(19) United States
(12) Patent Application Publication
(10) Pub. No.: US 2026/0193654 A1
(43) Pub. Date: Jul. 9, 2026

(54) COMPOSITIONS AND METHODS FOR REGULATING MAPT

(71) Applicant: VOYAGER THERAPEUTICS, INC., Lexington, MA (US)

(72) Inventors: Dinah Wen-Yee Sah, Hopkinton, MA (US); Kathrin Hultsch, Kulmbach (DE); Jochen Deckert, Bayreuth (DE); Wen Jin, Arlington, MA (US); Jinzhao Hou, Lexington, MA (US); Wencheng Liu, Cambridge, MA (US); Todd Carter, Winchester, MA (US); Cansu Colpan, North Billerica, MA (US); Elisabeth Knoll, Melrose, MA (US); Shiron Jessie Lee, Medford, MA (US); Rajeev Nambiar Sivasankaran, Acton, MA (US); Hechen Bao, Waltham, MA (US)

(21) Appl. No.: 19/135,319

(22) PCT Filed: Dec. 28, 2023
(86) PCT No.: PCT/US2023/086232

§ 371 Date: Jun. 3, 2025

Related U.S. Application Data
(60) Provisional application No. 63/477,736, filed on Dec. 29, 2022.

(51) Int. Cl. C12N 15/113; A61P 25/28; C12N 15/86

(52) U.S. Cl. CPC C12N 15/113; A61P 25/28; C12N 15/86; C12N 2310/14; C12N 2750/14143; C12N 2750/14151

(57) ABSTRACT

The disclosure relates to small interfering RNA (siRNA) molecules targeting MAPT and adeno-associated viral (AAV) particles encoding the same for treating tauopathies.

### RELATED APPLICATIONS

The application claims priority to U.S. Provisional Application No. 63/477,736 filed on Dec. 29, 2022; the entire contents of which are hereby incorporated by reference in their entirety.

### SEQUENCE LISTING

The present application is being filed along with a Sequence Listing in electronic format. The Sequence Listing file, entitled V2071-3010PCT_SL.xml, was created on Dec. 13, 2023 and is 2,478,152 bytes in size. The information in electronic format of the Sequence Listing is incorporated herein by reference in its entirety.

### FIELD OF THE DISCLOSURE

The present disclosure relates generally to compositions and methods for vectorized delivery of an agent, e.g., an RNA agent, that that inhibits a mutant, variant, or wild type human microtubule-associated protein tau (MAPT) gene, mRNA, and/or protein.

### BACKGROUND

Tauopathies, which may also be referred to herein as tau-associated diseases, are a heterogeneous group of neurodegenerative diseases characterized by the dysfunction and/or aggregation of the microtubule-associated protein tau (MAPT) which may also be referred to as Tau herein, which is encoded by the MAPT gene. Tau is normally a very soluble protein known to associate with microtubules based on the extent of its phosphorylation. In tauopathies, tau becomes hyperphosphorylated, misfolds and aggregates as neurofibrillary tangles (NFT) of paired helical filaments (PHF), twisted ribbons or straight filaments. These NFT are largely considered indicative of impending neuronal cell degeneration which can contribute to widespread neuronal cell loss, leading to a variety of behavioral and cognitive deficits, and can also be fatal. Currently, very limited treatment options are available for tauopathies, often with only symptomatic relief and supportive therapies available. As such, there is a medical need for improved compositions and methods of prevention, treatment, and diagnosis for diseases associated with aberrant tau expression, including tauopathies and related neurodegenerative disorders.

### SUMMARY

The present disclosure provides AAV particles encoding an agent for targeting MAPT, e.g., an RNA agent to modulate, e.g., inhibit, MAPT gene expression and/or MAPT protein production and methods of use thereof. In some embodiments, the agent for targeting MAPT, e.g., a MAPT gene, mRNA, or protein (e.g., a tau protein) is an RNA agent for targeting MAPT, e.g., a siRNA duplex for targeting MAPT. In some embodiments, the agent for targeting MAPT comprises a modulatory polynucleotide encoding a siRNA duplex for targeting MAPT. Methods for treating diseases associated MAPT expression, e.g., aberrant expression, such as tauopathies, including but not limited to Alzheimer's disease (AD), frontotemporal dementia (FTD), and/or Dravet syndrome (DS) in a subject in need thereof.

Accordingly, in one aspect, the present disclosure provides a short interfering ribonucleic acid (siRNA) for inhibiting expression of microtubule-associated protein tau (MAPT) which comprises a sense strand sequence and an antisense strand sequence, wherein the antisense sequence is complementary to at least 15, 16, 17, 18, 19, 20, or 21 contiguous nucleotides comprising 0, 1, 2, or 3 mismatches of a MAPT sequence comprising the nucleotide sequence of SEQ ID NO: 5024 or a nucleotide sequence provided in Table 3 or 19. In some embodiments, the antisense strand sequence comprises at least 15, 16, 17, 18, 19, 20, 21 contiguous nucleotides differing by 3, 2, 1, or 0 nucleotides from any one of the antisense strand sequences provided in Tables 3 4, 5, 9A, or 9B; and the sense strand sequence comprises at least 15, 16, 17, 18, 19, 20, 21 contiguous nucleotides differing by 3, 2, 1, or 0 nucleotides from any one of the sense strand sequences provided in Tables 3 4, 5, 9A, or 9B, wherein the sense strand sequence and the antisense strand sequence comprise a region of complementarity of at least 15 nucleotides. In some embodiments, the antisense strand sequence comprises at least 15, 16, 17, 18, 19, 20, 21 contiguous nucleotides from any one of the antisense strand sequences provided in Tables 3 4, 5, 9A, or 9B; and the sense strand sequence comprises at least 15, 16, 17, 18, 19, 20, 21 contiguous nucleotides from any one of the sense strand sequences provided in Tables 3 4, 5, 9A, or 9B, wherein the sense strand sequence and the antisense strand sequence comprise a region of complementarity of at least 15 nucleotides. In some embodiments, the antisense strand sequence comprises nucleotide sequence of any one of the antisense strand sequences provided in Tables 3 4, 5, 9A, or 9B; and the sense strand sequence comprises the nucleotide sequence of any one of the sense strand sequences provided in Tables 3 4, 5, 9A, or 9B, wherein the sense strand sequence and the antisense strand sequence comprise a region of complementarity of at least 15 nucleotides.

In yet another aspect, the present disclosure provides a short interfering ribonucleic acid (siRNA) for inhibiting expression of microtubule-associated protein tau (MAPT) which comprises a sense strand sequence and an antisense strand sequence, wherein: (i) the antisense strand sequence comprises at least 20 or 21 contiguous nucleotides of any one of SEQ ID NOs: 4908, 4920, 4924, 5057, 4916, 4912, 5080, 5104, or 5128; and (ii) the sense strand sequence comprises at least 20 or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 5054, 5060, 5064, 5066, 5070, 5074, 4914, 4934, 4954, 4974, 4994, 5014, 4910, 4930, 4950, 4970, 4990, 5010, 5077, 5084, 5088, 5092, 5096, 5098, 5101, 5108, 5112, 5116, 5120, 5122, 5125, 5132, 5136, 5140, 5144, or 5146; wherein the sense strand sequence and the antisense strand sequence comprise a region of complementarity of at least 15 nucleotides.

In yet another aspect, the present disclosure provides an adeno-associated virus (AAV) viral genome comprising a nucleotide sequence positioned between two inverted terminal repeats (ITRs), wherein the nucleotide sequence encodes an siRNA for inhibiting MAPT, wherein the siRNA comprises a sense strand sequence and an antisense strand sequence, wherein: (i) the antisense strand sequence comprises at least 19, 20, or 21 contiguous nucleotides of any one of SEQ ID NOs: 4908, 4920, 4924, 5057, 4916, 4912, 5080, 5104, or 5128; and (ii) the sense strand sequence comprises at least 19, 20, or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 5054, 5060, 5064, 5066, 5070, 5074, 4914, 4934, 4954, 4974, 4994, 5014, 4910, 4930, 4950, 4970, 4990, 5010, 5077, 5084, 5088, 5092, 5096, 5098, 5101, 5108, 5112, 5116, 5120, 5122, 5125, 5132, 5136, 5140, 5144, or 5146; wherein the sense strand sequence and the antisense strand sequence comprise a region of complementarity of at least 15 nucleotides.

In another aspect, the present disclosure provides a modulatory polynucleotide encoding an siRNA for targeting MAPT described herein. In some embodiments, the modulatory polynucleotide further comprises a 5′ flanking region, a loop region, and/or a 3′ flanking region.

In another aspect, the present disclosure provides an AAV particle comprising a promoter operably linked a nucleic acid encoding a modulatory polynucleotide comprising an siRNA for targeting MAPT described herein.

In another aspect, the present disclosure provides a method of making an AAV particle comprising an AAV capsid protein, e.g., an AAV capsid variant, encoding an siRNA for targeting MAPT described herein. In some embodiments, the method comprises providing a host cell comprising a viral genome encoding an siRNA described herein and incubating the host cell under conditions suitable to enclose the viral genome in the AAV capsid protein, e.g., an AAV capsid variant, thereby making the AAV particle.

In yet another aspect, the present disclosure provides a method of delivering an siRNA for inhibiting MAPT to a cell. In some embodiments, the method comprises administering an effective amount of an siRNA or an AAV particle encoding an siRNA described herein.

In yet another aspect, the present disclosure provides, a method of treating a subject having or diagnosed with having a genetic disorder e.g., a monogenic disorder or a polygenic disorder. The method comprising administering an effective amount of an siRNA or an AAV particle encoding an siRNA described herein.

In yet another aspect, the present disclosure provides, a method of treating a subject having or diagnosed with having a neurological disorder e.g., a neurodegenerative disorder. The method comprising administering an effective amount of an siRNA or an AAV particle encoding an siRNA described herein.

In yet another aspect, the present disclosure provides, a method of treating a subject having or diagnosed with having a disorder associated with tau expression, e.g., aberrant tau expression. The method comprising administering an effective amount of an siRNA or an AAV particle encoding an siRNA described herein.

In yet another aspect, the present disclosure provides, a method of treating a subject having or diagnosed with a tauopathy. The method comprising administering an effective amount of an siRNA or an AAV particle encoding an siRNA described herein.

In some embodiments, the genetic disorder, neurological disorder (e.g., neurodegenerative disorder), disorder associated with tau expression (e.g., aberrant tau expression), and/or tauopathy is Alzheimer's disease, Frontotemporal dementia (FTD), frontotemporal dementia and parkinsonism linked to chromosome 17 (FTDP-17), frontotemporal lobar degeneration (FTLD), Dravet syndrome, neurodegenerative disease, traumatic brain injury (TBI), chronic traumatic encephalopathy (CTE), progressive supranuclear palsy (PSP), Down's syndrome, Pick's disease, corticobasal degeneration (CBD), corticobasal syndrome, amyotrophic lateral sclerosis (ALS), Prion diseases, CJD, Multiple system atrophy, mild cognitive impairment, Tangle-only dementia, or Progressive subcortical gliosis.

### ENUMERATED EMBODIMENTS

1. A short interfering ribonucleic acid (siRNA) for inhibiting expression of microtubule-associated protein tau (MAPT) which comprises a sense strand sequence and an antisense strand sequence, wherein the antisense sequence is complementary to at least 15, 16, 17, 18, 19, 20, or 21 contiguous nucleotides comprising 0, 1, 2, or 3 mismatches of a MAPT sequence comprising the nucleotide sequence of SEQ ID NO: 5024 or a nucleotide sequence provided in Table 3 or 19.
2. The siRNA of embodiment 1, wherein the antisense strand is complementary to at least 15, 16, 17, 18, 19, 20, or 21, contiguous nucleotides with 0, 1, 2, or 3 mismatches to an exon of a MAPT sequence, wherein the exon comprises nucleotides 1277 to 1332 of SEQ ID NO: 5024, nucleotides 1712-1977 of SEQ ID NO: 5024, or nucleotides 2266-6644 of SEQ ID NO: 5024.
3. The siRNA of embodiment 1 or 2, wherein the antisense strand is complementary to at least 15, 16, 17, or 18, contiguous nucleotides with 0, 1, 2, or 3 mismatches to nucleotides 1300-1317, 1305-1322, 1860-1877, 2279-2297, 2286-2303, 2402-2419, 2581-2958, 2584-2601, 2633-2650, or 2634-2653 of SEQ ID NO: 5024.
4. The siRNA of any one of embodiments 1-3, wherein the sense strand comprises:
(i) at least 15, 16, 17, 18, 19, 20, or 21 contiguous nucleotides comprising 0, 1, 2, or 3 mismatches of a MAPT sequence comprising the nucleotide sequence of SEQ ID NO: 5024 or a nucleotide sequence provided in Table 3 or 19;
(ii) at least 15, 16, 17, 18, 19, 20, or 21, contiguous nucleotides with 0, 1, 2, or 3 mismatches to an exon of a MAPT sequence, wherein the exon comprises nucleotides 1277 to 1332 of SEQ ID NO: 5024, nucleotides 1712-1977 of SEQ ID NO: 5024, or nucleotides 2266-6644 of SEQ ID NO: 5024; or
(iii) at least 15, 16, 17, or 18, contiguous nucleotides with 0, 1, 2, or 3 mismatches to nucleotides 1300-1317, 1305-1322, 1860-1877, 2279-2297, 2286-2303, 2402-2419, 2581-2958, 2584-2601, 2633-2650, or 2634-2653 of SEQ ID NO: 5024.
   5. The siRNA of any one of embodiments 1-4, wherein the antisense strand sequence comprises at least 15, 16, 17, 18, 19, 20, 21 contiguous nucleotides differing by 3, 2, 1, or 0 nucleotides from any one of the antisense strand sequences provided in Tables 3 4, 5, 9A, or 9B.
   6. The siRNA of any one of embodiments 1-5, wherein the sense strand sequence comprises at least 15, 16, 17, 18, 19, 20, 21 contiguous nucleotides differing by 3, 2, 1, or 0 nucleotides from any one of the sense strand sequences provided in Table 4, 5, 9A, or 9B.
   7. The siRNA of any one of embodiments 1-6, wherein the antisense strand sequence comprises at least 15, 16, 17, 18, 19, 20, 21 contiguous nucleotides differing by 3, 2, 1, or 0 nucleotides from any one of the antisense strand sequences provided in Tables 3 4, 5, 9A, or 9B; and the sense strand sequence comprises at least 15, 16, 17, 18, 19, 20, 21 contiguous nucleotides differing by 3, 2, 1, or 0 nucleotides from any one of the sense strand sequences provided in Tables 3 4, 5, 9A, or 9B, wherein the sense strand sequence and the antisense strand sequence comprise a region of complementarity of at least 15 nucleotides.
   8. The siRNA of embodiment 7, wherein the region of complementarity is 15-30, 19-21, or 25-30 nucleotides in length, e.g., 17, 18, 20, 21, 22, 25, or 30 nucleotides in length.
   9. The siRNA of any one of embodiments 1-8, wherein:
(i) the antisense strand sequence comprises at least 15, 16, 17, 18, or 19 contiguous nucleotides differing by 3, 2, 1, or 0 nucleotides from the nucleotide sequence of SEQ ID NO: 4700 and the sense strand sequence comprises at least 15, 16, 17, 18, or 19 contiguous nucleotides differing by 3, 2, 1, or 0 nucleotides from the nucleotide sequence of any one of SEQ ID NOs: 4502-4505;
(ii) the antisense strand sequence comprises at least 15, 16, 17, 18, 19, 20, or 21 contiguous nucleotides differing by 3, 2, 1, or 0 nucleotides from the nucleotide sequence of SEQ ID NO: 4920 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4918, 4938, 4958, 4978, or 4998;
(iii) the antisense strand sequence comprises at least 15, 16, 17, 18, or 19 contiguous nucleotides differing by 3, 2, 1, or 0 nucleotides from the nucleotide sequence of SEQ ID NO: 4697 and the sense strand sequence comprises at least 15, 16, 17, 18, or 19 contiguous nucleotides differing by 3, 2, 1, or 0 nucleotides from the nucleotide sequence of any one of SEQ ID NOs: 4495-4499;
(iv) the antisense strand sequence comprises at least 15, 16, 17, 18, 19, 20, or 21 contiguous nucleotides differing by 3, 2, 1, or 0 nucleotides from the nucleotide sequence of SEQ ID NO: 4908 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4906, 4926, 4946, 4966, 4986, or 5006;
(v) the antisense strand sequence comprises at least 15, 16, 17, 18, or 19 contiguous nucleotides differing by 3, 2, 1, or 0 nucleotides from the nucleotide sequence of SEQ ID NO: 4690 and the sense strand sequence comprises at least 15, 16, 17, 18, or 19 contiguous nucleotides differing by 3, 2, 1, or 0 nucleotides from the nucleotide sequence of any one of SEQ ID NOs: 4480-4484;
(vi) the antisense strand sequence comprises at least 15, 16, 17, 18, 19, 20, or 21 contiguous nucleotides differing by 3, 2, 1, or 0 nucleotides from the nucleotide sequence of SEQ ID NO: 4924 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4922, 4942, 4962, 4982, 5002, or 5022;
(vii) the antisense strand sequence comprises at least 15, 16, 17, 18, or 19 contiguous nucleotides differing by 3, 2, 1, or 0 nucleotides from the nucleotide sequence of SEQ ID NO: 4694 and the sense strand sequence comprises at least 15, 16, 17, 18, or 19 contiguous nucleotides differing by 3, 2, 1, or 0 nucleotides from the nucleotide sequence of SEQ ID NO: 4492;
(viii) the antisense strand sequence comprises at least 15, 16, 17, 18, or 19 contiguous nucleotides differing by 3, 2, 1, or 0 nucleotides from the nucleotide sequence of SEQ ID NO: 4691 and the sense strand sequence comprises at least 15, 16, 17, 18, or 19 contiguous nucleotides differing by 3, 2, 1, or 0 nucleotides from the nucleotide sequence of any one of SEQ ID NOs: 4485-4489;
(ix) the antisense strand sequence comprises at least 15, 16, 17, 18, 19, 20, or 21 contiguous nucleotides differing by 3, 2, 1, or 0 nucleotides from the nucleotide sequence of SEQ ID NO: 4916 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4914, 4934, 4954, 4974, 4994, or 5014;
(x) the antisense strand sequence comprises at least 15, 16, 17, 18, or 19 contiguous nucleotides differing by 3, 2, 1, or 0 nucleotides from the nucleotide sequence of SEQ ID NO: 4701 and the sense strand sequence comprises at least 15, 16, 17, 18, or 19 contiguous nucleotides differing by 3, 2, 1, or 0 nucleotides from the nucleotide sequence of any one of SEQ ID NOs: 4506-4510;
(xi) the antisense strand sequence comprises at least 15, 16, 17, 18, 19, 20, or 21 contiguous nucleotides differing by 3, 2, 1, or 0 nucleotides from the nucleotide sequence of SEQ ID NO: 4912 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4910, 4930, 4950, 4970, 4990, or 5010;
(xii) the antisense strand sequence comprises at least 15, 16, 17, 18, or 19 contiguous nucleotides differing by 3, 2, 1, or 0 nucleotides from the nucleotide sequence of SEQ ID NO: 4687 and the sense strand sequence comprises at least 15, 16, 17, 18, or 19 contiguous nucleotides differing by 3, 2, 1, or 0 nucleotides from the nucleotide sequence of SEQ ID NO: 4477;
(xiii) the antisense strand sequence comprises at least 15, 16, 17, 18, or 19 contiguous nucleotides differing by 3, 2, 1, or 0 nucleotides from the nucleotide sequence of SEQ ID NO: 4712 and the sense strand sequence comprises at least 15, 16, 17, 18, or 19 contiguous nucleotides differing by 3, 2, 1, or 0 nucleotides from the nucleotide sequence of SEQ ID NO: 4521;
(xiv) the antisense strand sequence comprises at least 15, 16, 17, 18, or 19 contiguous nucleotides differing by 3, 2, 1, or 0 nucleotides from the nucleotide sequence of SEQ ID NO: 4696 and the sense strand sequence comprises at least 15, 16, 17, 18, or 19 contiguous nucleotides differing by 3, 2, 1, or 0 nucleotides from the nucleotide sequence of SEQ ID NO: 4494; or
(xv) the antisense strand sequence comprises at least 15, 16, 17, 18, or 19 contiguous nucleotides differing by 3, 2, 1, or 0 nucleotides from the nucleotide sequence of SEQ ID NO: 4718 and the sense strand sequence comprises at least 15, 16, 17, 18, or 19 contiguous nucleotides differing by 3, 2, 1, or 0 nucleotides from the nucleotide sequence of SEQ ID NO: 4527.
   10. The siRNA of any one of claims 1-9, wherein:
(i) the antisense strand sequence comprises at least 15, 16, 17, 18, or 19 contiguous nucleotides from the nucleotide sequence of SEQ ID NO: 4700 and the sense strand sequence comprises at least 15, 16, 17, 18, or 19 contiguous nucleotides from the nucleotide sequence of any one of SEQ ID NOs: 4502-4505;
(ii) the antisense strand sequence comprises at least 15, 16, 17, 18, 19, 20, or 21 contiguous nucleotides from the nucleotide sequence of SEQ ID NO: 4920 and the sense strand sequence comprises at least 15, 16, 17, 18, 19, 20, or 21 contiguous nucleotides differing from the nucleotide sequence of any one of SEQ ID NOs: 4918, 4938, 4958, 4978, or 4998;
(iii) the antisense strand sequence comprises at least 15, 16, 17, 18, or 19 contiguous nucleotides from the nucleotide sequence of SEQ ID NO: 4697 and the sense strand sequence comprises at least 15, 16, 17, 18, or 19 contiguous nucleotides from the nucleotide sequence of any one of SEQ ID NOs: 4495-4499;
(iv) the antisense strand sequence comprises at least 15, 16, 17, 18, 19, 20, or 21 contiguous nucleotides from the nucleotide sequence of SEQ ID NO: 4908 and the sense strand sequence comprises at least 15, 16, 17, 18, 19, 20, or 21 contiguous nucleotides from the nucleotide sequence of any one of SEQ ID NOs: 4906, 4926, 4946, 4966, 4986, or 5006;
(v) the antisense strand sequence comprises at least 15, 16, 17, 18, or 19 contiguous nucleotides from the nucleotide sequence of SEQ ID NO: 4690 and the sense strand sequence comprises at least 15, 16, 17, 18, or 19 contiguous nucleotides from the nucleotide sequence of any one of SEQ ID NOs: 4480-4484;
(vi) the antisense strand sequence comprises at least 15, 16, 17, 18, 19, 20, or 21 contiguous nucleotides from the nucleotide sequence of SEQ ID NO: 4924 and the sense strand sequence comprises at least 15, 16, 17, 18, 19, 20, or 21 contiguous nucleotides from the nucleotide sequence of any one of SEQ ID NOs: 4922, 4942, 4962, 4982, 5002, or 5022;
(vii) the antisense strand sequence comprises at least 15, 16, 17, 18, or 19 contiguous nucleotides from the nucleotide sequence of SEQ ID NO: 4694 and the sense strand sequence comprises at least 15, 16, 17, 18, or 19 contiguous nucleotides from the nucleotide sequence of SEQ ID NO: 4492; (viii) the antisense strand sequence comprises at least 15, 16, 17, 18, or 19 contiguous nucleotides from the nucleotide sequence of SEQ ID NO: 4691 and the sense strand sequence comprises at least 15, 16, 17, 18, or 19 contiguous nucleotides from the nucleotide sequence of any one of SEQ ID NOs: 4485-4489;
(ix) the antisense strand sequence comprises at least 15, 16, 17, 18, 19, 20, or 21 contiguous nucleotides from the nucleotide sequence of SEQ ID NO: 4916 and the sense strand sequence comprises at least 15, 16, 17, 18, 19, 20, or 21 contiguous nucleotides from the nucleotide sequence of any one of SEQ ID NOs: 4914, 4934, 4954, 4974, 4994, or 5014;
(x) the antisense strand sequence comprises at least 15, 16, 17, 18, or 19 contiguous nucleotides from the nucleotide sequence of SEQ ID NO: 4701 and the sense strand sequence comprises at least 15, 16, 17, 18, or 19 contiguous nucleotides from the nucleotide sequence of any one of SEQ ID NOs: 4506-4510;
(xi) the antisense strand sequence comprises at least 15, 16, 17, 18, 19, 20, or 21 contiguous nucleotides from the nucleotide sequence of SEQ ID NO: 4912 and the sense strand sequence comprises at least 15, 16, 17, 18, 19, 20, or 21 contiguous nucleotides from the nucleotide sequence of any one of SEQ ID NOs: 4910, 4930, 4950, 4970, 4990, or 5010;
(xii) the antisense strand sequence comprises at least 15, 16, 17, 18, or 19 contiguous nucleotides from the nucleotide sequence of SEQ ID NO: 4687 and the sense strand sequence comprises at least 15, 16, 17, 18, or 19 contiguous nucleotides from the nucleotide sequence of SEQ ID NO: 4477;
(xiii) the antisense strand sequence comprises at least 15, 16, 17, 18, or 19 contiguous nucleotides from the nucleotide sequence of SEQ ID NO: 4712 and the sense strand sequence comprises at least 15, 16, 17, 18, or 19 contiguous nucleotides from the nucleotide sequence of SEQ ID NO: 4521;
(xiv) the antisense strand sequence comprises at least 15, 16, 17, 18, or 19 contiguous nucleotides from the nucleotide sequence of SEQ ID NO: 4696 and the sense strand sequence comprises at least 15, 16, 17, 18, or 19 contiguous nucleotides from the nucleotide sequence of SEQ ID NO: 4494; or (xv) the antisense strand sequence comprises at least 15, 16, 17, 18, or 19 contiguous nucleotides from the nucleotide sequence of SEQ ID NO: 4718 and the sense strand sequence comprises at least 15, 16, 17, 18, or 19 contiguous nucleotides from the nucleotide sequence of SEQ ID NO: 4527.
   11. A short interfering ribonucleic acid (siRNA) for inhibiting expression of microtubule-associated protein tau (MAPT) which comprises a sense strand sequence and an antisense strand sequence, wherein:
(i) the antisense strand sequence comprises at least 20 or 21 contiguous nucleotides of any one of SEQ ID NOs: 4908, 4920, 4924, 5057, 4916, 4912, 5080, 5104, or 5128; and
(ii) the sense strand sequence comprises at least 20 or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 5054, 5060, 5064, 5066, 5070, 5074, 4914, 4934, 4954, 4974, 4994, 5014, 4910, 4930, 4950, 4970, 4990, 5010, 5077, 5084, 5088, 5092, 5096, 5098, 5101, 5108, 5112, 5116, 5120, 5122, 5125, 5132, 5136, 5140, 5144, or 5146;
wherein the sense strand sequence and the antisense strand sequence comprise a region of complementarity of at least 15 nucleotides.
   12. An adeno-associated virus (AAV) viral genome comprising a nucleotide sequence positioned between two inverted terminal repeats (ITRs), wherein the nucleotide sequence encodes an siRNA for inhibiting MAPT, wherein the siRNA comprises a sense strand sequence and an antisense strand sequence, wherein:
(i) the antisense strand sequence comprises at least 19, 20, or 21 contiguous nucleotides of any one of SEQ ID NOs: 4908, 4920, 4924, 5057, 4916, 4912, 5080, 5104, or 5128; and
(ii) the sense strand sequence comprises at least 19, 20, or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 5054, 5060, 5064, 5066, 5070, 5074, 4914, 4934, 4954, 4974, 4994, 5014, 4910, 4930, 4950, 4970, 4990, 5010, 5077, 5084, 5088, 5092, 5096, 5098, 5101, 5108, 5112, 5116, 5120, 5122, 5125, 5132, 5136, 5140, 5144, or 5146;
wherein the sense strand sequence and the antisense strand sequence comprise a region of complementarity of at least 15 nucleotides.
   13. The siRNA of any one of embodiments 8-11 or the AAV viral genome of embodiment 12, wherein the region of complementarity comprises at least 18, 19, or 20 nucleotides.
   14. The siRNA of any one of embodiments 1-10 or the AAV viral genome of embodiment 12 or 13, wherein the antisense strand sequence comprises at least 19, 20, or 21 contiguous nucleotides of any one of SEQ ID NOs: 4908, 4920, 4924, or 4912.
   15. The siRNA of any one of embodiments 1-10 or the AAV viral genome of any one of embodiments 12-14, wherein the sense strand sequence comprises at least 19, 20, or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 4910, 4930, 4950, 4970, 4990, or 5010.
   16. The siRNA of any one of embodiments 1-10 or the AAV viral genome of any one of embodiments 12-15, wherein:
(i) the antisense strand sequence comprises at least 19, 20, or 21 contiguous nucleotides from the nucleotide sequence of SEQ ID NO: 4908 and the sense strand sequence comprises at least 19, 20, or 21 contiguous nucleotides from the nucleotide sequence of any one of SEQ ID NOs: 4906, 4926, 4946, 4966, 4986, or 5006;
(ii) the antisense strand sequence comprises at least 19, 20, or 21 contiguous nucleotides from the nucleotide sequence of SEQ ID NO: 4920 and the sense strand sequence comprises at least 19, 20, or 21 contiguous nucleotides from the nucleotide sequence of any one of SEQ ID NOs: 4918, 4938, 4958, 4978, or 4998;
(iii) the antisense strand sequence comprises at least 19, 20, or 21 contiguous nucleotides from the nucleotide sequence of SEQ ID NO: 4924 and the sense strand sequence comprises at least 19, 20, or 21 contiguous nucleotides from the nucleotide sequence of any one of SEQ ID NOs: 4922, 4942, 4962, 4982, 5002, or 5022;
(iv) the antisense strand sequence comprises at least 19, 20, or 21 contiguous nucleotides from the nucleotide sequence of SEQ ID NO: 4912 and the sense strand sequence comprises at least 19, 20, or 21 contiguous nucleotides from the nucleotide sequence of any one of SEQ ID NOs: 4910, 4930, 4950, 4970, 4990, or 5010;
(v) the antisense strand sequence comprises at least 19, 20, or 21 contiguous nucleotides from the nucleotide sequence of SEQ ID NO: 4916 and the sense strand sequence comprises at least 19, 20, or 21 contiguous nucleotides from the nucleotide sequence of any one of SEQ ID NOs: 4914, 4934, 4954, 4974, 4994, or 5014;
(vi) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of any one of SEQ ID NOs: 5101, 5108, 5112, 5116, 5120, or 5122;
(vii) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of any one of SEQ ID NOs: 5125, 5132, 5136, 5140, 5144, or 5146;
(viii) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of any one of SEQ ID NOs: 5077, 5084, 5088, 5092, 5096, or 5098; or
(ix) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of any one of SEQ ID NOs: 5054, 5060, 5064, 5066, 5070, or 5074.
   17. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4908 and the sense strand sequence comprises at least 19, 20, or 21 contiguous nucleotides from the nucleotide sequence of SEQ ID NO: 4906.
   18. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4920 and the sense strand sequence comprises at least 19, 20, or 21 contiguous nucleotides from the nucleotide sequence of SEQ ID NO: 4918.
   19. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4924 and the sense strand sequence comprises at least 19, 20, or 21 contiguous nucleotides from the nucleotide sequence of SEQ ID NO: 4922.
   20. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4908 and the sense strand sequence comprises at least 19, 20, or 21 contiguous nucleotides from the nucleotide sequence of SEQ ID NO: 4966.
   21. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4912 and the sense strand sequence comprises at least 19, 20, or 21 contiguous nucleotides from the nucleotide sequence of SEQ ID NO: 4970.
   22. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4920 and the sense strand sequence comprises at least 19, 20, or 21 contiguous nucleotides from the nucleotide sequence of SEQ ID NO: 4978.
   23. The siRNA of any one of embodiments 1-10 or the AAV viral genome of any one of embodiments 12-16, wherein the antisense strand sequence comprises at least 19, 20, or 21 contiguous nucleotides from the nucleotide sequence of SEQ ID NO: 4924 and the sense strand sequence comprises at least 19, 20, or 21 contiguous nucleotides from the nucleotide sequence of SEQ ID NO: 4982.
   24. The siRNA of any one of embodiments 1-10 or the AAV viral genome of any one of embodiments 12-16, wherein:
(i) the antisense strand sequence comprises at least 19, 20, or 21 contiguous nucleotides from the nucleotide sequence of SEQ ID NO: 4912; and the sense strand sequence comprises at least 19, 20, or 21 contiguous nucleotides from the nucleotide sequence of SEQ ID NO: 4910;
(ii) the antisense strand sequence comprises at least 19, 20, or 21 contiguous nucleotides from the nucleotide sequence of SEQ ID NO: 4916; and the sense strand sequence comprises at least 19, 20, or 21 contiguous nucleotides from the nucleotide sequence of SEQ ID NO: 4914;
(iii) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of SEQ ID NO: 5054;
(iv) the antisense strand sequence comprises at least 19, 20, or 21 contiguous nucleotides from the nucleotide sequence of SEQ ID NO: 4908; and the sense strand sequence comprises at least 19, 20, or 21 contiguous nucleotides from the nucleotide sequence of SEQ ID NO: 4926;
(v) the antisense strand sequence comprises at least 19, 20, or 21 contiguous nucleotides from the nucleotide sequence of SEQ ID NO: 4912; and the sense strand sequence comprises at least 19, 20, or 21 contiguous nucleotides from the nucleotide sequence of SEQ ID NO: 4930;
(vi) the antisense strand sequence comprises at least 19, 20, or 21 contiguous nucleotides from the nucleotide sequence of SEQ ID NO: 4916; and the sense strand sequence comprises at least 19, 20, or 21 contiguous nucleotides from the nucleotide sequence of SEQ ID NO: 4934;
(vii) the antisense strand sequence comprises at least 19, 20, or 21 contiguous nucleotides from the nucleotide sequence of SEQ ID NO: 4920; and the sense strand sequence comprises at least 19, 20, or 21 contiguous nucleotides from the nucleotide sequence of SEQ ID NO: 4938;
(viii) the antisense strand sequence comprises at least 19, 20, or 21 contiguous nucleotides from the nucleotide sequence of SEQ ID NO: 4924; and the sense strand sequence comprises at least 19, 20, or 21 contiguous nucleotides from the nucleotide sequence of SEQ ID NO: 4942;
(ix) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of SEQ ID NO: 5060;
(x) the antisense strand sequence comprises at least 19, 20, or 21 contiguous nucleotides from the nucleotide sequence of SEQ ID NO: 4908; and the sense strand sequence comprises at least 19, 20, or 21 contiguous nucleotides from the nucleotide sequence of SEQ ID NO: 4946;
(xi) the antisense strand sequence comprises at least 19, 20, or 21 contiguous nucleotides from the nucleotide sequence of SEQ ID NO: 4912; and the sense strand sequence comprises at least 19, 20, or 21 contiguous nucleotides from the nucleotide sequence of SEQ ID NO: 4950;
(xii) the antisense strand sequence comprises at least 19, 20, or 21 contiguous nucleotides from the nucleotide sequence of SEQ ID NO: 4916; and the sense strand sequence comprises at least 19, 20, or 21 contiguous nucleotides from the nucleotide sequence of SEQ ID NO: 4954;
(xiii) the antisense strand sequence comprises at least 19, 20, or 21 contiguous nucleotides from the nucleotide sequence of SEQ ID NO: 4920; and the sense strand sequence comprises at least 19, 20, or 21 contiguous nucleotides from the nucleotide sequence of SEQ ID NO: 4958;
(xiv) the antisense strand sequence comprises at least 19, 20, or 21 contiguous nucleotides from the nucleotide sequence of SEQ ID NO: 4924; and the sense strand sequence comprises at least 19, 20, or 21 contiguous nucleotides from the nucleotide sequence of SEQ ID NO: 4962;
(xv) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of SEQ ID NO: 5064;
(xvi) the antisense strand sequence comprises at least 19, 20, or 21 contiguous nucleotides from the nucleotide sequence of SEQ ID NO: 4916; and the sense strand sequence comprises at least 19, 20, or 21 contiguous nucleotides from the nucleotide sequence of SEQ ID NO: 4974;
(xvii) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of SEQ ID NO: 5066;
(xviii) the antisense strand sequence comprises at least 19, 20, or 21 contiguous nucleotides from the nucleotide sequence of SEQ ID NO: 4908; and the sense strand sequence comprises at least 19, 20, or 21 contiguous nucleotides from the nucleotide sequence of SEQ ID NO: 4986;
(xix) the antisense strand sequence comprises at least 19, 20, or 21 contiguous nucleotides from the nucleotide sequence of SEQ ID NO: 4912; and the sense strand sequence comprises at least 19, 20, or 21 contiguous nucleotides from the nucleotide sequence of SEQ ID NO: 4990;
(xx) the antisense strand sequence comprises at least 19, 20, or 21 contiguous nucleotides from the nucleotide sequence of SEQ ID NO: 4916; and the sense strand sequence comprises at least 19, 20, or 21 contiguous nucleotides from the nucleotide sequence of SEQ ID NO: 4994;
(xxi) the antisense strand sequence comprises at least 19, 20, or 21 contiguous nucleotides from the nucleotide sequence of SEQ ID NO: 4920; and the sense strand sequence comprises at least 19, 20, or 21 contiguous nucleotides from the nucleotide sequence of SEQ ID NO: 4998;
(xxii) the antisense strand sequence comprises at least 19, 20, or 21 contiguous nucleotides from the nucleotide sequence of SEQ ID NO: 4924; and the sense strand sequence comprises at least 19, 20, or 21 contiguous nucleotides from the nucleotide sequence of SEQ ID NO: 5002;
(xxiii) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of SEQ ID NO: 5070;
(xxiv) the antisense strand sequence comprises at least 19, 20, or 21 contiguous nucleotides from the nucleotide sequence of SEQ ID NO: 4908; and the sense strand sequence comprises at least 19, 20, or 21 contiguous nucleotides from the nucleotide sequence of SEQ ID NO: 5006;
(xxv) the antisense strand sequence comprises at least 19, 20, or 21 contiguous nucleotides from the nucleotide sequence of SEQ ID NO: 4912; and the sense strand sequence comprises at least 19, 20, or 21 contiguous nucleotides from the nucleotide sequence of SEQ ID NO: 5010;
(xxvi) the antisense strand sequence comprises at least 19, 20, or 21 contiguous nucleotides from the nucleotide sequence of SEQ ID NO: 4916; and the sense strand sequence comprises at least 19, 20, or 21 contiguous nucleotides from the nucleotide sequence of SEQ ID NO: 5014;
(xxvii) the antisense strand sequence comprises at least 19, 20, or 21 contiguous nucleotides from the nucleotide sequence of SEQ ID NO: 4920; and the sense strand sequence comprises at least 19, 20, or 21 contiguous nucleotides from the nucleotide sequence of SEQ ID NO: 4958;
(xxviii) the antisense strand sequence comprises at least 19, 20, or 21 contiguous nucleotides from the nucleotide sequence of SEQ ID NO: 4924; and the sense strand sequence comprises at least 19, 20, or 21 contiguous nucleotides from the nucleotide sequence of SEQ ID NO: 5022;
(xxvii) the antisense strand sequence comprises at least 19, 20, or 21 contiguous nucleotides from the nucleotide sequence of SEQ ID NO: 5057; and the sense strand sequence comprises at least 19, 20, or 21 contiguous nucleotides from the nucleotide sequence of SEQ ID NO: 5074;
(xxix) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of SEQ ID NO: 5077;
(xxx) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of SEQ ID NO: 5084;
(xxxi) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of SEQ ID NO: 5088;
(xxxii) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of SEQ ID NO: 5092;
(xxxiii) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of SEQ ID NO: 5096;
(xxxiv) the antisense strand sequence comprises at least 19, 20, or 21 contiguous nucleotides from the nucleotide sequence of SEQ ID NO: 5080; and the sense strand sequence comprises at least 19, 20, or 21 contiguous nucleotides from the nucleotide sequence of SEQ ID NO: 5098;
(xxxv) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of SEQ ID NO: 5101;
(xxxvi) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of SEQ ID NO: 5108;
(xxxvii) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of SEQ ID NO: 5112;
(xxxviii) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of SEQ ID NO: 5116;
(xxxix) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of SEQ ID NO: 5120;
(xl) the antisense strand sequence comprises at least 19, 20, or 21 contiguous nucleotides from the nucleotide sequence of SEQ ID NO: 5104; and the sense strand sequence comprises at least 19, 20, or 21 contiguous nucleotides from the nucleotide sequence of SEQ ID NO: 5122;
(xli) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of SEQ ID NO: 5125;
(xlii) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of SEQ ID NO: 5132;
(xliii) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of SEQ ID NO: 5136;
(xliv) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of SEQ ID NO: 5140;
(xlv) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of SEQ ID NO: 5144; or (xlvi) the antisense strand sequence comprises at least 19, 20, or 21 contiguous nucleotides from the nucleotide sequence of SEQ ID NO: 5128; and the sense strand sequence comprises at least 19, 20, or 21 contiguous nucleotides from the nucleotide sequence of SEQ ID NO: 5146.
   25. The siRNA of any one of embodiments 1-11 or 13 or the AAV viral genome of any one of embodiments 12-24, wherein the antisense strand sequence comprises at least 20 contiguous nucleotides of any one of SEQ ID NOs: 4908, 4920, 4924, or 4912.
   26. The siRNA of any one of embodiments 1-11, 13, or 25 or the AAV viral genome of any one of embodiments 12-25, wherein the sense strand sequence comprises at least 20 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 4910, 4930, 4950, 4970, 4990, or 5010.
   27. The siRNA of any one of embodiments 1-11, 13, 25, or 26 or the AAV viral genome of any one of embodiments 12-26, wherein:
(i) the antisense strand sequence comprises at least 20 contiguous nucleotides from the nucleotide sequence of SEQ ID NO: 4908 and the sense strand sequence comprises at least 20 contiguous nucleotides from the nucleotide sequence of any one of SEQ ID NOs: 4906, 4926, 4946, 4966, 4986, or 5006;
(ii) the antisense strand sequence comprises at least 20 contiguous nucleotides from the nucleotide sequence of SEQ ID NO: 4920 and the sense strand sequence comprises at least 20 contiguous nucleotides from the nucleotide sequence of any one of SEQ ID NOs: 4918, 4938, 4958, 4978, or 4998;
(iii) the antisense strand sequence comprises at least 20 contiguous nucleotides from the nucleotide sequence of SEQ ID NO: 4924 and the sense strand sequence comprises at least 20 contiguous nucleotides from the nucleotide sequence of any one of SEQ ID NOs: 4922, 4942, 4962, 4982, 5002, or 5022;
(iv) the antisense strand sequence comprises at least 20 contiguous nucleotides from the nucleotide sequence of SEQ ID NO: 4912 and the sense strand sequence comprises at least 20 contiguous nucleotides from the nucleotide sequence of any one of SEQ ID NOs: 4910, 4930, 4950, 4970, 4990, or 5010;
(v) the antisense strand sequence comprises at least 20 contiguous nucleotides from the nucleotide sequence of SEQ ID NO: 4916 and the sense strand sequence comprises at least 20 contiguous nucleotides from the nucleotide sequence of any one of SEQ ID NOs: 4914, 4934, 4954, 4974, 4994, or 5014;
(vi) the antisense strand sequence comprises at least 20 contiguous nucleotides from the nucleotide sequence of SEQ ID NO: 5104; and the sense strand sequence comprises at least 20 contiguous nucleotides from the nucleotide sequence of any one of SEQ ID NOs: 5101, 5108, 5112, 5116, 5120, or 5122;
(vii) the antisense strand sequence comprises at least 20 contiguous nucleotides from the nucleotide sequence of SEQ ID NO: 5128; and the sense strand sequence comprises at least 20 contiguous nucleotides from the nucleotide sequence of any one of SEQ ID NOs: 5125, 5132, 5136, 5140, 5144, or 5146;
(viii) the antisense strand sequence comprises at least 20 contiguous nucleotides from the nucleotide sequence of SEQ ID NO: 5080; and the sense strand sequence comprises at least 20 contiguous nucleotides from the nucleotide sequence of any one of SEQ ID NOs: 5077, 5084, 5088, 5092, 5096, or 5098; or
(ix) the antisense strand sequence comprises at least 20 contiguous nucleotides from the nucleotide sequence of SEQ ID NO: 5057; and the sense strand sequence comprises at least 20 contiguous nucleotides from the nucleotide sequence of any one of SEQ ID NOs: 5054, 5060, 5064, 5066, 5070, or 5074.
   28. The siRNA of any one of embodiments 1-11, 13, or 25-27 or the AAV viral genome of any one of embodiments 11-17 or 25-27, wherein the antisense strand sequence comprises at least 20 contiguous nucleotides from the nucleotide sequence of SEQ ID NO: 4908 and the sense strand sequence comprises at least 20 contiguous nucleotides from the nucleotide sequence of SEQ ID NO: 4906.
   29. The siRNA of any one of embodiments 1-11, 13, or 25-27 or the AAV viral genome of any one of embodiments 11-16, 18, or 25-27, wherein the antisense strand sequence comprises at least 20 contiguous nucleotides from the nucleotide sequence of SEQ ID NO: 4920 and the sense strand sequence comprises at least 20 contiguous nucleotides from the nucleotide sequence of SEQ ID NO: 4918.
   30. The siRNA of any one of embodiments 1-11, 13, or 25-27 or the AAV viral genome of any one of embodiments 11-16, 19, or 25-27, wherein the antisense strand sequence comprises at least 20 contiguous nucleotides from the nucleotide sequence of SEQ ID NO: 4924 and the sense strand sequence comprises at least 20 contiguous nucleotides from the nucleotide sequence of SEQ ID NO: 4922.
   31. The siRNA of any one of embodiments 1-11, 13, or 25-27 or the AAV viral genome of any one of embodiments 11-16, 20, or 25-27, wherein the antisense strand sequence comprises at least 20 contiguous nucleotides from the nucleotide sequence of SEQ ID NO: 4908 and the sense strand sequence comprises at least 20 contiguous nucleotides from the nucleotide sequence of SEQ ID NO: 4966.
   32. The siRNA of any one of embodiments 1-11, 13, or 25-27 or the AAV viral genome of any one of embodiments 11-16, 21, or 25-27, wherein the antisense strand sequence comprises at least 20 contiguous nucleotides from the nucleotide sequence of SEQ ID NO: 4912 and the sense strand sequence comprises at least 20 contiguous nucleotides from the nucleotide sequence of SEQ ID NO: 4970.
   33. The siRNA of any one of embodiments 1-11, 13, or 25-27 or the AAV viral genome of any one of embodiments 11-16, 22, or 25-27, wherein the antisense strand sequence comprises at least 20 contiguous nucleotides from the nucleotide sequence of SEQ ID NO: 4920 and the sense strand sequence comprises at least 20 contiguous nucleotides from the nucleotide sequence of SEQ ID NO: 4978.
   34. The siRNA of any one of embodiments 1-11, 13, or 25-27 or the AAV viral genome of any one of embodiments 11-16, 23, or 25-27, wherein the antisense strand sequence comprises at least 20 contiguous nucleotides from the nucleotide sequence of SEQ ID NO: 4924 and the sense strand sequence comprises at least 20 contiguous nucleotides from the nucleotide sequence of SEQ ID NO: 4982.
   35. The siRNA of any one of embodiments 1-11, 13, or 25-27 or the AAV viral genome of any one of embodiments 11-16 or 24-27, wherein:
(i) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4910;
(ii) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4914;
(iii) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5054;
(iv) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 4926;
(v) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4930;
(vi) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4934;
(vii) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4938;
(viii) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 4942;
(ix) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5060;
(x) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 4946;
(xi) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4950;
(xii) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4954;
(xiii) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4958;
(xiv) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 4962;
(xv) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5064;
(xvi) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4974;
(xvii) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5066;
(xviii) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 4986;
(xix) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4990;
(xx) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4994;
(xxi) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4998;
(xxii) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 5002;
(xxiii) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5070;
(xxiv) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 5006;
(xxv) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 5010;
(xxvi) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 5014;
(xxvii) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4958;
(xxviii) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 5022;
(xxvii) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5074;
(xxix) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5077;
(xxx) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5084;
(xxxi) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5088;
(xxxii) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5092;
(xxxiii) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5096;
(xxxiv) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5098;
(xxxv) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5101;
(xxxvi) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5108;
(xxxvii) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5112;
(xxxviii) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5116;
(xxxix) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5120;
(xl) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5122;
(xli) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5125;
(xlii) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5132;
(xliii) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5136;
(xliv) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5140;
(xlv) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5144; or
(xlvi) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5146.
   36. The siRNA of any one of embodiments 1-11, 13, or 25-35 or the AAV viral genome of any one of embodiments 12-35, wherein the antisense strand sequence comprises the nucleotide sequence of any one of SEQ ID NOs: 4908, 4920, 4924, or 4912.
   37. The siRNA of any one of embodiments 1-11, 13, or 25-36 or the AAV viral genome of any one of embodiments 12-36, wherein the sense strand sequence comprises at the nucleotide sequence of any one of SEQ ID NOs: 4906, 4926, 4946, 4966, 4986, 5006, 4918, 4938, 4958, 4978, 4998, 4922, 4942, 4962, 4982, 5002, 5022, 4910, 4930, 4950, 4970, 4990, or 5010.
   38. The siRNA of any one of embodiments 1-11, 13, or 25-37 or the AAV viral genome of any one of embodiments 12-37, wherein:
(i) the antisense strand sequence comprises the nucleotide sequence of SEQ ID NO: 4908 and the sense strand sequence comprises the nucleotide sequence of any one of SEQ ID NOs: 4906, 4926, 4946, 4966, 4986, or 5006;
(ii) the antisense strand sequence comprises the nucleotide sequence of SEQ ID NO: 4920 and the sense strand sequence comprises the nucleotide sequence of any one of SEQ ID NOs: 4918, 4938, 4958, 4978, or 4998;
(iii) the antisense strand sequence comprises the nucleotide sequence of SEQ ID NO: 4924 and the sense strand sequence comprises the nucleotide sequence of any one of SEQ ID NOs: 4922, 4942, 4962, 4982, 5002, or 5022;
(iv) the antisense strand sequence comprises the nucleotide sequence of SEQ ID NO: 4912 and the sense strand sequence comprises the nucleotide sequence of any one of SEQ ID NOs: 4910, 4930, 4950, 4970, 4990, or 5010;
(v) the antisense strand sequence comprises the nucleotide sequence of SEQ ID NO: 4916 and the sense strand sequence comprises the nucleotide sequence of any one of SEQ ID NOs: 4914, 4934, 4954, 4974, 4994, or 5014;
(vi) the antisense strand sequence comprises the nucleotide sequence of SEQ ID NO: 5104; and the sense strand sequence comprises the nucleotide sequence of any one of SEQ ID NOs: 5101, 5108, 5112, 5116, 5120, or 5122;
(vii) the antisense strand sequence comprises the nucleotide sequence of SEQ ID NO: 5128; and the sense strand sequence comprises the nucleotide sequence of any one of SEQ ID NOs: 5125, 5132, 5136, 5140, 5144, or 5146;
(viii) the antisense strand sequence comprises the nucleotide sequence of SEQ ID NO: 5080; and the sense strand sequence comprises the nucleotide sequence of any one of SEQ ID NOs: 5077, 5084, 5088, 5092, 5096, or 5098; or
(ix) the antisense strand sequence comprises the nucleotide sequence of SEQ ID NO: 5057; and the sense strand sequence comprises the nucleotide sequence of any one of SEQ ID NOs: 5054, 5060, 5064, 5066, 5070, or 5074.
   39. The siRNA of any one of embodiments 1-11, 13, 25-28, or 36-39 or the AAV viral genome of any one of embodiments 11-17, 25-28, or 36-39, wherein the antisense strand sequence comprises the nucleotide sequence of SEQ ID NO: 4908 and the sense strand sequence comprises the nucleotide sequence of SEQ ID NO: 4906.
   40. The siRNA of any one of embodiments 1-11, 13, 25-27, 29, or 36-39 or the AAV viral genome of any one of embodiments 11-16, 18, 25-27, 29, or 36-39, wherein the antisense strand sequence comprises the nucleotide sequence of SEQ ID NO: 4920 and the sense strand sequence comprises the nucleotide sequence of SEQ ID NO: 4918.
   41. The siRNA of any one of embodiments 1-11, 13, 25-27, 30, or 36-39 or the AAV viral genome of any one of embodiments 11-16, 19, 25-27, 30, or 36-39, wherein the antisense strand sequence comprises the nucleotide sequence of SEQ ID NO: 4924 and the sense strand sequence comprises the nucleotide sequence of SEQ ID NO: 4922.
   42. The siRNA of any one of embodiments 1-11, 13, 25-27, 31, or 36-39 or the AAV viral genome of any one of embodiments 11-16, 19, 25-27, 31, or 36-39, wherein the antisense strand sequence comprises the nucleotide sequence of SEQ ID NO: 4908 and the sense strand sequence comprises the nucleotide sequence of SEQ ID NO: 4966.
   43. The siRNA of any one of embodiments 1-11, 13, 25-27, 32, or 36-39 or the AAV viral genome of any one of embodiments 11-16, 19, 25-27, 62, or 36-39, wherein the antisense strand sequence comprises the nucleotide sequence of SEQ ID NO: 4912 and the sense strand sequence comprises the nucleotide sequence of SEQ ID NO: 4970.
   44. The siRNA of any one of embodiments 1-11, 13, 25-27, 33, or 36-39 or the AAV viral genome of any one of embodiments 11-16, 19, 25-27, 33, or 36-39, wherein the antisense strand sequence comprises the nucleotide sequence of SEQ ID NO: 4920 and the sense strand sequence comprises the nucleotide sequence of SEQ ID NO: 4978.
   45. The siRNA of any one of embodiments 1-11, 13, 25-27, 34, or 36-39 or the AAV viral genome of any one of embodiments 11-16, 19, 25-27, 34, or 36-39, wherein the antisense strand sequence comprises the nucleotide sequence of SEQ ID NO: 4924 and the sense strand sequence comprises the nucleotide sequence of SEQ ID NO: 4982.
   46. The siRNA of any one of embodiments 1-11, 13, 25-27, 35, or 36-39 or the AAV viral genome of any one of embodiments 11-16, 19, 25-27, 35, or 36-39, wherein:
(i) the antisense strand sequence comprises the nucleotide sequence of SEQ ID NO: 4912; and the sense strand sequence comprises the nucleotide sequence of SEQ ID NO: 4910;
(ii) the antisense strand sequence comprises the nucleotide sequence of SEQ ID NO: 4916; and the sense strand sequence comprises the nucleotide sequence of SEQ ID NO: 4914;
(iii) the antisense strand sequence comprises the nucleotide sequence of SEQ ID NO: 5057; and the sense strand sequence comprises the nucleotide sequence of SEQ ID NO: 5054;
(iv) the antisense strand sequence comprises the nucleotide sequence of SEQ ID NO: 4908; and the sense strand sequence comprises the nucleotide sequence of SEQ ID NO: 4926;
(v) the antisense strand sequence comprises the nucleotide sequence of SEQ ID NO: 4912; and the sense strand sequence comprises the nucleotide sequence of SEQ ID NO: 4930;
(vi) the antisense strand sequence comprises the nucleotide sequence of SEQ ID NO: 4916; and the sense strand sequence comprises the nucleotide sequence of SEQ ID NO: 4934;
(vii) the antisense strand sequence comprises the nucleotide sequence of SEQ ID NO: 4920; and the sense strand sequence comprises the nucleotide sequence of SEQ ID NO: 4938;
(viii) the antisense strand sequence comprises the nucleotide sequence of SEQ ID NO: 4924; and the sense strand sequence comprises the nucleotide sequence of SEQ ID NO: 4942;
(ix) the antisense strand sequence comprises the nucleotide sequence of SEQ ID NO: 5057; and the sense strand sequence comprises the nucleotide sequence of SEQ ID NO: 5060;
(x) the antisense strand sequence comprises the nucleotide sequence of SEQ ID NO: 4908; and the sense strand sequence comprises the nucleotide sequence of SEQ ID NO: 4946;
(xi) the antisense strand sequence comprises the nucleotide sequence of SEQ ID NO: 4912; and the sense strand sequence comprises the nucleotide sequence of SEQ ID NO: 4950;
(xii) the antisense strand sequence comprises the nucleotide sequence of SEQ ID NO: 4916; and the sense strand sequence comprises the nucleotide sequence of SEQ ID NO: 4954;
(xiii) the antisense strand sequence comprises the nucleotide sequence of SEQ ID NO: 4920; and the sense strand sequence comprises the nucleotide sequence of SEQ ID NO: 4958;
(xiv) the antisense strand sequence comprises the nucleotide sequence of SEQ ID NO: 4924; and the sense strand sequence comprises the nucleotide sequence of SEQ ID NO: 4962;
(xv) the antisense strand sequence comprises the nucleotide sequence of SEQ ID NO: 5057; and the sense strand sequence comprises the nucleotide sequence of SEQ ID NO: 5064;
(xvi) the antisense strand sequence comprises the nucleotide sequence of SEQ ID NO: 4916; and the sense strand sequence comprises the nucleotide sequence of SEQ ID NO: 4974;
(xvii) the antisense strand sequence comprises the nucleotide sequence of SEQ ID NO: 5057; and the sense strand sequence comprises the nucleotide sequence of SEQ ID NO: 5066;
(xviii) the antisense strand sequence comprises the nucleotide sequence of SEQ ID NO: 4908; and the sense strand sequence comprises the nucleotide sequence of SEQ ID NO: 4986;
(xix) the antisense strand sequence comprises the nucleotide sequence of SEQ ID NO: 4912; and the sense strand sequence comprises the nucleotide sequence of SEQ ID NO: 4990;
(xx) the antisense strand sequence comprises the nucleotide sequence of SEQ ID NO: 4916; and the sense strand sequence comprises the nucleotide sequence of SEQ ID NO: 4994;
(xxi) the antisense strand sequence comprises the nucleotide sequence of SEQ ID NO: 4920; and the sense strand sequence comprises the nucleotide sequence of SEQ ID NO: 4998;
(xxii) the antisense strand sequence comprises the nucleotide sequence of SEQ ID NO: 4924; and the sense strand sequence comprises the nucleotide sequence of SEQ ID NO: 5002;
(xxiii) the antisense strand sequence comprises the nucleotide sequence of SEQ ID NO: 5057; and the sense strand sequence comprises the nucleotide sequence of SEQ ID NO: 5070;
(xxiv) the antisense strand sequence comprises the nucleotide sequence of SEQ ID NO: 4908; and the sense strand sequence comprises the nucleotide sequence of SEQ ID NO: 5006;
(xxv) the antisense strand sequence comprises the nucleotide sequence of SEQ ID NO: 4912; and the sense strand sequence comprises the nucleotide sequence of SEQ ID NO: 5010;
(xxvi) the antisense strand sequence comprises the nucleotide sequence of SEQ ID NO: 4916; and the sense strand sequence comprises the nucleotide sequence of SEQ ID NO: 5014;
(xxvii) the antisense strand sequence comprises the nucleotide sequence of SEQ ID NO: 4920; and the sense strand sequence comprises the nucleotide sequence of SEQ ID NO: 4958;
(xxviii) the antisense strand sequence comprises the nucleotide sequence of SEQ ID NO: 4924; and the sense strand sequence comprises the nucleotide sequence of SEQ ID NO: 5022;
(xxvii) the antisense strand sequence comprises the nucleotide sequence of SEQ ID NO: 5057; and the sense strand sequence comprises the nucleotide sequence of SEQ ID NO: 5074;
(xxix) the antisense strand sequence comprises the nucleotide sequence of SEQ ID NO: 5080; and the sense strand sequence comprises the nucleotide sequence of SEQ ID NO: 5077;
(xxx) the antisense strand sequence comprises the nucleotide sequence of SEQ ID NO: 5080; and the sense strand sequence comprises the nucleotide sequence of SEQ ID NO: 5084;
(xxxi) the antisense strand sequence comprises the nucleotide sequence of SEQ ID NO: 5080; and the sense strand sequence comprises the nucleotide sequence of SEQ ID NO: 5088;
(xxxii) the antisense strand sequence comprises the nucleotide sequence of SEQ ID NO: 5080; and the sense strand sequence comprises the nucleotide sequence of SEQ ID NO: 5092;
(xxxiii) the antisense strand sequence comprises the nucleotide sequence of SEQ ID NO: 5080; and the sense strand sequence comprises the nucleotide sequence of SEQ ID NO: 5096;
(xxxiv) the antisense strand sequence comprises the nucleotide sequence of SEQ ID NO: 5080; and the sense strand sequence comprises the nucleotide sequence of SEQ ID NO: 5098;
(xxxv) the antisense strand sequence comprises the nucleotide sequence of SEQ ID NO: 5104; and the sense strand sequence comprises the nucleotide sequence of SEQ ID NO: 5101;
(xxxvi) the antisense strand sequence comprises the nucleotide sequence of SEQ ID NO: 5104; and the sense strand sequence comprises the nucleotide sequence of SEQ ID NO: 5108;
(xxxvii) the antisense strand sequence comprises the nucleotide sequence of SEQ ID NO: 5104; and the sense strand sequence comprises the nucleotide sequence of SEQ ID NO: 5112;
(xxxviii) the antisense strand sequence comprises the nucleotide sequence of SEQ ID NO: 5104; and the sense strand sequence comprises the nucleotide sequence of SEQ ID NO: 5116;
(xxxix) the antisense strand sequence comprises the nucleotide sequence of SEQ ID NO: 5104; and the sense strand sequence comprises the nucleotide sequence of SEQ ID NO: 5120;
(xl) the antisense strand sequence comprises the nucleotide sequence of SEQ ID NO: 5104; and the sense strand sequence comprises the nucleotide sequence of SEQ ID NO: 5122;
(xli) the antisense strand sequence comprises the nucleotide sequence of SEQ ID NO: 5128; and the sense strand sequence comprises the nucleotide sequence of SEQ ID NO: 5125;
(xlii) the antisense strand sequence comprises the nucleotide sequence of SEQ ID NO: 5128; and the sense strand sequence comprises the nucleotide sequence of SEQ ID NO: 5132;
(xliii) the antisense strand sequence comprises the nucleotide sequence of SEQ ID NO: 5128; and the sense strand sequence comprises the nucleotide sequence of SEQ ID NO: 5136;
(xlv) the antisense strand sequence comprises the nucleotide sequence of SEQ ID NO: 5128; and the sense strand sequence comprises the nucleotide sequence of SEQ ID NO: 5140;
(xliv) the antisense strand sequence comprises the nucleotide sequence of SEQ ID NO: 5128; and the sense strand sequence comprises the nucleotide sequence of SEQ ID NO: 5144; or
(xlvi) the antisense strand sequence comprises the nucleotide sequence of SEQ ID NO: 5128; and the sense strand sequence comprises the nucleotide sequence of SEQ ID NO: 5146.
   47. The siRNA of any one of embodiments 1-11 or 13-46 or the AAV viral genome of any one of embodiments 12-46, wherein:
(i) the antisense strand sequence comprises the nucleotide sequence of SEQ ID NO: 4700 and the sense strand sequence comprises the nucleotide sequence of any one of SEQ ID NOs: 4502-4505;
(ii) the antisense strand sequence comprises the nucleotide sequence of SEQ ID NO: 4920 and the sense strand sequence comprises the nucleotide sequence of any one of SEQ ID NOs: 4918, 4938, 4958, 4978, or 4998;
(iii) the antisense strand sequence comprises the nucleotide sequence of SEQ ID NO: 4697 and the sense strand sequence comprises the nucleotide sequence of any one of SEQ ID NOs: 4495-4499;
(iv) the antisense strand sequence comprises the nucleotide sequence of SEQ ID NO: 4908 and the sense strand sequence comprises the nucleotide sequence of any one of SEQ ID NOs: 4906, 4926, 4946, 4966, 4986, or 5006;
(v) the antisense strand sequence comprises at least 15, 16, 17, 18, or 19 contiguous nucleotides from the nucleotide sequence of SEQ ID NO: 4690 and the sense strand sequence comprises at least 15, 16, 17, 18, or 19 contiguous nucleotides from the nucleotide sequence of any one of SEQ ID NOs: 4480-4484;
(vi) the antisense strand sequence comprises the nucleotide sequence of SEQ ID NO: 4924 and the sense strand sequence comprises the nucleotide sequence of any one of SEQ ID NOs: 4922, 4942, 4962, 4982, 5002, or 5022;
(vii) the antisense strand sequence comprises the nucleotide sequence of SEQ ID NO: 4694 and the sense strand sequence comprises the nucleotide sequence of SEQ ID NO: 4492;
(viii) the antisense strand sequence comprises the nucleotide sequence of SEQ ID NO: 4691 and the sense strand sequence comprises the nucleotide sequence of any one of SEQ ID NOs: 4485-4489;
(ix) the antisense strand sequence comprises the nucleotide sequence of SEQ ID NO: 4916 and the sense strand sequence comprises the nucleotide sequence of any one of SEQ ID NOs: 4914, 4934, 4954, 4974, 4994, or 5014;
(x) the antisense strand sequence comprises the nucleotide sequence of SEQ ID NO: 4701 and the sense strand sequence comprises the nucleotide sequence of any one of SEQ ID NOs: 4506-4510;
(xi) the antisense strand sequence comprises the nucleotide sequence of SEQ ID NO: 4912 and the sense strand sequence comprises the nucleotide sequence of any one of SEQ ID NOs: 4910, 4930, 4950, 4970, 4990, or 5010;
(xii) the antisense strand sequence comprises the nucleotide sequence of SEQ ID NO: 4687 and the sense strand sequence comprises the nucleotide sequence of SEQ ID NO: 4477;
(xiii) the antisense strand sequence comprises the nucleotide sequence of SEQ ID NO: 4712 and the sense strand sequence comprises the nucleotide sequence of SEQ ID NO: 4521;
(xiv) the antisense strand sequence comprises the nucleotide sequence of SEQ ID NO: 4696 and the sense strand sequence comprises the nucleotide sequence of SEQ ID NO: 4494; or
(xv) the antisense strand sequence comprises the nucleotide sequence of SEQ ID NO: 4718 and the sense strand sequence comprises the nucleotide sequence of SEQ ID NO: 4527.
   48. The siRNA of any one of embodiments 1-11 or 13-47 or the AAV viral genome of any one of embodiments 12-47, wherein
(i) the nucleotide sequence encoding the sense strand sequence comprises the nucleotide sequence of SEQ ID NO: 4420 and the nucleotide sequence encoding the antisense strand comprises the nucleotide sequence of SEQ ID NO: 4434;
(ii) the nucleotide sequence encoding the sense strand sequence comprises the nucleotide sequence of SEQ ID NO: 4905 and the nucleotide sequence encoding the antisense strand comprises the nucleotide sequence of SEQ ID NO: 4907;
(iii) the nucleotide sequence encoding the sense strand sequence comprises the nucleotide sequence of SEQ ID NO: 4421 and the nucleotide sequence encoding the antisense strand comprises the nucleotide sequence of SEQ ID NO: 4435;
(iv) the nucleotide sequence encoding the sense strand sequence comprises the nucleotide sequence of SEQ ID NO: 4909 and the nucleotide sequence encoding the antisense strand comprises the nucleotide sequence of SEQ ID NO: 4911;
(v) the nucleotide sequence encoding the sense strand sequence comprises the nucleotide sequence of SEQ ID NO: 4422 and the nucleotide sequence encoding the antisense strand comprises the nucleotide sequence of SEQ ID NO: 4436;
(vi) the nucleotide sequence encoding the sense strand sequence comprises the nucleotide sequence of SEQ ID NO: 4913 and the nucleotide sequence encoding the antisense strand comprises the nucleotide sequence of SEQ ID NO: 4915;
(vii) the nucleotide sequence encoding the sense strand sequence comprises the nucleotide sequence of SEQ ID NO: 4423 and the nucleotide sequence encoding the antisense strand comprises the nucleotide sequence of SEQ ID NO: 4437;
(viii) the nucleotide sequence encoding the sense strand sequence comprises the nucleotide sequence of SEQ ID NO: 4917 and the nucleotide sequence encoding the antisense strand comprises the nucleotide sequence of SEQ ID NO: 4919;
(ix) the nucleotide sequence encoding the sense strand sequence comprises the nucleotide sequence of SEQ ID NO: 4424 and the nucleotide sequence encoding the antisense strand comprises the nucleotide sequence of SEQ ID NO: 4438;
(x) the nucleotide sequence encoding the sense strand sequence comprises the nucleotide sequence of SEQ ID NO: 4921 and the nucleotide sequence encoding the antisense strand comprises the nucleotide sequence of SEQ ID NO: 4923;
(xi) the nucleotide sequence encoding the sense strand sequence comprises the nucleotide sequence of SEQ ID NO: 4410 and the nucleotide sequence encoding the antisense strand comprises the nucleotide sequence of SEQ ID NO: 4434;
(xii) the nucleotide sequence encoding the sense strand sequence comprises the nucleotide sequence of SEQ ID NO: 4925 and the nucleotide sequence encoding the antisense strand comprises the nucleotide sequence of SEQ ID NO: 4907;
(xiii) the nucleotide sequence encoding the sense strand sequence comprises the nucleotide sequence of SEQ ID NO: 4411 and the nucleotide sequence encoding the antisense strand comprises the nucleotide sequence of SEQ ID NO: 4435;
(xiv) the nucleotide sequence encoding the sense strand sequence comprises the nucleotide sequence of SEQ ID NO: 4929 and the nucleotide sequence encoding the antisense strand comprises the nucleotide sequence of SEQ ID NO: 4911;
(xv) the nucleotide sequence encoding the sense strand sequence comprises the nucleotide sequence of SEQ ID NO: 4412 and the nucleotide sequence encoding the antisense strand comprises the nucleotide sequence of SEQ ID NO: 4436;
(xvi) the nucleotide sequence encoding the sense strand sequence comprises the nucleotide sequence of SEQ ID NO: 4933 and the nucleotide sequence encoding the antisense strand comprises the nucleotide sequence of SEQ ID NO: 4915;
(xvii) the nucleotide sequence encoding the sense strand sequence comprises the nucleotide sequence of SEQ ID NO: 4413 and the nucleotide sequence encoding the antisense strand comprises the nucleotide sequence of SEQ ID NO: 4437;
(xviii) the nucleotide sequence encoding the sense strand sequence comprises the nucleotide sequence of SEQ ID NO: 4937 and the nucleotide sequence encoding the antisense strand comprises the nucleotide sequence of SEQ ID NO: 4419;
(xix) the nucleotide sequence encoding the sense strand sequence comprises the nucleotide sequence of SEQ ID NO: 4414 and the nucleotide sequence encoding the antisense strand comprises the nucleotide sequence of SEQ ID NO: 4438;
(xx) the nucleotide sequence encoding the sense strand sequence comprises the nucleotide sequence of SEQ ID NO: 4941 and the nucleotide sequence encoding the antisense strand comprises the nucleotide sequence of SEQ ID NO: 4923;
(xxi) the nucleotide sequence encoding the sense strand sequence comprises the nucleotide sequence of SEQ ID NO: 4415 and the nucleotide sequence encoding the antisense strand comprises the nucleotide sequence of SEQ ID NO: 4434;
(xxii) the nucleotide sequence encoding the sense strand sequence comprises the nucleotide sequence of SEQ ID NO: 4945 and the nucleotide sequence encoding the antisense strand comprises the nucleotide sequence of SEQ ID NO: 4907;
(xxiii) the nucleotide sequence encoding the sense strand sequence comprises the nucleotide sequence of SEQ ID NO: 4416 and the nucleotide sequence encoding the antisense strand comprises the nucleotide sequence of SEQ ID NO: 4435;
(xxiv) the nucleotide sequence encoding the sense strand sequence comprises the nucleotide sequence of SEQ ID NO: 4949 and the nucleotide sequence encoding the antisense strand comprises the nucleotide sequence of SEQ ID NO: 4911;
(xxv) the nucleotide sequence encoding the sense strand sequence comprises the nucleotide sequence of SEQ ID NO: 4417 and the nucleotide sequence encoding the antisense strand comprises the nucleotide sequence of SEQ ID NO: 4436;
(xxvi) the nucleotide sequence encoding the sense strand sequence comprises the nucleotide sequence of SEQ ID NO: 4953 and the nucleotide sequence encoding the antisense strand comprises the nucleotide sequence of SEQ ID NO: 4915;
(xxvii) the nucleotide sequence encoding the sense strand sequence comprises the nucleotide sequence of SEQ ID NO: 4418 and the nucleotide sequence encoding the antisense strand comprises the nucleotide sequence of SEQ ID NO: 4437;
(xxviii) the nucleotide sequence encoding the sense strand sequence comprises the nucleotide sequence of SEQ ID NO: 4957 and the nucleotide sequence encoding the antisense strand comprises the nucleotide sequence of SEQ ID NO: 4919;
(xxix) the nucleotide sequence encoding the sense strand sequence comprises the nucleotide sequence of SEQ ID NO: 4419 and the nucleotide sequence encoding the antisense strand comprises the nucleotide sequence of SEQ ID NO: 4438;
(xxx) the nucleotide sequence encoding the sense strand sequence comprises the nucleotide sequence of SEQ ID NO: 4961 and the nucleotide sequence encoding the antisense strand comprises the nucleotide sequence of SEQ ID NO: 4923;
(xxxi) the nucleotide sequence encoding the sense strand sequence comprises the nucleotide sequence of SEQ ID NO: 4420 and the nucleotide sequence encoding the antisense strand comprises the nucleotide sequence of SEQ ID NO: 4434;
(xxxii) the nucleotide sequence encoding the sense strand sequence comprises the nucleotide sequence of SEQ ID NO: 4965 and the nucleotide sequence encoding the antisense strand comprises the nucleotide sequence of SEQ ID NO: 4907;
(xxxii) the nucleotide sequence encoding the sense strand sequence comprises the nucleotide sequence of SEQ ID NO: 4421 and the nucleotide sequence encoding the antisense strand comprises the nucleotide sequence of SEQ ID NO: 4435;
(xxxiii) the nucleotide sequence encoding the sense strand sequence comprises the nucleotide sequence of SEQ ID NO: 4969 and the nucleotide sequence encoding the antisense strand comprises the nucleotide sequence of SEQ ID NO: 4911;
(xxxiv) the nucleotide sequence encoding the sense strand sequence comprises the nucleotide sequence of SEQ ID NO: 4422 and the nucleotide sequence encoding the antisense strand comprises the nucleotide sequence of SEQ ID NO: 4436;
(xxxv) the nucleotide sequence encoding the sense strand sequence comprises the nucleotide sequence of SEQ ID NO: 4973 and the nucleotide sequence encoding the antisense strand comprises the nucleotide sequence of SEQ ID NO: 4915;
(xxxvi) the nucleotide sequence encoding the sense strand sequence comprises the nucleotide sequence of SEQ ID NO: 4423 and the nucleotide sequence encoding the antisense strand comprises the nucleotide sequence of SEQ ID NO: 4437;
(xxxvii) the nucleotide sequence encoding the sense strand sequence comprises the nucleotide sequence of SEQ ID NO: 4977 and the nucleotide sequence encoding the antisense strand comprises the nucleotide sequence of SEQ ID NO: 4919;
(xxxviii) the nucleotide sequence encoding the sense strand sequence comprises the nucleotide sequence of SEQ ID NO: 4424 and the nucleotide sequence encoding the antisense strand comprises the nucleotide sequence of SEQ ID NO: 4438;
(xxxix) the nucleotide sequence encoding the sense strand sequence comprises the nucleotide sequence of SEQ ID NO: 498I and the nucleotide sequence encoding the antisense strand comprises the nucleotide sequence of SEQ ID NO: 4923;
(xl) the nucleotide sequence encoding the sense strand sequence comprises the nucleotide sequence of SEQ ID NO: 4425 and the nucleotide sequence encoding the antisense strand comprises the nucleotide sequence of SEQ ID NO: 4434;
(xli) the nucleotide sequence encoding the sense strand sequence comprises the nucleotide sequence of SEQ ID NO: 4985 and the nucleotide sequence encoding the antisense strand comprises the nucleotide sequence of SEQ ID NO: 4907;
(xlii) the nucleotide sequence encoding the sense strand sequence comprises the nucleotide sequence of SEQ ID NO: 4426 and the nucleotide sequence encoding the antisense strand comprises the nucleotide sequence of SEQ ID NO: 4435;
(xliii) the nucleotide sequence encoding the sense strand sequence comprises the nucleotide sequence of SEQ ID NO: 4989 and the nucleotide sequence encoding the antisense strand comprises the nucleotide sequence of SEQ ID NO: 4911;
(xliv) the nucleotide sequence encoding the sense strand sequence comprises the nucleotide sequence of SEQ ID NO: 4427 and the nucleotide sequence encoding the antisense strand comprises the nucleotide sequence of SEQ ID NO: 4436;
(xlv) the nucleotide sequence encoding the sense strand sequence comprises the nucleotide sequence of SEQ ID NO: 4993 and the nucleotide sequence encoding the antisense strand comprises the nucleotide sequence of SEQ ID NO: 4915;
(xlvi) the nucleotide sequence encoding the sense strand sequence comprises the nucleotide sequence of SEQ ID NO: 4428 and the nucleotide sequence encoding the antisense strand comprises the nucleotide sequence of SEQ ID NO: 4437;
(xlvii) the nucleotide sequence encoding the sense strand sequence comprises the nucleotide sequence of SEQ ID NO: 4997 and the nucleotide sequence encoding the antisense strand comprises the nucleotide sequence of SEQ ID NO: 4919;
(xlviii) the nucleotide sequence encoding the sense strand sequence comprises the nucleotide sequence of SEQ ID NO: 4429 and the nucleotide sequence encoding the antisense strand comprises the nucleotide sequence of SEQ ID NO: 4438;
(xlix) the nucleotide sequence encoding the sense strand sequence comprises the nucleotide sequence of SEQ ID NO: 5001 and the nucleotide sequence encoding the antisense strand comprises the nucleotide sequence of SEQ ID NO: 4923;
(l) the nucleotide sequence encoding the sense strand sequence comprises the nucleotide sequence of SEQ ID NO: 4430 and the nucleotide sequence encoding the antisense strand comprises the nucleotide sequence of SEQ ID NO: 4434;
(li) the nucleotide sequence encoding the sense strand sequence comprises the nucleotide sequence of SEQ ID NO: 5005 and the nucleotide sequence encoding the antisense strand comprises the nucleotide sequence of SEQ ID NO: 4907;
(lii) the nucleotide sequence encoding the sense strand sequence comprises the nucleotide sequence of SEQ ID NO: 4431 and the nucleotide sequence encoding the antisense strand comprises the nucleotide sequence of SEQ ID NO: 4435;
(liii) the nucleotide sequence encoding the sense strand sequence comprises the nucleotide sequence of SEQ ID NO: 5009 and the nucleotide sequence encoding the antisense strand comprises the nucleotide sequence of SEQ ID NO: 4911;
(liv) the nucleotide sequence encoding the sense strand sequence comprises the nucleotide sequence of SEQ ID NO: 4432 and the nucleotide sequence encoding the antisense strand comprises the nucleotide sequence of SEQ ID NO: 4436;
(lv) the nucleotide sequence encoding the sense strand sequence comprises the nucleotide sequence of SEQ ID NO: 5013 and the nucleotide sequence encoding the antisense strand comprises the nucleotide sequence of SEQ ID NO: 4915;
(lvi) the nucleotide sequence encoding the sense strand sequence comprises the nucleotide sequence of SEQ ID NO: 4433 and the nucleotide sequence encoding the antisense strand comprises the nucleotide sequence of SEQ ID NO: 4438;
(lvii) the nucleotide sequence encoding the sense strand sequence comprises the nucleotide sequence of SEQ ID NO: 5021 and the nucleotide sequence encoding the antisense strand comprises the nucleotide sequence of SEQ ID NO: 4923;
(lviii) the nucleotide sequence encoding the sense strand sequence comprises the nucleotide sequence of SEQ ID NO: 5053 and the nucleotide sequence encoding the antisense strand comprises the nucleotide sequence of SEQ ID NO: 5056;
(lix) the nucleotide sequence encoding the sense strand sequence comprises the nucleotide sequence of SEQ ID NO: 5059 and the nucleotide sequence encoding the antisense strand comprises the nucleotide sequence of SEQ ID NO: 5056;
(lx) the nucleotide sequence encoding the sense strand sequence comprises the nucleotide sequence of SEQ ID NO: 5063 and the nucleotide sequence encoding the antisense strand comprises the nucleotide sequence of SEQ ID NO: 5056;
(lxi) the nucleotide sequence encoding the sense strand sequence comprises the nucleotide sequence of SEQ ID NO: 5065 and the nucleotide sequence encoding the antisense strand comprises the nucleotide sequence of SEQ ID NO: 5056;
(lxii) the nucleotide sequence encoding the sense strand sequence comprises the nucleotide sequence of SEQ ID NO: 5069 and the nucleotide sequence encoding the antisense strand comprises the nucleotide sequence of SEQ ID NO: 5056;
(lxiii) the nucleotide sequence encoding the sense strand sequence comprises the nucleotide sequence of SEQ ID NO: 5073 and the nucleotide sequence encoding the antisense strand comprises the nucleotide sequence of SEQ ID NO: 5056;
(lxiv) the nucleotide sequence encoding the sense strand sequence comprises the nucleotide sequence of SEQ ID NO: 5076 and the nucleotide sequence encoding the antisense strand comprises the nucleotide sequence of SEQ ID NO: 5079;
(lxv) the nucleotide sequence encoding the sense strand sequence comprises the nucleotide sequence of SEQ ID NO: 5083 and the nucleotide sequence encoding the antisense strand comprises the nucleotide sequence of SEQ ID NO: 5079;
(lxvi) the nucleotide sequence encoding the sense strand sequence comprises the nucleotide sequence of SEQ ID NO: 5087 and the nucleotide sequence encoding the antisense strand comprises the nucleotide sequence of SEQ ID NO: 5079;
(lxvii) the nucleotide sequence encoding the sense strand sequence comprises the nucleotide sequence of SEQ ID NO: 5091 and the nucleotide sequence encoding the antisense strand comprises the nucleotide sequence of SEQ ID NO: 5079;
(lxviii) the nucleotide sequence encoding the sense strand sequence comprises the nucleotide sequence of SEQ ID NO: 5095 and the nucleotide sequence encoding the antisense strand comprises the nucleotide sequence of SEQ ID NO: 5079;
(lxix) the nucleotide sequence encoding the sense strand sequence comprises the nucleotide sequence of SEQ ID NO: 5097 and the nucleotide sequence encoding the antisense strand comprises the nucleotide sequence of SEQ ID NO: 5079;
(lxx) the nucleotide sequence encoding the sense strand sequence comprises the nucleotide sequence of SEQ ID NO: 5100 and the nucleotide sequence encoding the antisense strand comprises the nucleotide sequence of SEQ ID NO: 5103;
(lxxi) the nucleotide sequence encoding the sense strand sequence comprises the nucleotide sequence of SEQ ID NO: 5107 and the nucleotide sequence encoding the antisense strand comprises the nucleotide sequence of SEQ ID NO: 5103;
(lxxii) the nucleotide sequence encoding the sense strand sequence comprises the nucleotide sequence of SEQ ID NO: 5111 and the nucleotide sequence encoding the antisense strand comprises the nucleotide sequence of SEQ ID NO: 5103;
(lxxiii) the nucleotide sequence encoding the sense strand sequence comprises the nucleotide sequence of SEQ ID NO: 5115 and the nucleotide sequence encoding the antisense strand comprises the nucleotide sequence of SEQ ID NO: 5103;
(lxxiv) the nucleotide sequence encoding the sense strand sequence comprises the nucleotide sequence of SEQ ID NO: 5119 and the nucleotide sequence encoding the antisense strand comprises the nucleotide sequence of SEQ ID NO: 5103;
(lxxv) the nucleotide sequence encoding the sense strand sequence comprises the nucleotide sequence of SEQ ID NO: 5121 and the nucleotide sequence encoding the antisense strand comprises the nucleotide sequence of SEQ ID NO: 5103;
(lxxvi) the nucleotide sequence encoding the sense strand sequence comprises the nucleotide sequence of SEQ ID NO: 5124 and the nucleotide sequence encoding the antisense strand comprises the nucleotide sequence of SEQ ID NO: 5127;
(lxxvii) the nucleotide sequence encoding the sense strand sequence comprises the nucleotide sequence of SEQ ID NO: 5131 and the nucleotide sequence encoding the antisense strand comprises the nucleotide sequence of SEQ ID NO: 5127;
(lxxviii) the nucleotide sequence encoding the sense strand sequence comprises the nucleotide sequence of SEQ ID NO: 5135 and the nucleotide sequence encoding the antisense strand comprises the nucleotide sequence of SEQ ID NO: 5127;
(lxxix) the nucleotide sequence encoding the sense strand sequence comprises the nucleotide sequence of SEQ ID NO: 5139 and the nucleotide sequence encoding the antisense strand comprises the nucleotide sequence of SEQ ID NO: 5127;
(lxxx) the nucleotide sequence encoding the sense strand sequence comprises the nucleotide sequence of SEQ ID NO: 5143 and the nucleotide sequence encoding the antisense strand comprises the nucleotide sequence of SEQ ID NO: 5127; or
(lxxxi) the nucleotide sequence encoding the sense strand sequence comprises the nucleotide sequence of SEQ ID NO: 5145 and the nucleotide sequence encoding the antisense strand comprises the nucleotide sequence of SEQ ID NO: 5127.
   49. The siRNA of any one of embodiments 1-11, 13, 25-28, 36-39, 47, or 48, or the AAV viral genome of any one of embodiments 11-17, 25-28, 36-39, or 47, or 48, wherein the nucleotide sequence encoding the antisense strand sequence comprises the nucleotide sequence of SEQ ID NO: 4907 and the nucleotide sequence encoding the sense strand sequence comprises the nucleotide sequence of SEQ ID NO: 4905.
   50. The siRNA of any one of embodiments 1-11, 13, 25-27, 29, 36-39, 47, or 48 or the AAV viral genome of any one of embodiments 11-16, 18, 25-27, 29, 36-39, 47, or 48, wherein the nucleotide sequence encoding the antisense strand sequence comprises the nucleotide sequence of SEQ ID NO: 4919 and the nucleotide sequence encoding the sense strand sequence comprises the nucleotide sequence of SEQ ID NO: 4917.
   51. The siRNA of any one of embodiments 1-11, 13, 25-27, 30, 36-39, 47, or 48 or the AAV viral genome of any one of embodiments 11-16, 19, 25-27, 30, 36-39, 47, or 48, wherein the nucleotide sequence encoding the antisense strand sequence comprises the nucleotide sequence of SEQ ID NO: 4923 and the nucleotide sequence encoding the sense strand sequence comprises the nucleotide sequence of SEQ ID NO: 4921.
   52. The siRNA of any one of embodiments 1-11, 13, 25-27, 31, 36-39, 47, or 48 or the AAV viral genome of any one of embodiments 11-16, 19, 25-27, 31, 36-39, 47, or 48, wherein the nucleotide sequence encoding the antisense strand sequence comprises the nucleotide sequence of SEQ ID NO: 4907 and the nucleotide sequence encoding the sense strand sequence comprises the nucleotide sequence of SEQ ID NO: 4965.
   53. The siRNA of any one of embodiments 1-11, 13, 25-27, 32, 36-39, 47, or 48 or the AAV viral genome of any one of embodiments 11-16, 19, 25-27, 62, 36-39, 47, or 48, wherein the nucleotide sequence encoding the antisense strand sequence comprises the nucleotide sequence of SEQ ID NO: 4911 and the nucleotide sequence encoding the sense strand sequence comprises the nucleotide sequence of SEQ ID NO: 4969.
   54. The siRNA of any one of embodiments 1-11, 13, 25-27, 33, 36-39, 47, or 48 or the AAV viral genome of any one of embodiments 11-16, 19, 25-27, 33, 36-39, 47, or 48, wherein the nucleotide sequence encoding the antisense strand sequence comprises the nucleotide sequence of SEQ ID NO: 4919 and the nucleotide sequence encoding the sense strand sequence comprises the nucleotide sequence of SEQ ID NO: 4977.
   55. The siRNA of any one of embodiments 1-11, 13, 25-27, 34, 36-39, 47, or 48 or the AAV viral genome of any one of embodiments 11-16, 19, 25-27, 34, 36-39, 47, or 48, wherein the nucleotide sequence encoding the antisense strand sequence comprises the nucleotide sequence of SEQ ID NO: 4923 and the nucleotide sequence encoding the sense strand sequence comprises the nucleotide sequence of SEQ ID NO: 498I.
   56. The siRNA of any one of embodiments 1-11 or 13-55, or the AAV viral genome of any one of embodiments 12-55, wherein the sense strand sequence, the antisense strand sequence, or both the sense strand sequence and the antisense strand sequence comprise at least 15-30, 19-21, or 25-30 nucleotides in length, e.g., 17, 18, 20, 21, 22, 25, or 30 nucleotides in length.
   57. The siRNA of any one of embodiments 1-11 or 13-56 or the AAV viral genome of any one of embodiments 12-56, wherein the sense strand sequence, the antisense strand sequence, or both the sense strand sequence and the antisense strand sequence comprise an overhang, e.g., an overhang at the 5′ end of the sense strand sequence and/or at the 3′ end of the antisense strand sequence, of 1 or 2 nucleotides.
   58. The siRNA of any one of embodiments 1-11 or 13-57 or the AAV viral genome of any one of embodiments 12-57, wherein:
(i) the sense strand and the antisense strand comprise one mismatch; and/or
(ii) the antisense strand and the target sequence comprise one mismatch.
   59. A modulatory polynucleotide comprising the siRNA of any one of embodiments 1-12 or 13-58.
   60. The AAV viral genome of any one of embodiments 13-58, wherein the nucleotide sequence further encodes a modulatory polynucleotide comprising the sense strand sequence and the antisense strand sequence.
   61. The modulatory polynucleotide of embodiment 59, or the AAV viral genome of embodiment 60, wherein the modulatory polynucleotide further comprises one, two, three or all of:
(i) a 5′ flanking region;
(ii) a loop region; and
(iii) a 3′ flanking region.
   62. The modulatory polynucleotide of embodiment 61, or the AAV viral genome of embodiment 61, wherein:
(i) the 5′ flanking region comprises the nucleotide sequence of any one of SEQ ID NOs: 4878-4886; a nucleotide sequence at least 70%, 75%, 80%, 85%, 90%, 95% 96%, 97%, 98%, 99% identical thereto; a nucleotide sequence comprising one, two, three, four, five, six, or seven, but no more than ten different nucleotides relative to the nucleotide sequence of any of SEQ ID NOs: 4878-4886; or a nucleotide sequence comprising one, two, three, four, five, six, or seven, but no more than ten modifications relative to the nucleotide sequence of any of SEQ ID NOs: 4878-4886;
(ii) the loop region comprises the nucleotide sequence of any of SEQ ID NOs: 4887-4896; a nucleotide sequence at least 70%, 75%, 80%, 85%, 90%, 95% 96%, 97%, 98%, 99% identical thereto; a nucleotide sequence comprising one, two, three, or four, but no more than five different nucleotides relative to the nucleotide sequence of any of SEQ ID NOs: 4887-4896; or a nucleotide sequence comprising one, two, three, or four, but no more than five modifications relative to the nucleotide sequence of any of SEQ ID NOs: 4887-4896; and/or
(iii) the 3′ flanking region comprises the nucleotide sequence of any one of SEQ ID NOs: 4897-4904; a nucleotide sequence at least 70%, 75%, 80%, 85%, 90%, 95% 96%, 97%, 98%, 99% identical thereto; a nucleotide sequence comprising one, two, three, four, five, six, or seven, but no more than ten modifications relative to the nucleotide sequence of any of SEQ ID NOs: 4897-4904; or a nucleotide sequence comprising one, two, three, four, five, six, or seven, but no more than ten modifications relative to the nucleotide sequence of any of SEQ ID NOs: 4897-4904 63. The modulatory polynucleotide of embodiment 61 or 62 or the AAV viral genome of embodiment 61 or 62, wherein:
(i) the nucleotide sequence encoding the 5′ flanking region comprises the nucleotide sequence of any of SEQ ID NOs: 4353-4361, a nucleotide sequence at least 70%, 75%, 80%, 85%, 90%, 95% 96%, 97%, 98%, 99% identical thereto; a nucleotide sequence comprising one, two, three, four, five, six, or seven, but no more than ten different nucleotides relative to the nucleotide sequence of any of SEQ ID NOs: 4353-4361; or a nucleotide sequence comprising one, two, three, four, five, six, or seven, but no more than ten modifications relative to the nucleotide sequence of any of SEQ ID NOs: 4353-4361;
(ii) the nucleotide sequence encoding the loop region comprises the nucleotide sequence of any of SEQ ID NOs: 4362-4371; a nucleotide sequence at least 70%, 75%, 80%, 85%, 90%, 95% 96%, 97%, 98%, 99% identical thereto; a nucleotide sequence comprising one, two, three, or four, but no more than five different nucleotides relative to the nucleotide sequence of any of SEQ ID NOs: 4362-4371; or a nucleotide sequence comprising one, two, three, or four, but no more than five modifications relative to the nucleotide sequence of any of SEQ ID NOs: 4362-4371; and/or
(iii) the nucleotide sequence encoding the 3′ flanking region comprises the nucleotide sequence of any of SEQ ID NOs: 4372-4379; a nucleotide sequence at least 70%, 75%, 80%, 85%, 90%, 95% 96%, 97%, 98%, 99% identical thereto; a nucleotide sequence comprising one, two, three, four, five, six, or seven, but no more than ten different nucleotides relative to the nucleotide sequence of any of SEQ ID NOs: 4372-4379; or a nucleotide sequence comprising one, two, three, four, five, six, or seven, but no more than ten modifications relative to the nucleotide sequence of any of SEQ ID NOs: 4372-4379.
   64. The modulatory polynucleotide of any one of embodiments 59 or 61-63 or the AAV viral genome of any one of embodiments 60-63, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten different nucleotides relative to the nucleotide sequence of SEQ ID NO: 4879;
(ii) a loop region comprising the nucleotide sequence of SEQ ID NO: 4887, a nucleotide sequence at least 70%, 75%, 80%, 85%, 90%, 95% 96%, 97%, 98%, 99% identical to SEQ ID NO: 4887, a nucleotide sequence comprising one, two, three, or four, but no more than five different nucleotides relative to the nucleotide sequence of SEQ ID NO: 4887; or a nucleotide sequence comprising one, two, three, or four, but no more than five different nucleotides relative to the nucleotide sequence of SEQ ID NO: 4887; and
(iii) a 3′ flanking region of SEQ ID NO: 4898, a nucleotide sequence at least 70%, 75%, 80%, 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.
   65. The modulatory polynucleotide of any one of embodiments 59 or 61-63 or the AAV viral genome of any one of embodiments 60-63, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) a loop region comprising the nucleotide sequence of SEQ ID NO: 4888, a nucleotide sequence at least 70%, 75%, 80%, 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888; and
(iii) a 3′ flanking region of SEQ ID NO: 4898, a nucleotide sequence at least 70%, 75%, 80%, 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.
   66. The modulatory polynucleotide of any one of embodiments 59 or 61-63 or the AAV viral genome of any one of embodiments 60-63, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) a loop region comprising the nucleotide sequence of SEQ ID NO: 4887, a nucleotide sequence at least 70%, 75%, 80%, 85%, 90%, 95% 96%, 97%, 98%, 99% identical to SEQ ID NO: 4887, a nucleotide sequence comprising one, two, three, or four, but no more than five different nucleotides relative to the nucleotide sequence of SEQ ID NO: 4887; or a nucleotide sequence comprising one, two, three, or four, but no more than five modifications relative to the nucleotide sequence of SEQ ID NO: 4887; and
(iii) a 3′ flanking region of SEQ ID NO: 4899, a nucleotide sequence at least 70%, 75%, 80%, 85%, 90%, 95% 96%, 97%, 98%, 99% identical to SEQ ID NO: 4899, a nucleotide sequence comprising one, two, three, four, five, six, or seven, but no more than ten different nucleotides relative to the nucleotide sequence of SEQ ID NO: 4899, a nucleotide sequence comprising one, two, three, four, five, six, or seven, but no more than ten modifications relative to the nucleotide sequence of SEQ ID NO: 4899.
   67. The modulatory polynucleotide of any one of embodiments 59 or 61-63 or the AAV viral genome of any one of embodiments 60-63, wherein the modulatory polynucleotide comprises:
(i) a 5′ flanking region comprising the nucleotide sequence of SEQ ID NO: 4880, a nucleotide sequence at least 70%, 75%, 80%, 85%, 90%, 95% 96%, 97%, 98%, 99% identical to SEQ ID NO: 4880, a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) a loop region comprising the nucleotide sequence of SEQ ID NO: 4891, a nucleotide sequence at least 70%, 75%, 80%, 85%, 90%, 95% 96%, 97%, 98%, 99% identical to SEQ ID NO: 4891, a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891; and
(iii) a 3′ flanking region of SEQ ID NO: 4900, a nucleotide sequence at least 70%, 75%, 80%, 85%, 90%, 95% 96%, 97%, 98%, 99% identical to SEQ ID NO: 4900, a nucleotide sequence comprising one, two, three, four, five, six, or seven, but no more than ten different nucleotides relative to the nucleotide sequence of SEQ ID NO: 4900, or a nucleotide sequence comprising one, two, three, four, five, six, or seven, but no more than ten modifications relative to SEQ ID NO: 4900.
   68. The modulatory polynucleotide of any one of embodiments 59 or 61-67 or the AAV viral genome of any one of embodiments 62-67, wherein the modulatory polynucleotide comprises in 5′ to 3′ order:
(i) the 5′ flanking region, sense strand sequence, loop region, antisense strand sequence, and 3′ flanking region; or
(ii) the 5′ flanking region, antisense strand sequence, loop region, sense strand sequence, and 3′ flanking region.
   69. The modulatory polynucleotide of any one of embodiments 59, 61-63, 67, 68 or the AAV viral genome of any one of embodiments 60-63, 67, 68, where the modulatory polynucleotide comprises:
(i) a 5′ flanking region comprising the nucleotide sequence of SEQ ID NO: 4880; a nucleotide sequence at least 70%, 75%, 80%, 85%, 90%, 95% 96%, 97%, 98%, 99% identical to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) the sense strand sequence of SEQ ID NO: 4906;
(iii) a loop region comprising the nucleotide sequence of SEQ ID NO: 4891; a nucleotide sequence at least 70%, 75%, 80%, 85%, 90%, 95% 96%, 97%, 98%, 99% identical to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891;
(iv) the antisense strand sequence of SEQ ID NO: 4908; and
(v) a 3′ flanking region comprising the nucleotide sequence of SEQ ID NO: 4900; a nucleotide sequence at least 70%, 75%, 80%, 85%, 90%, 95% 96%, 97%, 98%, 99% identical to SEQ ID NO: 4900; a nucleotide sequence comprising one, two, three, four, five, six, or seven, but no more than ten different nucleotides relative to the nucleotide sequence of SEQ ID NO: 4900; or a nucleotide sequence comprising one, two, three, four, five, six, or seven, but no more than ten modifications relative to SEQ ID NO: 4900.
   70. The modulatory polynucleotide of any one of embodiments 59, 61-63, or 67-69, or the AAV viral genome of any one of embodiments 60-63 or 67-69, where the nucleotide sequence encoding the modulatory polynucleotide comprises:
(i) a 5′ flanking region comprising the nucleotide sequence of SEQ ID NO: 4355; a nucleotide sequence at least 70%, 75%, 80%, 85%, 90%, 95% 96%, 97%, 98%, 99% identical to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355;
(ii) the sense strand sequence of SEQ ID NO: 4905;
(iii) a loop region comprising the nucleotide sequence of SEQ ID NO: 4366; a nucleotide sequence at least 70%, 75%, 80%, 85%, 90%, 95% 96%, 97%, 98%, 99% identical to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366;
(iv) the antisense strand sequence of SEQ ID NO: 4907; and
(v) a 3′ flanking region comprising the nucleotide sequence of SEQ ID NO: 4375; a nucleotide sequence at least 70%, 75%, 80%, 85%, 90%, 95% 96%, 97%, 98%, 99% identical to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to SEQ ID NO: 4375.
   71. The modulatory polynucleotide of any one of embodiments 59, 61-63, 67, or 68 or the AAV viral genome of any one of embodiments 60-63, 67, or 68, where the modulatory polynucleotide comprises:
(i) a 5′ flanking region comprising the nucleotide sequence of SEQ ID NO: 4880; a nucleotide sequence at least 70%, 75%, 80%, 85%, 90%, 95% 96%, 97%, 98%, 99% identical to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) the sense strand sequence of SEQ ID NO: 4918;
(iii) a loop region comprising the nucleotide sequence of SEQ ID NO: 4891; a nucleotide sequence at least 70%, 75%, 80%, 85%, 90%, 95% 96%, 97%, 98%, 99% identical to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891;
(iv) the antisense strand sequence of SEQ ID NO: 4920; and
(v) a 3′ flanking region comprising the nucleotide sequence of SEQ ID NO: 4900; a nucleotide sequence at least 70%, 75%, 80%, 85%, 90%, 95% 96%, 97%, 98%, 99% identical to SEQ ID NO: 4900; a nucleotide sequence comprising one, two, three, four, five, six, or seven, but no more than ten different nucleotides relative to the nucleotide sequence of SEQ ID NO: 4900; or a nucleotide sequence comprising one, two, three, four, five, six, or seven, but no more than ten modifications relative to SEQ ID NO: 4900.
   72. The modulatory polynucleotide of any one of embodiments 59, 61-63, 67, 68, or 71 or the AAV viral genome of any one of embodiments 60-63, 67, 68 or 71, where the nucleotide sequence encoding the modulatory polynucleotide comprises:
(i) a 5′ flanking region comprising the nucleotide sequence of SEQ ID NO: 4355; a nucleotide sequence at least 70%, 75%, 80%, 85%, 90%, 95% 96%, 97%, 98%, 99% identical to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355;
(ii) the sense strand sequence of SEQ ID NO: 4917;
(iii) a loop region comprising the nucleotide sequence of SEQ ID NO: 4366; a nucleotide sequence at least 70%, 75%, 80%, 85%, 90%, 95% 96%, 97%, 98%, 99% identical to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366;
(iv) the antisense strand sequence of SEQ ID NO: 4919; and
(v) a 3′ flanking region comprising the nucleotide sequence of SEQ ID NO: 4375; a nucleotide sequence at least 70%, 75%, 80%, 85%, 90%, 95% 96%, 97%, 98%, 99% identical to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to SEQ ID NO: 4375.
   73. The modulatory polynucleotide of any one of embodiments 59, 61-63, 67, or 68 or the AAV viral genome of any one of embodiments 60-63, 67, or 68, where the modulatory polynucleotide comprises:
(i) a 5′ flanking region comprising the nucleotide sequence of SEQ ID NO: 4880; a nucleotide sequence at least 70%, 75%, 80%, 85%, 90%, 95% 96%, 97%, 98%, 99% identical to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) the sense strand sequence of SEQ ID NO: 4922;
(iii) a loop region comprising the nucleotide sequence of SEQ ID NO: 4891; a nucleotide sequence at least 70%, 75%, 80%, 85%, 90%, 95% 96%, 97%, 98%, 99% identical to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891;
(iv) the antisense strand sequence of SEQ ID NO: 4924; and
(v) a 3′ flanking region comprising the nucleotide sequence of SEQ ID NO: 4900; a nucleotide sequence at least 70%, 75%, 80%, 85%, 90%, 95% 96%, 97%, 98%, 99% identical to SEQ ID NO: 4900; a nucleotide sequence comprising one, two, three, four, five, six, or seven, but no more than ten different nucleotides relative to the nucleotide sequence of SEQ ID NO: 4900; or a nucleotide sequence comprising one, two, three, four, five, six, or seven, but no more than ten modifications relative to SEQ ID NO: 4900.
   74. The modulatory polynucleotide of any one of embodiments 59, 61-63, 67, 68, or 73 or the AAV viral genome of any one of embodiments 60-63, 67, 68, or 73, where the nucleotide sequence encoding the modulatory polynucleotide comprises:
(i) a 5′ flanking region comprising the nucleotide sequence of SEQ ID NO: 4355; a nucleotide sequence at least 70%, 75%, 80%, 85%, 90%, 95% 96%, 97%, 98%, 99% identical to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355;
(ii) the sense strand sequence of SEQ ID NO: 4921;
(iii) a loop region comprising the nucleotide sequence of SEQ ID NO: 4366; a nucleotide sequence at least 70%, 75%, 80%, 85%, 90%, 95% 96%, 97%, 98%, 99% identical to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366;
(iv) the antisense strand sequence of SEQ ID NO: 4923; and
(v) a 3′ flanking region comprising the nucleotide sequence of SEQ ID NO: 4375; a nucleotide sequence at least 70%, 75%, 80%, 85%, 90%, 95% 96%, 97%, 98%, 99% identical to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to SEQ ID NO: 4375.
   75. The modulatory polynucleotide of any one of embodiments 59, 60-63, 65, or 68 or the AAV viral genome of any one of embodiments 60-63, 65, or 68, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4966;
(iii) a loop region comprising the nucleotide sequence of SEQ ID NO: 4888, a nucleotide sequence at least 70%, 75%, 80%, 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4908; and
(v) a 3′ flanking region of SEQ ID NO: 4898, a nucleotide sequence at least 70%, 75%, 80%, 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.
   76. The modulatory polynucleotide of any one of embodiments 59, 61-63, 65, 68, or 76 or the AAV viral genome of any one of embodiments 60-63, 65, 68, or 76, wherein the nucleotide sequence encoding the modulatory polynucleotide comprises:
(i) a 5′ flanking region comprising the nucleotide sequence of SEQ ID NO: 4354, a nucleotide sequence at least 70%, 75%, 80%, 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4965;
(iii) a loop region comprising the nucleotide sequence of SEQ ID NO: 4363, a nucleotide a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4907; and
(v) a 3′ flanking region of SEQ ID NO: 4373, a nucleotide sequence at least 70%, 75%, 80%, 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.
   77. The modulatory polynucleotide of any one of embodiments 59, 61-63, 65, or 68 or the AAV viral genome of any one of embodiments 60-63, 65, or 68, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4970;
(iii) a loop region comprising the nucleotide sequence of SEQ ID NO: 4888, a nucleotide sequence at least 70%, 75%, 80%, 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4912; and (v) a 3′ flanking region of SEQ ID NO: 4898, a nucleotide sequence at least 70%, 75%, 80%, 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.
   78. The modulatory polynucleotide of any one of embodiments 59, 61-63, 65, 68, or 77 or the AAV viral genome of any one of embodiments 60-63, 65, 68, or 77, wherein the nucleotide sequence encoding the modulatory polynucleotide comprises:
(i) a 5′ flanking region comprising the nucleotide sequence of SEQ ID NO: 4354, a nucleotide sequence at least 70%, 75%, 80%, 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4969;
(iii) a loop region comprising the nucleotide sequence of SEQ ID NO: 4363, a nucleotide sequence at least 70%, 75%, 80%, 85%, 90%, 95% 96%, 97%, 98%, 99% identical to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4911; and
(v) a 3′ flanking region of SEQ ID NO: 4373, a nucleotide sequence at least 70%, 75%, 80%, 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.
   79. The modulatory polynucleotide of any one of embodiments 59, 61-63, 65, or 68 or the AAV viral genome of any one of embodiments 60-63, 65, or 68, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4978;
(iii) a loop region comprising the nucleotide sequence of SEQ ID NO: 4888, a nucleotide sequence at least 70%, 75%, 80%, 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4920; and
(v) a 3′ flanking region of SEQ ID NO: 4898, a nucleotide sequence at least 70%, 75%, 80%, 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.
   80. The modulatory polynucleotide of any one of embodiments 59, 61-63, 65, 68, or 79 or the AAV viral genome of any one of embodiments 60-63, 65, 68, or 79, wherein the nucleotide sequence encoding the modulatory polynucleotide comprises:
(i) a 5′ flanking region comprising the nucleotide sequence of SEQ ID NO: 4354, a nucleotide sequence at least 70%, 75%, 80%, 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4977;
(iii) a loop region comprising the nucleotide sequence of SEQ ID NO: 4363, a nucleotide sequence at least 70%, 75%, 80%, 85%, 90%, 95% 96%, 97%, 98%, 99% identical to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4919; and
(v) a 3′ flanking region of SEQ ID NO: 4373, a nucleotide sequence at least 70%, 75%, 80%, 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.
   81. The modulatory polynucleotide of any one of embodiments 59, 61-63, 65, or 68 or the AAV viral genome of any one of embodiments 60-63, 65, or 68, wherein the modulatory polynucleotide comprises:
(i) a 5′ flanking region comprising the nucleotide sequence of SEQ ID NO: 4879, a nucleotide sequence at least 70%, 75%, 80%, 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4982;
(iii) a loop region comprising the nucleotide sequence of SEQ ID NO: 4888, a nucleotide sequence at least 70%, 75%, 80%, 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4924; and
(v) a 3′ flanking region of SEQ ID NO: 4898, a nucleotide sequence at least 70%, 75%, 80%, 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.
   82. The modulatory polynucleotide of any one of embodiments 59, 61-63, 65, 68, or 81 or the AAV viral genome of any one of embodiments 60-63, 65, 68, or 81, wherein the nucleotide sequence encoding the modulatory polynucleotide comprises:
(i) a 5′ flanking region comprising the nucleotide sequence of SEQ ID NO: 4354, a nucleotide sequence at least 70%, 75%, 80%, 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4981;
(iii) a loop region comprising the nucleotide sequence of SEQ ID NO: 4363, a nucleotide a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4923; and
(v) a 3′ flanking region of SEQ ID NO: 4373, a nucleotide sequence at least 70%, 75%, 80%, 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.
   83. The modulatory polynucleotide of any one of embodiments 59 or 61-82 or the AAV viral genome of any one of embodiments 62-82, wherein the nucleotide sequence encoding the modulatory polynucleotide comprises the nucleotide sequence of the nucleotide sequence of any one of SEQ ID NOs: 4380-4409 or 5027-5050, or a nucleotide sequence at least 70%, 75%, 80%, 85%, 90%, 92%, 95%, 96%, 97%, 98%, or 99% identical thereto.
   84. The modulatory polynucleotide of any one of embodiments 59, 61-63, 67-70, or 83, or the AAV viral genome of any one of embodiments 60-63, 67-70, or 83, wherein the nucleotide sequence encoding the modulatory polynucleotide comprises the nucleotide sequence of the nucleotide sequence of SEQ ID NO: 4405, or a nucleotide sequence at least 70%, 75%, 80%, 85%, 90%, 92%, 95%, 96%, 97%, 98%, or 99% identical thereto.
   85. The modulatory polynucleotide of any one of embodiments 59, 61-63, 67, 68, 71, 72, or 83, or the AAV viral genome of any one of embodiments 60-63, 67, 68, 71, 72, or 83, wherein the nucleotide sequence encoding the modulatory polynucleotide comprises the nucleotide sequence of SEQ ID NO: 4408, or a nucleotide sequence at least 70%, 75%, 80%, 85%, 90%, 92%, 95%, 96%, 97%, 98%, or 99% identical thereto.
   86. The modulatory polynucleotide of any one of embodiments 59, 61-63, 67, 68, 73, 74, or 83, or the AAV viral genome of any one of embodiments 60-63, 67, 68, 73, 74, or 83, wherein the nucleotide sequence encoding the modulatory polynucleotide comprises the nucleotide sequence of SEQ ID NO: 4409, or a nucleotide sequence at least 70%, 75%, 80%, 85%, 90%, 92%, 95%, 96%, 97%, 98%, or 99% identical thereto.
   87. The modulatory polynucleotide of any one of embodiments 59, 61-63, 65, 68, 75, 76, or 83, or the AAV viral genome of any one of embodiments 60-63, 65, 68, 75, 76, or 83, wherein the nucleotide sequence encoding the modulatory polynucleotide comprises the nucleotide sequence of SEQ ID NO: 4390, or a nucleotide sequence at least 70%, 75%, 80%, 85%, 90%, 92%, 95%, 96%, 97%, 98%, or 99% identical thereto.
   88. The modulatory polynucleotide of any one of embodiments 59, 61-63, 65, 68, 77, 78, or 83, or the AAV viral genome of any one of embodiments 60-63, 65, 68, 77, 78, or 83, wherein the nucleotide sequence encoding the modulatory polynucleotide comprises the nucleotide sequence of SEQ ID NO: 4391, or a nucleotide sequence at least 70%, 75%, 80%, 85%, 90%, 92%, 95%, 96%, 97%, 98%, or 99% identical thereto.
   89. The modulatory polynucleotide of any one of embodiments 59, 61-63, 65, 68, 79, 80, or 83, or the AAV viral genome of any one of embodiments 60-63, 65, 68, 79, 80, or 83, wherein the nucleotide sequence encoding the modulatory polynucleotide comprises the nucleotide sequence of SEQ ID NO: 4393, or a nucleotide sequence at least 70%, 75%, 80%, 85%, 90%, 92%, 95%, 96%, 97%, 98%, or 99% identical thereto.
   90. The modulatory polynucleotide of any one of embodiments 59, 61-63, 65, 68, 81, 82, or 83, or the AAV viral genome of any one of embodiments 60-63, 65, 68, 81, 82, or 83, wherein the nucleotide sequence encoding the modulatory polynucleotide comprises the nucleotide sequence of SEQ ID NO: 4394, or a nucleotide sequence at least 70%, 75%, 80%, 85%, 90%, 92%, 95%, 96%, 97%, 98%, or 99% identical thereto.
   91. An adeno-associated virus (AAV) viral genome comprising a promoter operably linked to a nucleic acid encoding the modulatory polynucleotide of any one of embodiments 59-90 or the siRNA of any one of embodiments 1-11 or 13-58.
   92. The AAV viral genome of any one of embodiments 12-58 or 60-90, wherein the AAV viral genome further comprises a promoter operably linked to the nucleotide sequence encoding the sense strand sequence and the antisense strand sequence or the modulatory polynucleotide.
   93. The AAV viral genome of embodiment 92, wherein the promoter is a ubiquitous promoter.
   94. The AAV viral genome of embodiment 92, wherein the promoter is a cell or tissue specific promoter.
   95. The AAV viral genome of any one of embodiments 92-94, wherein the promoter is chosen from human elongation factor 1α-subunit (EF1α), cytomegalovirus (CMV) immediate-early enhancer and/or promoter, chicken β-actin (CBA) and its derivative CAG, B glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light chain (NFL) or neurofilament heavy chain (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a functional fragment, e.g., a truncation, or a functional variant thereof.
   96. The AAV viral genome of any one of embodiments 92-95, wherein the promoter comprises a CBA promoter or a variant thereof.
   97. The AAV viral genome of any one of embodiments 92-95, wherein the promoter comprises the nucleotide sequence of any one of the nucleotide sequences provided in Table 2; a nucleotide sequence comprising at least one, two, three, four, five, six, or seven but no more than ten modifications, e.g., substitutions, insertions, or deletions, relative to any one of the nucleotide sequences provided in Table 2; or a nucleotide sequence with at least 80%, 85%, 90%, 92%, 95%, 96%, 97%, 98%, or 99% sequence identity to any one of the nucleotide sequences provided in Table 2.
   98. The AAV viral genome of any one of embodiments 92-97, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence with at least 80%, 85%, 90%, 92%, 95%, 96%, 97%, 98%, or 99% sequence identity to SEQ ID NO: 5199.
   99. The AAV viral genome of any one of embodiments 92-97, wherein the AAV viral genome further comprises a polyA signal sequence.
   100. The AAV viral genome of embodiment 99, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476.
   101. The AAV viral genome of any one of embodiments 26-32, wherein the AAV viral genome further comprises an inverted terminal repeat (ITR) sequence.
   102. The AAV viral genome of embodiment 101, wherein the ITR comprises the nucleotide sequence of SEQ ID NO: 5197, 5200, 4469, or 4470; a nucleotide sequence comprising at least one, two, three, four, five, six, or seven but no more than ten modifications, e.g., substitutions, insertions, or deletions, relative to SEQ ID NO: 5197, 5200, 4469, or 4470; or a nucleotide sequence with at least 80%, 85%, 90%, 92%, 95%, 96%, 97%, 98%, or 99% sequence identity to SEQ ID NO: 5197, 5200, 4469, or 4470.
   103. The AAV viral genome of any one of embodiments 12-58 or 60-102, wherein the AAV viral genome comprises an ITR sequence positioned 5′ relative to the nucleic acid encoding the modulatory polynucleotide or siRNA.
   104. The AAV viral genome of embodiment 103, wherein the ITR positioned 5′ relative to the nucleic acid encoding the modulator polynucleotide or siRNA comprises the nucleotide sequence of SEQ ID NO: 5197 or 4469; a nucleotide sequence comprising at least one, two, three, four, five, six, or seven but no more than ten modifications, e.g., substitutions, insertions, or deletions, relative to SEQ ID NO: 5197 or 4469; or a nucleotide sequence with at least 80%, 85%, 90%, 92%, 95%, 96%, 97%, 98%, or 99% sequence identity to SEQ ID NO: 5197 or 4469.
   105. The AAV viral genome of any one of embodiments 12-58 or 60-104, wherein the AAV viral genome comprises an ITR sequence positioned 3′ relative to the nucleic acid encoding the modulatory polynucleotide or siRNA.
   106. The AAV viral genome of embodiment 103, wherein the ITR positioned 3′ relative to the nucleic acid encoding the modulator polynucleotide or siRNA comprises the nucleotide sequence of SEQ ID NO: 5200 or 4470; a nucleotide sequence comprising at least one, two, three, four, five, six, or seven but no more than ten modifications, e.g., substitutions, insertions, or deletions, relative to SEQ ID NO: 5200 or 4470; or a nucleotide sequence with at least 80%, 85%, 90%, 92%, 95%, 96%, 97%, 98%, or 99% sequence identity to SEQ ID NO: 5200 or 4470.
   107. The AAV viral genome particle of any one of embodiments 12-58 or 60-106, wherein the AAV viral genome comprises an ITR sequence positioned 5′ relative to the nucleic acid encoding the modulatory polynucleotide or siRNA and an ITR sequence positioned 3′ relative to the nucleic acid encoding the modulatory polynucleotide or siRNA.
   108. The AAV viral genome of any one of embodiments 105-107, wherein:
(i) the ITR positioned 5′ relative to the nucleic acid encoding the modulatory polynucleotide or siRNA comprises the nucleotide sequence of SEQ ID NO: 5197; a nucleotide sequence at least 80%, 85%, 90%, 92% 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197; and
(ii) the ITR positioned 3′ relative to the nucleic acid encoding the modulatory polynucleotide or siRNA comprises the nucleotide sequence of SEQ ID NO: 5200; a nucleotide sequence at least 80%, 85%, 90%, 92% 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   109. The AAV viral genome of any one of embodiments 105-107, wherein:
(i) the ITR positioned 5′ relative to the nucleic acid encoding the modulatory polynucleotide or siRNA comprises the nucleotide sequence of SEQ ID NO: 4469; a nucleotide sequence at least 80%, 85%, 90%, 92%, 95%, 96%, 97%, 98%, or 99% identical to SEQ ID NO: 4469; or a nucleotide sequence comprising at least one, two, three, four, five, six, or seven but no more than ten modifications, e.g., substitutions, insertions, or deletions, relative to SEQ ID NO: 4469; and
(ii) the ITR positioned 3′ relative to the nucleic acid encoding the modulatory polynucleotide or siRNA comprises the nucleotide sequence of SEQ ID NO: 4470; a nucleotide sequence at least 80%, 85%, 90%, 92%, 95%, 96%, 97%, 98%, or 99% identical to SEQ ID NO: 4470; or a nucleotide sequence comprising at least one, two, three, four, five, six, or seven but no more than ten modifications, e.g., substitutions, insertions, or deletions, relative to SEQ ID NO: 4470.
   110. The AAV viral genome of any one of embodiments 12-58 or 60-109, wherein the AAV viral genome further comprises an enhancer, a Kozak sequence, an intron region, and/or an exon region.
   111. The AAV viral genome of embodiment 110, wherein the enhancer comprises:
(i) a CMVie enhancer or variant thereof; or
(ii) the nucleotide sequence of SEQ ID NO: 4471 or 4472; a nucleotide sequence comprising at least one, two, three, four, five, six, or seven but no more than ten modifications, e.g., substitutions, insertions, or deletions, relative to SEQ ID NO: 4471 or 4472; or a nucleotide sequence with at least 80%, 85%, 90%, 92%, 95%, 96%, 97%, 98%, or 99% sequence identity to SEQ ID NO: 4471 or 4472.
   112. The AAV viral genome of embodiment 110 or 111, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence with at least 80%, 85%, 90%, 92%, 95%, 96%, 97%, 98%, or 99% sequence identity to SEQ ID NO: 4471.
   113. The AAV viral genome of any one of embodiments 12-58 or 60-112, which comprises a CMVie enhancer or variant thereof; and a CBA promoter or variant thereof; optionally wherein the CMVie enhancer or variant thereof is present 5′ relative to the CBA promoter or variant thereof.
   114. The AAV viral genome of any one of embodiments 110-113, wherein the enhancer and promoter comprise the nucleotide sequence of SEQ ID NO: 4473 or 4474; a nucleotide sequence comprising at least one, two, three, four, five, six, or seven but no more than ten modifications, e.g., substitutions, insertions, or deletions, relative to SEQ ID NO: 4473 or 4474; or a nucleotide sequence with at least 80%, 85%, 90%, 92%, 95%, 96%, 97%, 98%, or 99% sequence identity to SEQ ID NO: 4473 or 4474.
   115. The AAV viral genome of any one of embodiments 110-114, wherein the intron comprises:
(i) a human beta globin intron (hBG intron) or variant thereof; or
(ii) the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence with at least 80%, 85%, 90%, 92%, 95%, 96%, 97%, 98%, or 99% sequence identity to SEQ ID NO: 4475.
   116. The AAV viral genome of any one of embodiments 12-58 or 60-115, wherein the AAV viral genome further comprises a nucleotide sequence encoding a miR binding site, e.g., a miR binding site that modulates, e.g., reduces expression of the payload encoded by the viral genome in a cell or tissue where the corresponding miRNA is expressed.
   117. The AAV viral genome of embodiment 116, wherein the encoded miR binding site modulates, e.g., reduces expression of the encoded payload in a cell or tissue of the DRG, liver, heart, hematopoietic lineage, or a combination thereof.
   118. The AAV viral genome of any one of embodiments 12-58 or 60-117, wherein the AAV viral genome comprises at least 1-5 copies of the encoded miR binding site, e.g., at least 1, 2, 3, 4, or 5 copies, optionally wherein the at least 1-5 copies are continuous (e.g., not separated by a spacer) or the at least 1-5 copies are separated by a spacer.
   119. The AAV viral genome of any one of embodiments 12-58 or 60-118, wherein the AAV viral genome is self-complementary.
   120. The AAV viral genome of any one of embodiments 12-58 or 60-118, wherein the AAV viral genome is single stranded.
   121. The AAV viral genome of any one of embodiments 12-58 or 60-120, wherein the AAV viral genome further comprises a nucleotide sequence encoding a Rep protein, e.g., a non-structural protein, wherein the Rep protein comprises a Rep78 protein, a Rep68, Rep52 protein, and/or a Rep40 protein (e.g., a Rep78 and a Rep52 protein).
   122. The AAV viral genome of any one of embodiments 12-58 or 60-121, wherein the AAV viral genome comprises in 5′ to 3′ order:
(i) an ITR;
(ii) an enhancer (e.g., a CMVie enhancer or variant thereof);
(iii) a promoter (e.g., a CBA promoter or variant thereof);
(iv) an intron (e.g., an hBG intron or variant thereof);
(v) a nucleic acid sequence encoding a modulatory polynucleotide (e.g. the modulatory polynucleotide of any one of embodiments 59-90);
(vi) a polyA signal sequence; and
(vii) an ITR.
   123. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises any one of SEQ ID NOs: 4380-4409 or 5027-5050, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   124. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises any one of SEQ ID NOs: 4405, 4408, 4409, 4390, 4391, 4393, or 4394, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   125. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4405, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   126. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4408, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   127. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4409, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   128. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4390, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   129. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4391, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   130. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4393, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   131. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4394, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.
   132. The AAV viral genome of any one of embodiments 12-58 or 60-122, wherein the AAV viral genome comprises in 5′ to 3′ order:
(i) an ITR, wherein the ITR comprises the nucleotide sequence of SEQ ID NO: 4469; a nucleotide sequence at least 80%, 85%, 90%, 92%, 95%, 96%, 97%, 98%, or 99% identical to SEQ ID NO: 4469; or a nucleotide sequence comprising at least one, two, three, four, five, six, or seven but no more than ten modifications, e.g., substitutions, insertions, or deletions, relative to SEQ ID NO: 4469;
(ii) an enhancer, wherein the enhancer comprises the nucleotide sequence of SEQ ID NO: 4472; a nucleotide sequence comprising at least one, two, three, four, five, six, or seven but no more than ten modifications, e.g., substitutions, insertions, or deletions, relative to SEQ ID NO: 4472; or a nucleotide sequence at least 80%, 85%, 90%, 92%, 95%, 96%, 97%, 98%, or 99% identical to SEQ ID NO: 4472;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises any one of SEQ ID NOs: 4380-4409 or 5027-5050, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 4470; a nucleotide sequence at least 80%, 85%, 90%, 92%, 95%, 96%, 97%, 98%, or 99% identical to SEQ ID NO: 4470; or a nucleotide sequence comprising at least one, two, three, four, five, six, or seven but no more than ten modifications, e.g., substitutions, insertions, or deletions, relative to SEQ ID NO: 4470.
   133. The AAV viral genome of any one of embodiments 12-58 or 60-132, which comprises the nucleotide sequence of any one of SEQ ID NOs: 4439-4468 or 5149-5196, or a nucleotide sequence at least 70%, 75%, 80%, 85%, 90%, 92%, 95%, 96%, 97%, 98%, or 99% identical to any one of SEQ ID NOs: 4439-4468 or 5149-5196.
   134. The AAV viral genome of any one of embodiments 12-58, 60-96, 98-108, or 110-133, which comprises the nucleotide sequence of any one of SEQ ID NOs: 5149-5196, or a nucleotide sequence at least 70%, 75%, 80%, 85%, 90%, 92%, 95%, 96%, 97%, 98%, or 99% identical to any one of SEQ ID NOs: 5149-5196.
   135. The AAV viral genome of any one of embodiments 12-58, 60-96, 98-108, 110-133, or 134, which comprises the nucleotide sequence of any one of SEQ ID NOs: 5173, 5195, 5196, 5182, 5183, 5184, or 5185, or a nucleotide sequence at least 70%, 75%, 80%, 85%, 90%, 92%, 95%, 96%, 97%, 98%, or 99% identical to any one of SEQ ID NOs: 5173, 5195, 5196, 5182, 5183, 5184, or 5185.
   136. The AAV viral genome of any one of embodiments 12-58, 60-96, 98-108, 110-125, or 133-135, which comprises the nucleotide sequence of SEQ ID NO: 5173, or a nucleotide sequence at least 70%, 75%, 80%, 85%, 90%, 92%, 95%, 96%, 97%, 98%, or 99% identical to SEQ ID NO: 5173.
   137. The AAV viral genome of any one of embodiments 12-58, 60-96, 98-108, 110-124, 126, or 133-135, which comprises the nucleotide sequence of SEQ ID NO: 5195, or a nucleotide sequence at least 70%, 75%, 80%, 85%, 90%, 92%, 95%, 96%, 97%, 98%, or 99% identical to SEQ ID NO: 5195.
   138. The AAV viral genome of any one of embodiments 12-58, 60-96, 98-108, 110-124, 127, or 133-135, which comprises the nucleotide sequence of SEQ ID NO: 5196, or a nucleotide sequence at least 70%, 75%, 80%, 85%, 90%, 92%, 95%, 96%, 97%, 98%, or 99% identical to SEQ ID NO: 5196.
   139. The AAV viral genome of any one of embodiments 12-58, 60-96, 98-108, 110-124, 128, or 133-135, which comprises the nucleotide sequence of SEQ ID NO: 5182, or a nucleotide sequence at least 70%, 75%, 80%, 85%, 90%, 92%, 95%, 96%, 97%, 98%, or 99% identical to SEQ ID NO: 5182.
   140. The AAV viral genome of any one of embodiments 12-58, 60-96, 98-108, 110-124, 129, or 133-135, which comprises the nucleotide sequence of SEQ ID NO: 5183, or a nucleotide sequence at least 70%, 75%, 80%, 85%, 90%, 92%, 95%, 96%, 97%, 98%, or 99% identical to SEQ ID NO: 5183.
   141. The AAV viral genome of any one of embodiments 12-58, 60-96, 98-108, 110-124, 130, or 133-135, which comprises the nucleotide sequence of SEQ ID NO: 5184, or a nucleotide sequence at least 70%, 75%, 80%, 85%, 90%, 92%, 95%, 96%, 97%, 98%, or 99% identical to SEQ ID NO: 5184.
   142. The AAV viral genome of any one of embodiments 12-58, 60-96, 98-108, 110-124, 131, or 133-135, which comprises the nucleotide sequence of SEQ ID NO: 5185, or a nucleotide sequence at least 70%, 75%, 80%, 85%, 90%, 92%, 95%, 96%, 97%, 98%, or 99% identical to SEQ ID NO: 5185.
   143. The AAV viral genome of any one of embodiments 12-58, 60-96, 98-107, 109-122, or 133, which comprises the nucleotide sequence of any one of SEQ ID NOs: 4439-4468, or a nucleotide sequence at least 70%, 75%, 80%, 85%, 90%, 92%, 95%, 96%, 97%, 98%, or 99% identical to any one of SEQ ID NOs: 4439-4468.
   144. An AAV particle comprising the AAV viral genome of any one of embodiments 12-58 or 60-143, an AAV viral genome encoding the modulatory polynucleotide of any one of embodiments 59-90, or an AAV viral genome encoding the siRNA of any one of embodiments 11 or 13-58, and an AAV capsid protein.
   145. The AAV particle of embodiment 48, wherein the AAV capsid protein comprises an AAV5 capsid protein, an AAV9 capsid protein, VOY101 capsid protein, a PHPeB capsid protein, a PHP.B capsid protein, a PHP.N capsid protein, an AAV1 capsid protein, a VOY9P39 capsid protein, an AAV2 capsid protein, an AAVrh 10 capsid protein, or a variant thereof (e.g., an AAV9 capsid protein or a variant thereof or an AAV5 capsid protein or variant thereof).
   146. The AAV particle of embodiment 144 or 145, wherein the AAV capsid protein comprises an AAV9 capsid or variant thereof.
   147. The AAV particle of embodiment 144 or 145, wherein the AAV capsid protein comprises an AAV5 capsid or variant thereof.
   148. The AAV particle of any one of embodiments 144-147, wherein:
(i) the AAV capsid protein comprises an amino acid sequence provided in Table 1 or an amino acid sequence at least 70%, 75%, 80%, 85%, 90%, 92%, 95%, 97%, 98%, or 99% identical thereto; or
(ii) the nucleotide sequence encoding the AAV capsid protein comprises any one of the nucleotide sequences in Table 1, or a nucleotide sequence at least 70%, 75%, 80%, 85%, 90%, 92%, 95%, 97%, 98%, or 99% identical thereto.
   149. A vector comprising the AAV viral genome of any one of embodiments 12-58 or 60-143, a nucleotide sequence encoding the modulatory polynucleotide of any one of embodiments 59-90, or a nucleotide sequence encoding the siRNA of any one of embodiments 11 or 13-58.
   150. A cell comprising the AAV particle of any one of embodiments 144-148, the AAV viral genome of any one of embodiments 12-58 or 60-143, the modulatory polynucleotide of any one of embodiments 59-90, or the siRNA of any one of embodiments 11 or 13-58.
   151. The cell of embodiment 150, wherein the cell is a mammalian cell or an insect cell.
   152. The cell of embodiment 150 or 151, wherein the cell is a cell of a brain region or a spinal cord region (e.g., a CNS cell).
   153. The cell of any one of embodiments 150-152, wherein the cell is a cell of the cortex, hippocampus, thalamus, or brainstem.
   154. The cell of any one of embodiments 150-153, wherein the cell is a neuron, a glial cell, or an oligodendrocyte.
   155. A method of making an AAV particle, comprising:
(i) providing a host cell comprising the AAV viral genome of any one of embodiments 12-58 or 60-143, an AAV viral genome encoding the modulatory polynucleotide of any one of embodiments 59-90, or a viral genome encoding the siRNA of any one of embodiments 11 or 13-58;
(ii) incubating the host cell under conditions suitable to enclose the AAV viral genome in an AAV capsid protein;
thereby making the AAV particle.
   156. The method of embodiment 155, further comprising, prior to step (i), introducing a first nucleic acid molecule comprising the viral genome into the host cell.
   157. The method of embodiment 155 or 156, wherein the host cell comprises a second nucleic acid encoding the AAV capsid protein.
   158. The method of embodiment 157, wherein the second nucleic acid molecule is introduced into the host cell prior to, concurrently with, or after the first nucleic acid molecule.
   159. A pharmaceutical composition comprising the AAV particle of any one of embodiments 144-148, an AAV particle comprising the AAV viral genome of any one of embodiments 12-58 or 60-143, the modulatory polynucleotide of any one of embodiments 59-90, or the siRNA of any one of embodiments 11 or 13-58, and a pharmaceutically acceptable excipient.
   160. A method of delivering an siRNA for inhibiting MAPT expression (e.g., MAPT gene, mRNA, or protein expression) to a cell, comprising administering an effective amount of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, thereby delivering the siRNA for inhibiting MAPT expression to the cell.
   161. The method of embodiment 160, wherein the cell is a cell of a brain region or a spinal cord region.
   162. The method of embodiment 160 or 161, wherein the cell is a cell of the cortex, hippocampus, thalamus, or brainstem.
   163. The method of any one of embodiments 160-162, wherein the cell is a neuron, an astrocyte, a glial cell, or an oligodendrocyte.
   164. The method of any one of embodiments 160-163, wherein delivery decreases (e.g., inhibits) MAPT mRNA levels, e.g., by at least 40%, 44%, 45%, 50%, 55%, 60%, 64%, 65%, 70%, 75%, 76%, 79%, 80%, 84%, 85%, 87%, or 90%, e.g., in a cell of a brain region (e.g., cortex, hippocampus, or brain stem), e.g., as compared to a reference level (e.g., the mRNA levels in a cell that has not been delivered or prior to delivering the AAV particle, modulatory polynucleotide, siRNA, or pharmaceutical composition), e.g., when measured by an assay (e.g., an assay as described in Example 6).
   165. The method of any one of embodiments 160-164, wherein delivery decreases (e.g., inhibits) MAPT protein levels, e.g., by at least 15%, 17%, 20%, 25%, 30%, 35%, 37%, 40%, 44%, 45%, 46%, 50%, 53%, 55%, 60%, 63%, 64%, 65%, 70%, 72%, 74%, or 75%, e.g., in a cell of a brain region (e.g., cortex, hippocampus, thalamus, or brain stem), e.g., as compared to a reference level (e.g., the protein levels in a cells that has not been delivered or prior to delivering the AAV particle, modulatory polynucleotide, siRNA, or pharmaceutical composition), e.g., when measured by an assay (e.g., an assay as described in Example 6).
   166. The method of any one of embodiments 160-165, wherein the cell is in a subject.
   167. The method of embodiment 166, wherein the subject has or has been diagnosed with having a genetic disorder (e.g., a monogenic disorder or a polygenic disorder).
   168. The method of embodiment 166 or 167, wherein the subject has or has been diagnosed with having a neurological disorder (e.g., a neurodegenerative disorder).
   169. The method of any one of embodiments 166-168, wherein the subject has or has been diagnosed with having a disorder associated with tau expression, e.g., aberrant tau expression.
   170. The method of any one of embodiments 166-169, wherein the subject has or has been diagnosed with having a tauopathy.
   171. A method of treating a subject having or diagnosed with having a genetic disorder, e.g., a monogenic disorder or a polygenic disorder, comprising administering to the subject an effective amount of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, thereby treating the subject having or diagnosed with having the genetic disorder, e.g., the monogenic disorder or the polygenic disorder.
   172. A method of treating a subject having or diagnosed with having a neurological disorder (e.g., a neurodegenerative disorder), comprising administering to the subject an effective amount of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, thereby treating the subject having or diagnosed with having the neurological disorder (e.g., the neurodegenerative disorder).
   173. A method of treating a subject having or diagnosed with having a disorder associated with tau expression, e.g., aberrant tau expression, comprising administering to the subject an effective amount of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, thereby treating the subject having or diagnosed with having the disorder associate with tau expression, e.g., aberrant tau expression.
   174. A method of treating a subject having or diagnosed with having a tauopathy, comprising administering to the subject an effective amount of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, thereby treating the subject having or diagnosed with having the tauopathy.
   175. The method of any one of embodiments 167-174, wherein the genetic disorder, neurological disorder (e.g., neurodegenerative disorder), disorder associated with tau expression (e.g., aberrant tau expression), and/or tauopathy is Alzheimer's disease (AD), Frontotemporal dementia (FTD), frontotemporal dementia and parkinsonism linked to chromosome 17 (FTDP-17), frontotemporal lobar degeneration (FTLD), Dravet syndrome (DS), neurodegenerative disease, traumatic brain injury (TBI), chronic traumatic encephalopathy (CTE), progressive supranuclear palsy (PSP), Down's syndrome, Pick's disease, corticobasal degeneration (CBD), corticobasal syndrome, amyotrophic lateral sclerosis (ALS), Prion diseases, CJD, Multiple system atrophy, mild cognitive impairment, Tangle-only dementia, or Progressive subcortical gliosis.
   176. The method of any one of embodiments 171-175, wherein treating comprises prevention of progression of the genetic disorder, neurological disorder (e.g., neurodegenerative disorder), disorder associated with tau expression (e.g., aberrant tau expression), and/or tauopathy in the subject.
   177. The method of any one of embodiments 171-176, further comprising performing a blood test, an imaging test (e.g., a PET scan or a PET scan in combination with biomarker, e.g., serum biomarker staining), a CNS biopsy sample, or an aqueous cerebral spinal fluid biopsy, optionally wherein the blood test, imaging test, biopsy sample, or aqueous cerebral spinal fluid biopsy is performed prior to, during, or after treatment with the AAV particle, modulatory polynucleotide, siRNA, or pharmaceutical composition.
   178. The method of any one of embodiments 160-177, wherein administration decreases, e.g., reduces or inhibits, expression of a MAPT gene, mRNA, and/or protein.
   179. The method of embodiments 178, wherein the MAPT gene, mRNA, and/or protein is a wild-type MAPT gene, mRNA, or protein.
   180. The method of embodiment 178 or 179, wherein the MAPT gene, mRNA, and/or protein is a mutated MAPT gene, mRNA, or protein (e.g., comprises at least 1 mutation).
   181. The method of any one of embodiments 171-180, wherein administration of the AAV particle, modulatory polynucleotide, siRNA, or pharmaceutical composition results in a reduction and/or prevention of tau pathology, e.g., a decrease in a biomarker of tau pathology (e.g., ¹⁸F-flortaucipir, plasma ptau 181, or ¹⁸F-PM-PBB3), e.g., as measured by a PET scan or PET scan in combination with Braak neuropathological staging and/or serum biomarker staining, e.g., as compared to a reference (e.g., a subject that as not received the AAV particle, modulatory polynucleotide, siRNA, or pharmaceutical composition).
   182. The method of any one of embodiments 171-181, wherein the administration decreases MAPT mRNA levels in the subject, e.g., by at least 40%, 44%, 45%, 50%, 55%, 60%, 64%, 65%, 70%, 75%, 76%, 79%, 80%, 84%, 85%, 87%, or 90%, e.g., in a cell of a brain region (e.g., cortex, hippocampus, or brain stem), e.g., as compared to a reference level (e.g., the mRNA levels in a subject that has not received or prior to receiving the AAV particle, modulatory polynucleotide, siRNA, or pharmaceutical composition), e.g., when measured by an assay (e.g., an assay as described in Example 6).
   183. The method of any one of embodiments 171-182, wherein the administration decreases MAPT protein levels in the subject, e.g., by at least 15%, 17%, 20%, 25%, 30%, 35%, 37%, 40%, 44%, 45%, 46%, 50%, 53%, 55%, 60%, 63%, 64%, 65%, 70%, 72%, 74%, or 75%, e.g., in a cell of a brain region (e.g., cortex, hippocampus, thalamus, or brain stem), e.g., as compared to a reference level (e.g., the protein levels in a subject that has not received or prior to receiving the AAV particle, modulatory polynucleotide, siRNA, or pharmaceutical composition), e.g., when measured by an assay (e.g., an assay as described in Example 6).
   184. The method of any one of embodiments 166-183, wherein the subject is a human.
   185. The method of any one of embodiments 166-184, wherein the pharmaceutical composition, AAV particle, modulatory polynucleotide, or siRNA are administered to the subject intravenously, intramuscularly, via intraparenchymal administration, intracerebroventricularly, via intra-cisterna magna (ICM) injection, intrathecally, via focused ultrasound (FUS), e.g., coupled with the intravenous administration of microbubbles (FUS-MB), or MRI-guided FUS coupled with intravenous administration.
   186. The method of any one of embodiments 166-185, wherein the pharmaceutical composition, AAV particle, modulatory polynucleotide, or siRNA are administered to the subject intravenously.
   187. The method of any one of embodiments 166-186, wherein the pharmaceutical composition, AAV particle, modulatory polynucleotide, or siRNA are administered to the subject via intra-cisterna magna injection (ICM).
   188. The method of any one of embodiments 166-187, wherein the pharmaceutical composition, AAV particle, modulatory polynucleotide, or siRNA are administered to the subject intrathecally. 189. The pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58 for use in a method of delivering an siRNA for inhibiting MAPT expression to a cell.
   190. The pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, for use in the manufacture of a medicament.
   191. The pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, for use in the treatment of a genetic disorder, neurological disorder (e.g., neurodegenerative disorder), disorder associated with tau expression (e.g., aberrant tau expression), and/or tauopathy.
   192. Use of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, in the manufacture of a medicament for delivering an siRNA for inhibiting MAPT expression to a cell.
   193. Use of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, in the manufacture of a medicament.
   194. Use of the pharmaceutical composition of embodiment 159, the AAV particle of any one of embodiments 144-159, an AAV particle comprising the AAV viral genome of any one of embodiments 12-58 or 60-143, the modulatory polynucleotide of any one of embodiments 59-90, or the siRNA of any one of embodiments 11 or 13-58, in the manufacture of a medicament for treating a genetic disorder, neurological disorder (e.g., neurodegenerative disorder), disorder associated with tau expression (e.g., aberrant tau expression), and/or tauopathy.

### BRIEF DESCRIPTION OF THE DRAWINGS

FIG. 1 is a graph depicting the relative R/F activity as % over non-target control (NTC) on the Y-axis post-transfection with the modulatory polynucleotide constructs targeting MAPT indicated on the X-axis (from left to right: VOYTaumiR-102-530 (SEQ ID NO: 4380), VOYTaumiR-102-556 (SEQ ID NO: 4381), VOYTaumiR-102-579 (SEQ ID NO: 4383), VOYTaumiR-102-586 (SEQ ID NO: 4384), VOYTaumiR-104-530 (SEQ ID NO: 4385), VOYTaumiR-104-556 (SEQ ID NO: 4386), VOYTaumiR-104-579 (SEQ ID NO: 4388), VOYTaumiR-104-586 (SEQ ID NO: 4389), VOYTaumiR-109-530 (SEQ ID NO: 4390), VOYTaumiR-109-556 (SEQ ID NO: 4391), VOYTaumiR-109-579 (SEQ ID NO: 4393), VOYTaumiR-109-586 (SEQ ID NO: 4394), VOYTaumiR-114-530 (SEQ ID NO: 4395), VOYTaumiR-114-556 (SEQ ID NO: 4396), VOYTaumiR-114-579 (SEQ ID NO: 4398), VOYTaumiR-114-586 (SEQ ID NO: 4399), VOYTaumiR-116-530 (SEQ ID NO: 4400), VOYTaumiR-116-556 (SEQ ID NO: 4401), VOYTaumiR-116-579 (SEQ ID NO: 4403), VOYTaumiR-116-586 (SEQ ID NO: 4404), VOYTaumiR-127-530 (SEQ ID NO: 4405), VOYTaumiR-127-556 (SEQ ID NO: 4406), VOYTaumiR-127-579 (SEQ ID NO: 4408), and VOYTaumiR-127-586 (SEQ ID NO: 4409)).

FIG. 2 is a graph showing the fold-change in MAPT mRNA remaining for each vectorized modulatory polynucleotide relative to the non-target control (fold change relative to NTC) on the Y-axis post-transfection with the modulatory polynucleotide constructs targeting MAPT indicated on the X-axis (from left to right: mock, non-target control (NTC), VOYTaumiR-127-530 (SEQ ID NO: 4405), VOYTaumiR-127-556 (SEQ ID NO: 4406), VOYTaumiR-127-579 (SEQ ID NO: 4408), VOYTaumiR-127-586 (SEQ ID NO: 4409), VOYTaumiR-116-530 (SEQ ID NO: 4400), VOYTaumiR-116-556 (SEQ ID NO: 4401), VOYTaumiR-116-579 (SEQ ID NO: 4403), VOYTaumiR-116-586 (SEQ ID NO: 4404), VOYTaumiR-114-530 (SEQ ID NO: 4395), VOYTaumiR-114-556 (SEQ ID NO: 4396), VOYTaumiR-114-579 (SEQ ID NO: 4398), VOYTaumiR-114-586 (SEQ ID NO: 4399), VOYTaumiR-109-530 (SEQ ID NO: 4390), VOYTaumiR-109-556 (SEQ ID NO: 4391), VOYTaumiR-109-579 (SEQ ID NO: 4393), VOYTaumiR-109-586 (SEQ ID NO: 4394), VOYTaumiR-104-530 (SEQ ID NO: 4385), VOYTaumiR-104-556 (SEQ ID NO: 4386), VOYTaumiR-104-579 (SEQ ID NO: 4388), VOYTaumiR-104-586 (SEQ ID NO: 4389), VOYTaumiR-102-530 (SEQ ID NO: 4380), VOYTaumiR-102-556 (SEQ ID NO: 4381), VOYTaumiR-102-579 (SEQ ID NO: 4383), and VOYTaumiR-102-586 (SEQ ID NO: 4384)).

FIG. 3 is a graph showing the fold-change in MAPT mRNA remaining for each vectorized modulatory polynucleotide relative to the non-target control (fold change relative to NTC) on the Y-axis post-transfection with increasing concentrations (0.05 μg, 0.16 μg, 0.50 μg, or 1.50 μg per well) of the modulatory polynucleotides on the X-axis (from left to right: mock, non-target control (NTC), VOYTaumiR-127-530 (SEQ ID NO: 4405), VOYTaumiR-127-556 (SEQ ID NO: 4406), VOYTaumiR-127-579 (SEQ ID NO: 4408), VOYTaumiR-127-586 (SEQ ID NO: 4409), VOYTaumiR-109-530 (SEQ ID NO: 4390), VOYTaumiR-109-556 (SEQ ID NO: 4391), VOYTaumiR-109-579 (SEQ ID NO: 4393), VOYTaumiR-109-586 (SEQ ID NO: 4394), VOYTaumiR-116-579 (SEQ ID NO: 4403), VOYTaumiR-104-556 (SEQ ID NO: 4386), and VOYTaumiR-104-586 (SEQ ID NO: 4389)).

FIG. 4 is a graph depicting the fold-change in MAPT mRNA remaining for each vectorized modulatory polynucleotide relative to the non-target control (fold change relative to NTC) on the Y-axis post-transfection with the indicated concentrations (0.5 μg or 1.0 μg) of the modulatory polynucleotides on the X-axis (from left to right: mock, GFP, NTC, MAPT antisense oligonucleotide (ASO) positive control, VOYTaumiR-127-530 (SEQ ID NO: 4405), VOYTaumiR-109-530 (SEQ ID NO: 4390), VOYTaumiR-102-582 (SEQ ID NO: 5027), VOYTaumiR-104-582 (SEQ ID NO: 5031), VOYTaumiR-109-582 (SEQ ID NO: 5035), VOYTaumiR-114-582 (SEQ ID NO: 5039), VOYTaumiR-127-582 (SEQ ID NO: 5047), VOYTaumiR-102-587 (SEQ ID NO: 5028), VOYTaumiR-109-587 (SEQ ID NO: 5036), VOYTaumiR-114-587 (SEQ ID NO: 5040), VOYTaumiR-104-552 (SEQ ID NO: 5033), VOYTaumiR-104-549 (SEQ ID NO: 5034), and VOYTaumiR-116-549 (SEQ ID NO: 5046).

FIGS. 5A-5E are a series of graphs depicting the vector genome copies per diploid cells in the cortex (FIG. 5A), hippocampus (FIG. 5B), brainstem (FIG. 5C), thalamus (FIG. 5D), or liver (FIG. 5E) of mice following intravenous injection of the vehicle control or AAV particles comprising the VOY9P39 capsid encoding the VOYTaumiR-127-530 modulatory polynucleotide (VOY9P39_127-530) at a dose of 3e12 vg/kg, 1e13 vg/kg, or 3e13 vg/kg. Statistical significance was evaluated with a one-way ANOVA with Tukey's multiple comparisons post-hoc test: *, **, ***, and **** indicate p<0.05, 0.005, 0.0005 and 0.0001, respectively. Data are shown as the group mean±SEM. The numbers above the bars indicate the group mean in vg/dg (biodistribution).

FIGS. 6A-6D are a series of graphs depicting the fold-change in MAPT mRNA levels remaining post-treatment in each treatment group relative to the vehicle on the Y-axis, in the cortex (FIG. 6A), hippocampus (FIG. 6B), thalamus (FIG. 6C), or brainstem (FIG. 6D) of mice following intravenous injection of AAV particles comprising the payload and capsid combinations indicated on the X-axis, which are from left to right: vehicle control, VOY9P39 capsid and mCherry reporter control at 3e13 vg/kg, VOY9P39 capsid and non-target control (NTC) at 3e13 vg/kg, VOY9P39 capsid and VOYTaumiR-127-530 modulatory polynucleotide (127-530) at 3e12 vg/kg, VOY9P39 capsid and VOYTaumiR-127-530 modulatory polynucleotide (127-530) at 1e13 vg/kg, VOY9P39 capsid and VOYTaumiR-127-530 modulatory polynucleotide (127-530) at 3e13 vg/kg, VOY101 capsid and mCherry reporter control at 3e13 vg/kg, and VOY101 capsid and VOYTaumiR-127-530 modulatory polynucleotide (127-530) at 3e13 vg/kg. Statistical significance was evaluated with a one-way ANOVA with Tukey's multiple comparison with p-value as shown on the graphs.

FIGS. 7A-7D are a series of graphs depicting the MAPT protein levels remaining post-treatment in each treatment group relative to the vehicle in the cortex (FIG. 7A), hippocampus (FIG. 7B), thalamus (FIG. 7C), or brainstem (FIG. 7D) of mice following intravenous injection of the vehicle control (vehicle); or AAV particles comprising the VOY9P39 capsid encoding VOYTaumiR-127-530 modulatory polynucleotide (127-530) administered at a dose of 3e12 vg/kg, 1e13 vg/kg, or 3e13 vg/kg. The percentages above the bars represent the percent knockdown of tau protein following treatment. Statistical significance was evaluated with a one-way ANOVA with Tukey's multiple comparison.

FIGS. 8A-8D are a series of graphs depicting the MAPT mRNA levels remaining post-treatment in each treatment group relative to the vehicle in the cortex (FIG. 8A), hippocampus (FIG. 8B), thalamus (FIG. 8C), or brainstem (FIG. 8D) of mice following intravenous injection of the vehicle control (vehicle); AAV particles comprising the VOY9P39 capsid encoding the non-target control (NTC) at dose of 1e13 vg/kg (C9P39-NTC_1E13); AAV particles comprising the VOY9P39 capsid encoding the VOYTaumiR-127-530 modulatory polynucleotide at a dose of 1e12 vg/kg (C9P39-127.530_1E12) or 1e13 vg/kg (C9P39-127.530_1E13); or AAV particles comprising the VOY9P39 capsid encoding the VOYTaumiR-127-579 modulatory polynucleotide at a dose of 1e13 vg/kg (C9P39-127.579_1E13). Statistical significance was evaluated with a one-way ANOVA with Tukey's multiple comparison with p-value as shown on the graphs.

### DETAILED DESCRIPTION

Described herein, inter alia, are compositions comprising isolated, e.g., recombinant, viral particles, e.g., AAV particles, for delivery, e.g., vectorized delivery, of an RNA agent for targeting MAPT (e.g., a siRNA duplex for targeting MAPT) and methods of making and using the same. In some embodiments, the RNA agent for targeting MAPT is siRNA duplex that modulates, e.g., inhibits MAPT expression, e.g., a siRNA duplex for targeting MAPT as described herein. Generally, the recombinant AAV particles will include a viral genome comprising a nucleotide sequence, e.g., encoding a transgene encoding an RNA agent for targeting MAPT (e.g., a siRNA duplex for targeting MAPT) and an AAV capsid protein, e.g., an AAV capsid variant described herein.

Mutations in the MAPT gene were identified in the autosomal dominantly inherited tauopathy frontotemporal dementia and parkinsonism linked to chromosome 17 (FTDP-17), which demonstrates that mutations in the MAPT gene and/or tau protein could lead to neurodegenerative changes in the brain. Mutant tau is considered more amyloidogenic than wild-type tau, meaning it is more likely to become hyperphosphorylated and to aggregate as NFT (Hutton, M. et al., 1998, Nature 393(6686):702-5), and these NFT can be composed of pathological forms of tau including, but not limited to, hyperphosphorylated, misfolded and aggregated tau species, each of which has yielded a set of avidly pursued targets. Hyperphosphorylation of tau depresses its binding to microtubules and microtubule assembly/stability activity. Further, hyperphosphorylation of tau renders it prone to misfolding and aggregation. Without wishing to be bound by theory, it is believed in some embodiments that administration of an siRNA targeting MAPT (e.g., a mutated MAPT gene or a wild-type MAPT gene), e.g., a siRNA for targeting MAPT described herein, and vectorized forms thereof, can reduce tau, e.g., pathological tau, protein expression, aggregation, hyperphosphorylation, and/or misfolding, thereby modulating, e.g., reducing or slowing, pathology associated with aberrant tau expression and/or tauopathies (e.g., Alzheimer's disease (AD), frontotemporal dementia (FTD), and/or Dravet syndrome (DS)).

Without wishing to be bound by theory, it is believed in some embodiments, that the use of an AAV particle or plurality of AAV particles for the vectorized delivery of an RNA agent for targeting MAPT (e.g., a siRNA duplex for targeting MAPT) described herein would lead to increased exposure in the central nervous system (CNS), and robust expression of the RNA agent targeting MAPT in the subject, e.g., a subject having or diagnosed with having a disease associated with expression of tau or a neurological disorder described herein (e.g., a tauopathy).

### I. COMPOSITIONS

According to the present disclosure, compositions for delivering a functional RNA agent for targeting MAPT by adeno-associated virus particles (AAVs), e.g., an AAV particle comprising an AAV capsid polypeptide are provided. In some embodiments, an AAV particle, e.g., an AAV particle comprising a an AAV capsid polypeptide, as described herein, or plurality of particles, may be provided, e.g., delivered, via any of several routes of administration (e.g., via intravenous administration), to a cell, tissue, organ, or organism, in vivo, ex vivo, or in vitro

### Adeno-Associated Viruses (AAVs) and AAV Particles

Described herein, inter alia, are compositions comprising isolated, e.g., recombinant, viral particles, e.g., AAV particles, for delivery, e.g., vectorized delivery, of a polynucleotide, e.g., a MAPT targeting siRNA, and methods of making and using the same.

In some embodiments, an adeno-associated virus (AAV) comprises a small non-enveloped icosahedral capsid virus of the Parvoviridae family and is characterized by a single stranded DNA viral genome. The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety. In some embodiments, AAV is capable of replication in vertebrate hosts including, but not limited to, human, primate, bovine, canine, equine, and ovine species

In some embodiments, an AAV particle is used as a biological tool due to a relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload, e.g., a polynucleotide encoding a siRNA for targeting MAPT.

In some embodiments, the AAV particle is a naturally occurring (e.g., wild-type) AAV or a recombinant AAV. In some embodiments, the wild-type AAV viral genome is a linear, single-stranded DNA (ssDNA) molecule approximately 5,000 nucleotides (nt) in length. In some embodiments, inverted terminal repeats (ITRs) cap the viral genome at both the 5′ and the 3′ end, providing origins of replication for the viral genome. In some embodiments, an AAV viral genome typically comprises two ITR sequences. These ITRs have a characteristic T-shaped hairpin structure defined by a self-complementary region (145nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In some embodiments, the AAV particle, e.g., an AAV particle (e.g., ssAAVs) described herein comprises a viral genome that is self-complementary (scAAV). In some embodiments, the ssAAV comprises nucleic acid molecules, e.g., DNA strands, that anneal together to form double stranded DNA. In some embodiments, a scAAV allows for rapid expression in a transduced cell as it bypasses second strand synthesis.

In some embodiments, the wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are used for replication and packaging, while the capsid proteins are assembled to create the protein shell of the AAV, or AAV capsid polypeptide, e.g., an AAV capsid variant. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9 (SEQ ID NO: 138) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the “AAV capsid” protein. While not wishing to be bound by theory, the AAV capsid protein typically comprises a molar ratio of 1:1:10 of VP1:VP2:VP3. In some embodiments, a viral genome of a wild-type, e.g., naturally occurring, AAV can be modified to replace the rep/cap sequences with a nucleic acid comprising a transgene encoding a payload, e.g., an antibody molecule, wherein the genetic element comprises at least one ITR region. In some embodiments, the viral genome of a recombinant AAV comprises two ITR regions, e.g., a 5′ITR or a 3′ITR. In some embodiments, the rep/cap sequences can be provided in trans during production to generate AAV particles. In some embodiments, the viral genome of an AAV is comprised in an AAV vector, which further encodes a capsid protein e.g., a structural protein, wherein the capsid protein comprises a VP1 polypeptide, a VP2 polypeptide, and/or a VP3 polypeptide; and/or a Rep protein, e.g., a non-structural protein, wherein the Rep protein comprises a Rep78 protein, a Rep68, Rep52 protein, and/or a Rep40 protein.

In some embodiments, AAV particles of the present disclosure are recombinant AAV particles which are replication defective and lacking the nucleotide sequences encoding functional Rep and Cap proteins. In some embodiments, these defective AAV particles may lack most or all parental coding sequences and carry only one or two AAV ITR sequences and the nucleic acid of interest for delivery to a cell, a tissue, an organ, or an organism.

Methods for producing and/or modifying AAV particles are disclosed in the art such as pseudotyped AAV particles (PCT Patent Publication Nos. WO200028004; WO200123001; WO2004112727; WO2005005610; and WO2005072364, the content of each of which is incorporated herein by reference in its entirety).

As described herein, the AAV particles of the disclosure comprising an AAV capsid polypeptide, and a viral genome are capable of providing, e.g., delivering, a transgene to a cell, e.g., mammalian cell ad/or a subject. In some embodiments, the recombinant AAV particles of the present disclosure are capable of vectorized delivery of a siRNA (e.g., an siRNA for targeting MAPT).

AAV Capsids and Variants thereof.

In some embodiments, an AAV particle, e.g., an AAV particle for the vectorized delivery of an siRNA described herein (e.g., an siRNA for targeting MAPT), may comprise an AAV capsid polypeptide, e.g., an AAV capsid variant.

In some embodiments, the AAV capsid polypeptide, e.g., AAV capsid variant, allows for blood brain barrier penetration following intravenous administration. In some embodiments, the AAV capsid, e.g., AAV capsid variant, allows for blood brain barrier penetration following focused ultrasound (FUS), e.g., coupled with the intravenous administration of microbubbles (FUS-MB), or MRI-guided FUS coupled with intravenous administration. In some embodiments the AAV capsid, e.g., AAV capsid variant allows for increased distribution to a brain region. In some embodiments, the brain region comprises a frontal cortex, sensory cortex, motor cortex, caudate, dentate nucleus, cerebellar cortex, cerebral cortex, brain stem, hippocampus, thalamus, putamen, or a combination thereof. In some embodiments, the AAV capsid, e.g., AAV capsid variant allows for preferential transduction in a brain region relative to the transduction in the dorsal root ganglia (DRG).

In some embodiments the AAV capsid polypeptide, e.g., AAV capsid variant allows for increased distribution to a spinal cord region. In some embodiments, the spinal region comprises a cervical spinal cord region, thoracic spinal cord region, and/or lumbar spinal cord region.

In some embodiments, the AAV capsid polypeptide, e.g., an AAV capsid variant comprises a VOY101 capsid polypeptide, a PHPeB capsid polypeptide, an AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAV9 K449R capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 1, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide comprises any one of the nucleotide sequences in Table 1, or a nucleotide sequence substantially identical (e.g., having at least 70%, 75%, 80%, 85%, 90%, 92%, 95%, 97%, 98%, or 99% sequence identity) thereto.

TABLE 1
Exemplary full length capsid sequences

| | SEQ | |
|---|---|---|
| | ID | |
| Description | NO: | Sequence Information |
| VOY101 | 1 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9/hu.14 | 11 | MAADGYLPDWLEDNLSEGIREWWALKPGAPQPKANQQHQDNARGLVLPGYKYLGPGNGL |
| K449R | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 138 | MAADGYLPDWLEDNLSEGIREWWALKPGAPQPKANQQHQDNARGLVLPGYKYLGPGNGL |
| WT | | DKGEPVNAADAAALEHDKAYDQQLKAGDNPYLKYNHADAEFQERLKEDTSFGGNLGRAV |
| (amino acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 137 | ATGGCTGCCGATGGTTATCTTCCAGATTGGCTCGAGGACAACCTTAGTGAAGGAATTCG |
| WT | | CGAGTGGTGGGCTTTGAAACCTGGAGCCCCTCAACCCAAGGCAAATCAACAACATCAAG |
| (DNA) | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| PHP.eB | 3 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| PHP.B | 12 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| PHP.N | 13 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQTL |
| | | AVPFSNPAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAVrh10 | | MAADGYLPDWLEDNLSEGIREWWDLKPGAPKPKANQQKQDDGRGLVLPGYKYLGPFNGL |
| (amino acid) | 14 | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEPSPQRSPDSSTGIGKKGQQPAKKRLNFGQT |
| | | GDSESVPDPQPIGEPPAGPSGLGSGTMAAGGGAPMADNNEGADGVGSSSGNWHCDSTWL |
| | | GDRVITTSTRTWALPTYNNHLYKQISNGTSGGSTNDNTYFGYSTPWGYFDFNRFHCHFS |
| | | PRDWQRLINNNWGFRPKRLNFKLFNIQVKEVTQNEGTKTIANNLTSTIQVFTDSEYQLP |
| | | YVLGSAHQGCLPPFPADVFMIPQYGYLTLNNGSQAVGRSSFYCLEYFPSQMLRTGNNFE |
| | | FSYQFEDVPFHSSYAHSQSLDRLMNPLIDQYLYYLSRTQSTGGTAGTQQLLFSQAGPNN |
| | | MSAQAKNWLPGPCYRQQRVSTTLSQNNNSNFAWTGATKYHLNGRDSLVNPGVAMATHKD |
| | | DEERFFPSSGVLMFGKQGAGKDNVDYSSVMLTSEEEIKTTNPVATEQYGVVADNLQQQN |
| | | AAPIVGAVNSQGALPGMVWQNRDVYLQGPIWAKIPHTDGNFHPSPLMGGFGLKHPPPQI |
| | | LIKNTPVPADPPTTFSQAKLASFITQYSTGQVSVEIEWELQKENSKRWNPEIQYTSNYY |
| | | KSTNVDFAVNTDGTYSEPRPIGTRYLTRNL |
| AAVrh10 | 15 | ATGGCTGCCGATGGTTATCTTCCAGATTGGCTCGAGGACAACCTCTCTGAGGGCATTCG |
| (DNA) | | CGAGTGGTGGGACTTGAAACCTGGAGCCCCGAAACCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAGAGACCGGTAGAGCCATCACCCCAGCGTTCTCCAGACTCCTCTA |
| | | CGGGCATCGGCAAGAAAGGCCAGCAGCCCGCGAAAAAGAGACTCAACTTTGGGCAGACT |
| | | GGCGACTCAGAGTCAGTGCCCGACCCTCAACCAATCGGAGAACCCCCCGCAGGCCCCTC |
| | | TGGTCTGGGATCTGGTACAATGGCTGCAGGCGGTGGCGCTCCAATGGCAGACAATAACG |
| | | AAGGCGCCGACGGAGTGGGTAGTTCCTCAGGAAATTGGCATTGCGATTCCACATGGCTG |
| | | GGCGACAGAGTCATCACCACCAGCACCCGAACCTGGGCCCTCCCCACCTACAACAACCA |
| | | CCTCTACAAGCAAATCTCCAACGGGACTTCGGGAGGAAGCACCAACGACAACACCTACT |
| | | TCGGCTACAGCACCCCCTGGGGGTATTTTGACTTTAACAGATTCCACTGCCACTTCTCA |
| | | CCACGTGACTGGCAGCGACTCATCAACAACAACTGGGGATTCCGGCCCAAGAGACTCAA |
| | | CTTCAAGCTCTTCAACATCCAGGTCAAGGAGGTCACGCAGAATGAAGGCACCAAGACCA |
| | | TCGCCAATAACCTTACCAGCACGATTCAGGTCTTTACGGACTCGGAATACCAGCTCCCG |
| | | TACGTCCTCGGCTCTGCGCACCAGGGCTGCCTGCCTCCGTTCCCGGCGGACGTCTTCAT |
| | | GATTCCTCAGTACGGGTACCTGACTCTGAACAATGGCAGTCAGGCCGTGGGCCGTTCCT |
| | | CCTTCTACTGCCTGGAGTACTTTCCTTCTCAAATGCTGAGAACGGGCAACAACTTTGAG |
| | | TTCAGCTACCAGTTTGAGGACGTGCCTTTTCACAGCAGCTACGCGCACAGCCAAAGCCT |
| | | GGACCGGCTGATGAACCCCCTCATCGACCAGTACCTGTACTACCTGTCTCGGACTCAGT |
| | | CCACGGGAGGTACCGCAGGAACTCAGCAGTTGCTATTTTCTCAGGCCGGGCCTAATAAC |
| | | ATGTCGGCTCAGGCCAAAAACTGGCTACCCGGGCCCTGCTACCGGCAGCAACGCGTCTC |
| | | CACGACACTGTCGCAAAATAACAACAGCAACTTTGCCTGGACCGGTGCCACCAAGTATC |
| | | ATCTGAATGGCAGAGACTCTCTGGTAAATCCCGGTGTCGCTATGGCAACCCACAAGGAC |
| | | GACGAAGAGCGATTTTTTCCGTCCAGCGGAGTCTTAATGTTTGGGAAACAGGGAGCTGG |
| | | AAAAGACAACGTGGACTATAGCAGCGTTATGCTAACCAGTGAGGAAGAAATTAAAACCA |
| | | CCAACCCAGTGGCCACAGAACAGTACGGCGTGGTGGCCGATAACCTGCAACAGCAAAAC |
| | | GCCGCTCCTATTGTAGGGGCCGTCAACAGTCAAGGAGCCTTACCTGGCATGGTCTGGCA |
| | | GAACCGGGACGTGTACCTGCAGGGTCCTATCTGGGCCAAGATTCCTCACACGGACGGAA |
| | | ACTTTCATCCCTCGCCGCTGATGGGAGGCTTTGGACTGAAACACCCGCCTCCTCAGATC |
| | | CTGATTAAGAATACACCTGTTCCCGCGGATCCTCCAACTACCTTCAGTCAAGCTAAGCT |
| | | GGCGTCGTTCATCACGCAGTACAGCACCGGACAGGTCAGCGTGGAAATTGAATGGGAGC |
| | | TGCAGAAAGAAAACAGCAAACGCTGGAACCCAGAGATTCAATACACTTCCAACTACTAC |
| | | AAATCTACAAATGTGGACTTTGCTGTTAACACAGATGGCACTTATTCTGAGCCTCGCCC |
| | | CATCGGCACCCGTTACCTCACCCGTAATCTGTAA |
| AAV2 | 16 | MAADGYLPDWLEDTLSEGIRQWWKLKPGPPPPKPAERHKDDSRGLVLPGYKYLGPFNGL |
| (amino acid) | | DKGEPVNEADAAALEHDKAYDRQLDSGDNPYLKYNHADAEFQERLKEDTSFGGNLGRAV |
| | | FQAKKRVLEPLGLVEEPVKTAPGKKRPVEHSPVEPDSSSGTGKAGQQPARKRLNFGQTG |
| | | DADSVPDPQPLGQPPAAPSGLGTNTMATGSGAPMADNNEGADGVGNSSGNWHCDSTWMG |
| | | DRVITTSTRTWALPTYNNHLYKQISSQSGASNDNHYFGYSTPWGYFDFNRFHCHFSPRD |
| | | WQRLINNNWGFRPKRLNFKLFNIQVKEVTQNDGTTTIANNLTSTVQVFTDSEYQLPYVL |
| | | GSAHQGCLPPFPADVFMVPQYGYLTLNNGSQAVGRSSFYCLEYFPSQMLRTGNNFTFSY |
| | | TFEDVPFHSSYAHSQSLDRLMNPLIDQYLYYLSRTNTPSGTTTQSRLQFSQAGASDIRD |
| | | QSRNWLPGPCYRQQRVSKTSADNNNSEYSWTGATKYHLNGRDSLVNPGPAMASHKDDEE |
| | | KFFPQSGVLIFGKQGSEKTNVDIEKVMITDEEEIRTTNPVATEQYGSVSTNLQRGNRQA |
| | | ATADVNTQGVLPGMVWQDRDVYLQGPIWAKIPHTDGHFHPSPLMGGFGLKHPPPQILIK |
| | | NTPVPANPSTTFSAAKFASFITQYSTGQVSVEIEWELQKENSKRWNPEIQYTSNYNKSV |
| | | NVDFTVDTNGVYSEPRPIGTRYLTRNL |
| AAV2 | 17 | ATGGCTGCCGATGGTTATCTTCCAGATTGGCTCGAGGACACTCTCTCTGAAGGAATAAG |
| (DNA) | | ACAGTGGTGGAAGCTCAAACCTGGCCCACCACCACCAAAGCCCGCAGAGCGGCATAAGG |
| | | ACGACAGCAGGGGTCTTGTGCTTCCTGGGTACAAGTACCTCGGACCCTTCAACGGACTC |
| | | GACAAGGGAGAGCCGGTCAACGAGGCAGACGCCGCGGCCCTCGAGCACGACAAAGCCTA |
| | | CGACCGGCAGCTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGACGAGCAGTC |
| | | TTCCAGGCGAAAAAGAGGGTTCTTGAACCTCTGGGCCTGGTTGAGGAACCTGTTAAGAC |
| | | GGCTCCGGGAAAAAAGAGGCCGGTAGAGCACTCTCCTGTGGAGCCAGACTCCTCCTCGG |
| | | GAACCGGAAAGGCGGGCCAGCAGCCTGCAAGAAAAAGATTGAATTTTGGTCAGACTGGA |
| | | GACGCAGACTCAGTACCTGACCCCCAGCCTCTCGGACAGCCACCAGCAGCCCCCTCTGG |
| | | TCTGGGAACTAATACGATGGCTACAGGCAGTGGCGCACCAATGGCAGACAATAACGAGG |
| | | GCGCCGACGGAGTGGGTAATTCCTCGGGAAATTGGCATTGCGATTCCACATGGATGGGC |
| | | GACAGAGTCATCACCACCAGCACCCGAACCTGGGCCCTGCCCACCTACAACAACCACCT |
| | | CTACAAACAAATTTCCAGCCAATCAGGAGCCTCGAACGACAATCACTACTTTGGCTACA |
| | | GCACCCCTTGGGGGTATTTTGACTTCAACAGATTCCACTGCCACTTTTCACCACGTGAC |
| | | TGGCAAAGACTCATCAACAACAACTGGGGATTCCGACCCAAGAGACTCAACTTCAAGCT |
| | | CTTTAACATTCAAGTCAAAGAGGTCACGCAGAATGACGGTACGACGACGATTGCCAATA |
| | | ACCTTACCAGCACGGTTCAGGTGTTTACTGACTCGGAGTACCAGCTCCCGTACGTCCTC |
| | | GGCTCGGCGCATCAAGGATGCCTCCCGCCGTTCCCAGCAGACGTCTTCATGGTGCCACA |
| | | GTATGGATACCTCACCCTGAACAACGGGAGTCAGGCAGTAGGACGCTCTTCATTTTACT |
| | | GCCTGGAGTACTTTCCTTCTCAGATGCTGCGTACCGGAAACAACTTTACCTTCAGCTAC |
| | | ACTTTTGAGGACGTTCCTTTCCACAGCAGCTACGCTCACAGCCAGAGTCTGGACCGTCT |
| | | CATGAATCCTCTCATCGACCAGTACCTGTATTACTTGAGCAGAACAAACACTCCAAGTG |
| | | GAACCACCACGCAGTCAAGGCTTCAGTTTTCTCAGGCCGGAGCGAGTGACATTCGGGAC |
| | | CAGTCTAGGAACTGGCTTCCTGGACCCTGTTACCGCCAGCAGCGAGTATCAAAGACATC |
| | | TGCGGATAACAACAACAGTGAATACTCGTGGACTGGAGCTACCAAGTACCACCTCAATG |
| | | GCAGAGACTCTCTGGTGAATCCGGGCCCGGCCATGGCAAGCCACAAGGACGATGAAGAA |
| | | AAGTTTTTTCCTCAGAGCGGGGTTCTCATCTTTGGGAAGCAAGGCTCAGAGAAAACAAA |
| | | TGTGGACATTGAAAAGGTCATGATTACAGACGAAGAGGAAATCAGGACAACCAATCCCG |
| | | TGGCTACGGAGCAGTATGGTTCTGTATCTACCAACCTCCAGAGAGGCAACAGACAAGCA |
| | | GCTACCGCAGATGTCAACACACAAGGCGTTCTTCCAGGCATGGTCTGGCAGGACAGAGA |
| | | TGTGTACCTTCAGGGGCCCATCTGGGCAAAGATTCCACACACGGACGGACATTTTCACC |
| | | CCTCTCCCCTCATGGGTGGATTCGGACTTAAACACCCTCCTCCACAGATTCTCATCAAG |
| | | AACACCCCGGTACCTGCGAATCCTTCGACCACCTTCAGTGCGGCAAAGTTTGCTTCCTT |
| | | CATCACACAGTACTCCACGGGACAGGTCAGCGTGGAGATCGAGTGGGAGCTGCAGAAGG |
| | | AAAACAGCAAACGCTGGAATCCCGAAATTCAGTACACTTCCAACTACAACAAGTCTGTT |
| | | AATGTGGACTTTACTGTGGACACTAATGGCGTGTATTCAGAGCCTCGCCCCATTGGCAC |
| | | CAGATACCTGACTCGTAATCTGTAA |
| AAV1 | 18 | MAADGYLPDWLEDNLSEGIREWWDLKPGAPKPKANQQKQDDGRGLVLPGYKYLGPFNGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDFNRFHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWTGASKYNLNGRESIINPGTAMASHKDDE |
| | | DKFFPMSGVMIFGKESAGASNTALDNVMITDEEEIKATNPVATERFGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| AAVI | 19 | atggctgccgatggttatcttccagattggctcgaggacaacctctctgagggcattcg |
| (DNA) | | cgagtggtgggacttgaaacctggagccccgaagcccaaagccaaccagcaaaagcagg |
| | | acgacggccggggtctggtgcttcctggctacaagtacctcggacccttcaacggactc |
| | | gacaagggggagcccgtcaacgcggcggacgcagcggccctcgagcacgacaaggccta |
| | | cgaccagcagctcaaagcgggtgacaatccgtacctgcggtataaccacgccgacgccg |
| | | agtttcaggagcgtctgcaagaagatacgtcttttgggggcaacctcgggcgagcagtc |
| | | ttccaggccaagaagcgggttctcgaacctctcggtctggttgaggaaggcgctaagac |
| | | ggctcctggaaagaaacgtccggtagagcagtcgccacaagagccagactectcctcgg |
| | | gcatcggcaagacaggccagcagcccgctaaaaagagactcaattttggtcagactggc |
| | | gactcagagtcagtccccgatccacaacctctcggagaacctccagcaacccccgctgc |
| | | tgtgggacctactacaatggcttcaggcggtggcgcaccaatggcagacaataacgaag |
| | | gcgccgacggagtgggtaatgcctcaggaaattggcattgcgattccacatggctgggc |
| | | gacagagtcatcaccaccagcacccgcacctgggccttgcccacctacaataaccacct |
| | | ctacaagcaaatctccagtgcttcaacgggggccagcaacgacaaccactacttcggct |
| | | acagcaccccctgggggtattttgatttcaacagattccactgccacttttcaccacgt |
| | | gactggcagcgactcatcaacaacaattggggattccggcccaagagactcaacttcaa |
| | | actcttcaacatccaagtcaaggaggtcacgacgaatgatggcgtcacaaccatcgcta |
| | | ataaccttaccagcacggttcaagtcttctcggactcggagtaccagcttccgtacgtc |
| | | ctcggctctgcgcaccagggctgcctccctccgttcccggcggacgtgttcatgattcc |
| | | gcaatacggctacctgacgctcaacaatggcagccaagccgtgggacgttcatcctttt |
| | | actgcctggaatatttcccttctcagatgctgagaacgggcaacaactttaccttcagc |
| | | tacacctttgaggaagtgcctttccacagcagctacgcgcacagccagagcctggaccg |
| | | gctgatgaatcctctcatcgaccaatacctgtattacctgaacagaactcaaaatcagt |
| | | ccggaagtgcccaaaacaaggacttgctgtttagccgtgggtctccagctggcatgtct |
| | | gttcagcccaaaaactggctacctggaccctgttatcggcagcagcgcgtttctaaaac |
| | | aaaaacagacaacaacaacagcaattttacctggactggtgcttcaaaatataacctca |
| | | atgggcgtgaatccatcatcaaccctggcactgctatggcctcacacaaagacgacgaa |
| | | gacaagttctttcccatgagcggtgtcatgatttttggaaaagagagcgccggagcttc |
| | | aaacactgcattggacaatgtcatgattacagacgaagaggaaattaaagccactaacc |
| | | ctgtggccaccgaaagatttgggaccgtggcagtcaatttccagagcagcagcacagac |
| | | cctgcgaccggagatgtgcatgctatgggagcattacctggcatggtgtggcaagatag |
| | | agacgtgtacctgcagggtcccatttgggccaaaattcctcacacagatggacactttc |
| | | acccgtctcctcttatgggcggctttggactcaagaacccgcctcctcagatcctcatc |
| | | aaaaacacgcctgttcctgcgaatcctccggcggagttttcagctacaaagtttgcttc |
| | | attcatcacccaatactccacaggacaagtgagtgtggaaattgaatgggagctgcaga |
| | | aagaaaacagcaagcgctggaatcccgaagtgcagtacacatccaattatgcaaaatct |
| | | gccaacgttgattttactgtggacaacaatggactttatactgagcctcgccccattgg |
| | | cacccgttaccttacccgtcccctgtaa |
| AAV5 | 20 | MSFVDHPPDWLEEVGEGLREFLGLEAGPPKPKPNQQHQDQARGLVLPGYNYLGPGNGLD |
| (amino acid) | | RGEPVNRADEVAREHDISYNEQLEAGDNPYLKYNHADAEFQEKLADDTSFGGNLGKAVF |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDFNRFHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRTGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMTNNLQGSNTYALENTMIFNS |
| | | QPANPGTTATYLEGNMLITSESETQPVNRVAYNVGGQMATNNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |
| AAV5 | 21 | atgtcttttgttgatcaccctccagattggttggaagaagttggtgaaggtcttcgcga |
| (DNA) | | gtttttgggccttgaagcgggcccaccgaaaccaaaacccaatcagcagcatcaagatc |
| | | aagcccgtggtcttgtgctgcctggttataactatctcggacccggaaacggtctcgat |
| | | cgaggagagcctgtcaacagggcagacgaggtcgcgcgagagcacgacatctcgtacaa |
| | | cgagcagcttgaggcgggagacaacccctacctcaagtacaaccacgcggacgccgagt |
| | | ttcaggagaagctcgccgacgacacatccttcgggggaaacctcggaaaggcagtcttt |
| | | caggccaagaaaagggttctcgaaccttttggcctggttgaagagggtgctaagacggc |
| | | ccctaccggaaagcggatagacgaccactttccaaaaagaaagaaggcccggaccgaag |
| | | aggactccaagccttccacctcgtcagacgccgaagctggacccagcggatcccagcag |
| | | ctgcaaatcccagcccaaccagcctcaagtttgggagctgatacaatgtctgcgggagg |
| | | tggcggcccattgggcgacaataaccaaggtgccgatggagtgggcaatgcctcgggag |
| | | attggcattgcgattccacgtggatgggggacagagtcgtcaccaagtccacccgaacc |
| | | tgggtgctgcccagctacaacaaccaccagtaccgagagatcaaaagcggctccgtcga |
| | | cggaagcaacgccaacgcctactttggatacagcaccccctgggggtactttgacttta |
| | | accgcttccacagccactggagcccccgagactggcaaagactcatcaacaactactgg |
| | | ggcttcagaccccggtccctcagagtcaaaatcttcaacattcaagtcaaagaggtcac |
| | | ggtgcaggactccaccaccaccatcgccaacaacctcacctccaccgtccaagtgttta |
| | | cggacgacgactaccagctgccctacgtcgtcggcaacgggaccgagggatgcctgccg |
| | | gccttccctccgcaggtctttacgctgccgcagtacggttacgcgacgctgaaccgcga |
| | | caacacagaaaatcccaccgagaggagcagcttcttctgcctagagtactttcccagca |
| | | agatgctgagaacgggcaacaactttgagtttacctacaactttgaggaggtgcccttc |
| | | cactccagcttcgctcccagtcagaacctcttcaagctggccaacccgctggtggacca |
| | | gtacttgtaccgcttcgtgagcacaaataacactggcggagtccagttcaacaagaacc |
| | | tggccgggagatacgccaacacctacaaaaactggttcccggggcccatgggccgaacc |
| | | cagggctggaacctgggctccggggtcaaccgcgccagtgtcagcgccttcgccacgac |
| | | caataggatggagctcgagggcgcgagttaccaggtgcccccgcagccgaacggcatga |
| | | ccaacaacctccagggcagcaacacctatgccctggagaacactatgatcttcaacagc |
| | | cagccggcgaacccgggcaccaccgccacgtacctcgagggcaacatgctcatcaccag |
| | | cgagagcgagacgcagccggtgaaccgcgtggcgtacaacgtcggcgggcagatggcca |
| | | ccaacaaccagagctccaccactgccccegcgaccggcacgtacaacctccaggaaatc |
| | | gtgcccggcagcgtgtggatggagagggacgtgtacctccaaggacccatctgggccaa |
| | | gatcccagagacgggggcgcactttcacccctctccggccatgggcggattcggactca |
| | | aacacccaccgcccatgatgctcatcaagaacacgcctgtgcccggaaatatcaccagc |
| | | ttctcggacgtgcccgtcagcagcttcatcacccagtacagcaccgggcaggtcaccgt |
| | | ggagatggagtgggagctcaagaaggaaaactccaagaggtggaacccagagatccagt |
| | | acacaaacaactacaacgacccccagtttgtggactttgccccggacagcaccggggaa |
| | | tacagaaccaccagacctatcggaacccgataccttacccgacccctttaa |
| TTD-001 | 3636 | MAADGYLPDWLEDNLSEGIREWWALKPGAPQPKANQQHQDNARGLVLPGYKYLGPGNGL |
| 9mer peptide | | DKGEPVNAADAAALEHDKAYDQQLKAGDNPYLKYNHADAEFQERLKEDTSFGGNLGRAV |
| underlined, | | FQAKKRLLEPLGLVEEAAKTAPGKKRPVEQSPQEPDSSAGIGKSGAQPAKKRLNFGQTG |
| starts at | | DTESVPDPQPIGEPPAAPSGVGSLTMASGGGAPVADNNEGADGVGSSSGNWHCDSQWLG |
| position 587 | | DRVITTSTRTWALPTYNNHLYKQISNSTSGGSSNDNAYFGYSTPWGYFDFNRFHCHFSP |
| (immediately | | RDWQRLINNNWGFRPKRLNFKLFNIQVKEVTDNNGVKTIANNLTSTVQVFTDSDYQLPY |
| subsequent to | | VLGSAHEGCLPPFPADVFMIPQYGYLTLNDGSQAVGRSSFYCLEYFPSQMLRTGNNFQF |
| position 586); | | SYEFENVPFHSSYAHSQSLDRLMNPLIDQYLYYLSKTINGSGQNQQTLKFSVAGPSNMA |
| 743 aa | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSPLNG |
| | | AVHLYAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| TTD-001 | 3623 | ATGGCTGCCGATGGTTATCTTCCAGATTGGCTCGAGGACAACCTTAGTGAAGGAATTCG |
| 9mer peptide | | CGAGTGGTGGGCTTTGAAACCTGGAGCCCCTCAACCCAAGGCAAATCAACAACATCAAG |
| underlined | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTCCGCTTAATGGT |
| | | GCCGTCCATCTTTATGCTCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| VOY9P39 | 5147 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTG |
| | | STTGWAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| VOY9P39 | 5148 | ATGGCTGCCGATGGTTATCTTCCAGATTGGCTCGAGGACAACCTTAGTGAAGGAATTCG |
| (DNA) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGACGGAACAGGA |
| | | AGCACAACAGGATGGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |

In some embodiments, the AAV capsid polypeptide, e.g., the AAV capsid variant, comprises the amino acid sequence of any of SEQ ID NOs: 1, 3, 11, 138, 12, 13, 14, 16, 18, 20, 5147, 3636, or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the amino acid sequence of any of SEQ ID NOs: 1, 3, 11, 138, 12, 13, 14, 16, 18, 20, or 3636. In some embodiments, the AAV capsid polypeptide, e.g., the AAV capsid variant, comprises an amino acid sequence encoded by the nucleotide sequence of any of SEQ ID NOs: 137, 15, 17, 19, 21, 3623, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding the AAV capsid polypeptide, e.g., the AAV capsid variant, comprises the nucleotide sequence of any of SEQ ID NOs: 137, 15, 17, 19, 21, 5148, or 3623, or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises a nucleotide sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the nucleotide sequence of any of SEQ ID NOs: 137, 15, 17, 19, 21, or 3623.

In some embodiments, the AAV capsid polypeptide, e.g., the AAV capsid variant, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 138. In some embodiments, the AAV capsid polypeptide, e.g., the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the AAV capsid polypeptide, e.g., the AAV capsid variant, comprises substitution at position K449, e.g., a K449R substitution, numbered according to SEQ ID NO: 138.

In some embodiments, the AAV capsid polypeptide, e.g., the AAV capsid variant, comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (conservative substitutions), relative to the amino acid sequence of SEQ ID NO: 11, optionally wherein position 449 is not R.

In some embodiments, the capsid polypeptide, comprises the amino acid sequence of SEQ ID NO: 1 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 1.

In some embodiments, the capsid polypeptide, comprises the amino acid sequence of SEQ ID NO: 3 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 3.

In some embodiments, the capsid polypeptide, comprises the amino acid sequence of SEQ ID NO: 5147 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 5147.

In some embodiments, an AAV capsid variant disclosed herein comprises a modification in loop VIII of AAV9, e.g., at positions between 580-599, e.g., at positions 587, 588, 589, and/or 590, numbered relative to SEQ ID NO: 5, 8, 138 or 3636-3647. In some embodiments, loop (e.g., loop VIII) is used interchangeably herein with the term variable region (e.g., variable region VIII), or VR (e.g., VR-VIII). In some embodiments loop VIII comprises positions 580-599 (e.g., amino acids VATNHQSAQAQAQTGWVQNQ (SEQ ID NO: 1195)), numbered according to SEQ ID NO: 138. In some embodiments, loop VIII comprises positions 582-593 (e.g., amino acids TNHQSAQAQAQT (SEQ ID NO: 1196)), numbered according to SEQ ID NO: 138. In some embodiments loop VIII comprises positions 587-593 (e.g., amino acids AQAQAQT (SEQ ID NO: 1197)), numbered according to SEQ ID NO: 138. In some embodiments loop VIII comprises positions 587-590 (e.g., amino acids AQAQ (SEQ ID NO: 5026)), numbered according to SEQ ID NO: 138. In some embodiments, loop VIII or variable region VIII (VR-VIII) is as described in DiMattia et al. “Structural Insights into the Unique Properties of the Adeno-Associated Virus Serotype 9,” Journal of Virology, 12(86):6947-6958 (the contents of which are hereby incorporated by reference in their entirety), e.g., comprising positions 581-593 (e.g., ATNHQSAQAQAQT (SEQ ID NO: 1198)), numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262). In some embodiments, the peptide is present immediately subsequent to position 588, relative to a reference sequence numbered according to SEQ ID NO: 138. In some embodiments, the capsid polypeptide comprises the amino acid substitutions of A587D and Q588G, numbered according to SEQ ID NO: 138.

In some embodiments, the AAV capsid polypeptide, e.g., the AAV capsid variant, comprises the amino acid substitution of K449R, numbered according to SEQ ID NO: 138; and a peptide comprising the amino acid sequence of TLAVPFK (SEQ ID NO: 1262), wherein the peptide is present immediately subsequent to position 588, relative to a reference sequence numbered according to SEQ ID NO: 138.

In some embodiments, the AAV capsid polypeptide, e.g., the AAV capsid variant, comprises the amino acid substitution of K449R, numbered according to SEQ ID NO: 138; an peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises at least 3, 4, 5, 6, 7, 8, or 9 consecutive amino acids from the amino acid sequence of PLNGAVHLY (SEQ ID NO: 3648). In some embodiments, the AAV capsid polypeptide, e.g., the AAV capsid variant, comprises the amino acid sequence of PLNGAVHLY (SEQ ID NO: 3648), an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648), or an amino acid sequence comprising at least one, two, or three but no more than four different amino acids relative to the amino acid sequence of PLNGAVHLY (SEQ ID NO: 3648), optionally wherein position 7 is H. In some embodiments, the amino acid sequence is present in loop VIII. In some embodiments, the amino acid sequence is present immediately subsequent to position 586, relative to a reference sequence numbered according to the amino acid sequence of SEQ ID NO: 138. In some embodiments, the amino acid sequence replaces positions replaces positions 587 and 588 (e.g., A587 and Q 588), numbered according to SEQ ID NO: 138. In some embodiments, the amino acid sequence is present at immediately subsequent to position 586 and replaces positions 587 and 588 (e.g., A587 and Q 588), numbered according to SEQ ID NO: 138.

In some embodiments, the 3 consecutive amino acids comprise PLN. In some embodiments, the 4 consecutive amino acids comprise PLNG (SEQ ID NO: 3678). In some embodiments, the 5 consecutive amino acids comprise PLNGA (SEQ ID NO: 3679). In some embodiments, the 6 consecutive amino acids comprise PLNGAV (SEQ ID NO: 3680). In some embodiments, the 7 consecutive amino acids comprise PLNGAVH (SEQ ID NO: 3681). In some embodiments, the 8 consecutive amino acids comprise PLNGAVHL (SEQ ID NO: 3682). In some embodiments, the 9 consecutive amino acids comprise PLNGAVHLY (SEQ ID NO: 3648).

In some embodiments, the four consecutive amino acids comprise NGAV (SEQ ID NO: 3683). In some embodiments, the four consecutive amino acids comprise GAVH (SEQ ID NO: 3684). In some embodiments, the five consecutive amino acids comprise NGAVH (SEQ ID NO: 3685). In some embodiments, the five consecutive amino acids comprise GAVHL (SEQ ID NO: 3686). In some embodiments, the five consecutive amino acids comprise AVHLY (SEQ ID NO: 3687). In some embodiments, the six consecutive amino acids comprise NGAVHL (SEQ ID NO: 3688). In some embodiments, the seven consecutive amino acids comprise NGAVHLY (SEQ ID NO: 3689).

In some embodiments, the AAV capsid polypeptide, e.g., the AAV capsid variant (e.g., an AAV capsid variant described herein), comprises an amino acid sequence encoded by the nucleotide sequence of CCGCTTAATGGTGCCGTCCATCTTTAT (SEQ ID NO: 3660), or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of CCGCTTAATGGTGCCGTCCATCTTTAT (SEQ ID NO: 3660). In some embodiments, the AAV capsid polypeptide, e.g., the AAV capsid variant (e.g., an AAV capsid variant described herein), comprises an amino acid sequence encoded by a nucleotide sequence comprising at least one, two, three, four, five, six, or seven, but no more than ten different nucleotides relative to the nucleotide sequence of CCGCTTAATGGTGCCGTCCATCTTTAT (SEQ ID NO: 3660).

In some embodiments, the AAV capsid polypeptide, e.g., the AAV capsid variant, comprises the amino acid P at position 587, the amino acid L at position 588, and the amino acid sequence NGAVHLY (SEQ ID NO: 3689), which is present immediately subsequent to position 588, corresponding to or numbered according to SEQ ID NO: 3636.

In some embodiments, the AAV capsid polypeptide, e.g., the AAV capsid variant, e.g., an AAV capsid variant described herein, comprises the amino acid sequence of SEQ ID NO: 3636, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises the amino acid sequence of SEQ ID NO: 3636, or an amino acid sequence at least 95% identical thereto. In some embodiments, the AAV capsid polypeptide, e.g., the AAV capsid variant, e.g., an AAV capsid variant described herein, comprises the amino acid sequence of SEQ ID NO: 3636, or an amino acid sequence at least 99% identical thereto.

In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 3636. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 3636.

In some embodiments, the AAV capsid polypeptide, e.g., the AAV capsid variant, comprises the amino acid sequence corresponding to positions 138-743, e.g., a VP2, of SEQ ID NO: 3636, or a sequence with at least 80% (e.g., at least about 85, 90, 95, 96, 97, 98, or 99%) sequence identity thereto. In some embodiments, the AAV capsid variant comprises the amino acid sequence corresponding to positions 203-743, e.g., a VP3, of SEQ ID NO: 3636, or a sequence with at least 80% (e.g., at least about 85, 90, 95, 96, 97, 98, or 99%) sequence identity thereto. In some embodiments, the AAV capsid polypeptide, e.g., the AAV capsid variant, comprises the amino acid sequence corresponding to positions 138-736, e.g., a VP2, of SEQ ID NO: 138, or a sequence with at least 80% (e.g., at least about 85, 90, 95, 96, 97, 98, or 99%) sequence identity thereto. In some embodiments, the AAV capsid variant comprises the amino acid sequence corresponding to positions 203-736, e.g., a VP3, of SEQ ID NO: 138, or a sequence with at least 80% (e.g., at least about 85, 90, 95, 96, 97, 98, or 99%) sequence identity thereto. In some embodiments, the amino acid sequence is present immediately subsequent to position 586, numbered according to any one of SEQ ID NO: 138 or 3636, optionally wherein the amino acid replaces positions 587 and 588 (e.g., A587 and Q588), numbered according to SEQ ID NO: 138. In some embodiments, the amino acid sequence corresponds to positions 587-595 of SEQ ID NO: 3636.

In some embodiments, the AAV capsid polypeptide, e.g., the AAV capsid variant, comprises one or two, but no more than three different amino acids (e.g., substitutions, e.g., conservative substitutions) relative to the amino acid sequence of PLNGAVHLY (SEQ ID NO: 3648), wherein the AAV capsid variant comprises (a) a VP1 protein comprising the amino acid sequence of SEQ ID NO: 138 or 3636; (b) a VP2 protein comprising the amino acid sequence of positions 138-736 of SEQ ID NO: 138 or positions 138-743 of SEQ ID NO: 5, 8, or 3636; (c) a VP3 protein comprising the amino acid sequence of positions 203-736 of SEQ ID NO: 138 or positions 203-743 of SEQ ID NO: 5, 8, or 3636; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to position 586, numbered according to any one of SEQ ID NO: 138 or 3636, optionally wherein the amino acid replaces positions 587 and 588 (e.g., A587 and Q588), numbered according to SEQ ID NO: 138. In some embodiments, the amino acid sequence corresponds to positions 587-595 of SEQ ID NO: 3636.

In some embodiments, the AAV capsid polypeptide, e.g., the AAV capsid variant, comprises one or two, but no more than three different amino acids (e.g., substitutions, e.g., conservative substitutions) relative to the amino acid sequence of PLNGAVHLY (SEQ ID NO: 3648), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to SEQ ID NO: 3636. In some embodiments, the amino acid sequence is present immediately subsequent to position 586, numbered according to any one of SEQ ID NO: 138 or 3636, optionally wherein the amino acid replaces positions 587 and 588 (e.g., A587 and Q588), numbered according to SEQ ID NO: 138. In some embodiments, the amino acid sequence corresponds to positions 587-595 of SEQ ID NO: 3636.

In some embodiments, the AAV capsid polypeptide, e.g., the AAV capsid variant, comprises an amino acid sequence comprising at least 5, 6, 7, 8, or 9 consecutive amino acids from the amino acid sequence of PLNGAVHLY (SEQ ID NO: 3648), wherein: (i) the 5 consecutive amino acids comprise PLNGA (SEQ ID NO: 3679); (ii) the 6 consecutive amino acids comprise PLNGAV (SEQ ID NO: 3680); (iii) the 7 consecutive amino acids comprise PLNGAVH (SEQ ID NO: 3681); (iv) the 8 consecutive amino acids comprise PLNGAVHL (SEQ ID NO: 3682); or (v) the 9 consecutive amino acids comprise PLNGAVHLY (SEQ ID NO: 3648), wherein the AAV capsid variant comprises: (a) a VP1 protein comprising the amino acid sequence of SEQ ID NO: 138 or SEQ ID NO: 3636; (b) a VP2 protein comprising the amino acid sequence of positions 138-736 of SEQ ID NO: 138 or positions 138-743 of SEQ ID NO: 3636; (c) a VP3 protein comprising the amino acid sequence of positions 203-736 of SEQ ID NO: 138 or positions 203-743 of SEQ ID NO: 3636; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to position 586, numbered according to any one of SEQ ID NO: 138 or 3636, optionally wherein the amino acid replaces positions 587 and 588 (e.g., A587 and Q588), numbered according to SEQ ID NO: 138. In some embodiments, the amino acid sequence corresponds to positions 587-595 of SEQ ID NO: 3636.

In some embodiments, the AAV capsid polypeptide, e.g., the AAV capsid variant, comprises an amino acid sequence comprising at least 5, 6, 7, 8, or 9 consecutive amino acids from the amino acid sequence of PLNGAVHLY (SEQ ID NO: 3648), wherein: (i) the 5 consecutive amino acids comprise PLNGA (SEQ ID NO: 3679); (ii) the 6 consecutive amino acids comprise PLNGAV (SEQ ID NO: 3680); (iii) the 7 consecutive amino acids comprise PLNGAVH (SEQ ID NO: 3681); (iv) the 8 consecutive amino acids comprise PLNGAVHL (SEQ ID NO: 3682); or (v) the 9 consecutive amino acids comprise PLNGAVHLY (SEQ ID NO: 3648), wherein the AAV capsid variant comprises the amino acid sequence of any one of SEQ ID NOs: 5, 8, or 3636 or an amino acid sequence with at least 90% (e.g., at least about 95, 96, 97, 98, or 99%) sequence identity to any one of SEQ ID NO: 3636. In some embodiments, the amino acid sequence is present immediately subsequent to position 586, numbered according to any one of SEQ ID NO: 138 or 3636, optionally wherein the amino acid replaces positions 587 and 588 (e.g., A587 and Q588), numbered according to SEQ ID NO: 138. In some embodiments, the amino acid sequence corresponds to positions 587-595 of SEQ ID NO: 3636.

In some embodiments, the nucleotide sequence encoding an AAV capsid polypeptide, e.g., an AAV capsid variant, described herein comprises the nucleotide sequence of SEQ ID NO: 3623, or a nucleotide sequence with at least 80% (e.g., at least about 85, 90, 95, 96, 97, 98, or 99%) sequence identity thereto. In some embodiments, nucleotide sequence encoding the AAV capsid variant comprises a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 3623. In some embodiments, the nucleotide sequence encoding the AAV capsid variant comprises a nucleotide sequence comprising at least one, two or three, but not more than 30, 20 or 10 different nucleotides, relative to the nucleotide sequence of SEQ ID NO: 3623. In some embodiments, the nucleic acid sequence encoding an AAV capsid polypeptide, e.g., an AAV capsid variant, described herein is codon optimized.

In some embodiments, an AAV capsid polypeptide, e.g., an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid polypeptide, e.g., an AAV capsid variant, of the present disclosure has decreased tropism for the liver. In some embodiments, an AAV capsid variant comprises a modification, e.g., substitution (e.g., conservative substitution), insertion, or deletion, that results in reduced tropism (e.g., de-targeting) and/or activity in the liver. In some embodiments, the reduced tropism in the liver is compared to an otherwise similar capsid that does not comprise the modification, e.g., a wild-type capsid polypeptide. In some embodiments, an AAV capsid variant described comprises a modification, e.g., substitution (e.g., conservative substitution), insertion, or deletion, that results in one or more of the following properties: (1) reduced tropism in the liver; (2) de-targeted expression in the liver; (3) reduced activity in the liver; and/or (4) reduced binding to galactose. In some embodiments, the reduction in any one, or all of properties (1)-(3) is compared to an otherwise similar AAV capsid variant that does not comprise the modification. Exemplary modifications are provided in WO 2018/119330; Pulicherla et al. (2011) Mol. Ther. 19(6): 1070-1078; Adachi et al. (2014) Nature Communications 5(3075), DOI: 10.1038/ncomms4075; and Bell et al. (2012) J. Virol. 86(13): 7326-33; the contents of which are hereby incorporated by reference in their entirety. In some embodiments, the AAV capsid variant comprises a modification e.g., substitution (e.g., conservative substitution), insertion, or deletion, at position N470 (e.g., N470A), D271 (e.g., D271A), N272 (e.g., N272A), Y446 (e.g., Y446A), N498 (e.g., N498Y or N498I), W503 (e.g., W503R or W503A), L620 (e.g., L620F), or a combination thereof, relative to a reference sequence numbered according to SEQ ID NO: 138. In some embodiments, the AAV capsid variant comprises one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498 (e.g., Y or I), and amino acid other than W at position 503 (e.g., R or A), and amino acid other than L at position 620 (e.g., F), relative to a reference sequence numbered according to SEQ ID NO: 138. In some embodiments, the AAV capsid variant comprises a modification e.g., substitution (e.g., conservative substitution), insertion, or deletion, at position N470 (e.g., N470A), D271 (e.g., D271A), N272 (e.g., N272A), Y446 (e.g., Y446A), and W503 (e.g., W503R or W03A), relative to a reference sequence numbered according to SEQ ID NO: 138. In some embodiments, the AAV capsid variant comprises a modification e.g., substitution (e.g., conservative substitution), insertion, or deletion, at N498 (e.g., N498Y) and L620 (e.g., L620F).

In some embodiments, an AAV capsid variant comprised herein comprises a modification as described in Adachi et al. (2014) Nature Communications 5(3075), DOI: 10.1038/ncomms4075, the contents of which are hereby incorporated by reference in its entirety. Exemplary modifications that alter or do not alter tissue transduction in at least the brain, liver, heart, lung, and/or kidney can be found in Supplementary Data 2 showing the AAV Barcode-Seq data obtained with AAV9-AA-VBCLib of Adachi et al. (supra), the contents of which are hereby incorporated by reference in its entirety.

In some embodiments, an, AAV capsid polypeptide, e.g., an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968):973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

Also provided herein are polynucleotide sequences encoding any of the AAV capsid variants described above and AAV particles, vectors, and cells comprising the same

### Viral Genome

In some aspects, an AAV particle of the present disclosure, e.g., an AAV particle comprising an AAV capsid polypeptide, e.g., an AAV capsid variant described herein, comprises a viral genome which encodes a an agent for targeting MAPT, e.g., a siRNA duplex for targeting MAPT or a modulatory polynucleotide encoding a siRNA duplex for targeting MAPT.

In some embodiments, an AAV particle as described herein comprising an AAV capsid polypeptide, e.g., AAV capsid variant, described herein, may be used for the delivery of a viral genome to a tissue (e.g., CNS, DRG, and/or muscle). In some embodiments, an AAV particle comprising an AAV capsid polypeptide, e.g., an AAV capsid variant, described herein can be used for delivery of a viral genome to a tissue or cell, e.g., CNS, DRG, or muscle cell or tissue. In some embodiments, an AAV particle of the present disclosure is a recombinant AAV particle. In some embodiments, an AAV particle of the present disclosure is an isolated AAV particle.

In some embodiments, the AAV particles described herein are used to deliver the agent to target MAPT (e.g., a MAPT targeting siRNA duplex or a modulatory polynucleotide encoding a siRNA duplex to cells of the CNS, after intravenous delivery.

In some embodiments, a viral genome of an AAV particle comprising an AAV capsid polypeptide, e.g., an AAV capsid variant, as described herein, comprises a nucleic acid comprising a transgene encoding the agent to target MAPT (e.g., a MAPT targeting siRNA duplex or a modulatory polynucleotide encoding a siRNA duplex (e.g., a siRNA duplex for targeting MAPT)). In some embodiments, the viral genome comprises an inverted terminal repeat (ITR) sequence. In some embodiments, the viral genome comprises two ITR sequences, e.g., one at the 5′ end of the viral genome (e.g., 5′ relative to the encoded payload) and one at the 3′ end of the viral genome (e.g., 3′ relative to the encoded payload). In some embodiments, a viral genome of the AAV particles described herein (e.g., comprising an AAV capsid variant described herein) may comprise a regulatory element (e.g., promoter), untranslated regions (UTR), a miR binding site a polyadenylation sequence (polyA), a filler or stuffer sequence, an intron, and/or a linker sequence, e.g., for enhancing transgene expression.

In some embodiments, the viral genome components are selected and/or engineered for expression of an agent for targeting MAPT (e.g., a MAPT targeting siRNA duplex or a modulatory polynucleotide encoding a siRNA duplex (e.g., a siRNA duplex for targeting MAPT)) in a target tissue (e.g., a CNS tissue, e.g., a brain tissue or a spinal cord tissue), or a target cell (e.g., a cell of the CNS, e.g., a brain cell or a spinal cord cell).

### Viral Genome Component: Inverted Terminal Repeats (ITRs)

In some embodiments, the viral genome comprises an ITR and a transgene encoding a payload. In some embodiment, the viral genome has two ITRs. In some embodiments, the two ITRs flank the nucleotide sequence encoding the payload at the 5′ and 3′ ends. In some embodiments, the ITRs function as origins of replication comprising recognition sites for replication. In some embodiments, the ITRs comprise sequence regions which can be complementary and symmetrically arranged. In some embodiments, the ITRs incorporated into viral genomes as described herein may be comprised of naturally occurring polynucleotide sequences or recombinantly derived polynucleotide sequences.

In some embodiments, the ITR may be of the same serotype as the capsid polypeptide, e.g., capsid variant, selected from any of the known serotypes, or a variant thereof. In some embodiments, the ITR may be of a different serotype than the capsid. In one embodiment, the viral genome comprises two ITR sequence regions, wherein the ITRs are of the same serotype as one another. In another embodiment, the viral genome comprises two ITR sequence regions, wherein the ITRs are of different serotypes. Non-limiting examples include zero, one or both of the ITRs having the same serotype as the capsid. In one embodiment both ITRs of the viral genome of the AAV particle are AAV2 ITRs.

In some embodiments, the ITR comprises the nucleotide sequence of SEQ ID NO: 5197, or a nucleotide sequence at least 70%, 75%, 80%, 85%, 90%, 92%, 95%, 96%, 97%, 98%, or 99% identical thereto. In some embodiments, the ITR comprises the nucleotide sequence of SEQ ID NO: 5200, or a nucleotide sequence at least 70%, 75%, 80%, 85%, 90%, 92%, 95%, 96%, 97%, 98%, or 99% identical thereto. In some embodiments, an AAV viral genome comprises an ITR comprising the nucleotide sequence of SEQ ID NO: 5197, or a nucleotide sequence at least 70%, 75%, 80%, 85%, 90%, 92%, 95%, 96%, 97%, 98%, or 99% identical thereto, and an ITR comprising the nucleotide sequence of SEQ ID NO: 5200, or a nucleotide sequence at least 70%, 75%, 80%, 85%, 90%, 92%, 95%, 96%, 97%, 98%, or 99% identical thereto.

In some embodiments, the ITR comprises the nucleotide sequence of SEQ ID NO: 4469, or a nucleotide sequence at least 70%, 75%, 80%, 85%, 90%, 92%, 95%, 96%, 97%, 98%, or 99% identical thereto. In some embodiments, the ITR comprises the nucleotide sequence of SEQ ID NO: 4470, or a nucleotide sequence at least 70%, 75%, 80%, 85%, 90%, 92%, 95%, 96%, 97%, 98%, or 99% identical thereto. In some embodiments, an AAV viral genome comprises an ITR comprising the nucleotide sequence of SEQ ID NO: 4469, or a nucleotide sequence at least 70%, 75%, 80%, 85%, 90%, 92%, 95%, 96%, 97%, 98%, or 99% identical thereto, and an ITR comprising the nucleotide sequence of SEQ ID NO: 4470, or a nucleotide sequence at least 70%, 75%, 80%, 85%, 90%, 92%, 95%, 96%, 97%, 98%, or 99% identical thereto.

### Viral Genome Component: Promoters

In some embodiments, the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid variant described herein comprises a viral genome comprising a nucleic acid comprising a transgene encoding a payload, wherein the transgene is operably linked to a promoter. In some embodiments, the promoter is a species-specific promoter, an inducible promoter, a tissue-specific promoter, or a cell cycle-specific promoter (e.g., a promoter as described in Parr et al., Nat. Med. 3:1145-9(1997); the contents of which are herein incorporated by reference in their entirety).

In some embodiments, the promoter may be naturally occurring or non-naturally occurring. Non-limiting examples of promoters include those derived from viruses, plants, mammals, or humans. In some embodiments, the promoters may be those derived from human cells or systems. In some embodiments, the promoter may be truncated or mutated, e.g., a promoter variant.

In some embodiments, the promoter is a ubiquitous promoter, e.g., capable of expression in multiple tissues. In some embodiments the promoter is a human elongation factor 1α-subunit (EF1α) promoter, the cytomegalovirus (CMV) immediate-early enhancer and/or promoter, the chicken β-actin (CBA) promoter and its derivative CAG, B glucuronidase (GUSB) promoter, or ubiquitin C (UBC) promoter. In some embodiments, the promoter is a cell or tissue specific promoter, e.g., capable of expression in tissues or cells of the central or peripheral nervous systems, targeted regions within (e.g., frontal cortex), and/or sub-sets of cells therein (e.g., excitatory neurons). In some embodiments, the promoter is a cell-type specific promoters capable of expression of a payload in excitatory neurons (e.g., glutamatergic), inhibitory neurons (e.g., GABA-crgic), neurons of the sympathetic or parasympathetic nervous system, sensory neurons, neurons of the dorsal root ganglia, motor neurons, or supportive cells of the nervous systems such as microglia, glial cells, astrocytes, oligodendrocytes, and/or Schwann cells.

In some embodiments, the promoter is a liver specific promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., αMHC, cTnT, and CMV-MLC2k).

In some embodiments, the promoter is a tissue-specific promoter for payload expression in a tissue or cell of the central nervous system. In some embodiments, the promoter is a synapsin (Syn) promoter, glutamate vesicular transporter (VGLUT) promoter, vesicular GABA transporter (VGAT) promoter, parvalbumin (PV) promoter, sodium channel Nav 1.8 promoter, tyrosine hydroxylase (TH) promoter, choline acetyltransferase (ChaT) promoter, methyl-CpG binding protein 2 (MeCP2) promoter, Ca2+/calmodulin-dependent protein kinase II (CaMKII) promoter, metabotropic glutamate receptor 2 (mGluR2) promoter, neurofilament light chain (NFL) or heavy chain (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter, or a fragment thereof. In some embodiments, the promoter is a cell-type specific promoter capable of expression in an astrocyte, e.g., a glial fibrillary acidic protein (GFAP) promoter and a EAAT2 promoter, or a fragment thereof. In some embodiments, the promoter is a cell-type specific promoter capable of expression in an oligodendrocyte, e.g., a myelin basic protein (MBP) promoter or a fragment thereof.

In some embodiments, the promoter is a GFAP promoter. In some embodiments, the promoter is a synapsin (syn or syn1) promoter, or a fragment thereof.

In some embodiments, the promoter comprises a CBA promoter or a variant thereof, e.g., a functional variant thereof. In some embodiments, the promoter comprises the nucleotide sequence of SEQ ID NO: 5199, or a nucleotide sequence at least 70%, 75%, 80%, 85%, 90%, 92%, 95%, 96%, 97%, 98%, or 99% identical thereto.

In some embodiments, the AAV viral genome comprises an enhancer. In some embodiments, the enhancer comprises a CMVie enhancer, or a variant thereof. In some embodiments, the enhancer comprises the nucleotide sequence of SEQ ID NO: 4471, or a nucleotide sequence at least 70%, 75%, 80%, 85%, 90%, 92%, 95%, 96%, 97%, 98%, or 99% identical thereto. In some embodiments, the enhancer comprises the nucleotide sequence of SEQ ID NO: 4472, or a nucleotide sequence at least 70%, 75%, 80%, 85%, 90%, 92%, 95%, 96%, 97%, 98%, or 99% identical thereto.

In some embodiments, the AAV viral genome comprises an enhancer and a promoter. In some embodiments, the enhancer comprises a CMVie enhancer and the promoter comprises a CBA promoter. In some embodiments, the enhancer and the promoter comprise the nucleotide sequence of SEQ ID NO: 4474, or a nucleotide sequence at least 70%, 75%, 80%, 85%, 90%, 92%, 95%, 96%, 97%, 98%, or 99% identical thereto.

In some embodiments, the promoter comprises an insulin promoter or a fragment thereof.

In some embodiments, the viral genome comprises a ubiquitous promoter. Non-limiting examples of ubiquitous promoters include CMV, CBA (including derivatives CAG, CB6, CBh, etc.), EF-1α, PGK, UBC, GUSB (hGBp), and UCOE (promoter of HNRPA2B1-CBX3). In some embodiments, the viral genome comprises an EF-1α promoter or EF-1α promoter variant, e.g., as provided in Table 2. In some embodiments, the EF-1α promoter comprises the nucleotide sequence of any one of SEQ ID NOs: 1874-1889 or any of the sequences provided in Table 40, a nucleotide sequence comprising at least one, two, or three but no more than four modifications, e.g., substitutions, relative to the nucleotide sequence of SEQ ID NOs: 1874-1889 or any of the sequences provided in Table 40, or a nucleotide sequence with at least 80% (e.g., 85%, 90%, 95%, 96%, 97%, 98%, or 99%) sequence identity to any one of SEQ ID NOs: 1874-1889 or any of the sequences provided in Table 2.

TABLE 2
Exemplary Promoter Variants

| | | SEQ ID |
|---|---|---|
| Description | Sequences | NO: |
| EF1a Promoter | CGTGAGGCTCCGGTGCCCGTCAGTGGGCAGAGCGCACATCGCCCACAGTCCCCG | 1874 |
| (intron | AGAAGTTGGGGGGAGGGGTCGGCAATTGAACCGGTGCCTAGAGAAGGTGGCGCG | |
| underlined) | GGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACG | |
| | GGTTTGCCGCCAGAACACAGGTAAGTGCCGTGTGTGGTTCCCGCGGGCCTGGCC | |
| | TCTTTACGGGTTATGGCCCTTGCGTGCCTTGAATTACTTCCACCTGGCTGCAGT | |
| | ACGTGATTCTTGATCCCGAGCTTCGGGTTGGAAGTGGGTGGGAGAGTTCGAGGC | |
| | CTTGCGCTTAAGGAGCCCCTTCGCCTCGTGCTTGAGTTGAGGCCTGGCCTGGGC | |
| | GCTGGGGCCGCCGCGTGCGAATCTGGTGGCACCTTCGCGCCTGTCTCGCTGCTT | |
| | TCGATAAGTCTCTAGCCATTTAAAATTTTTGATGACCTGCTGCGACGCTTTTTT | |
| | TCTGGCAAGATAGTCTTGTAAATGCGGGCCAAGATCTGCACACTGGTATTTCGG | |
| | TTTTTGGGGCCGCGGGCGGCGACGGGGCCCGTGCGTCCCAGCGCACATGTTCGG | |
| | CGAGGCGGGGCCTGCGAGCGCGGCCACCGAGAATCGGACGGGGGTAGTCTCAAG | |
| | CTGGCCGGCCTGCTCTGGTGCCTGGCCTCGCGCCGCCGTGTATCGCCCCGCCCT | |
| | GGGCGGCAAGGCTGGCCCGGTCGGCACCAGTTGCGTGAGCGGAAAGATGGCCGC | |
| | TTCCCGGCCCTGCTGCAGGGAGCTCAAAATGGAGGACGCGGCGCTCGGGAGAGC | |
| | GGGCGGGTGAGTCACCCACACAAAGGAAAAGGGCCTTTCCGTCCTCAGCCGTCG | |
| | CTTCATGTGACTCCACGGAGTACCGGGCGCCGTCCAGGCACCTCGATTAGTTCT | |
| | CGAGCTTTTGGAGTACGTCGTCTTTAGGTTGGGGGGAGGGGTTTTATGCGATGG | |
| | AGTTTCCCCACACTGAGTGGGTGGAGACTGAAGTTAGGCCAGCTTGGCACTTGA | |
| | TGTAATTCTCCTTGGAATTTGCCCTTTTTGAGTTTGGATCTTGGTTCATTCTCA | |
| | AGCCTCAGACAGTGGTTCAAAGTTTTTTTCTTCCATTTCAGGTGTCGTGA | |
| miniEFla | GCCCGTCAGTGGGCAGAGCGCACATCGCCCACAGTCCCCGAGAAGTTGGGGGGA | 1875 |
| | GGGGTCGGCAATTGAACCGGTGCCTAGAGAAGGTGGCGCGGGGTAAACTGGGAA | |
| | AGTGATGTCGTGTACTGGCTCCGCCTTTTTCCCGAGGGTGGGGGAGAACCGTAT | |
| | ATAAGTGCAGTAGTCGCCGTGAACGTTCTTTTTCGCAACGGGTTTGCCGCCAGA | |
| | ACACGCGTAAG | |
| Promoter | GCATG | |
| Variant 1 | | |
| Promoter | GGTGGAGAAGAGCATG | 1876 |
| Variant 2 | | |
| Promoter | GTCATCACTGAGGTGGAGAAGAGCATG | 1877 |
| Variant 3 | | |
| Promoter | CGTGAG | |
| Variant 4 | | |
| Promoter | GT | |
| Variant 5 | | |
| Promoter | GCTCCGGT | |
| Variant 6 | | |
| Promoter | GCCCGTCAGTGGGCAGAGCGCACATCGCCCACAGTCCCCGAGAAGTTGGGGGGA | 1878 |
| Variant 19 | GGGGTCGGCAATTGAACCGGTGCCTAGAGAAGGTGGCGCGGGGTAAACTGGGAA | |
| | AGTGATGTCGTGTACTGGCTCCGCCTTTTTCCCGAGGGTGGGGGAGAACCGTAT | |
| | ATAAGTGCAGTAGTCGCCGTGAACGTTCTTTTTCGCAACGGGTTTGCCGCCAGA | |
| | ACACAG | |
| Promoter | GCCCGTCAGTGGGCAGAGCGCACATCGCCCACAGTCCCCGAGAAGTTGGGGGGA | 1879 |
| Variant 20 | GGGGTCGGCAATTGAACCGGTGCCTAGAGAAGGTGGCGCGGGGTAAACTGGGAA | |
| | AGTGATGTCGTGTACTGGCTCCGCCTTTTTCCCGAGGGTGGGGGAGAACCGTAT | |
| | ATAAGTGCAGTAGTCGCCGTGAACGTTCTTTTTCGCAACGGGTTTGCCGCCAGA | |
| | ACACGC | |
| Promoter | GTAAG | |
| Variant 7 | | |
| Promoter | GTGCCCGTCAGTGGGCAGAGCGCACATCGCCCACAGTCCCCGAGAAGTTGGGGG | 1880 |
| Variant 8 | GAGGGGTCGGCAATTGAACCGGTGCCTAGAGAAGGTGGCGCGGGGTAAACTGGG | |
| | AAAGTGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCCGCCA | |
| | GAACACGCGTAAG | |
| Promoter | GCTCCGGTGCCCGTCAGTGGGCAGAGCGCACATCGCCCACAGTCCCCGAGAAGT | 1881 |
| Variant 9 | TGGGGGGAGGGGTCGGCAATTGAACCGGTGCCTAGAGAAGGTGGCGCGGGGTAA | |
| | ACTGGGAAAGTGATGTCGTGTACTGGCTCCGCCTTTTTCCCGAGGGTGGGGGAG | |
| | AACCGTATATAAGTGCAGTAGTCGCCGTGAACGTTCTTTTTCGCAACGGGTTTG | |
| | CCGCCAGAACACGCGTAAG | |
| Promoter | CGTGAGGCTCCGGTGCCCGTCAGTGGGCAGAGCGCACATCGCCCACAGTCCCCG | 1882 |
| Variant 10 | AGAAGTTGGGGGGAGGGGTCGGCAATTGAACCGGTGCCTAGAGAAGGTGGCGCG | |
| | GGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACG | |
| | GGTTTGCCGCCAGAACACGCGTAAG | |
| Promoter | CGTGAGGCTCCGGTGCCCGTCAGTGGGCAGAGCGCACATCGCCCACAGTCCCCG | 1883 |
| Variant 11 | AGAAGTTGGGGGGAGGGGTCGGCAATTGAACCGGTGCCTAGAGAAGGTGGCGCG | |
| | GGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACG | |
| | GGTTTGCCGCCAGAACACAG | |
| Promoter | GCATGCGTGAGGCTCCGGTGCCCGTCAGTGGGCAGAGCGCACATCGCCCACAGT | 1884 |
| Variant | CCCCGAGAAGTTGGGGGGAGGGGTCGGCAATTGAACCGGTGCCTAGAGAAGGTG | |
| 12 | GCGCGGGGTAAACTGGGAAAGTGATGTCGTGTACTGGCTCCGCCTTTTTCCCGA | |
| | GGGTGGGGGAGAACCGTATATAAGTGCAGTAGTCGCCGTGAACGTTCTTTTTCG | |
| | CAACGGGTTTGCCGCCAGAACACGCGTAAG | |
| Promoter | GCATGCGTGAGGCTCCGGTGCCCGTCAGTGGGCAGAGCGCACATCGCCCACAGT | 1885 |
| Variant 13 | CCCCGAGAAGTTGGGGGGAGGGGTCGGCAATTGAACCGGTGCCTAGAGAAGGTG | |
| | GCGCGGGGTAAACTGGGAAAGTGATGTCGTGTACTGGCTCCGCCTTTTTCCCGA | |
| | GGGTGGGGGAGAACCGTATATAAGTGCAGTAGTCGCCGTGAACGTTCTTTTTCG | |
| | CAACGGGTTTGCCGCCAGAACACAG | |
| Promoter | GGTGGAGAAGAGCATGCGTGAGGCTCCGGTGCCCGTCAGTGGGCAGAGCGCACA | 1886 |
| Variant 14 | TCGCCCACAGTCCCCGAGAAGTTGGGGGGAGGGGTCGGCAATTGAACCGGTGCC | |
| | TAGAGAAGGTGGCGCGGGGTAAACTGGGAAAGTGATGTCGTGTACTGGCTCCGC | |
| | CTTTTTCCCGAGGGTGGGGGAGAACCGTATATAAGTGCAGTAGTCGCCGTGAAC | |
| | GTTCTTTTTCGCAACGGGTTTGCCGCCAGAACACGCGTAAG | |
| Promoter | GGTGGAGAAGAGCATGCGTGAGGCTCCGGTGCCCGTCAGTGGGCAGAGCGCACA | 1887 |
| Variant 15 | TCGCCCACAGTCCCCGAGAAGTTGGGGGGAGGGGTCGGCAATTGAACCGGTGCC | |
| | TAGAGAAGGTGGCGCGGGGTAAACTGGGAAAGTGATGTCGTGTACTGGCTCCGC | |
| | CTTTTTCCCGAGGGTGGGGGAGAACCGTATATAAGTGCAGTAGTCGCCGTGAAC | |
| | GTTCTTTTTCGCAACGGGTTTGCCGCCAGAACACAG | |
| Promoter | GTCATCACTGAGGTGGAGAAGAGCATGCGTGAGGCTCCGGTGCCCGTCAGTGGG | 1888 |
| Variant 16 | CAGAGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCGTGT | |
| | ACTGGCTCCGCCTTTTTCCCGAGGGTGGGGGAGAACCGTATATAAGTGCAGTAG | |
| | TCGCCGTGAACGTTCTTTTTCGCAACGGGTTTGCCGCCAGAACACGCGTAAG | |
| Promoter | GTCATCACTGAGGTGGAGAAGAGCATGCGTGAGGCTCCGGTGCCCGTCAGTGGG | 1889 |
| Variant | CAGAGCGCACATCGCCCACAGTCCCCGAGAAGTTGGGGGGAGGGGTCGGCAATT | |
| 18 | GAACCGGTGCCTAGAGAAGGTGGCGCGGGGTAAACTGGGAAAGTGATGTCGTGT | |
| | ACTGGCTCCGCCTTTTTCCCGAGGGTGGGGGAGAACCGTATATAAGTGCAGTAG | |
| | TCGCCGTGAACGTTCTTTTTCGCAACGGGTTTGCCGCCAGAACACAG | |

### Viral Genome Component: Introns

In some embodiments, the viral genome comprises an element to enhance the payload target specificity and expression (See e.g., Powell et al. Viral Expression Cassette Elements to Enhance Transgene Target Specificity and Expression in Gene Therapy, Discov. Med, 2015, 19(102):49-57; the contents of which are herein incorporated by reference in their entirety), such as an intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps). In some embodiments, the intron comprises a human beta-globin intron or a variant thereof.

In some embodiments, the intron comprises the nucleotide sequence of SEQ ID NO: 4475, or a nucleotide sequence at least 70%, 75%, 80%, 85%, 90%, 92%, 95%, 96%, 97%, 98%, or 99% identical thereto.

### Viral Genome Component: Untranslated Regions (UTRs)

In some embodiments, a wild type untranslated region (UTR) of a gene is transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the disclosure to enhance expression in hepatic cell lines or liver.

In some embodiments, the viral genome encoding a transgene described herein (e.g., a transgene encoding a GBA protein) comprises a Kozak sequence. While not wishing to be bound by theory, wild-type 5′ untranslated regions (UTRs) include features that play roles in translation initiation. Kozak sequences, which are commonly known to be involved in the process by which the ribosome initiates translation of many genes, are usually included in 5′ UTRs. Kozak sequences have the consensus CCR (A/G) CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

While not wishing to be bound by theory, wild-type 3′ UTRs are known to have stretches of adenosines and uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in their entirety): Class I AREs, such as, but not limited to, c-Myc and MyoD, contain several dispersed copies of an AUUUA motif within U-rich regions. Class II AREs, such as, but not limited to, GM-CSF and TNF-a, possess two or more overlapping UUAUUUA (U/A) (U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo (dT) sequence for templated addition of a poly-A tail.

Any UTR from any gene known in the art may be incorporated into the viral genome of the AAV particle. These UTRs, or portions thereof, may be placed in the same orientation as in the gene from which they were selected or they may be altered in orientation or location. In some embodiments, the UTR used in the viral genome of the AAV particle may be inverted, shortened, lengthened, or made with one or more other 5′ UTRs or 3′ UTRs known in the art. As used herein, the term “altered,” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTR, which is not a variant of a wild type UTR.

In some embodiments, the viral genome of the AAV particle comprises UTRs which have been selected from a family of transcripts whose proteins share a common function, structure, feature, or property.

### Viral Genome Component: miR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed. In some embodiments, the miR binding site modulates, e.g., reduces, expression of the encoded GBA protein in a cell or tissue of the DRG, liver, hematopoietic lineage, or a combination thereof.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleic acid sequence encoding a payload), e.g., before the polyA sequence, 5′-UTR region of the viral genome (e.g., 5′ relative to the nucleic acid sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, the encoded miR binding site series comprises 4 copies of a miR binding site. In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer is about 8 nucleotides in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complementary), to the miR in the host cell. In some embodiments, the sequence complementary to the nucleotide sequence encoding the miR binding site comprises at least 1, 2, 3, 4, or 5 mismatches or no more than 6, 7, 8, 9, or 10 mismatches relative to the corresponding miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complementary to the miR in the host cell.

In some embodiments, the encoded miR binding site or the encoded miR binding site series is about 10 to about 125 nucleotides in length, e.g., about 10 to 50 nucleotides, 10 to 100 nucleotides, 50 to 100 nucleotides, 50 to 125 nucleotides, or 100 to 125 nucleotides in length. In some embodiments, an encoded miR binding site or the encoded miR binding site series is about 7 to about 28 nucleotides in length, e.g., about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., full complementary or partially complementary) to the seed sequence of a miRNA (e.g., a miR122, a miR 142, a miR-1, a miR183).

In some embodiments, the encoded miR binding site is complementary (e.g., fully complementary or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 1865), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1865, e.g., wherein the modification can result in a mismatch between the encoded miR binding site and the corresponding miRNA. In some embodiments, the viral genome comprises at least 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCACACTCCA (SEQ ID NO: 1866), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1866, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR 122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 5025), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 5025, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully complementary or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site is complementary (e.g., fully complementary or partially complementary) to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 1869), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1842, e.g., wherein the modification can result in a mismatch between the encoded miR binding site and the corresponding miRNA. In some embodiments, the viral genome comprises at least 3, 4, or 5 copies of an encoded miR-142-3p binding site, e.g., an encoded miR-142-3p binding site series. In some embodiments, the at least 3, 4, or 5 copies (e.g., 4 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary (e.g., fully complementary or partially complementary) to a miR expressed in expressed in a DRG neuron. In some embodiments, the encoded miR binding site comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 1847), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1847, e.g., wherein the modification can result in a mismatch between the encoded miR binding site and the corresponding miRNA. In some embodiments, the sequence complementary (e.g., fully complementary or partially complementary) to the seed sequence corresponds to the double underlined of the encoded miR-183 binding site sequence. In some embodiments, the viral genome comprises at least comprises at least 3, 4, or 5 copies (e.g., 4 copies) of the encoded miR183 binding site, e.g. an encoded miR183 binding site. In some embodiments, the viral genome comprises at least comprises 4 copies of the encoded miR183 binding site, e.g. an encoded miR183 binding site comprising 4 copies of a miR183 binding site. In some embodiments, the at least 3, 4, or 5 copies (e.g., 4 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the encoded miR183 binding site series comprises the nucleotide sequence of SEQ ID NO: 1849, or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1849.

In some embodiments, the encoded miR binding site or encoded miR binding site series comprises a miR182 binding site sequence. In some embodiments, the encoded miR182 binding site comprises, the nucleotide sequence of AGTGTGAGTTCTACCATTGCCAAA (SEQ ID NO: 1867), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1867, e.g., wherein the modification can result in a mismatch between the encoded miR binding site and the corresponding miRNA. In some embodiments, the viral genome comprises at least 3, 4, or 5 copies of the encoded miR182 binding site, e.g., an encoded miR182 binding site series. In some embodiments, the at least 3, 4, or 5 copies (e.g., 4 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA.

In some embodiments, the encoded miR binding site or encoded miR binding site series comprises a miR96 binding site sequence. In some embodiments, the encoded miR96 binding site comprises the nucleotide sequence of AGCAAAAATGTGCTAGTGCCAAA (SEQ ID NO: 1868), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1868, e.g., wherein the modification can result in a mismatch between the encoded miR binding site and the corresponding miRNA. In some embodiments, the viral genome comprises at least 3, 4, or 5 copies of the encoded miR96 binding site, e.g., an encoded miR96 binding site series. In some embodiments, the at least 3, 4, or 5 copies (e.g., 4 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA.

In some embodiments, the encoded miR binding site series comprises a miR122 binding site, a miR142 binding site, a miR183 binding site, a miR182 binding site, a miR 96 binding site, or a combination thereof. In some embodiments, the encoded miR binding site series comprises at least 3, 4, or 5 copies of a miR122 binding site, a miR-1, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, an encoded miR binding site series comprises at least 3-5 copies (e.g., 4 copies) of a combination of at least two, three, four, five, or all of a miR122 binding site, a miR-1 a miR142 binding site, a miR183 binding site, a miR182 binding site, a miR96 binding site, wherein each of the miR binding sites within the series are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

### Viral Genome Component: Filler Sequence

In some embodiments, the viral genome of an AAV particle described herein comprises an element to improve packaging efficiency and expression, such as a filler or stuffer sequence. Non-limiting examples of filler or stuffer sequences include albumin and/or alpha-1 antitrypsin. Any known viral, mammalian, or plant sequence may be manipulated for use as a filler sequence. In some embodiments, the filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides.

### Viral Genome Component: Polyadenylation Sequence

The viral genome comprises a polyadenylation (polyA) sequence. In some embodiments, the viral genome of the AAV particle (e.g., an AAV particle comprising an AAV capsid variant, described herein) comprises a polyadenylation sequence between the 3′ end of the nucleotide sequence encoding the payload and the 5′ end of the 3′ITR. In some embodiments, the polyA sequence comprises the nucleotide sequence of SEQ ID NO: 4476, or a nucleotide sequence at least 70%, 75%, 80%, 85%, 90%, 92%, 95%, 96%, 97%, 98%, or 99% identical thereto.

### Payloads

In some embodiments, an AAV particle, e.g., an AAV particle for the vectorized delivery of a siRNA, e.g., a MAPT targeting siRNA described herein, comprises a payload. In some embodiments, an AAV particle, e.g., an AAV particle for the vectorized delivery of a siRNA described herein (e.g., a MAPT targeting siRNA), comprises a viral genome encoding a payload. In some embodiments, the viral genome comprises a promoter operably linked to a nucleic acid comprising a transgene encoding a payload. In some embodiments, the payload comprises a siRNA or a modulatory polynucleotide comprising or encoding an siRNA duplex.

In some aspects, the present disclosure relates to a composition containing or comprising a nucleic acid sequence encoding a MAPT targeting siRNA or functional fragment or variants thereof and methods of administering the composition in vitro or in vivo in a subject, e.g., a humans and/or an animal model of disease, e.g., a disease related to expression of MAPT.

AAV particles of the present disclosure may comprise a nucleic acid sequence encoding at least one payload. As used herein, payload or payload region refers to one or more polynucleotides or polynucleotide regions encoded by or within a viral genome or an expression product of such polynucleotide or polynucleotide region, e.g., a transgene encoding a siRNA duplex, a modulatory polynucleotide, or a gene editing system for inhibiting expression of MAPT, or fragment or variant thereof. The payload may comprise any nucleic acid known in the art that is useful for the expression (by supplementation of the MAPT siRNA or gene replacement using a modulatory nucleic acid) of the MAPT targeting siRNA in a target cell transduced or contacted with the AAV particle carrying the payload.

### MAPT

AAV particle viral genomes of the present disclosure may comprise a payload region, which when expressed, results in the silencing, suppression, and/or reduction of the MAPT gene expression, corresponding transcript expression, and/or protein production.

The present disclosure provides small interfering RNA (siRNA) duplexes (and modulatory polynucleotides comprising or encoding them) that target MAPT transcript to modulate, e.g., inhibit or silence MAPT gene expression, mRNA production, and/or protein production. In some embodiments, the MAPT gene is a wild type gene. In some embodiments, the MAPT gene comprises at least one, e.g., one or more mutations, e.g., a mutated MAPT gene. In some embodiments, the MAPT gene to be targeted by a siRNA duplex as described herein comprises a mutation that results in the production of a tau protein comprising one, two, or all of a mutation at position 272, 301, and/or 406, e.g., G272V, P301L, and/or R406W.

In some embodiments, the MAPT gene to be targeted by a siRNA described herein is provided in Table 3. In some embodiments, a siRNA for targeting MAPT described herein targets a MAPT gene comprising a nucleotide sequence provided in Table 3 or a fragment or variant thereof (e.g., the nucleotide sequence of any one of SEQ ID NOs: 3944-3951 or a nucleotide sequence at least 70%, 75%, 80%, 85%, 90%, 95%, 97%, or 99% identical thereto). In some embodiments, the siRNA for targeting MAPT as described herein modulates the level of a protein comprising the amino acid sequence of any one of SEQ ID Nos. 3936-3943, or a fragment or variant thereof (e.g., an amino acid sequence at least 70%, 75%, 80%, 85%, 90%, 95%, 97%, or 99% identical to SEQ ID NOs: 3936-3943).

TABLE 3
Exemplary MAPT Sequences

| | | | SEQ |
|---|---|---|---|
| ID | Sequence | Type | ID NO |
| MAPT SEQ-001 | NP_058519.3 | PRT | 3936 |
| MAPT SEQ-002 | NP_005901.2 | PRT | 3937 |
| MAPT SEQ-003 | NP_058518.1 | PRT | 3938 |
| MAPT SEQ-004 | NP_058525.1 | PRT | 3939 |
| MAPT SEQ-005 | NP_001116539.1 | PRT | 3940 |
| MAPT SEQ-006 | NP_001116538.2 | PRT | 3941 |
| MAPT SEQ-007 | NP_001190180.1 | PRT | 3942 |
| MAPT SEQ-008 | NP_001190181.1 | PRT | 3943 |
| MAPT SEQ-009 | NM_016835.4 | DNA | 3944 |
| MAPT SEQ-010 | NM_005910.5 | DNA | 3945 |
| MAPT SEQ-011 | NM_016834.4 | DNA | 3946 |
| MAPT SEQ-012 | NM_016841.4 | DNA | 3947 |
| MAPT SEQ-013 | NM_001123067.3 | DNA | 3948 |
| MAPT SEQ-014 | NM_001123066.3 | DNA | 3949 |
| MAPT SEQ-015 | NM_001203251.1 | DNA | 3950 |
| MAPT SEQ-016 | NM_001203252.1 | DNA | 3951 |
| MAPT SEQ-017 | NM_001123066.4 | DNA | 5024 |

### Modulatory Polynucleotides

In some embodiments, modulatory polynucleotides, e.g., RNA or DNA molecules, may be used to treat a neurodegenerative disease, e.g., a tauopathy, e.g., Alzheimer's disease (AD) and frontotemporal dementia (FTD). In some embodiments, the tauopathy may be a neurodegenerative disease. In some embodiments, the tauopathy may be a non-neurodegenerative disease without neurodegeneration. In some embodiments, the tauopathy is a familial tauopathy (e.g., genetic) or a sporadic tauopathy (e.g., idiopathic). In some embodiments, a modulatory polynucleotide described herein, e.g., an RNA or DNA molecule, is used to treat epilepsy or Dravet syndrome (DS).

In some embodiments, the modulatory polynucleotides may comprise a nucleic acid sequence encoding a siRNA molecule. In some embodiments, the modulatory polynucleotides may comprise a nucleic acid sequence encoding at least 1, 2, 3, 4, 5, 6, 7, 8, or 9 siRNA molecules. In some embodiments the modulatory polynucleotide comprises a siRNA molecule, e.g., an siRNA molecule described herein.

siRNA Molecules for Targeting MAPT

The present disclosure relates to RNA interference (RNAi) agents, e.g., a siRNA, that modulate (e.g., inhibit) expression of MAPT. In some embodiments, an siRNA molecule described herein modulates, reduces and/or silences, e.g., inhibits a MAPT gene, mRNA, and or protein expression. In some embodiments, a siRNA molecule described herein modulates, reduces, and/or silences, e.g., inhibits, MAPT transcript expression, gene expression and/or protein production in cortical neurons, thereby ameliorating symptoms of Alzheimer's disease (AD) and/or frontotemporal dementia (FTD). In some embodiments, a siRNA molecule described herein modulates, reduces, and/or silences, e.g., inhibits, MAPT transcript expression, gene expression and/or protein production in interneurons and/or hippocampal neurons, thereby ameliorating symptoms of Dravet syndrome (DS). In some embodiments, the hippocampal neuron is a dentate gyrus granule cell neuron.

RNAi (also known as post-transcriptional gene silencing (PTGS), quelling, or co-suppression) is a post-transcriptional gene silencing process in which RNA molecules, in a sequence-specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs, and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs-containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA-mediated downregulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes (e.g., as described in Elbashir S M et al., Nature, 2001, 411, 494-498, the contents of which are hereby incorporated by reference in their entirety).

In vitro synthetized siRNA molecules may be introduced into cells to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target RNA transcript and an antisense strand that is complementary to the target RNA transcript offers much more advantage in terms of efficiency for target RNA destruction compared to the use of the single strand (ss)-siRNAs (e.g., antisense strand RNA or antisense oligonucleotides). In many cases, it requires higher concentration of the ss-siRNA to achieve the effective gene silencing potency of the corresponding duplex.

While not wishing to be bound by theory, a vector genome may encode a pri-miRNA or pre-miRNA, which when expressed in the target cells is processed via the cell's endogenous pathway to a miRNA duplex. Once the miRNA duplex unwinds, the guide strand may bind to its complementary mRNA substrate and recruit the RNA-induced silencing complex, which leads to the cleavage of the substrate mRNA. Hence, delivery of an AAV particle comprising a vector genome encoding an RNAi precursor (pre- or pri-miR), enters the processing pathway at a different step than would occur with direct administration of an shRNA or siRNA, for example.

Any of the foregoing molecules may be encoded by an AAV vector or vector genome.

In some embodiments, a nucleic acid sequence encoding such siRNA molecules, or a single strand of the siRNA molecules, is inserted into the vector genome of an AAV particle and introduced into cells, specifically neurons, including interneurons, and/or other surrounding cells in the central nervous system.

According to the present disclosure, siRNA molecules (e.g., siRNA duplexes or encoded dsRNA) that target the MAPT transcript may be designed which can modulate, e.g., suppress, inhibit, or silence MAPT gene expression, mRNA expression and/or protein expression. In some embodiments, the siRNA molecule is able to modulate, e.g., inhibit, expression of a wild type MAPT gene, mRNA, and/or protein. In some embodiments, the siRNA molecule is able to modulate, e.g., inhibit, expression of a MAPT gene, mRNA, and/or protein comprising at least one mutation, e.g., a mutated MAPT gene, mRNA, and/or protein. In some embodiments, the mutation is a mutation associate with a tauopathy, e.g., frontotemporal dementia and parkinsonism linked to chromosome 17 (FTDP-17). In some embodiments, the MAPT is a human MAPT. In some embodiments, the MAPT sequence, e.g., the MAPT sequence targeted by an siRNA described herein, comprises the nucleotide sequence of NM_001123066.4, which comprises the nucleotide sequence of:

(SEQ ID NO: 5024)
GCAGTCACCGCCACCCACCAGCTCCGGCACCAACAGCAGCGCCGCTGCCACCGCCCACCTTCTGCCGCCGCCACCACAGC
CACCTTCTCCTCCTCCGCTGTCCTCTCCCGTCCTCGCCTCTGTCGACTATCAGGTGAACTTTGAACCAGGATGGCTGAGC
CCCGCCAGGAGTTCGAAGTGATGGAAGATCACGCTGGGACGTACGGGTTGGGGGACAGGAAAGATCAGGGGGGCTACACC
ATGCACCAAGACCAAGAGGGTGACACGGACGCTGGCCTGAAAGAATCTCCCCTGCAGACCCCCACTGAGGACGGATCTGA
GGAACCGGGCTCTGAAACCTCTGATGCTAAGAGCACTCCAACAGCGGAAGATGTGACAGCACCCTTAGTGGATGAGGGAG
CTCCCGGCAAGCAGGCTGCCGCGCAGCCCCACACGGAGATCCCAGAAGGAACCACAGCTGAAGAAGCAGGCATTGGAGAC
ACCCCCAGCCTGGAAGACGAAGCTGCTGGTCACGTGACCCAAGAGCCTGAAAGTGGTAAGGTGGTCCAGGAAGGCTTCCT
CCGAGAGCCAGGCCCCCCAGGTCTGAGCCACCAGCTCATGTCCGGCATGCCTGGGGCTCCCCTCCTGCCTGAGGGCCCCA
GAGAGGCCACACGCCAACCTTCGGGGACAGGACCTGAGGACACAGAGGGCGGCCGCCACGCCCCTGAGCTGCTCAAGCAC
CAGCTTCTAGGAGACCTGCACCAGGAGGGGCCGCCGCTGAAGGGGGCAGGGGGCAAAGAGAGGCCGGGGAGCAAGGAGGA
GGTGGATGAAGACCGCGACGTCGATGAGTCCTCCCCCCAAGACTCCCCTCCCTCCAAGGCCTCCCCAGCCCAAGATGGGC
GGCCTCCCCAGACAGCCGCCAGAGAAGCCACCAGCATCCCAGGCTTCCCAGCGGAGGGTGCCATCCCCCTCCCTGTGGAT
TTCCTCTCCAAAGTTTCCACAGAGATCCCAGCCTCAGAGCCCGACGGGCCCAGTGTAGGGCGGGCCAAAGGGCAGGATGC
CCCCCTGGAGTTCACGTTTCACGTGGAAATCACACCCAACGTGCAGAAGGAGCAGGCGCACTCGGAGGAGCATTTGGGAA
GGGCTGCATTTCCAGGGGCCCCTGGAGAGGGGCCAGAGGCCCGGGGCCCCTCTTTGGGAGAGGACACAAAAGAGGCTGAC
CTTCCAGAGCCCTCTGAAAAGCAGCCTGCTGCTGCTCCGCGGGGGAAGCCCGTCAGCCGGGTCCCTCAACTCAAAGCTCG
CATGGTCAGTAAAAGCAAAGACGGGACTGGAAGCGATGACAAAAAAGCCAAGACATCCACACGTTCCTCTGCTAAAACCT
TGAAAAATAGGCCTTGCCTTAGCCCCAAACACCCCACTCCTGGTAGCTCAGACCCTCTGATCCAACCCTCCAGCCCTGCT
GTGTGCCCAGAGCCACCTTCCTCTCCTAAATACGTCTCTTCTGTCACTTCCCGAACTGGCAGTTCTGGAGCAAAGGAGAT
GAAACTCAAGGGGGCTGATGGTAAAACGAAGATCGCCACACCGCGGGGAGCAGCCCCTCCAGGCCAGAAGGGCCAGGCCA
ACGCCACCAGGATTCCAGCAAAAACCCCGCCCGCTCCAAAGACACCACCCAGCTCTGCGACTAAGCAAGTCCAGAGAAGA
CCACCCCCTGCAGGGCCCAGATCTGAGAGAGGTGAACCTCCAAAATCAGGGGATCGCAGCGGCTACAGCAGCCCCGGCTC
CCCAGGCACTCCCGGCAGCCGCTCCCGCACCCCGTCCCTTCCAACCCCACCCACCCGGGAGCCCAAGAAGGTGGCAGTGG
TCCGTACTCCACCCAAGTCGCCGTCTTCCGCCAAGAGCCGCCTGCAGACAGCCCCCGTGCCCATGCCAGACCTGAAGAAT
GTCAAGTCCAAGATCGGCTCCACTGAGAACCTGAAGCACCAGCCGGGAGGCGGGAAGGTGCAGATAATTAATAAGAAGCT
GGATCTTAGCAACGTCCAGTCCAAGTGTGGCTCAAAGGATAATATCAAACACGTCCCGGGAGGCGGCAGTGTGCAAATAG
TCTACAAACCAGTTGACCTGAGCAAGGTGACCTCCAAGTGTGGCTCATTAGGCAACATCCATCATAAACCAGGAGGTGGC
CAGGTGGAAGTAAAATCTGAGAAGCTTGACTTCAAGGACAGAGTCCAGTCGAAGATTGGGTCCCTGGACAATATCACCCA
CGTCCCTGGCGGAGGAAATAAAAAGATTGAAACCCACAAGCTGACCTTCCGCGAGAACGCCAAAGCCAAGACAGACCACG
GGGCGGAGATCGTGTACAAGTCGCCAGTGGTGTCTGGGGACACGTCTCCACGGCATCTCAGCAATGTCTCCTCCACCGGC
AGCATCGACATGGTAGACTCGCCCCAGCTCGCCACGCTAGCTGACGAGGTGTCTGCCTCCCTGGCCAAGCAGGGTTTGTG
ATCAGGCCCCTGGGGCGGTCAATAATTGTGGAGAGGAGAGAATGAGAGAGTGTGGAAAAAAAAAGAATAATGACCCGGCC
CCCGCCCTCTGCCCCCAGCTGCTCCTCGCAGTTCGGTTAATTGGTTAATCACTTAACCTGCTTTTGTCACTCGGCTTTGG
CTCGGGACTTCAAAATCAGTGATGGGAGTAAGAGCAAATTTCATCTTTCCAAATTGATGGGTGGGCTAGTAATAAAATAT
TTAAAAAAAAACATTCAAAAACATGGCCACATCCAACATTTCCTCAGGCAATTCCTTTTGATTCTTTTTTCTTCCCCCTC
CATGTAGAAGAGGGAGAAGGAGAGGCTCTGAAAGCTGCTTCTGGGGGATTTCAAGGGACTGGGGGTGCCAACCACCTCTG
GCCCTGTTGTGGGGGTGTCACAGAGGCAGTGGCAGCAACAAAGGATTTGAAACTTGGTGTGTTCGTGGAGCCACAGGCAG
ACGATGTCAACCTTGTGTGAGTGTGACGGGGGTTGGGGTGGGGGGGGAGGCCACGGGGGAGGCCGAGGCAGGGGCTGGGC
AGAGGGGAGAGGAAGCACAAGAAGTGGGAGTGGGAGAGGAAGCCACGTGCTGGAGAGTAGACATCCCCCTCCTTGCCGCT
GGGAGAGCCAAGGCCTATGCCACCTGCAGCGTCTGAGCGGCCGCCTGTCCTTGGTGGCCGGGGGTGGGGGCCTGCTGTGG
GTCAGTGTGCCACCCTCTGCAGGGCAGCCTGTGGGAGAAGGGACAGCGGGTAAAAAGAGAAGGCAAGCTGGCAGGAGGGT
GGCACTTCGTGGATGACCTCCTTAGAAAAGACTGACCTTGATGTCTTGAGAGCGCTGGCCTCTTCCTCCCTCCCTGCAGG
GTAGGGGGCCTGAGTTGAGGGGCTTCCCTCTGCTCCACAGAAACCCTGTTTTATTGAGTTCTGAAGGTTGGAACTGCTGC
CATGATTTTGGCCACTTTGCAGACCTGGGACTTTAGGGCTAACCAGTTCTCTTTGTAAGGACTTGTGCCTCTTGGGAGAC
GTCCACCCGTTTCCAAGCCTGGGCCACTGGCATCTCTGGAGTGTGTGGGGGTCTGGGAGGCAGGTCCCGAGCCCCCTGTC
CTTCCCACGGCCACTGCAGTCACCCCGTCTGCGCCGCTGTGCTGTTGTCTGCCGTGAGAGCCCAATCACTGCCTATACCC
CTCATCACACGTCACAATGTCCCGAATTCCCAGCCTCACCACCCCTTCTCAGTAATGACCCTGGTTGGTTGCAGGAGGTA
CCTACTCCATACTGAGGGTGAAATTAAGGGAAGGCAAAGTCCAGGCACAAGAGTGGGACCCCAGCCTCTCACTCTCAGTT
CCACTCATCCAACTGGGACCCTCACCACGAATCTCATGATCTGATTCGGTTCCCTGTCTCCTCCTCCCGTCACAGATGTG
AGCCAGGGCACTGCTCAGCTGTGACCCTAGGTGTTTCTGCCTTGTTGACATGGAGAGAGCCCTTTCCCCTGAGAAGGCCT
GGCCCCTTCCTGTGCTGAGCCCACAGCAGCAGGCTGGGTGTCTTGGTTGTCAGTGGTGGCACCAGGATGGAAGGGCAAGG
CACCCAGGGCAGGCCCACAGTCCCGCTGTCCCCCACTTGCACCCTAGCTTGTAGCTGCCAACCTCCCAGACAGCCCAGCC
CGCTGCTCAGCTCCACATGCATAGTATCAGCCCTCCACACCCGACAAAGGGGAACACACCCCCTTGGAAATGGTTCTTTT
CCCCCAGTCCCAGCTGGAAGCCATGCTGTCTGTTCTGCTGGAGCAGCTGAACATATACATAGATGTTGCCCTGCCCTCCC
CATCTGCACCCTGTTGAGTTGTAGTTGGATTTGTCTGTTTATGCTTGGATTCACCAGAGTGACTATGATAGTGAAAAGAA
AAAAAAAAAAAAAAAAGGACGCATGTATCTTGAAATGCTTGTAAAGAGGTTTCTAACCCACCCTCACGAGGTGTCTCTCA
CCCCCACACTGGGACTCGTGTGGCCTGTGTGGTGCCACCCTGCTGGGGCCTCCCAAGTTTTGAAAGGCTTTCCTCAGCAC
CTGGGACCCAACAGAGACCAGCTTCTAGCAGCTAAGGAGGCCGTTCAGCTGTGACGAAGGCCTGAAGCACAGGATTAGGA
CTGAAGCGATGATGTCCCCTTCCCTACTTCCCCTTGGGGCTCCCTGTGTCAGGGCACAGACTAGGTCTTGTGGCTGGTCT
GGCTTGCGGCGCGAGGATGGTTCTCTCTGGTCATAGCCCGAAGTCTCATGGCAGTCCCAAAGGAGGCTTACAACTCCTGC
ATCACAAGAAAAAGGAAGCCACTGCCAGCTGGGGGGATCTGCAGCTCCCAGAAGCTCCGTGAGCCTCAGCCACCCCTCAG
ACTGGGTTCCTCTCCAAGCTCGCCCTCTGGAGGGGCAGCGCAGCCTCCCACCAAGGGCCCTGCGACCACAGCAGGGATTG
GGATGAATTGCCTGTCCTGGATCTGCTCTAGAGGCCCAAGCTGCCTGCCTGAGGAAGGATGACTTGACAAGTCAGGAGAC
ACTGTTCCCAAAGCCTTGACCAGAGCACCTCAGCCCGCTGACCTTGCACAAACTCCATCTGCTGCCATGAGAAAAGGGAA
GCCGCCTTTGCAAAACATTGCTGCCTAAAGAAACTCAGCAGCCTCAGGCCCAATTCTGCCACTTCTGGTTTGGGTACAGT
TAAAGGCAACCCTGAGGGACTTGGCAGTAGAAATCCAGGGCCTCCCCTGGGGCTGGCAGCTTCGTGTGCAGCTAGAGCTT
TACCTGAAAGGAAGTCTCTGGGCCCAGAACTCTCCACCAAGAGCCTCCCTGCCGTTCGCTGAGTCCCAGCAATTCTCCTA
AGTTGAAGGGATCTGAGAAGGAGAAGGAAATGTGGGGTAGATTTGGTGGTGGTTAGAGATATGCCCCCCTCATTACTGCC
AACAGTTTCGGCTGCATTTCTTCACGCACCTCGGTTCCTCTTCCTGAAGTTCTTGTGCCCTGCTCTTCAGCACCATGGGC
CTTCTTATACGGAAGGCTCTGGGATCTCCCCCTTGTGGGGCAGGCTCTTGGGGCCAGCCTAAGATCATGGTTTAGGGTGA
TCAGTGCTGGCAGATAAATTGAAAAGGCACGCTGGCTTGTGATCTTAAATGAGGACAATCCCCCCAGGGCTGGGCACTCC
TCCCCTCCCCTCACTTCTCCCACCTGCAGAGCCAGTGTCCTTGGGTGGGCTAGATAGGATATACTGTATGCCGGCTCCTT
CAAGCTGCTGACTCACTTTATCAATAGTTCCATTTAAATTGACTTCAGTGGTGAGACTGTATCCTGTTTGCTATTGCTTG
TTGTGCTATGGGGGGAGGGGGGAGGAATGTGTAAGATAGTTAACATGGGCAAAGGGAGATCTTGGGGTGCAGCACTTAAA
CTGCCTCGTAACCCTTTTCATGATTTCAACCACATTTGCTAGAGGGAGGGAGCAGCCACGGAGTTAGAGGCCCTTGGGGT
TTCTCTTTTCCACTGACAGGCTTTCCCAGGCAGCTGGCTAGTTCATTCCCTCCCCAGCCAGGTGCAGGCGTAGGAATATG
GACATCTGGTTGCTTTGGCCTGCTGCCCTCTTTCAGGGGTCCTAAGCCCACAATCATGCCTCCCTAAGACCTTGGCATCC
TTCCCTCTAAGCCGTTGGCACCTCTGTGCCACCTCTCACACTGGCTCCAGACACACAGCCTGTGCTTTTGGAGCTGAGAT
CACTCGCTTCACCCTCCTCATCTTTGTTCTCCAAGTAAAGCCACGAGGTCGGGGCGAGGGCAGAGGTGATCACCTGCGTG
TCCCATCTACAGACCTGCAGCTTCATAAAACTTCTGATTTCTCTTCAGCTTTGAAAAGGGTTACCCTGGGCACTGGCCTA
GAGCCTCACCTCCTAATAGACTTAGCCCCATGAGTTTGCCATGTTGAGCAGGACTATTTCTGGCACTTGCAAGTCCCATG
ATTTCTTCGGTAATTCTGAGGGTGGGGGGAGGGACATGAAATCATCTTAGCTTAGCTTTCTGTCTGTGAATGTCTATATA
GTGTATTGTGTGTTTTAACAAATGATTTACACTGACTGTTGCTGTAAAAGTGAATTTGGAAATAAAGTTATTACTCTGAT
TAAA.

In some embodiments, an siRNA molecule of the present disclosure comprises a sense strand and a complementary antisense strand in which both strands are hybridized together to form a duplex structure. The antisense strand may have sufficient complementarity to the MAPT transcript sequence to direct target-specific RNAi to the target mRNA, e.g., the siRNA molecule has a sequence sufficient to trigger the destruction of the target transcript by the RNAi machinery or process.

In some embodiments, an siRNA molecule of the present disclosure comprises a sense strand and a corresponding antisense strand. In some embodiments, the antisense strand is complementary, e.g., substantially complementary or fully complementary to at least a portion of a MAPT gene, e.g., a MAPT gene as described in Table 3 or 19, or a variant thereof (e.g., a human MAPT). In some embodiments, the antisense strand is complementary, e.g., partially complementary or fully complementary, to a region of the MAPT transcript, e.g., a region between nucleotide 1 and 6850 on the MAPT transcript sequence, e.g., as described in Table 19. As a non-limiting example, the region of complementary comprises nucleotides 1-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500, 4500-4550, 4550-4600, 4600-4650, 4650-4700, 4700-4750, 4750-4800, 4800-4850, 4850-4900, 4900-4950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, or 6800-6850 of the MAPT transcript sequence, e.g., as described in Table 3 or 19, e.g., SEQ ID NO: 5024.

In some embodiments, the antisense strand sequence comprises a region of complementarity to the target sequence (e.g., a MAPT target), which is 15-30, 19-21, or 25-30 nucleotides in length, e.g., 15, 16, 17, 18, 19, 20, 21, 22, 23, 24, 25, 26, 27, 28, 29, or 30 nucleotides in length. In some embodiments, the antisense strand sequence is fully complementary to the target sequence (e.g., 100% complementary). In some embodiments, the antisense strand sequence is substantially complementary to the target sequence. In some embodiments, the antisense strand sequence comprises a region of complementarity to a portion of a the target sequence (e.g., a MAPT sequence provided in Table 3 or 19 or a variant thereof), e.g., a portion of at least 15, 16, 17, 18, 19, 20, or 21 contiguous nucleotides that comprises one, two, or three mismatches relative to the target sequence. In some embodiments, the antisense strand sequence is complementary to at least 15, 16, 17, or 18, contiguous nucleotides with 0, 1, 2, or 3 mismatches to nucleotides 1300-1317, 1305-1322, 1860-1877, 2279-2297, 2286-2303, 2402-2419, 2581-2958, 2584-2601, 2633-2650, or 2634-2653 of SEQ ID NO: 5024. In some embodiments, the antisense strand sequence is complementary to at least 15, 16, 17, 18, 19, 20, or 21, contiguous nucleotides with 0, 1, 2, or 3 mismatches to a portion of the target sequence present in an exon (e.g., an exon comprising nucleotides 1277 to 1332 of SEQ ID NO: 5024, an exon comprising nucleotides 1712-1977 of SEQ ID NO: 5024, or an exon comprising nucleotides 2266-6644 of SEQ ID NO: 5024) of a coding sequence of a MAPT mRNA, e.g., a MAPT sequence of SEQ ID NO: 5024, e.g., a coding sequence comprising nucleotides 151 to 2481 of SEQ ID NO: 5024.

In some embodiments, the sense strand sequence comprises at least 15, 16, 17, 18, 19, 20, or 21 contiguous nucleotides with 0, 1, 2, or 3 mismatches of the corresponding portion of a MAPT sequence, e.g., a MAPT sequence provided in Table 3 or 19, or a variant thereof. In some embodiments, the sense strand sequence or a portion thereof corresponds to positions 1300-1317, 1305-1322, 1860-1877, 2279-2297, 2286-2303, 2402-2419, 2581-2958, 2584-2601, 2633-2650, or 2634-2653 of SEQ ID NO: 5024. In some embodiments, the sense strand sequence comprises a nucleotide sequence comprising at least 15, 16, 17, 18, 19, 20, or 21, contiguous nucleotides with 0, 1, 2, or 3 mismatches of the corresponding portion of an exon (e.g., an exon comprising nucleotides 1277 to 1332 of SEQ ID NO: 5024, an exon comprising nucleotides 1712-1977 of SEQ ID NO: 5024, or an exon comprising nucleotides 2266-6644 of SEQ ID NO: 5024) of a coding sequence of a MAPT mRNA, e.g., a MAPT sequence of SEQ ID NO: 5024, e.g., a coding sequence comprising nucleotides 151 to 2481 of SEQ ID NO: 5024. In some embodiments, the sense strand sequence comprises at least 15, 16, 17, or 18 contiguous nucleotides with 0, 1, 2, or 3 mismatches from nucleotides 1300-1317, 1305-1322, 1860-1877, 2279-2297, 2286-2303, 2402-2419, 2581-2958, 2584-2601, 2633-2650, or 2634-2653 of SEQ ID NO: 5024.

In some embodiments, the sense strand sequence and the antisense strand sequence of an siRNA described herein comprise a region of complementarity, which is 15-30, 19-21, or 25-30 nucleotides in length, e.g., 17, 18, 20, 21, 22, 25, or 30 nucleotides in length. In some embodiments, the sense strand, antisense strand, or both comprise at least 15-30, 19-21, or 25-30 nucleotides in length, e.g., 17, 18, 20, 21, 22, 25, or 30 nucleotides in length. In some embodiments, the sense strand, antisense strand, or both the sense strand comprise one or two nucleotide overhangs, e.g., at the 5′ end of the sense strand and the 3′ end of the antisense strand or at the 5′ end of the antisense strand and the 3′ end of the sense strand. In some embodiments, the sense strand and the antisense strand comprise one mismatch. In some embodiments, the antisense strand sequence comprises one mismatch with the target sequence.

In some embodiments, the siRNA molecules of the present disclosure can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications. In some embodiments, the overhanging nucleotides at the 3′-end of each of the sense and the antisense strand is a dTdT overhang. In some embodiments, the sense strand, antisense strand, or both the sense strand and the antisense strand does not comprise a dTdT overhang.

In some embodiments, the siRNA molecule comprises an antisense (e.g., guide) strand sequence. In some embodiments, the antisense strand sequence comprises at least 15, 16, 17, 18, 19, 20, 21 contiguous nucleotides differing by 3, 2, 1, or 0 nucleotides from any one of the antisense strand sequences provided in Tables 4, 5, 9A, or 9B. In some embodiments, the antisense sequence comprises the nucleotide sequence of any one of the antisense strand sequences provided in Tables 4, 5, 9A, or 9B, or a nucleotide sequence 70%, 80%, 85%, 90%, 95% identical thereto. In some embodiments, the antisense strand sequence comprises at least 15, 16, 17, 18, 19, or more than 19 consecutive nucleotides of a nucleotide sequence of any one of the antisense strand sequences provided in Tables 4, 5, 9A, or 9B. In some embodiments, the antisense sequence comprises nucleotides 1 to 19, 1 to 18, 1 to 17, 1 to 16, 2 to 19, or 2 to 18, 2 to 17 of any one of the antisense strand sequences provided in Tables 4, 5, 9A, or 9B. In some embodiments, the antisense sequence may comprise a dTdT overhang, e.g., at the 3′ end of the antisense strand sequence. In some embodiments, the antisense sequence does not comprise a dTdT overhang, e.g., does not comprise a dTdT overhang at the 3′ end of the sequence. In some embodiments, the antisense strand sequence is a DNA sequence (e.g., comprises T in place of U in any one of the sequences provided in Tables 4 and 5). In some embodiments, the antisense strand sequence is an RNA sequence (e.g., comprises U in place of T in any one of the sequences provided in Table 9A or 9B).

In some embodiments, the siRNA molecule comprises a sense (e.g., passenger) strand sequence. In some embodiments, the sense strand sequence comprises at least 15, 16, 17, 18, 19, 20, 21 contiguous nucleotides differing by 3, 2, 1, or 0 nucleotides from any one of the sense strand sequences provided in Tables 4, 5, 9A, or 9B. In some embodiments, the sense sequence comprises the nucleotide sequence of any one of the sense strand sequences provided in Tables 4, 5, 9A, or 9B, or a nucleotide sequence 70%, 80%, 85%, 90%, 95% identical thereto. In some embodiments, the sense strand sequence comprises at least 15, 16, 17, 18, 19, or more than 19 consecutive nucleotides of a nucleotide sequence of any one of the sense strand sequences provided in Tables 4, 5, 9A, or 9B. In some embodiments, the sense sequence comprises nucleotides 1 to 19, 1 to 18, 1 to 17, 1 to 162 to 19, or 2 to 18, 2 to 17 of any one of the sense strand sequences provided in Tables 4, 5, 9A, or 9B. In some embodiments, the sense sequence may comprise a dTdT overhang, e.g. at the 3′ end of the sense strand sequence. In some embodiments, the sense sequence does not comprise a dTdT overhang, e.g., does not comprise the dTdT overhang at the 3′ end of the sequence. In some embodiments, the sense strand sequence is a DNA sequence (e.g., comprises T in place of U in any one of the sequences provided in Tables 4 and 5). In some embodiments, the sense strand sequence is an RNA sequence (e.g., comprises U in place of T in any one of the sequences provided in Table 9A or 9B).

In some embodiments, the siRNA comprises an antisense strand sequence comprising a nucleotide sequence comprising at least 15, 16, 17, 18, 19, 20, or 21 contiguous nucleotides differing by no more than 3, 2, 1, or 0 nucleotides from the antisense strand sequences provided in Tables 4, 5, 9A, or 9B; and a sense strand sequence comprising a nucleotide sequence comprising at least 15, 16, 17, 18, 19, 20, or 21 contiguous nucleotides differing by no more than 3, 2, 1, or 0 nucleotides from the sense strand sequences provided in Tables 4, 5, 9A, or 9B, optionally wherein the sense strand sequence and the antisense strand sequence comprise a region of complementarity of at least 15 nucleotides. In some embodiments, the siRNA molecules of the present disclosure may comprise a siRNA duplex as described in Table 4, 5 9A, or 9B, or a variant, e.g., functional variant, thereof.

In some embodiments, the dTdT nucleotides of the sense strand and/or the antisense strand of the siRNA provided in Table 4 or herein may be replaced with any two nucleotides (e.g., UU, UC, AA, AG, AC, etc.). Alternatively, the dTdT nucleotides of the sense strand and/or the antisense strand of Table 4 or provided herein may be replaced with a single nucleotide (e.g., U, A, C, or G).

TABLE 4
Exemplary Sense and Antisense Strand Sequences of MAPT dsRNA

| | | Sense | SS | | | AS |
|---|---|---|---|---|---|---|
| siRNA | | Strand | SEQ | | | SEQ |
| Duplex | | Sequence | ID | | Antisense Strand | ID |
| ID | SS ID | (5′-3′) | NO | AS ID | Sequence (5′-3′) | NO |
| D-001 | SS-001 | GCUUUGGCUCGG | 4143 | AS-001 | UGAAGUCCCGAGCC | 3952 |
| | | GACUUCCdTdT | | | AAAGCdTdT | |
| D-002 | SS-002 | CGACAUGGUAG | 4144 | AS-002 | UGGCGAGUCUACCA | 3953 |
| | | ACUCGCCCdTdT | | | UGUCGdTdT | |
| D-003 | SS-003 | CUCCUCGCAGUU | 4145 | AS-003 | UUAACCGAACUGCG | 3954 |
| | | CGGUUACdTdT | | | AGGAGdTdT | |
| D-004 | SS-004 | GGCUUUGGCUCG | 4146 | AS-004 | UAAGUCCCGAGCCA | 3955 |
| | | GGACUUCdTdT | | | AAGCCdTdT | |
| D-005 | SS-005 | GGCUUUGGCUCG | 4147 | AS-004 | UAAGUCCCGAGCCA | 3955 |
| | | GGACUUAdTdT | | | AAGCCdTdT | |
| D-006 | SS-006 | GGCUUUGGCUCG | 4148 | AS-004 | UAAGUCCCGAGCCA | 3955 |
| | | GGACUUUdTdT | | | AAGCCdTdT | |
| D-007 | SS-007 | GGCUUUGGCACG | 4149 | AS-004 | UAAGUCCCGAGCCA | 3955 |
| | | GGACUUCdTdT | | | AAGCCdTdT | |
| D-008 | SS-008 | GGCUUUGGCUCG | 4150 | AS-004 | UAAGUCCCGAGCCA | 3955 |
| | | GGACUUGdTdT | | | AAGCCdTdT | |
| D-009 | SS-009 | GCAUCGACAUGG | 4151 | AS-005 | UAGUCUACCAUGUC | 3956 |
| | | UAGACUCdTdT | | | GAUGCdTdT | |
| D-010 | SS-010 | GCAUCGACAUGG | 4152 | AS-005 | UAGUCUACCAUGUC | 3956 |
| | | UAGACUAdTdT | | | GAUGCdTdT | |
| D-011 | SS-011 | GCAUCGACAUGG | 4153 | AS-005 | UAGUCUACCAUGUC | 3956 |
| | | UAGACUUdTdT | | | GAUGCdTdT | |
| D-012 | SS-012 | GCAUCGACAAGG | 4154 | AS-005 | UAGUCUACCAUGUC | 3956 |
| | | UAGACUCdTdT | | | GAUGCdTdT | |
| D-013 | SS-013 | GCAUCGACAUGG | 4155 | AS-005 | UAGUCUACCAUGUC | 3956 |
| | | UAGACUGdTdT | | | GAUGCdTdT | |
| D-014 | SS-014 | UGCUCCUCGCAG | 4156 | AS-006 | UACCGAACUGCGAG | 3957 |
| | | UUCGGUCdTdT | | | GAGCAdTdT | |
| D-015 | SS-015 | CACCCAAGUCGC | 4157 | AS-007 | UAAGACGGCGACUU | 3958 |
| | | CGUCUUCdTdT | | | GGGUGdTdT | |
| D-016 | SS-016 | CCUCGCAGUUCG | 4158 | AS-008 | UAUUAACCGAACUG | 3959 |
| | | GUUAAUCdTdT | | | CGAGGdTdT | |
| D-017 | SS-017 | UCCUCGCAGUUC | 4159 | AS-009 | UUUAACCGAACUGC | 3960 |
| | | GGUUAACdTdT | | | GAGGAdTdT | |
| D-018 | SS-018 | AGCUGACCUUCC | 4160 | AS-010 | UUCUCGCGGAAGGU | 3961 |
| | | GCGAGACdTdT | | | CAGCUdTdT | |
| D-019 | SS-019 | GACUGGAAGCG | 4161 | AS-011 | UUUGUCAUCGCUUC | 3962 |
| | | AUGACAACdTdT | | | CAGUCdTdT | |
| D-020 | SS-020 | GACUGGAAGCG | 4162 | AS-011 | UUUGUCAUCGCUUC | 3962 |
| | | AUGACAAAdTdT | | | CAGUCdTdT | |
| D-021 | SS-021 | GACUGGAAGCG | 4163 | AS-011 | UUUGUCAUCGCUUC | 3962 |
| | | AUGACAAUdTdT | | | CAGUCdTdT | |
| D-022 | SS-022 | GACUGGAAGGG | 4164 | AS-011 | UUUGUCAUCGCUUC | 3962 |
| | | AUGACAACdTdT | | | CAGUCdTdT | |
| D-023 | SS-023 | GACUGGAAGCG | 4165 | AS-011 | UUUGUCAUCGCUUC | 3962 |
| | | AUGACAAGdTdT | | | CAGUCdTdT | |
| D-024 | SS-024 | AGCUCGCAUGGU | 4166 | AS-012 | UUUACUGACCAUGC | 3963 |
| | | CAGUAACdTdT | | | GAGCUdTdT | |
| D-025 | SS-025 | AGUCUACAAACC | 4167 | AS-013 | UUCAACUGGUUUGU | 3964 |
| | | AGUUGACdTdT | | | AGACUdTdT | |
| D-026 | SS-026 | GCUCCUCGCAGU | 4168 | AS-014 | UAACCGAACUGCGA | 3965 |
| | | UCGGUUCdTdT | | | GGAGCdTdT | |
| D-027 | SS-027 | GCUCCUCGCAGU | 4169 | AS-014 | UAACCGAACUGCGA | 3965 |
| | | UCGGUUGdTdT | | | GGAGCdTdT | |
| D-028 | SS-028 | GCUCCUCGCAGU | 4170 | AS-014 | UAACCGAACUGCGA | 3965 |
| | | UCGGUUUdTdT | | | GGAGCdTdT | |
| D-029 | SS-029 | GCUCCUCGCUGU | 4171 | AS-014 | UAACCGAACUGCGA | 3965 |
| | | UCGGUUCdTdT | | | GGAGCdTdT | |
| D-030 | SS-030 | CUUCCGCGAGAA | 4172 | AS-015 | UUUGGCGUUCUCGC | 3966 |
| | | CGCCAACdTdT | | | GGAAGdTdT | |
| D-031 | SS-031 | CUUCCGCGAGAA | 4173 | AS-015 | UUUGGCGUUCUCGC | 3966 |
| | | CGCCAAAdTdT | | | GGAAGdTdT | |
| D-032 | SS-032 | CUUCCGCGAGAA | 4174 | AS-015 | UUUGGCGUUCUCGC | 3966 |
| | | CGCCAAUdTdT | | | GGAAGdTdT | |
| D-033 | SS-033 | CUUCCGCGACAA | 4175 | AS-015 | UUUGGCGUUCUCGC | 3966 |
| | | CGCCAACdTdT | | | GGAAGdTdT | |
| D-034 | SS-034 | CUUCCGCGAGAA | 4176 | AS-015 | UUUGGCGUUCUCGC | 3966 |
| | | CGCCAAGdTdT | | | GGAAGdTdT | |
| D-035 | SS-035 | AAAUCAGUGAU | 4177 | AS-016 | UUACUCCCAUCACU | 3967 |
| | | GGGAGUACdTdT | | | GAUUUdTdT | |
| D-036 | SS-036 | GUCGCCGUCUUC | 4178 | AS-017 | UUUGGCGGAAGACG | 3968 |
| | | CGCCAACdTdT | | | GCGACdTdT | |
| D-037 | SS-037 | AUGGUAAAACG | 4179 | AS-018 | UCGAUCUUCGUUUU | 3969 |
| | | AAGAUCGCdTdT | | | ACCAUdTdT | |
| D-038 | SS-038 | ACGAAGAUCGCC | 4180 | AS-019 | UCGGUGUGGCGAUC | 3970 |
| | | ACACCGCdTdT | | | UUCGUdTdT | |
| D-039 | SS-039 | CGCAGUUCGGUU | 4181 | AS-020 | UCCAAUUAACCGAA | 3971 |
| | | AAUUGGCdTdT | | | CUGCGdTdT | |
| D-040 | SS-040 | GUACUCCACCCA | 4182 | AS-021 | UGCGACUUGGGUGG | 3972 |
| | | AGUCGCCdTdT | | | AGUACdTdT | |
| D-041 | SS-041 | CUCGGCUUUGGC | 4183 | AS-022 | UUCCCGAGCCAAAG | 3973 |
| | | UCGGGACdTdT | | | CCGAGdTdT | |
| D-042 | SS-042 | GCCCCUGGGGCG | 4184 | AS-023 | UAUUGACCGCCCCA | 3974 |
| | | GUCAAUCdTdT | | | GGGGCdTdT | |
| D-043 | SS-043 | CUCGCAGUUCGG | 4185 | AS-024 | UAAUUAACCGAACU | 3975 |
| | | UUAAUUCdTdT | | | GCGAGdTdT | |
| D-044 | SS-044 | UCGCCGUCUUCC | 4186 | AS-025 | UCUUGGCGGAAGAC | 3976 |
| | | GCCAAGCdTdT | | | GGCGAdTdT | |
| D-045 | SS-045 | GACGGGACUGG | 4187 | AS-026 | UAUCGCUUCCAGUC | 3977 |
| | | AAGCGAUCdTdT | | | CCGUCdTdT | |
| D-046 | SS-046 | UGGUAAAACGA | 4188 | AS-027 | UGCGAUCUUCGUUU | 3978 |
| | | AGAUCGCCdTdT | | | UACCAdTdT | |
| D-047 | SS-047 | GUAAAACGAAG | 4189 | AS-028 | UUGGCGAUCUUCGU | 3979 |
| | | AUCGCCACdTdT | | | UUUACdTdT | |
| D-048 | SS-048 | ACAAUAUCACCC | 4190 | AS-029 | UGGACGUGGGUGAU | 3980 |
| | | ACGUCCCdTdT | | | AUUGUdTdT | |
| D-049 | SS-049 | CCUUCCGCGAGA | 4191 | AS-030 | UUGGCGUUCUCGCG | 3981 |
| | | ACGCCACdTdT | | | GAAGGdTdT | |
| D-050 | SS-050 | GGGGGCUGAUG | 4192 | AS-031 | UGUUUUACCAUCAG | 3982 |
| | | GUAAAACCdTdT | | | CCCCCdTdT | |
| D-051 | SS-051 | GCCGUCUUCCGC | 4193 | AS-032 | UCUCUUGGCGGAAG | 3983 |
| | | CAAGAGCdTdT | | | ACGGCdTdT | |
| D-052 | SS-052 | CACCGGCAGCAU | 4194 | AS-033 | UAUGUCGAUGCUGC | 3984 |
| | | CGACAUCdTdT | | | CGGUGdTdT | |
| D-053 | SS-053 | GUAAAAGCAAA | 4195 | AS-034 | UUCCCGUCUUUGCU | 3985 |
| | | GACGGGACdTdT | | | UUUACdTdT | |
| D-054 | SS-054 | AGGUUUCUAACC | 4196 | AS-035 | UAGGGUGGGUUAGA | 3986 |
| | | CACCCUCdTdT | | | AACCUdTdT | |
| D-055 | SS-055 | UUCCGCGAGAAC | 4197 | AS-036 | UUUUGGCGUUCUCG | 3987 |
| | | GCCAAACdTdT | | | CGGAAdTdT | |
| D-056 | SS-056 | GACCUUCCGCGA | 4198 | AS-037 | UGCGUUCUCGCGGA | 3988 |
| | | GAACGCCdTdT | | | AGGUCdTdT | |
| D-057 | SS-057 | CGCCACGCUAGC | 4199 | AS-038 | UUCGUCAGCUAGCG | 3989 |
| | | UGACGACdTdT | | | UGGCGdTdT | |
| D-058 | SS-058 | ACCUGCGUGUCC | 4200 | AS-039 | UUAGAUGGGACACG | 3990 |
| | | CAUCUACdTdT | | | CAGGUdTdT | |
| D-059 | SS-059 | CCCCAGCUCGCC | 4201 | AS-040 | UUAGCGUGGCGAGC | 3991 |
| | | ACGCUACdTdT | | | UGGGGdTdT | |
| D-060 | SS-060 | GCCGUUCAGCUG | 4202 | AS-041 | UUCGUCACAGCUGA | 3992 |
| | | UGACGACdTdT | | | ACGGCdTdT | |
| D-061 | SS-061 | GCUGAUGGUAA | 4203 | AS-042 | UCUUCGUUUUACCA | 3993 |
| | | AACGAAGCdTdT | | | UCAGCdTdT | |
| D-062 | SS-062 | ACGAGGUGUCUC | 4204 | AS-043 | UGGGUGAGAGACAC | 3994 |
| | | UCACCCCdTdT | | | CUCGUdTdT | |
| D-063 | SS-063 | GGGCCAGCCUAA | 4205 | AS-044 | UAUGAUCUUAGGCU | 3995 |
| | | GAUCAUCdTdT | | | GGCCCdTdT | |
| D-064 | SS-064 | UCUAACCCACCC | 4206 | AS-045 | UUCGUGAGGGUGGG | 3996 |
| | | UCACGACdTdT | | | UUAGAdTdT | |
| D-065 | SS-065 | UAAAGAGGUUU | 4207 | AS-046 | UGGGUUAGAAACCU | 3997 |
| | | CUAACCCCdTdT | | | CUUUAdTdT | |
| D-066 | SS-066 | CUCACCUCCUAA | 4208 | AS-047 | UAGUCUAUUAGGAG | 3998 |
| | | UAGACUCdTdT | | | GUGAGdTdT | |
| D-067 | SS-067 | GGAUAUACUGU | 4209 | AS-048 | UCCGGCAUACAGUA | 3999 |
| | | AUGCCGGCdTdT | | | UAUCCdTdT | |
| D-068 | SS-068 | CACCCUCACGAG | 4210 | AS-049 | UAGACACCUCGUGA | 4000 |
| | | GUGUCUCdTdT | | | GGGUGdTdT | |
| D-069 | SS-069 | CACGCUAGCUGA | 4211 | AS-050 | UACCUCGUCAGCUA | 4001 |
| | | CGAGGUCdTdT | | | GCGUGdTdT | |
| D-070 | SS-070 | UCCCAUGAUUUC | 4212 | AS-051 | UACCGAAGAAAUCA | 4002 |
| | | UUCGGUCdTdT | | | UGGGAdTdT | |
| D-071 | SS-071 | UUGAGUUGUAG | 4213 | AS-052 | UAAUCCAACUACAA | 4003 |
| | | UUGGAUUCdTdT | | | CUCAAdTdT | |
| D-072 | SS-072 | CUGCGUGUCCCA | 4214 | AS-053 | UUGUAGAUGGGACA | 4004 |
| | | UCUACACdTdT | | | CGCAGdTdT | |
| D-073 | SS-073 | GUUGAGUUGUA | 4215 | AS-054 | UAUCCAACUACAAC | 4005 |
| | | GUUGGAUCdTdT | | | UCAACdTdT | |
| D-074 | SS-074 | GGUGGUGGUUA | 4216 | AS-055 | UAUAUCUCUAACCA | 4006 |
| | | GAGAUAUCdTdT | | | CCACCdTdT | |
| D-075 | SS-075 | GGGCUAGAUAG | 4217 | AS-056 | UGUAUAUCCUAUCU | 4007 |
| | | GAUAUACCdTdT | | | AGCCCdTdT | |
| D-076 | SS-076 | AGGAUUCCAGCA | 4218 | AS-057 | UGGUUUUUGCUGGA | 4008 |
| | | AAAACCCdTdT | | | AUCCUdTdT | |
| D-077 | SS-077 | GGCCCCUGGGGC | 4219 | AS-058 | UUUGACCGCCCCAG | 4009 |
| | | GGUCAACdTdT | | | GGGCCdTdT | |
| D-078 | SS-078 | GAUUAGGACUG | 4220 | AS-059 | UAUCGCUUCAGUCC | 4010 |
| | | AAGCGAUCdTdT | | | UAAUCdTdT | |
| D-079 | SS-079 | CUCCCAAGUUUU | 4221 | AS-060 | UCCUUUCAAAACUU | 4011 |
| | | GAAAGGCdTdT | | | GGGAGdTdT | |
| D-080 | SS-080 | GCUGGUCUGGCU | 4222 | AS-061 | UGCCGCAAGCCAGA | 4012 |
| | | UGCGGCCdTdT | | | CCAGCdTdT | |
| D-081 | SS-081 | GUUCCACUCAUC | 4223 | AS-062 | UCAGUUGGAUGAGU | 4013 |
| | | CAACUGCdTdT | | | GGAACdTdT | |
| D-082 | SS-082 | CACCUCCUAAUA | 4224 | AS-063 | UUAAGUCUAUUAGG | 4014 |
| | | GACUUACdTdT | | | AGGUGdTdT | |
| D-083 | SS-083 | UUCUAACCCACC | 4225 | AS-064 | UCGUGAGGGUGGGU | 4015 |
| | | CUCACGCdTdT | | | UAGAAdTdT | |
| D-084 | SS-084 | AAGCCCACAAUC | 4226 | AS-065 | UAGGCAUGAUUGUG | 4016 |
| | | AUGCCUCdTdT | | | GGCUUdTdT | |
| D-085 | SS-085 | ACCUUCCGCGAG | 4227 | AS-066 | UGGCGUUCUCGCGG | 4017 |
| | | AACGCCCdTdT | | | AAGGUdTdT | |
| D-086 | SS-086 | GCUGGUCACGUG | 4228 | AS-067 | UUUGGGUCACGUGA | 4018 |
| | | ACCCAACdTdT | | | CCAGCdTdT | |
| D-087 | SS-087 | UAACCCACCCUC | 4229 | AS-068 | UCCUCGUGAGGGUG | 4019 |
| | | ACGAGGCdTdT | | | GGUUAdTdT | |
| D-088 | SS-088 | CUUACAACUCCU | 4230 | AS-069 | UUGAUGCAGGAGUU | 4020 |
| | | GCAUCACdTdT | | | GUAAGdTdT | |
| D-089 | SS-089 | UGGGACUUUAG | 4231 | AS-070 | UGUUAGCCCUAAAG | 4021 |
| | | GGCUAACCdTdT | | | UCCCAdTdT | |
| D-090 | SS-090 | GCCACAGGCAGA | 4232 | AS-071 | UACAUCGUCUGCCU | 4022 |
| | | CGAUGUCdTdT | | | GUGGCdTdT | |
| D-091 | SS-091 | GCCUCUUGGGAG | 4233 | AS-072 | UGGACGUCUCCCAA | 4023 |
| | | ACGUCCCdTdT | | | GAGGCdTdT | |
| D-092 | SS-092 | GAGAUAUGCCCC | 4234 | AS-073 | UAUGAGGGGGGCAU | 4024 |
| | | CCUCAUCdTdT | | | AUCUCdTdT | |
| D-093 | SS-093 | UCCCAAGUUUUG | 4235 | AS-074 | UGCCUUUCAAAACU | 4025 |
| | | AAAGGCCdTdT | | | UGGGAdTdT | |
| D-094 | SS-094 | AGUUAUUACUC | 4236 | AS-075 | UUUAAUCAGAGUAA | 4026 |
| | | UGAUUAACdTdT | | | UAACUdTdT | |
| D-095 | SS-095 | UAUGCCCCCCUC | 4237 | AS-076 | UAGUAAUGAGGGGG | 4027 |
| | | AUUACUCdTdT | | | GCAUAdTdT | |
| D-096 | SS-096 | CAAAAUCAGGG | 4238 | AS-077 | UUGCGAUCCCCUGA | 4028 |
| | | GAUCGCACdTdT | | | UUUUGdTdT | |
| D-097 | SS-097 | CAGACCACGGGG | 4239 | AS-078 | UUCUCCGCCCCGUG | 4029 |
| | | CGGAGACdTdT | | | GUCUGdTdT | |
| D-098 | SS-098 | CUUGGGUGGGC | 4240 | AS-079 | UCUAUCUAGCCCAC | 4030 |
| | | UAGAUAGCdTdT | | | CCAAGdTdT | |
| D-099 | SS-099 | CUCGCCACGCUA | 4241 | AS-080 | UGUCAGCUAGCGUG | 4031 |
| | | GCUGACCdTdT | | | GCGAGdTdT | |
| D-100 | SS-100 | UGUUGAGUUGU | 4242 | AS-081 | UUCCAACUACAACU | 4032 |
| | | AGUUGGACdTdT | | | CAACAdTdT | |
| D-101 | SS-101 | UAUACAUAGAU | 4243 | AS-082 | UGGGCAACAUCUAU | 4033 |
| | | GUUGCCCCdTdT | | | GUAUAdTdT | |
| D-102 | SS-102 | AACCCACCCUCA | 4244 | AS-083 | UACCUCGUGAGGGU | 4034 |
| | | CGAGGUCdTdT | | | GGGUUdTdT | |
| D-103 | SS-103 | GUUGAGCAGGA | 4245 | AS-084 | UGAAAUAGUCCUGC | 4035 |
| | | CUAUUUCCdTdT | | | UCAACdTdT | |
| D-104 | SS-104 | CCACGCUAGCUG | 4246 | AS-085 | UCCUCGUCAGCUAG | 4036 |
| | | ACGAGGCdTdT | | | CGUGGdTdT | |
| D-105 | SS-105 | CUCCUAAUAGAC | 4247 | AS-086 | UGGCUAAGUCUAUU | 4037 |
| | | UUAGCCCdTdT | | | AGGAGdTdT | |
| D-106 | SS-106 | GAUUUCUUCGG | 4248 | AS-087 | UAGAAUUACCGAAG | 4038 |
| | | UAAUUCUCdTdT | | | AAAUCdTdT | |
| D-107 | SS-107 | GUCCCAAAGGAG | 4249 | AS-088 | UGUAAGCCUCCUUU | 4039 |
| | | GCUUACCdTdT | | | GGGACdTdT | |
| D-108 | SS-108 | GGUGGUUAGAG | 4250 | AS-089 | UGGCAUAUCUCUAA | 4040 |
| | | AUAUGCCCdTdT | | | CCACCdTdT | |
| D-109 | SS-109 | GGCUAGAUAGG | 4251 | AS-090 | UAGUAUAUCCUAUC | 4041 |
| | | AUAUACUCdTdT | | | UAGCCdTdT | |
| D-110 | SS-110 | AUUCACCAGAGU | 4252 | AS-091 | UAUAGUCACUCUGG | 4042 |
| | | GACUAUCdTdT | | | UGAAUdTdT | |
| D-111 | SS-111 | GAUAUACUGUA | 4253 | AS-092 | UGCCGGCAUACAGU | 4043 |
| | | UGCCGGCCdTdT | | | AUAUCdTdT | |
| D-112 | SS-112 | GACCACAGCAGG | 4254 | AS-093 | UCCAAUCCCUGCUG | 4044 |
| | | GAUUGGCdTdT | | | UGGUCdTdT | |
| D-113 | SS-113 | GAGCCACAGGCA | 4255 | AS-094 | UAUCGUCUGCCUGU | 4045 |
| | | GACGAUCdTdT | | | GGCUCdTdT | |
| D-114 | SS-114 | CUAGCUGACGAG | 4256 | AS-095 | UAGACACCUCGUCA | 4046 |
| | | GUGUCUCdTdT | | | GCUAGdTdT | |
| D-115 | SS-115 | AAGGGGGCUGA | 4257 | AS-096 | UUUUACCAUCAGCC | 4047 |
| | | UGGUAAACdTdT | | | CCCUUdTdT | |
| D-116 | SS-116 | AUCAGGGGAUC | 4258 | AS-097 | UCCGCUGCGAUCCC | 4048 |
| | | GCAGCGGCdTdT | | | CUGAUdTdT | |
| D-117 | SS-117 | CCCAGCUCGCCA | 4259 | AS-098 | UCUAGCGUGGCGAG | 4049 |
| | | CGCUAGCdTdT | | | CUGGGdTdT | |
| D-118 | SS-118 | CGUUCAGCUGUG | 4260 | AS-099 | UCUUCGUCACAGCU | 4050 |
| | | ACGAAGCdTdT | | | GAACGdTdT | |
| D-119 | SS-119 | CACUUGCAAGUC | 4261 | AS-100 | UUCAUGGGACUUGC | 4051 |
| | | CCAUGACdTdT | | | AAGUGdTdT | |
| D-120 | SS-120 | UGGGCUAGAUA | 4262 | AS-101 | UUAUAUCCUAUCUA | 4052 |
| | | GGAUAUACdTdT | | | GCCCAdTdT | |
| D-121 | SS-121 | UUUAUGCUUGG | 4263 | AS-102 | UGGUGAAUCCAAGC | 4053 |
| | | AUUCACCCdTdT | | | AUAAAdTdT | |
| D-122 | SS-122 | CCAAAAUCAGGG | 4264 | AS-103 | UGCGAUCCCCUGAU | 4054 |
| | | GAUCGCCdTdT | | | UUUGGdTdT | |
| D-123 | SS-123 | CCGUUCAGCUGU | 4265 | AS-104 | UUUCGUCACAGCUG | 4055 |
| | | GACGAACdTdT | | | AACGGdTdT | |
| D-124 | SS-124 | GGCCACAUCCAA | 4266 | AS-105 | UGAAAUGUUGGAUG | 4056 |
| | | CAUUUCCdTdT | | | UGGCCdTdT | |
| D-125 | SS-125 | UUUAUCAAUAG | 4267 | AS-106 | UAAUGGAACUAUUG | 4057 |
| | | UUCCAUUCdTdT | | | AUAAAdTdT | |
| D-126 | SS-126 | GGCGGAGAUCG | 4268 | AS-107 | UUUGUACACGAUCU | 4058 |
| | | UGUACAACdTdT | | | CCGCCdTdT | |
| D-127 | SS-127 | UCGCCACGCUAG | 4269 | AS-108 | UCGUCAGCUAGCGU | 4059 |
| | | CUGACGCdTdT | | | GGCGAdTdT | |
| D-128 | SS-128 | GUCUGGCUUGCG | 4270 | AS-109 | UUCGCGCCGCAAGC | 4060 |
| | | GCGCGACdTdT | | | CAGACdTdT | |
| D-129 | SS-129 | ACCUCCUAAUAG | 4271 | AS-110 | UCUAAGUCUAUUAG | 4061 |
| | | ACUUAGCdTdT | | | GAGGUdTdT | |
| D-130 | SS-130 | UGUCCCAUCUAC | 4272 | AS-111 | UAGGUCUGUAGAUG | 4062 |
| | | AGACCUCdTdT | | | GGACAdTdT | |
| D-131 | SS-131 | CGGUAAUUCUG | 4273 | AS-112 | UCCACCCUCAGAAU | 4063 |
| | | AGGGUGGCdTdT | | | UACCGdTdT | |
| D-132 | SS-132 | UUUCUUCGGUA | 4274 | AS-113 | UUCAGAAUUACCGA | 4064 |
| | | AUUCUGACdTdT | | | AGAAAdTdT | |
| D-133 | SS-133 | GCACCCUGUUGA | 4275 | AS-114 | UUACAACUCAACAG | 4065 |
| | | GUUGUACdTdT | | | GGUGCdTdT | |
| D-134 | SS-134 | AUAUACUGUAU | 4276 | AS-115 | UAGCCGGCAUACAG | 4066 |
| | | GCCGGCUCdTdT | | | UAUAUdTdT | |
| D-135 | SS-135 | ACUUCUGGUUU | 4277 | AS-116 | UUGUACCCAAACCA | 4067 |
| | | GGGUACACdTdT | | | GAAGUdTdT | |
| D-136 | SS-136 | GAAGCACAGGA | 4278 | AS-117 | UGUCCUAAUCCUGU | 4068 |
| | | UUAGGACCdTdT | | | GCUUCdTdT | |
| D-137 | SS-137 | CUUUCAGGGGUC | 4279 | AS-118 | UGCUUAGGACCCCU | 4069 |
| | | CUAAGCCdTdT | | | GAAAGdTdT | |
| D-138 | SS-138 | GAUAUGCCCCCC | 4280 | AS-119 | UUAAUGAGGGGGGC | 4070 |
| | | UCAUUACdTdT | | | AUAUCdTdT | |
| D-139 | SS-139 | CUAAGGAGGCCG | 4281 | AS-120 | UGCUGAACGGCCUC | 4071 |
| | | UUCAGCCdTdT | | | CUUAGdTdT | |
| D-140 | SS-140 | GGAACACACCCC | 4282 | AS-121 | UUCCAAGGGGGUGU | 4072 |
| | | CUUGGACdTdT | | | GUUCCdTdT | |
| D-141 | SS-141 | CCUAAGCCCACA | 4283 | AS-122 | UCAUGAUUGUGGGC | 4073 |
| | | AUCAUGCdTdT | | | UUAGGdTdT | |
| D-142 | SS-142 | CUGGGGCCUCCC | 4284 | AS-123 | UAAACUUGGGAGGC | 4074 |
| | | AAGUUUCdTdT | | | CCCAGdTdT | |
| D-143 | SS-143 | UGUCCCCCACUU | 4285 | AS-124 | UGGGUGCAAGUGGG | 4075 |
| | | GCACCCCdTdT | | | GGACAdTdT | |
| D-144 | SS-144 | AAGUUAUUACU | 4286 | AS-125 | UUAAUCAGAGUAAU | 4076 |
| | | CUGAUUACdTdT | | | AACUUdTdT | |
| D-145 | SS-145 | AAUAGACUUAG | 4287 | AS-126 | UCAUGGGGCUAAGU | 4077 |
| | | CCCCAUGCdTdT | | | CUAUUdTdT | |
| D-146 | SS-146 | CAGUAAUGACCC | 4288 | AS-127 | UCAACCAGGGUCAU | 4078 |
| | | UGGUUGCdTdT | | | UACUGdTdT | |
| D-147 | SS-147 | GGAGGAAUGUG | 4289 | AS-128 | UUAUCUUACACAUU | 4079 |
| | | UAAGAUACdTdT | | | CCUCCdTdT | |
| D-148 | SS-148 | CAAUCAUGCCUC | 4290 | AS-129 | UCUUAGGGAGGCAU | 4080 |
| | | CCUAAGCdTdT | | | GAUUGdTdT | |
| D-149 | SS-149 | UCCACUCAUCCA | 4291 | AS-130 | UCCCAGUUGGAUGA | 4081 |
| | | ACUGGGCdTdT | | | GUGGAdTdT | |
| D-150 | SS-150 | AGGUGCAGGCG | 4292 | AS-131 | UAUUCCUACGCCUG | 4082 |
| | | UAGGAAUCdTdT | | | CACCUdTdT | |
| D-151 | SS-151 | GAUAGGAUAUA | 4293 | AS-132 | UCAUACAGUAUAUC | 4083 |
| | | CUGUAUGCdTdT | | | CUAUCdTdT | |
| D-152 | SS-152 | UCCUAAGCCCAC | 4294 | AS-133 | UAUGAUUGUGGGCU | 4084 |
| | | AAUCAUCdTdT | | | UAGGAdTdT | |
| D-153 | SS-153 | GGCCGUUCAGCU | 4295 | AS-134 | UCGUCACAGCUGAA | 4085 |
| | | GUGACGCdTdT | | | CGGCCdTdT | |
| D-154 | SS-154 | UGGCCACAUCCA | 4296 | AS-135 | UAAAUGUUGGAUGU | 4086 |
| | | ACAUUUCdTdT | | | GGCCAdTdT | |
| D-155 | SS-155 | AGCAGGACUAU | 4297 | AS-136 | UGCCAGAAAUAGUC | 4087 |
| | | UUCUGGCCdTdT | | | CUGCUdTdT | |
| D-156 | SS-156 | AUAUGGACAUC | 4298 | AS-137 | UGCAACCAGAUGUC | 4088 |
| | | UGGUUGCCdTdT | | | CAUAUdTdT | |
| D-157 | SS-157 | ACUUGCACCCUA | 4299 | AS-138 | UACAAGCUAGGGUG | 4089 |
| | | GCUUGUCdTdT | | | CAAGUdTdT | |
| D-158 | SS-158 | GUAAAGAGGUU | 4300 | AS-139 | UGGUUAGAAACCUC | 4090 |
| | | UCUAACCCdTdT | | | UUUACdTdT | |
| D-159 | SS-159 | GACUCACUUUAU | 4301 | AS-140 | UCUAUUGAUAAAGU | 4091 |
| | | CAAUAGCdTdT | | | GAGUCdTdT | |
| D-160 | SS-160 | AGCUGCUGACUC | 4302 | AS-141 | UUAAAGUGAGUCAG | 4092 |
| | | ACUUUACdTdT | | | CAGCUdTdT | |
| D-161 | SS-161 | AAGGGUUACCCU | 4303 | AS-142 | UGUGCCCAGGGUAA | 4093 |
| | | GGGCACCdTdT | | | CCCUUdTdT | |
| D-162 | SS-162 | UGUAAAGAGGU | 4304 | AS-143 | UGUUAGAAACCUCU | 4094 |
| | | UUCUAACCdTdT | | | UUACAdTdT | |
| D-163 | SS-163 | UGGCUUGCGGCG | 4305 | AS-144 | UUCCUCGCGCCGCA | 4095 |
| | | CGAGGACdTdT | | | AGCCAdTdT | |
| D-164 | SS-164 | GCCACGCUAGCU | 4306 | AS-145 | UCUCGUCAGCUAGC | 4096 |
| | | GACGAGCdTdT | | | GUGGCdTdT | |
| D-165 | SS-165 | CCCAAAGGAGGC | 4307 | AS-146 | UUUGUAAGCCUCCU | 4097 |
| | | UUACAACdTdT | | | UUGGGdTdT | |
| D-166 | SS-166 | UGUUGUGCUAU | 4308 | AS-147 | UUCCCCCCAUAGCA | 4098 |
| | | GGGGGGACdTdT | | | CAACAdTdT | |
| D-167 | SS-167 | GGGAACACACCC | 4309 | AS-148 | UCCAAGGGGGUGUG | 4099 |
| | | CCUUGGCdTdT | | | UUCCCdTdT | |
| D-168 | SS-168 | UUCCAAAUUGA | 4310 | AS-149 | UCCACCCAUCAAUU | 4100 |
| | | UGGGUGGCdTdT | | | UGGAAdTdT | |
| D-169 | SS-169 | ACCGGCAGCAUC | 4311 | AS-150 | UCAUGUCGAUGCUG | 4101 |
| | | GACAUGCdTdT | | | CCGGUdTdT | |
| D-170 | SS-170 | CUGGCUUGCGGC | 4312 | AS-151 | UCCUCGCGCCGCAA | 4102 |
| | | GCGAGGCdTdT | | | GCCAGdTdT | |
| D-171 | SS-171 | GUGUAAGAUAG | 4313 | AS-152 | UAUGUUAACUAUCU | 4103 |
| | | UUAACAUCdTdT | | | UACACdTdT | |
| D-172 | SS-172 | AGGAUUAGGAC | 4314 | AS-153 | UCGCUUCAGUCCUA | 4104 |
| | | UGAAGCGCdTdT | | | AUCCUdTdT | |
| D-173 | SS-173 | AAUGUGUAAGA | 4315 | AS-154 | UUUAACUAUCUUAC | 4105 |
| | | UAGUUAACdTdT | | | ACAUUdTdT | |
| D-174 | SS-174 | GGCCAGCCUAAG | 4316 | AS-155 | UCAUGAUCUUAGGC | 4106 |
| | | AUCAUGCdTdT | | | UGGCCdTdT | |
| D-175 | SS-175 | AGUUCCAUUUA | 4317 | AS-156 | UGUCAAUUUAAAUG | 4107 |
| | | AAUUGACCdTdT | | | GAACUdTdT | |
| D-176 | SS-176 | UCCAUUUAAAU | 4318 | AS-157 | UGAAGUCAAUUUAA | 4108 |
| | | UGACUUCCdTdT | | | AUGGAdTdT | |
| D-177 | SS-177 | CCCACCCUCACG | 4319 | AS-158 | UACACCUCGUGAGG | 4109 |
| | | AGGUGUCdTdT | | | GUGGGdTdT | |
| D-178 | SS-178 | CAUCUUAGCUUA | 4320 | AS-159 | UGAAAGCUAAGCUA | 4110 |
| | | GCUUUCCdTdT | | | AGAUGdTdT | |
| D-179 | SS-179 | ACUUAGCCCCAU | 4321 | AS-160 | UAAACUCAUGGGGC | 4111 |
| | | GAGUUUCdTdT | | | UAAGUdTdT | |
| D-180 | SS-180 | UCAUGGUUUAG | 4322 | AS-161 | UGAUCACCCUAAAC | 4112 |
| | | GGUGAUCCdTdT | | | CAUGAdTdT | |
| D-181 | SS-181 | GCCUGAAGCACA | 4323 | AS-162 | UUAAUCCUGUGCUU | 4113 |
| | | GGAUUACdTdT | | | CAGGCdTdT | |
| D-182 | SS-182 | CAUACUGAGGG | 4324 | AS-163 | UAAUUUCACCCUCA | 4114 |
| | | UGAAAUUCdTdT | | | GUAUGdTdT | |
| D-183 | SS-183 | AAGAUCAUGGU | 4325 | AS-164 | UACCCUAAACCAUG | 4115 |
| | | UUAGGGUCdTdT | | | AUCUUdTdT | |
| D-184 | SS-184 | GGGAUCUCCCCC | 4326 | AS-165 | UCCACAAGGGGGAG | 4116 |
| | | UUGUGGCdTdT | | | AUCCCdTdT | |
| D-185 | SS-185 | GAGGAAUGUGU | 4327 | AS-166 | UCUAUCUUACACAU | 4117 |
| | | AAGAUAGCdTdT | | | UCCUCdTdT | |
| D-186 | SS-186 | AUCAUGGUUUA | 4328 | AS-167 | UAUCACCCUAAACC | 4118 |
| | | GGGUGAUCdTdT | | | AUGAUdTdT | |
| D-187 | SS-187 | CAGCAUCGACAU | 4329 | AS-168 | UUCUACCAUGUCGA | 4119 |
| | | GGUAGACdTdT | | | UGCUGdTdT | |
| D-188 | SS-188 | GGUGCAGGCGU | 4330 | AS-169 | UUAUUCCUACGCCU | 4120 |
| | | AGGAAUACdTdT | | | GCACCdTdT | |
| D-189 | SS-189 | AGGAAUGUGUA | 4331 | AS-170 | UACUAUCUUACACA | 4121 |
| | | AGAUAGUCdTdT | | | UUCCUdTdT | |
| D-190 | SS-190 | CAUGGUUUAGG | 4332 | AS-171 | UUGAUCACCCUAAA | 4122 |
| | | GUGAUCACdTdT | | | CCAUGdTdT | |
| D-191 | SS-191 | UCCACCCAAGUC | 4333 | AS-172 | UGACGGCGACUUGG | 4123 |
| | | GCCGUCCdTdT | | | GUGGAdTdT | |
| D-192 | SS-192 | UCAGUAAUGACC | 4334 | AS-173 | UAACCAGGGUCAUU | 4124 |
| | | CUGGUUCdTdT | | | ACUGAdTdT | |
| D-193 | SS-193 | CUGGGACUUUA | 4335 | AS-174 | UUUAGCCCUAAAGU | 4125 |
| | | GGGCUAACdTdT | | | CCCAGdTdT | |
| D-194 | SS-194 | CAGGCGUAGGA | 4336 | AS-175 | UUCCAUAUUCCUAC | 4126 |
| | | AUAUGGACdTdT | | | GCCUGdTdT | |
| D-195 | SS-195 | GCAGGCGUAGG | 4337 | AS-176 | UCCAUAUUCCUACG | 4127 |
| | | AAUAUGGCdTdT | | | CCUGCdTdT | |
| D-196 | SS-196 | AGCCACGAGGUC | 4338 | AS-177 | UCGCCCCGACCUCG | 4128 |
| | | GGGGCGCdTdT | | | UGGCUdTdT | |
| D-197 | SS-197 | AAUCAUGCCUCC | 4339 | AS-178 | UUCUUAGGGAGGCA | 4129 |
| | | CUAAGACdTdT | | | UGAUUdTdT | |
| D-198 | SS-198 | UAAGCCGUUGGC | 4340 | AS-179 | UAGAGGUGCCAACG | 4130 |
| | | ACCUCUCdTdT | | | GCUUAdTdT | |
| D-199 | SS-199 | AUCGACAUGGU | 4341 | AS-180 | UCGAGUCUACCAUG | 4131 |
| | | AGACUCGCdTdT | | | UCGAUdTdT | |
| D-200 | SS-200 | UCUUCGGUAAU | 4342 | AS-181 | UCCUCAGAAUUACC | 4132 |
| | | UCUGAGGCdTdT | | | GAAGAdTdT | |
| D-201 | SS-201 | AAGUCGCCAGUG | 4343 | AS-182 | UAGACACCACUGGC | 4133 |
| | | GUGUCUCdTdT | | | GACUUdTdT | |
| D-202 | SS-202 | AUAAAUUGAAA | 4344 | AS-183 | UCGUGCCUUUUCAA | 4134 |
| | | AGGCACGCdTdT | | | UUUAUdTdT | |
| D-203 | SS-203 | GUUAGAGGCCCU | 4345 | AS-184 | UACCCCAAGGGCCU | 4135 |
| | | UGGGGUCdTdT | | | CUAACdTdT | |
| D-204 | SS-204 | AAUCAGGGGAU | 4346 | AS-185 | UCGCUGCGAUCCCC | 4136 |
| | | CGCAGCGCdTdT | | | UGAUUdTdT | |
| D-205 | SS-205 | UAAGAUAGUUA | 4347 | AS-186 | UCCCAUGUUAACUA | 4137 |
| | | ACAUGGGCdTdT | | | UCUUAdTdT | |
| D-206 | SS-206 | UGUAAGAUAGU | 4348 | AS-187 | UCAUGUUAACUAUC | 4138 |
| | | UAACAUGCdTdT | | | UUACAdTdT | |
| D-207 | SS-207 | CGGCCGCCUGUC | 4349 | AS-188 | UACCAAGGACAGGC | 4139 |
| | | CUUGGUCdTdT | | | GGCCGdTdT | |
| D-208 | SS-208 | UUAGAGGCCCUU | 4350 | AS-189 | UAACCCCAAGGGCC | 4140 |
| | | GGGGUUCdTdT | | | UCUAAdTdT | |
| D-209 | SS-209 | GGGAUCGCAGCG | 4351 | AS-190 | UUGUAGCCGCUGCG | 4141 |
| | | GCUACACdTdT | | | AUCCCdTdT | |
| D-210 | SS-210 | CACGAGGUCGGG | 4352 | AS-191 | UCCUCGCCCCGACC | 4142 |
| | | GCGAGGCdTdT | | | UCGUGdTdT | |

TABLE 5
Exemplary sense and antisense strand sequence for targeting MAPT

| | | | SS | | | AS |
|---|---|---|---|---|---|---|
| SIRNA | | | SEQ | | | SEQ |
| Duplex | | Sense Strand | ID | | Antisense Strand | ID |
| ID | SS ID | Sequence (5′-3′) | NO | AS ID | Sequence (5′-3′) | NO |
| D-001 | SS-001 | GCUUUGGCUCGG | 4477 | AS-001 | UGAAGUCCCGAGCC | 4687 |
| | | GACUUCC | | | AAAGC | |
| D-002 | SS-002 | CGACAUGGUAG | 4478 | AS-002 | UGGCGAGUCUACCA | 4688 |
| | | ACUCGCCC | | | UGUCG | |
| D-003 | SS-003 | CUCCUCGCAGUU | 4479 | AS-003 | UUAACCGAACUGCG | 4689 |
| | | CGGUUAC | | | AGGAG | |
| D-004 | SS-004 | GGCUUUGGCUCG | 4480 | AS-004 | UAAGUCCCGAGCCA | 4690 |
| | | GGACUUC | | | AAGCC | |
| D-005 | SS-005 | GGCUUUGGCUCG | 4481 | AS-004 | UAAGUCCCGAGCCA | 4690 |
| | | GGACUUA | | | AAGCC | |
| D-006 | SS-006 | GGCUUUGGCUCG | 4482 | AS-004 | UAAGUCCCGAGCCA | 4690 |
| | | GGACUUU | | | AAGCC | |
| D-007 | SS-007 | GGCUUUGGCACG | 4483 | AS-004 | UAAGUCCCGAGCCA | 4690 |
| | | GGACUUC | | | AAGCC | |
| D-008 | SS-008 | GGCUUUGGCUCG | 4484 | AS-004 | UAAGUCCCGAGCCA | 4690 |
| | | GGACUUG | | | AAGCC | |
| D-009 | SS-009 | GCAUCGACAUGG | 4485 | AS-005 | UAGUCUACCAUGUC | 4691 |
| | | UAGACUC | | | GAUGC | |
| D-010 | SS-010 | GCAUCGACAUGG | 4486 | AS-005 | UAGUCUACCAUGUC | 4691 |
| | | UAGACUA | | | GAUGC | |
| D-011 | SS-011 | GCAUCGACAUGG | 4487 | AS-005 | UAGUCUACCAUGUC | 4691 |
| | | UAGACUU | | | GAUGC | |
| D-012 | SS-012 | GCAUCGACAAGG | 4488 | AS-005 | UAGUCUACCAUGUC | 4691 |
| | | UAGACUC | | | GAUGC | |
| D-013 | SS-013 | GCAUCGACAUGG | 4489 | AS-005 | UAGUCUACCAUGUC | 4691 |
| | | UAGACUG | | | GAUGC | |
| D-014 | SS-014 | UGCUCCUCGCAG | 4490 | AS-006 | UACCGAACUGCGAG | 4692 |
| | | UUCGGUC | | | GAGCA | |
| D-015 | SS-015 | CACCCAAGUCGC | 4491 | AS-007 | UAAGACGGCGACUU | 4693 |
| | | CGUCUUC | | | GGGUG | |
| D-016 | SS-016 | CCUCGCAGUUCG | 4492 | AS-008 | UAUUAACCGAACUG | 4694 |
| | | GUUAAUC | | | CGAGG | |
| D-017 | SS-017 | UCCUCGCAGUUC | 4493 | AS-009 | UUUAACCGAACUGC | 4695 |
| | | GGUUAAC | | | GAGGA | |
| D-018 | SS-018 | AGCUGACCUUCC | 4494 | AS-010 | UUCUCGCGGAAGGU | 4696 |
| | | GCGAGAC | | | CAGCU | |
| D-019 | SS-019 | GACUGGAAGCG | 4495 | AS-011 | UUUGUCAUCGCUUC | 4697 |
| | | AUGACAAC | | | CAGUC | |
| D-020 | SS-020 | GACUGGAAGCG | 4496 | AS-011 | UUUGUCAUCGCUUC | 4697 |
| | | AUGACAAA | | | CAGUC | |
| D-021 | SS-021 | GACUGGAAGCG | 4497 | AS-011 | UUUGUCAUCGCUUC | 4697 |
| | | AUGACAAU | | | CAGUC | |
| D-022 | SS-022 | GACUGGAAGGG | 4498 | AS-011 | UUUGUCAUCGCUUC | 4697 |
| | | AUGACAAC | | | CAGUC | |
| D-023 | SS-023 | GACUGGAAGCG | 4499 | AS-011 | UUUGUCAUCGCUUC | 4697 |
| | | AUGACAAG | | | CAGUC | |
| D-024 | SS-024 | AGCUCGCAUGGU | 4500 | AS-012 | UUUACUGACCAUGC | 4698 |
| | | CAGUAAC | | | GAGCU | |
| D-025 | SS-025 | AGUCUACAAACC | 4501 | AS-013 | UUCAACUGGUUUGU | 4699 |
| | | AGUUGAC | | | AGACU | |
| D-026 | SS-026 | GCUCCUCGCAGU | 4502 | AS-014 | UAACCGAACUGCGA | 4700 |
| | | UCGGUUC | | | GGAGC | |
| D-027 | SS-027 | GCUCCUCGCAGU | 4503 | AS-014 | UAACCGAACUGCGA | 4700 |
| | | UCGGUUG | | | GGAGC | |
| D-028 | SS-028 | GCUCCUCGCAGU | 4504 | AS-014 | UAACCGAACUGCGA | 4700 |
| | | UCGGUUU | | | GGAGC | |
| D-029 | SS-029 | GCUCCUCGCUGU | 4505 | AS-014 | UAACCGAACUGCGA | 4700 |
| | | UCGGUUC | | | GGAGC | |
| D-030 | SS-030 | CUUCCGCGAGAA | 4506 | AS-015 | UUUGGCGUUCUCGC | 4701 |
| | | CGCCAAC | | | GGAAG | |
| D-031 | SS-031 | CUUCCGCGAGAA | 4507 | AS-015 | UUUGGCGUUCUCGC | 4701 |
| | | CGCCAAA | | | GGAAG | |
| D-032 | SS-032 | CUUCCGCGAGAA | 4508 | AS-015 | UUUGGCGUUCUCGC | 4701 |
| | | CGCCAAU | | | GGAAG | |
| D-033 | SS-033 | CUUCCGCGACAA | 4509 | AS-015 | UUUGGCGUUCUCGC | 4701 |
| | | CGCCAAC | | | GGAAG | |
| D-034 | SS-034 | CUUCCGCGAGAA | 4510 | AS-015 | UUUGGCGUUCUCGC | 4701 |
| | | CGCCAAG | | | GGAAG | |
| D-035 | SS-035 | AAAUCAGUGAU | 4511 | AS-016 | UUACUCCCAUCACU | 4702 |
| | | GGGAGUAC | | | GAUUU | |
| D-036 | SS-036 | GUCGCCGUCUUC | 4512 | AS-017 | UUUGGCGGAAGACG | 4703 |
| | | CGCCAAC | | | GCGAC | |
| D-037 | SS-037 | AUGGUAAAACG | 4513 | AS-018 | UCGAUCUUCGUUUU | 4704 |
| | | AAGAUCGC | | | ACCAU | |
| D-038 | SS-038 | ACGAAGAUCGCC | 4514 | AS-019 | UCGGUGUGGCGAUC | 4705 |
| | | ACACCGC | | | UUCGU | |
| D-039 | SS-039 | CGCAGUUCGGUU | 4515 | AS-020 | UCCAAUUAACCGAA | 4706 |
| | | AAUUGGC | | | CUGCG | |
| D-040 | SS-040 | GUACUCCACCCA | 4516 | AS-021 | UGCGACUUGGGUGG | 4707 |
| | | AGUCGCC | | | AGUAC | |
| D-041 | SS-041 | CUCGGCUUUGGC | 4517 | AS-022 | UUCCCGAGCCAAAG | 4708 |
| | | UCGGGAC | | | CCGAG | |
| D-042 | SS-042 | GCCCCUGGGGCG | 4518 | AS-023 | UAUUGACCGCCCCA | 4709 |
| | | GUCAAUC | | | GGGGC | |
| D-043 | SS-043 | CUCGCAGUUCGG | 4519 | AS-024 | UAAUUAACCGAACU | 4710 |
| | | UUAAUUC | | | GCGAG | |
| D-044 | SS-044 | UCGCCGUCUUCC | 4520 | AS-025 | UCUUGGCGGAAGAC | 4711 |
| | | GCCAAGC | | | GGCGA | |
| D-045 | SS-045 | GACGGGACUGG | 4521 | AS-026 | UAUCGCUUCCAGUC | 4712 |
| | | AAGCGAUC | | | CCGUC | |
| D-046 | SS-046 | UGGUAAAACGA | 4522 | AS-027 | UGCGAUCUUCGUUU | 4713 |
| | | AGAUCGCC | | | UACCA | |
| D-047 | SS-047 | GUAAAACGAAG | 4523 | AS-028 | UUGGCGAUCUUCGU | 4714 |
| | | AUCGCCAC | | | UUUAC | |
| D-048 | SS-048 | ACAAUAUCACCC | 4524 | AS-029 | UGGACGUGGGUGAU | 4715 |
| | | ACGUCCC | | | AUUGU | |
| D-049 | SS-049 | CCUUCCGCGAGA | 4525 | AS-030 | UUGGCGUUCUCGCG | 4716 |
| | | ACGCCAC | | | GAAGG | |
| D-050 | SS-050 | GGGGGCUGAUG | 4526 | AS-031 | UGUUUUACCAUCAG | 4717 |
| | | GUAAAACC | | | CCCCC | |
| D-051 | SS-051 | GCCGUCUUCCGC | 4527 | AS-032 | UCUCUUGGCGGAAG | 4718 |
| | | CAAGAGC | | | ACGGC | |
| D-052 | SS-052 | CACCGGCAGCAU | 4528 | AS-033 | UAUGUCGAUGCUGC | 4719 |
| | | CGACAUC | | | CGGUG | |
| D-053 | SS-053 | GUAAAAGCAAA | 4529 | AS-034 | UUCCCGUCUUUGCU | 4720 |
| | | GACGGGAC | | | UUUAC | |
| D-054 | SS-054 | AGGUUUCUAACC | 4530 | AS-035 | UAGGGUGGGUUAGA | 4721 |
| | | CACCCUC | | | AACCU | |
| D-055 | SS-055 | UUCCGCGAGAAC | 4531 | AS-036 | UUUUGGCGUUCUCG | 4722 |
| | | GCCAAAC | | | CGGAA | |
| D-056 | SS-056 | GACCUUCCGCGA | 4532 | AS-037 | UGCGUUCUCGCGGA | 4723 |
| | | GAACGCC | | | AGGUC | |
| D-057 | SS-057 | CGCCACGCUAGC | 4533 | AS-038 | UUCGUCAGCUAGCG | 4724 |
| | | UGACGAC | | | UGGCG | |
| D-058 | SS-058 | ACCUGCGUGUCC | 4534 | AS-039 | UUAGAUGGGACACG | 4725 |
| | | CAUCUAC | | | CAGGU | |
| D-059 | SS-059 | CCCCAGCUCGCC | 4535 | AS-040 | UUAGCGUGGCGAGC | 4726 |
| | | ACGCUAC | | | UGGGG | |
| D-060 | SS-060 | GCCGUUCAGCUG | 4536 | AS-041 | UUCGUCACAGCUGA | 4727 |
| | | UGACGAC | | | ACGGC | |
| D-061 | SS-061 | GCUGAUGGUAA | 4537 | AS-042 | UCUUCGUUUUACCA | 4728 |
| | | AACGAAGC | | | UCAGC | |
| D-062 | SS-062 | ACGAGGUGUCUC | 4538 | AS-043 | UGGGUGAGAGACAC | 4729 |
| | | UCACCCC | | | CUCGU | |
| D-063 | SS-063 | GGGCCAGCCUAA | 4539 | AS-044 | UAUGAUCUUAGGCU | 4730 |
| | | GAUCAUC | | | GGCCC | |
| D-064 | SS-064 | UCUAACCCACCC | 4540 | AS-045 | UUCGUGAGGGUGGG | 4731 |
| | | UCACGAC | | | UUAGA | |
| D-065 | SS-065 | UAAAGAGGUUU | 4541 | AS-046 | UGGGUUAGAAACCU | 4732 |
| | | CUAACCCC | | | CUUUA | |
| D-066 | SS-066 | CUCACCUCCUAA | 4542 | AS-047 | UAGUCUAUUAGGAG | 4733 |
| | | UAGACUC | | | GUGAG | |
| D-067 | SS-067 | GGAUAUACUGU | 4543 | AS-048 | UCCGGCAUACAGUA | 4734 |
| | | AUGCCGGC | | | UAUCC | |
| D-068 | SS-068 | CACCCUCACGAG | 4544 | AS-049 | UAGACACCUCGUGA | 4735 |
| | | GUGUCUC | | | GGGUG | |
| D-069 | SS-069 | CACGCUAGCUGA | 4545 | AS-050 | UACCUCGUCAGCUA | 4736 |
| | | CGAGGUC | | | GCGUG | |
| D-070 | SS-070 | UCCCAUGAUUUC | 4546 | AS-051 | UACCGAAGAAAUCA | 4737 |
| | | UUCGGUC | | | UGGGA | |
| D-071 | SS-071 | UUGAGUUGUAG | 4547 | AS-052 | UAAUCCAACUACAA | 4738 |
| | | UUGGAUUC | | | CUCAA | |
| D-072 | SS-072 | CUGCGUGUCCCA | 4548 | AS-053 | UUGUAGAUGGGACA | 4739 |
| | | UCUACAC | | | CGCAG | |
| D-073 | SS-073 | GUUGAGUUGUA | 4549 | AS-054 | UAUCCAACUACAAC | 4740 |
| | | GUUGGAUC | | | UCAAC | |
| D-074 | SS-074 | GGUGGUGGUUA | 4550 | AS-055 | UAUAUCUCUAACCA | 4741 |
| | | GAGAUAUC | | | CCACC | |
| D-075 | SS-075 | GGGCUAGAUAG | 4551 | AS-056 | UGUAUAUCCUAUCU | 4742 |
| | | GAUAUACC | | | AGCCC | |
| D-076 | SS-076 | AGGAUUCCAGCA | 4552 | AS-057 | UGGUUUUUGCUGGA | 4743 |
| | | AAAACCC | | | AUCCU | |
| D-077 | SS-077 | GGCCCCUGGGGC | 4553 | AS-058 | UUUGACCGCCCCAG | 4744 |
| | | GGUCAAC | | | GGGCC | |
| D-078 | SS-078 | GAUUAGGACUG | 4554 | AS-059 | UAUCGCUUCAGUCC | 4745 |
| | | AAGCGAUC | | | UAAUC | |
| D-079 | SS-079 | CUCCCAAGUUUU | 4555 | AS-060 | UCCUUUCAAAACUU | 4746 |
| | | GAAAGGC | | | GGGAG | |
| D-080 | SS-080 | GCUGGUCUGGCU | 4556 | AS-061 | UGCCGCAAGCCAGA | 4747 |
| | | UGCGGCC | | | CCAGC | |
| D-081 | SS-081 | GUUCCACUCAUC | 4557 | AS-062 | UCAGUUGGAUGAGU | 4748 |
| | | CAACUGC | | | GGAAC | |
| D-082 | SS-082 | CACCUCCUAAUA | 4558 | AS-063 | UUAAGUCUAUUAGG | 4749 |
| | | GACUUAC | | | AGGUG | |
| D-083 | SS-083 | UUCUAACCCACC | 4559 | AS-064 | UCGUGAGGGUGGGU | 4750 |
| | | CUCACGC | | | UAGAA | |
| D-084 | SS-084 | AAGCCCACAAUC | 4560 | AS-065 | UAGGCAUGAUUGUG | 4751 |
| | | AUGCCUC | | | GGCUU | |
| D-085 | SS-085 | ACCUUCCGCGAG | 4561 | AS-066 | UGGCGUUCUCGCGG | 4752 |
| | | AACGCCC | | | AAGGU | |
| D-086 | SS-086 | GCUGGUCACGUG | 4562 | AS-067 | UUUGGGUCACGUGA | 4753 |
| | | ACCCAAC | | | CCAGC | |
| D-087 | SS-087 | UAACCCACCCUC | 4563 | AS-068 | UCCUCGUGAGGGUG | 4754 |
| | | ACGAGGC | | | GGUUA | |
| D-088 | SS-088 | CUUACAACUCCU | 4564 | AS-069 | UUGAUGCAGGAGUU | 4755 |
| | | GCAUCAC | | | GUAAG | |
| D-089 | SS-089 | UGGGACUUUAG | 4565 | AS-070 | UGUUAGCCCUAAAG | 4756 |
| | | GGCUAACC | | | UCCCA | |
| D-090 | SS-090 | GCCACAGGCAGA | 4566 | AS-071 | UACAUCGUCUGCCU | 4757 |
| | | CGAUGUC | | | GUGGC | |
| D-091 | SS-091 | GCCUCUUGGGAG | 4567 | AS-072 | UGGACGUCUCCCAA | 4758 |
| | | ACGUCCC | | | GAGGC | |
| D-092 | SS-092 | GAGAUAUGCCCC | 4568 | AS-073 | UAUGAGGGGGGCAU | 4759 |
| | | CCUCAUC | | | AUCUC | |
| D-093 | SS-093 | UCCCAAGUUUUG | 4569 | AS-074 | UGCCUUUCAAAACU | 4760 |
| | | AAAGGCC | | | UGGGA | |
| D-094 | SS-094 | AGUUAUUACUC | 4570 | AS-075 | UUUAAUCAGAGUAA | 4761 |
| | | UGAUUAAC | | | UAACU | |
| D-095 | SS-095 | UAUGCCCCCCUC | 4571 | AS-076 | UAGUAAUGAGGGGG | 4762 |
| | | AUUACUC | | | GCAUA | |
| D-096 | SS-096 | CAAAAUCAGGG | 4572 | AS-077 | UUGCGAUCCCCUGA | 4763 |
| | | GAUCGCAC | | | UUUUG | |
| D-097 | SS-097 | CAGACCACGGGG | 4573 | AS-078 | UUCUCCGCCCCGUG | 4764 |
| | | CGGAGAC | | | GUCUG | |
| D-098 | SS-098 | CUUGGGUGGGC | 4574 | AS-079 | UCUAUCUAGCCCAC | 4765 |
| | | UAGAUAGC | | | CCAAG | |
| D-099 | SS-099 | CUCGCCACGCUA | 4575 | AS-080 | UGUCAGCUAGCGUG | 4766 |
| | | GCUGACC | | | GCGAG | |
| D-100 | SS-100 | UGUUGAGUUGU | 4576 | AS-081 | UUCCAACUACAACU | 4767 |
| | | AGUUGGAC | | | CAACA | |
| D-101 | SS-101 | UAUACAUAGAU | 4577 | AS-082 | UGGGCAACAUCUAU | 4768 |
| | | GUUGCCCC | | | GUAUA | |
| D-102 | SS-102 | AACCCACCCUCA | 4578 | AS-083 | UACCUCGUGAGGGU | 4769 |
| | | CGAGGUC | | | GGGUU | |
| D-103 | SS-103 | GUUGAGCAGGA | 4579 | AS-084 | UGAAAUAGUCCUGC | 4770 |
| | | CUAUUUCC | | | UCAAC | |
| D-104 | SS-104 | CCACGCUAGCUG | 4580 | AS-085 | UCCUCGUCAGCUAG | 4771 |
| | | ACGAGGC | | | CGUGG | |
| D-105 | SS-105 | CUCCUAAUAGAC | 4581 | AS-086 | UGGCUAAGUCUAUU | 4772 |
| | | UUAGCCC | | | AGGAG | |
| D-106 | SS-106 | GAUUUCUUCGG | 4582 | AS-087 | UAGAAUUACCGAAG | 4773 |
| | | UAAUUCUC | | | AAAUC | |
| D-107 | SS-107 | GUCCCAAAGGAG | 4583 | AS-088 | UGUAAGCCUCCUUU | 4774 |
| | | GCUUACC | | | GGGAC | |
| D-108 | SS-108 | GGUGGUUAGAG | 4584 | AS-089 | UGGCAUAUCUCUAA | 4775 |
| | | AUAUGCCC | | | CCACC | |
| D-109 | SS-109 | GGCUAGAUAGG | 4585 | AS-090 | UAGUAUAUCCUAUC | 4776 |
| | | AUAUACUC | | | UAGCC | |
| D-110 | SS-110 | AUUCACCAGAGU | 4586 | AS-091 | UAUAGUCACUCUGG | 4777 |
| | | GACUAUC | | | UGAAU | |
| D-111 | SS-111 | GAUAUACUGUA | 4587 | AS-092 | UGCCGGCAUACAGU | 4778 |
| | | UGCCGGCC | | | AUAUC | |
| D-112 | SS-112 | GACCACAGCAGG | 4588 | AS-093 | UCCAAUCCCUGCUG | 4779 |
| | | GAUUGGC | | | UGGUC | |
| D-113 | SS-113 | GAGCCACAGGCA | 4589 | AS-094 | UAUCGUCUGCCUGU | 4780 |
| | | GACGAUC | | | GGCUC | |
| D-114 | SS-114 | CUAGCUGACGAG | 4590 | AS-095 | UAGACACCUCGUCA | 4781 |
| | | GUGUCUC | | | GCUAG | |
| D-115 | SS-115 | AAGGGGGCUGA | 4591 | AS-096 | UUUUACCAUCAGCC | 4782 |
| | | UGGUAAAC | | | CCCUU | |
| D-116 | SS-116 | AUCAGGGGAUC | 4592 | AS-097 | UCCGCUGCGAUCCC | 4783 |
| | | GCAGCGGC | | | CUGAU | |
| D-117 | SS-117 | CCCAGCUCGCCA | 4593 | AS-098 | UCUAGCGUGGCGAG | 4784 |
| | | CGCUAGC | | | CUGGG | |
| D-118 | SS-118 | CGUUCAGCUGUG | 4594 | AS-099 | UCUUCGUCACAGCU | 4785 |
| | | ACGAAGC | | | GAACG | |
| D-119 | SS-119 | CACUUGCAAGUC | 4595 | AS-100 | UUCAUGGGACUUGC | 4786 |
| | | CCAUGAC | | | AAGUG | |
| D-120 | SS-120 | UGGGCUAGAUA | 4596 | AS-101 | UUAUAUCCUAUCUA | 4787 |
| | | GGAUAUAC | | | GCCCA | |
| D-121 | SS-121 | UUUAUGCUUGG | 4597 | AS-102 | UGGUGAAUCCAAGC | 4788 |
| | | AUUCACCC | | | AUAAA | |
| D-122 | SS-122 | CCAAAAUCAGGG | 4598 | AS-103 | UGCGAUCCCCUGAU | 4789 |
| | | GAUCGCC | | | UUUGG | |
| D-123 | SS-123 | CCGUUCAGCUGU | 4599 | AS-104 | UUUCGUCACAGCUG | 4790 |
| | | GACGAAC | | | AACGG | |
| D-124 | SS-124 | GGCCACAUCCAA | 4600 | AS-105 | UGAAAUGUUGGAUG | 4791 |
| | | CAUUUCC | | | UGGCC | |
| D-125 | SS-125 | UUUAUCAAUAG | 4601 | AS-106 | UAAUGGAACUAUUG | 4792 |
| | | UUCCAUUC | | | AUAAA | |
| D-126 | SS-126 | GGCGGAGAUCG | 4602 | AS-107 | UUUGUACACGAUCU | 4793 |
| | | UGUACAAC | | | CCGCC | |
| D-127 | SS-127 | UCGCCACGCUAG | 4603 | AS-108 | UCGUCAGCUAGCGU | 4794 |
| | | CUGACGC | | | GGCGA | |
| D-128 | SS-128 | GUCUGGCUUGCG | 4604 | AS-109 | UUCGCGCCGCAAGC | 4795 |
| | | GCGCGAC | | | CAGAC | |
| D-129 | SS-129 | ACCUCCUAAUAG | 4605 | AS-110 | UCUAAGUCUAUUAG | 4796 |
| | | ACUUAGC | | | GAGGU | |
| D-130 | SS-130 | UGUCCCAUCUAC | 4606 | AS-111 | UAGGUCUGUAGAUG | 4797 |
| | | AGACCUC | | | GGACA | |
| D-131 | SS-131 | CGGUAAUUCUG | 4607 | AS-112 | UCCACCCUCAGAAU | 4798 |
| | | AGGGUGGC | | | UACCG | |
| D-132 | SS-132 | UUUCUUCGGUA | 4608 | AS-113 | UUCAGAAUUACCGA | 4799 |
| | | AUUCUGAC | | | AGAAA | |
| D-133 | SS-133 | GCACCCUGUUGA | 4609 | AS-114 | UUACAACUCAACAG | 4800 |
| | | GUUGUAC | | | GGUGC | |
| D-134 | SS-134 | AUAUACUGUAU | 4610 | AS-115 | UAGCCGGCAUACAG | 4801 |
| | | GCCGGCUC | | | UAUAU | |
| D-135 | SS-135 | ACUUCUGGUUU | 4611 | AS-116 | UUGUACCCAAACCA | 4802 |
| | | GGGUACAC | | | GAAGU | |
| D-136 | SS-136 | GAAGCACAGGA | 4612 | AS-117 | UGUCCUAAUCCUGU | 4803 |
| | | UUAGGACC | | | GCUUC | |
| D-137 | SS-137 | CUUUCAGGGGUC | 4613 | AS-118 | UGCUUAGGACCCCU | 4804 |
| | | CUAAGCC | | | GAAAG | |
| D-138 | SS-138 | GAUAUGCCCCCC | 4614 | AS-119 | UUAAUGAGGGGGGC | 4805 |
| | | UCAUUAC | | | AUAUC | |
| D-139 | SS-139 | CUAAGGAGGCCG | 4615 | AS-120 | UGCUGAACGGCCUC | 4806 |
| | | UUCAGCC | | | CUUAG | |
| D-140 | SS-140 | GGAACACACCCC | 4616 | AS-121 | UUCCAAGGGGGUGU | 4807 |
| | | CUUGGAC | | | GUUCC | |
| D-141 | SS-141 | CCUAAGCCCACA | 4617 | AS-122 | UCAUGAUUGUGGGC | 4808 |
| | | AUCAUGC | | | UUAGG | |
| D-142 | SS-142 | CUGGGGCCUCCC | 4618 | AS-123 | UAAACUUGGGAGGC | 4809 |
| | | AAGUUUC | | | CCCAG | |
| D-143 | SS-143 | UGUCCCCCACUU | 4619 | AS-124 | UGGGUGCAAGUGGG | 4810 |
| | | GCACCCC | | | GGACA | |
| D-144 | SS-144 | AAGUUAUUACU | 4620 | AS-125 | UUAAUCAGAGUAAU | 4811 |
| | | CUGAUUAC | | | AACUU | |
| D-145 | SS-145 | AAUAGACUUAG | 4621 | AS-126 | UCAUGGGGCUAAGU | 4812 |
| | | CCCCAUGC | | | CUAUU | |
| D-146 | SS-146 | CAGUAAUGACCC | 4622 | AS-127 | UCAACCAGGGUCAU | 4813 |
| | | UGGUUGC | | | UACUG | |
| D-147 | SS-147 | GGAGGAAUGUG | 4623 | AS-128 | UUAUCUUACACAUU | 4814 |
| | | UAAGAUAC | | | CCUCC | |
| D-148 | SS-148 | CAAUCAUGCCUC | 4624 | AS-129 | UCUUAGGGAGGCAU | 4815 |
| | | CCUAAGC | | | GAUUG | |
| D-149 | SS-149 | UCCACUCAUCCA | 4625 | AS-130 | UCCCAGUUGGAUGA | 4816 |
| | | ACUGGGC | | | GUGGA | |
| D-150 | SS-150 | AGGUGCAGGCG | 4626 | AS-131 | UAUUCCUACGCCUG | 4817 |
| | | UAGGAAUC | | | CACCU | |
| D-151 | SS-151 | GAUAGGAUAUA | 4627 | AS-132 | UCAUACAGUAUAUC | 4818 |
| | | CUGUAUGC | | | CUAUC | |
| D-152 | SS-152 | UCCUAAGCCCAC | 4628 | AS-133 | UAUGAUUGUGGGCU | 4819 |
| | | AAUCAUC | | | UAGGA | |
| D-153 | SS-153 | GGCCGUUCAGCU | 4629 | AS-134 | UCGUCACAGCUGAA | 4820 |
| | | GUGACGC | | | CGGCC | |
| D-154 | SS-154 | UGGCCACAUCCA | 4630 | AS-135 | UAAAUGUUGGAUGU | 4821 |
| | | ACAUUUC | | | GGCCA | |
| D-155 | SS-155 | AGCAGGACUAU | 4631 | AS-136 | UGCCAGAAAUAGUC | 4822 |
| | | UUCUGGCC | | | CUGCU | |
| D-156 | SS-156 | AUAUGGACAUC | 4632 | AS-137 | UGCAACCAGAUGUC | 4823 |
| | | UGGUUGCC | | | CAUAU | |
| D-157 | SS-157 | ACUUGCACCCUA | 4633 | AS-138 | UACAAGCUAGGGUG | 4824 |
| | | GCUUGUC | | | CAAGU | |
| D-158 | SS-158 | GUAAAGAGGUU | 4634 | AS-139 | UGGUUAGAAACCUC | 4825 |
| | | UCUAACCC | | | UUUAC | |
| D-159 | SS-159 | GACUCACUUUAU | 4635 | AS-140 | UCUAUUGAUAAAGU | 4826 |
| | | CAAUAGC | | | GAGUC | |
| D-160 | SS-160 | AGCUGCUGACUC | 4636 | AS-141 | UUAAAGUGAGUCAG | 4827 |
| | | ACUUUAC | | | CAGCU | |
| D-161 | SS-161 | AAGGGUUACCCU | 4637 | AS-142 | UGUGCCCAGGGUAA | 4828 |
| | | GGGCACC | | | CCCUU | |
| D-162 | SS-162 | UGUAAAGAGGU | 4638 | AS-143 | UGUUAGAAACCUCU | 4829 |
| | | UUCUAACC | | | UUACA | |
| D-163 | SS-163 | UGGCUUGCGGCG | 4639 | AS-144 | UUCCUCGCGCCGCA | 4830 |
| | | CGAGGAC | | | AGCCA | |
| D-164 | SS-164 | GCCACGCUAGCU | 4640 | AS-145 | UCUCGUCAGCUAGC | 4831 |
| | | GACGAGC | | | GUGGC | |
| D-165 | SS-165 | CCCAAAGGAGGC | 4641 | AS-146 | UUUGUAAGCCUCCU | 4832 |
| | | UUACAAC | | | UUGGG | |
| D-166 | SS-166 | UGUUGUGCUAU | 4642 | AS-147 | UUCCCCCCAUAGCA | 4833 |
| | | GGGGGGAC | | | CAACA | |
| D-167 | SS-167 | GGGAACACACCC | 4643 | AS-148 | UCCAAGGGGGUGUG | 4834 |
| | | CCUUGGC | | | UUCCC | |
| D-168 | SS-168 | UUCCAAAUUGA | 4644 | AS-149 | UCCACCCAUCAAUU | 4835 |
| | | UGGGUGGC | | | UGGAA | |
| D-169 | SS-169 | ACCGGCAGCAUC | 4645 | AS-150 | UCAUGUCGAUGCUG | 4836 |
| | | GACAUGC | | | CCGGU | |
| D-170 | SS-170 | CUGGCUUGCGGC | 4646 | AS-151 | UCCUCGCGCCGCAA | 4837 |
| | | GCGAGGC | | | GCCAG | |
| D-171 | SS-171 | GUGUAAGAUAG | 4647 | AS-152 | UAUGUUAACUAUCU | 4838 |
| | | UUAACAUC | | | UACAC | |
| D-172 | SS-172 | AGGAUUAGGAC | 4648 | AS-153 | UCGCUUCAGUCCUA | 4839 |
| | | UGAAGCGC | | | AUCCU | |
| D-173 | SS-173 | AAUGUGUAAGA | 4649 | AS-154 | UUUAACUAUCUUAC | 4840 |
| | | UAGUUAAC | | | ACAUU | |
| D-174 | SS-174 | GGCCAGCCUAAG | 4650 | AS-155 | UCAUGAUCUUAGGC | 4841 |
| | | AUCAUGC | | | UGGCC | |
| D-175 | SS-175 | AGUUCCAUUUA | 4651 | AS-156 | UGUCAAUUUAAAUG | 4842 |
| | | AAUUGACC | | | GAACU | |
| D-176 | SS-176 | UCCAUUUAAAU | 4652 | AS-157 | UGAAGUCAAUUUAA | 4843 |
| | | UGACUUCC | | | AUGGA | |
| D-177 | SS-177 | CCCACCCUCACG | 4653 | AS-158 | UACACCUCGUGAGG | 4844 |
| | | AGGUGUC | | | GUGGG | |
| D-178 | SS-178 | CAUCUUAGCUUA | 4654 | AS-159 | UGAAAGCUAAGCUA | 4845 |
| | | GCUUUCC | | | AGAUG | |
| D-179 | SS-179 | ACUUAGCCCCAU | 4655 | AS-160 | UAAACUCAUGGGGC | 4846 |
| | | GAGUUUC | | | UAAGU | |
| D-180 | SS-180 | UCAUGGUUUAG | 4656 | AS-161 | UGAUCACCCUAAAC | 4847 |
| | | GGUGAUCC | | | CAUGA | |
| D-181 | SS-181 | GCCUGAAGCACA | 4657 | AS-162 | UUAAUCCUGUGCUU | 4848 |
| | | GGAUUAC | | | CAGGC | |
| D-182 | SS-182 | CAUACUGAGGG | 4658 | AS-163 | UAAUUUCACCCUCA | 4849 |
| | | UGAAAUUC | | | GUAUG | |
| D-183 | SS-183 | AAGAUCAUGGU | 4659 | AS-164 | UACCCUAAACCAUG | 4850 |
| | | UUAGGGUC | | | AUCUU | |
| D-184 | SS-184 | GGGAUCUCCCCC | 4660 | AS-165 | UCCACAAGGGGGAG | 4851 |
| | | UUGUGGC | | | AUCCC | |
| D-185 | SS-185 | GAGGAAUGUGU | 4661 | AS-166 | UCUAUCUUACACAU | 4852 |
| | | AAGAUAGC | | | UCCUC | |
| D-186 | SS-186 | AUCAUGGUUUA | 4662 | AS-167 | UAUCACCCUAAACC | 4853 |
| | | GGGUGAUC | | | AUGAU | |
| D-187 | SS-187 | CAGCAUCGACAU | 4663 | AS-168 | UUCUACCAUGUCGA | 4854 |
| | | GGUAGAC | | | UGCUG | |
| D-188 | SS-188 | GGUGCAGGCGU | 4664 | AS-169 | UUAUUCCUACGCCU | 4855 |
| | | AGGAAUAC | | | GCACC | |
| D-189 | SS-189 | AGGAAUGUGUA | 4665 | AS-170 | UACUAUCUUACACA | 4856 |
| | | AGAUAGUC | | | UUCCU | |
| D-190 | SS-190 | CAUGGUUUAGG | 4666 | AS-171 | UUGAUCACCCUAAA | 4857 |
| | | GUGAUCAC | | | CCAUG | |
| D-191 | SS-191 | UCCACCCAAGUC | 4667 | AS-172 | UGACGGCGACUUGG | 4858 |
| | | GCCGUCC | | | GUGGA | |
| D-192 | SS-192 | UCAGUAAUGACC | 4668 | AS-173 | UAACCAGGGUCAUU | 4859 |
| | | CUGGUUC | | | ACUGA | |
| D-193 | SS-193 | CUGGGACUUUA | 4669 | AS-174 | UUUAGCCCUAAAGU | 4860 |
| | | GGGCUAAC | | | CCCAG | |
| D-194 | SS-194 | CAGGCGUAGGA | 4670 | AS-175 | UUCCAUAUUCCUAC | 4861 |
| | | AUAUGGAC | | | GCCUG | |
| D-195 | SS-195 | GCAGGCGUAGG | 4671 | AS-176 | UCCAUAUUCCUACG | 4862 |
| | | AAUAUGGC | | | CCUGC | |
| D-196 | SS-196 | AGCCACGAGGUC | 4672 | AS-177 | UCGCCCCGACCUCG | 4863 |
| | | GGGGCGC | | | UGGCU | |
| D-197 | SS-197 | AAUCAUGCCUCC | 4673 | AS-178 | UUCUUAGGGAGGCA | 4864 |
| | | CUAAGAC | | | UGAUU | |
| D-198 | SS-198 | UAAGCCGUUGGC | 4674 | AS-179 | UAGAGGUGCCAACG | 4865 |
| | | ACCUCUC | | | GCUUA | |
| D-199 | SS-199 | AUCGACAUGGU | 4675 | AS-180 | UCGAGUCUACCAUG | 4866 |
| | | AGACUCGC | | | UCGAU | |
| D-200 | SS-200 | UCUUCGGUAAU | 4676 | AS-181 | UCCUCAGAAUUACC | 4867 |
| | | UCUGAGGC | | | GAAGA | |
| D-201 | SS-201 | AAGUCGCCAGUG | 4677 | AS-182 | UAGACACCACUGGC | 4868 |
| | | GUGUCUC | | | GACUU | |
| D-202 | SS-202 | AUAAAUUGAAA | 4678 | AS-183 | UCGUGCCUUUUCAA | 4869 |
| | | AGGCACGC | | | UUUAU | |
| D-203 | SS-203 | GUUAGAGGCCCU | 4679 | AS-184 | UACCCCAAGGGCCU | 4870 |
| | | UGGGGUC | | | CUAAC | |
| D-204 | SS-204 | AAUCAGGGGAU | 4680 | AS-185 | UCGCUGCGAUCCCC | 4871 |
| | | CGCAGCGC | | | UGAUU | |
| D-205 | SS-205 | UAAGAUAGUUA | 4681 | AS-186 | UCCCAUGUUAACUA | 4872 |
| | | ACAUGGGC | | | UCUUA | |
| D-206 | SS-206 | UGUAAGAUAGU | 4682 | AS-187 | UCAUGUUAACUAUC | 4873 |
| | | UAACAUGC | | | UUACA | |
| D-207 | SS-207 | CGGCCGCCUGUC | 4683 | AS-188 | UACCAAGGACAGGC | 4874 |
| | | CUUGGUC | | | GGCCG | |
| D-208 | SS-208 | UUAGAGGCCCUU | 4684 | AS-189 | UAACCCCAAGGGCC | 4875 |
| | | GGGGUUC | | | UCUAA | |
| D-209 | SS-209 | GGGAUCGCAGCG | 4685 | AS-190 | UUGUAGCCGCUGCG | 4876 |
| | | GCUACAC | | | AUCCC | |
| D-210 | SS-210 | CACGAGGUCGGG | 4686 | AS-191 | UCCUCGCCCCGACC | 4877 |
| | | GCGAGGC | | | UCGUG | |
| D-211 | SS-211 | GACUGGAAGCG | 4906 | AS-192 | UUUGUCAUCGCUUC | 4908 |
| | | AUGACAAUCU | | | CAGUCUU | |
| D-212 | SS-212 | CUUCCGCGAGAA | 4910 | AS-193 | UUUGGCGUUCUCGC | 4912 |
| | | CGCCAAUCU | | | GGAAGUU | |
| D-213 | SS-213 | GCAUCGACAUGG | 4914 | AS-194 | UAGUCUACCAUGUC | 4916 |
| | | UAGACUUCU | | | GAUGCUU | |
| D-214 | SS-214 | GCUCCUCGCAGU | 4918 | AS-195 | UAACCGAACUGCGA | 4920 |
| | | UCGGUUUCU | | | GGAGCUU | |
| D-215 | SS-215 | GGCUUUGGCUCG | 4922 | AS-196 | UAAGUCCCGAGCCA | 4924 |
| | | GGACUUUCU | | | AAGCCUU | |
| D-216 | SS-216 | GACUGGAAGCG | 4926 | AS-192 | UUUGUCAUCGCUUC | 4908 |
| | | AUGACAACCC | | | CAGUCUU | |
| D-217 | SS-217 | CUUCCGCGAGAA | 4930 | AS-193 | UUUGGCGUUCUCGC | 4912 |
| | | CGCCAACCC | | | GGAAGUU | |
| D-218 | SS-218 | GCAUCGACAUGG | 4934 | AS-194 | UAGUCUACCAUGUC | 4916 |
| | | UAGACUCCC | | | GAUGCUU | |
| D-219 | SS-219 | GCUCCUCGCAGU | 4938 | AS-195 | UAACCGAACUGCGA | 4920 |
| | | UCGGUUCCC | | | GGAGCUU | |
| D-220 | SS-220 | GGCUUUGGCUCG | 4942 | AS-196 | UAAGUCCCGAGCCA | 4924 |
| | | GGACUUCCC | | | AAGCCUU | |
| D-221 | SS-221 | GACUGGAAGCG | 4946 | AS-192 | UUUGUCAUCGCUUC | 4908 |
| | | AUGACAAACC | | | CAGUCUU | |
| D-222 | SS-222 | CUUCCGCGAGAA | 4950 | AS-193 | UUUGGCGUUCUCGC | 4912 |
| | | CGCCAAACC | | | GGAAGUU | |
| D-223 | SS-223 | GCAUCGACAUGG | 4954 | AS-194 | UAGUCUACCAUGUC | 4916 |
| | | UAGACUACC | | | GAUGCUU | |
| D-224 | SS-224 | GCUCCUCGCAGU | 4958 | AS-195 | UAACCGAACUGCGA | 4920 |
| | | UCGGUUGCC | | | GGAGCUU | |
| D-225 | SS-225 | GGCUUUGGCUCG | 4962 | AS-196 | UAAGUCCCGAGCCA | 4924 |
| | | GGACUUACC | | | AAGCCUU | |
| D-226 | SS-226 | GACUGGAAGCG | 4966 | AS-192 | UUUGUCAUCGCUUC | 4908 |
| | | AUGACAAUCC | | | CAGUCUU | |
| D-227 | SS-227 | CUUCCGCGAGAA | 4970 | AS-193 | UUUGGCGUUCUCGC | 4912 |
| | | CGCCAAUCC | | | GGAAGUU | |
| D-228 | SS-228 | GCAUCGACAUGG | 4974 | AS-194 | UAGUCUACCAUGUC | 4916 |
| | | UAGACUUCC | | | GAUGCUU | |
| D-229 | SS-229 | GCUCCUCGCAGU | 4978 | AS-195 | UAACCGAACUGCGA | 4920 |
| | | UCGGUUUCC | | | GGAGCUU | |
| D-230 | SS-230 | GGCUUUGGCUCG | 4982 | AS-196 | UAAGUCCCGAGCCA | 4924 |
| | | GGACUUUCC | | | AAGCCUU | |
| D-231 | SS-231 | GACUGGAAGGG | 4986 | AS-192 | UUUGUCAUCGCUUC | 4908 |
| | | AUGACAACCC | | | CAGUCUU | |
| D-232 | SS-232 | CUUCCGCGACAA | 4990 | AS-193 | UUUGGCGUUCUCGC | 4912 |
| | | CGCCAACCC | | | GGAAGUU | |
| D-233 | SS-233 | GCAUCGACAAGG | 4994 | AS-194 | UAGUCUACCAUGUC | 4916 |
| | | UAGACUCCC | | | GAUGCUU | |
| D-234 | SS-234 | GCUCCUCGCUGU | 4998 | AS-195 | UAACCGAACUGCGA | 4920 |
| | | UCGGUUCCC | | | GGAGCUU | |
| D-235 | SS-235 | GGCUUUGGCACG | 5002 | AS-196 | UAAGUCCCGAGCCA | 4924 |
| | | GGACUUCCC | | | AAGCCUU | |
| D-236 | SS-236 | GACUGGAAGCG | 5006 | AS-192 | UUUGUCAUCGCUUC | 4908 |
| | | AUGACAAGCC | | | CAGUCUU | |
| D-237 | SS-237 | CUUCCGCGAGAA | 5010 | AS-193 | UUUGGCGUUCUCGC | 4912 |
| | | CGCCAAGCC | | | GGAAGUU | |
| D-238 | SS-238 | GCAUCGACAUGG | 5014 | AS-194 | UAGUCUACCAUGUC | 4916 |
| | | UAGACUGCC | | | GAUGCUU | |
| D-239 | SS-239 | GGCUUUGGCUCG | 5022 | AS-196 | UAAGUCCCGAGCCA | 4924 |
| | | GGACUUGCC | | | AAGCCUU | |
| D-240 | SS-240 | CCUCGCAGUUCG | 5052 | AS-008 | UAUUAACCGAACUG | 4694 |
| | | GUUAAUU | | | CGAGG | |
| D-241 | SS-241 | CCUCGCAGUUCG | 5054 | AS-197 | UAUUAACCGAACUG | 5057 |
| | | GUUAAUUCU | | | CGAGGUU | |
| D-242 | SS-242 | CCUCGCAGUUCG | 5060 | AS-197 | UAUUAACCGAACUG | 5057 |
| | | GUUAAUCCC | | | CGAGGUU | |
| D-243 | SS-243 | CCUCGCAGUUCG | 5062 | AS-008 | UAUUAACCGAACUG | 4694 |
| | | GUUAAUA | | | CGAGG | |
| D-244 | SS-244 | CCUCGCAGUUCG | 5064 | AS-197 | UAUUAACCGAACUG | 5057 |
| | | GUUAAUACC | | | CGAGGUU | |
| D-245 | SS-245 | CCUCGCAGUUCG | 5066 | AS-197 | UAUUAACCGAACUG | 5057 |
| | | GUUAAUUCC | | | CGAGGUU | |
| D-246 | SS-246 | CCUCGCAGUACG | 5068 | AS-008 | UAUUAACCGAACUG | 4694 |
| | | GUUAAUC | | | CGAGG | |
| D-247 | SS-247 | CCUCGCAGUACG | 5070 | AS-197 | UAUUAACCGAACUG | 5057 |
| | | GUUAAUCCC | | | CGAGGUU | |
| D-248 | SS-248 | CCUCGCAGUUCG | 5072 | AS-008 | UAUUAACCGAACUG | 4694 |
| | | GUUAAUG | | | CGAGG | |
| D-249 | SS-249 | CCUCGCAGUUCG | 5074 | AS-197 | UAUUAACCGAACUG | 5057 |
| | | GUUAAUGCC | | | CGAGGUU | |
| D-250 | SS-250 | GCUUUGGCUCGG | 5077 | AS-198 | UGAAGUCCCGAGCC | 5080 |
| | | GACUUCCCC | | | AAAGCUU | |
| D-251 | SS-251 | GCUUUGGCUCGG | 5082 | AS-001 | UGAAGUCCCGAGCC | 4687 |
| | | GACUUCA | | | AAAGC | |
| D-252 | SS-252 | GCUUUGGCUCGG | 5084 | AS-198 | UGAAGUCCCGAGCC | 5080 |
| | | GACUUCACC | | | AAAGCUU | |
| D-253 | SS-253 | GCUUUGGCUCGG | 5086 | AS-001 | UGAAGUCCCGAGCC | 4687 |
| | | GACUUCU | | | AAAGC | |
| D-254 | SS-254 | GCUUUGGCUCGG | 5088 | AS-198 | UGAAGUCCCGAGCC | 5080 |
| | | GACUUCUCC | | | AAAGCUU | |
| D-255 | SS-255 | GCUUUGGCUGG | 5090 | AS-001 | UGAAGUCCCGAGCC | 4687 |
| | | GGACUUCC | | | AAAGC | |
| D-256 | SS-256 | GCUUUGGCUGG | 5092 | AS-198 | UGAAGUCCCGAGCC | 5080 |
| | | GGACUUCCCC | | | AAAGCUU | |
| D-257 | SS-257 | GCUUUGGCUCGG | 5094 | AS-001 | UGAAGUCCCGAGCC | 4687 |
| | | GACUUCG | | | AAAGC | |
| D-258 | SS-258 | GCUUUGGCUCGG | 5096 | AS-198 | UGAAGUCCCGAGCC | 5080 |
| | | GACUUCGCC | | | AAAGCUU | |
| D-259 | SS-259 | GCUUUGGCUCGG | 5098 | AS-198 | UGAAGUCCCGAGCC | 5080 |
| | | GACUUCUCU | | | AAAGCUU | |
| D-260 | SS-260 | AGCUGACCUUCC | 5101 | AS-199 | UUCUCGCGGAAGGU | 5104 |
| | | GCGAGACCC | | | CAGCUUU | |
| D-261 | SS-261 | AGCUGACCUUCC | 5106 | AS-010 | UUCUCGCGGAAGGU | 4696 |
| | | GCGAGAA | | | CAGCU | |
| D-262 | SS-262 | AGCUGACCUUCC | 5108 | AS-199 | UUCUCGCGGAAGGU | 5104 |
| | | GCGAGAACC | | | CAGCUUU | |
| D-263 | SS-263 | AGCUGACCUUCC | 5110 | AS-010 | UUCUCGCGGAAGGU | 4696 |
| | | GCGAGAU | | | CAGCU | |
| D-264 | SS-264 | AGCUGACCUUCC | 5112 | AS-199 | UUCUCGCGGAAGGU | 5104 |
| | | GCGAGAUCC | | | CAGCUUU | |
| D-265 | SS-265 | AGCUGACCUACC | 5114 | AS-010 | UUCUCGCGGAAGGU | 4696 |
| | | GCGAGAC | | | CAGCU | |
| D-266 | SS-266 | AGCUGACCUACC | 5116 | AS-199 | UUCUCGCGGAAGGU | 5104 |
| | | GCGAGACCC | | | CAGCUUU | |
| D-267 | SS-267 | AGCUGACCUUCC | 5118 | AS-010 | UUCUCGCGGAAGGU | 4696 |
| | | GCGAGAG | | | CAGCU | |
| D-268 | SS-268 | AGCUGACCUUCC | 5120 | AS-199 | UUCUCGCGGAAGGU | 5104 |
| | | GCGAGAGCC | | | CAGCUUU | |
| D-269 | SS-269 | AGCUGACCUUCC | 5122 | AS-199 | UUCUCGCGGAAGGU | 5104 |
| | | GCGAGAUCU | | | CAGCUUU | |
| D-270 | SS-270 | GCCGUCUUCCGC | 5125 | AS-200 | UCUCUUGGCGGAAG | 5128 |
| | | CAAGAGCCC | | | ACGGCUU | |
| D-271 | SS-271 | GCCGUCUUCCGC | 5130 | AS-032 | UCUCUUGGCGGAAG | 4718 |
| | | CAAGAGA | | | ACGGC | |
| D-272 | SS-272 | GCCGUCUUCCGC | 5132 | AS-200 | UCUCUUGGCGGAAG | 5128 |
| | | CAAGAGACC | | | ACGGCUU | |
| D-273 | SS-273 | GCCGUCUUCCGC | 5134 | AS-032 | UCUCUUGGCGGAAG | 4718 |
| | | CAAGAGU | | | ACGGC | |
| D-274 | SS-274 | GCCGUCUUCCGC | 5136 | AS-200 | UCUCUUGGCGGAAG | 5128 |
| | | CAAGAGUCC | | | ACGGCUU | |
| D-275 | SS-275 | GCCGUCUUCGGC | 5138 | AS-032 | UCUCUUGGCGGAAG | 4718 |
| | | CAAGAGC | | | ACGGC | |
| D-276 | SS-276 | GCCGUCUUCGGC | 5140 | AS-200 | UCUCUUGGCGGAAG | 5128 |
| | | CAAGAGCCC | | | ACGGCUU | |
| D-277 | SS-277 | GCCGUCUUCCGC | 5142 | AS-032 | UCUCUUGGCGGAAG | 4718 |
| | | CAAGAGG | | | ACGGC | |
| D-278 | SS-278 | GCCGUCUUCCGC | 5144 | AS-200 | UCUCUUGGCGGAAG | 5128 |
| | | CAAGAGGCC | | | ACGGCUU | |
| D-279 | SS-279 | GCCGUCUUCCGC | 5146 | AS-200 | UCUCUUGGCGGAAG | 5128 |
| | | CAAGAGUCU | | | ACGGCUU | |

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4700. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4502-4505. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4700 and the sense strand sequence comprises at least 15, 16, 17, 18, or 19 contiguous nucleotides differing by 3, 2, 1, or 0 nucleotides from the nucleotide sequence of any one of SEQ ID NOs: 4502-4505.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4920. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 4918, 4938, 4958, 4978, or 4998. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4920 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4918, 4938, 4958, 4978, or 4998.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4920. In some embodiments, the antisense strand comprises at least 20 or 21 contiguous nucleotides of SEQ ID NO: 4920. In some embodiments, the antisense strand comprises the nucleotide sequence of SEQ ID NO: 4920. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4918. In some embodiments, the sense strand sequence comprises at least 20 or 21 contiguous nucleotides of SEQ ID NO: 4918. In some embodiments, the sense strand sequence comprises the nucleotide sequence of SEQ ID NO: 4918. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4920 and the sense strand sequence comprises at least 15, 16, 17, 18, 19, 20, or 21 contiguous nucleotides differing by 3, 2, 1, or 0 nucleotides from the nucleotide sequence of SEQ ID NO: 4918. In some embodiments, the antisense strand sequence comprises at least 20 or 21 contiguous nucleotides of SEQ ID NO: 4920; and the sense strand sequence comprises at least 20 or 21 contiguous nucleotides of SEQ ID NO: 4918. In some embodiments, the antisense strand sequence comprises the nucleotide sequence of SEQ ID NO: 4920; and the sense strand comprises the nucleotide sequence of SEQ ID NO: 4918.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4919. In some embodiments, the nucleotide sequence encoding the antisense strand comprises at least 20 or 21 contiguous nucleotides of SEQ ID NO: 4919. In some embodiments, the nucleotide sequence encoding the antisense strand comprises the nucleotide sequence of SEQ ID NO: 4919. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4917. In some embodiments, the nucleotide sequence encoding the sense strand sequence comprises at least 20 or 21 contiguous nucleotides of SEQ ID NO: 4917. In some embodiments, the nucleotide sequence encoding the sense strand sequence comprises the nucleotide sequence of SEQ ID NO: 4917. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4919 and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4917. In some embodiments, the nucleotide sequence encoding the antisense strand sequence comprises at least 20 or 21 contiguous nucleotides of SEQ ID NO: 4919; and the nucleotide sequence encoding the sense strand sequence comprises at least 20 or 21 contiguous nucleotides of SEQ ID NO: 4917. In some embodiments, the nucleotide sequence encoding the antisense strand sequence comprises the nucleotide sequence of SEQ ID NO: 4919; and the nucleotide sequence encoding the sense strand comprises the nucleotide sequence of SEQ ID NO: 4917.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4920. In some embodiments, the antisense strand comprises at least 20 or 21 contiguous nucleotides of SEQ ID NO: 4920. In some embodiments, the antisense strand comprises the nucleotide sequence of SEQ ID NO: 4920. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4978. In some embodiments, the sense strand sequence comprises at least 20 or 21 contiguous nucleotides of SEQ ID NO: 4978. In some embodiments, the sense strand sequence comprises the nucleotide sequence of SEQ ID NO: 4978. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4920; and the sense strand sequence comprises at least 15, 16, 17, 18, 19, 20, or 21 contiguous nucleotides differing by 3, 2, 1, or 0 nucleotides from the nucleotide sequence of SEQ ID NO: 4978. In some embodiments, the antisense strand sequence comprises at least 20 or 21 contiguous nucleotides of SEQ ID NO: 4920; and the sense strand sequence comprises at least 20 or 21 contiguous nucleotides of SEQ ID NO: 4978. In some embodiments, the antisense strand sequence comprises the nucleotide sequence of SEQ ID NO: 4920; and the sense strand comprises the nucleotide sequence of SEQ ID NO: 4978.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4919. In some embodiments, the nucleotide sequence encoding the antisense strand comprises at least 20 or 21 contiguous nucleotides of SEQ ID NO: 4919. In some embodiments, the nucleotide sequence encoding the antisense strand comprises the nucleotide sequence of SEQ ID NO: 4919. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4977. In some embodiments, the nucleotide sequence encoding the sense strand sequence comprises at least 20 or 21 contiguous nucleotides of SEQ ID NO: 4977. In some embodiments, the nucleotide sequence encoding the sense strand sequence comprises the nucleotide sequence of SEQ ID NO: 4977. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4919; and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4977. In some embodiments, the nucleotide sequence encoding the antisense strand sequence comprises at least 20 or 21 contiguous nucleotides of SEQ ID NO: 4919; and the nucleotide sequence encoding the sense strand sequence comprises at least 20 or 21 contiguous nucleotides of SEQ ID NO: 4977. In some embodiments, the nucleotide sequence encoding the antisense strand sequence comprises the nucleotide sequence of SEQ ID NO: 4919; and the nucleotide sequence encoding the sense strand comprises the nucleotide sequence of SEQ ID NO: 4977.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4697. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4495-4499. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4697 and the sense strand sequence comprises at least 15, 16, 17, 18, or 19 contiguous nucleotides differing by 3, 2, 1, or 0 nucleotides from the nucleotide sequence of any one of SEQ ID NOs: 4495-4499.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4908. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 4906, 4926, 4946, 4966, 4986, or 5006. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4908 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4906, 4926, 4946, 4966, 4986, or 5006.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4908. In some embodiments, the antisense strand comprises at least 20 or 21 contiguous nucleotides of SEQ ID NO: 4908. In some embodiments, the antisense strand comprises the nucleotide sequence of SEQ ID NO: 4908. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4906. In some embodiments, the sense strand sequence comprises at least 20 or 21 contiguous nucleotides of SEQ ID NO: 4906. In some embodiments, the sense strand sequence comprises the nucleotide sequence of SEQ ID NO: 4906. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4908; and the sense strand sequence comprises at least 15, 16, 17, 18, 19, 20, or 21 contiguous nucleotides differing by 3, 2, 1, or 0 nucleotides from the nucleotide sequence of SEQ ID NO: 4906. In some embodiments, the antisense strand sequence comprises at least 20 or 21 contiguous nucleotides of SEQ ID NO: 4908; and the sense strand sequence comprises at least 20 or 21 contiguous nucleotides of SEQ ID NO: 4906. In some embodiments, the antisense strand sequence comprises the nucleotide sequence of SEQ ID NO: 4908; and the sense strand comprises the nucleotide sequence of SEQ ID NO: 4906.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4907. In some embodiments, the nucleotide sequence encoding the antisense strand comprises at least 20 or 21 contiguous nucleotides of SEQ ID NO: 4907. In some embodiments, the nucleotide sequence encoding the antisense strand comprises the nucleotide sequence of SEQ ID NO: 4907. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4905. In some embodiments, the nucleotide sequence encoding the sense strand sequence comprises at least 20 or 21 contiguous nucleotides of SEQ ID NO: 4905. In some embodiments, the nucleotide sequence encoding the sense strand sequence comprises the nucleotide sequence of SEQ ID NO: 4905. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4907; and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4905. In some embodiments, the nucleotide sequence encoding the antisense strand sequence comprises at least 20 or 21 contiguous nucleotides of SEQ ID NO: 4907; and the nucleotide sequence encoding the sense strand sequence comprises at least 20 or 21 contiguous nucleotides of SEQ ID NO: 4905. In some embodiments, the nucleotide sequence encoding the antisense strand sequence comprises the nucleotide sequence of SEQ ID NO: 4907; and the nucleotide sequence encoding the sense strand comprises the nucleotide sequence of SEQ ID NO: 4905.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4908. In some embodiments, the antisense strand comprises at least 20 or 21 contiguous nucleotides of SEQ ID NO: 4908. In some embodiments, the antisense strand comprises the nucleotide sequence of SEQ ID NO: 4908. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4966. In some embodiments, the sense strand sequence comprises at least 20 or 21 contiguous nucleotides of SEQ ID NO: 4966. In some embodiments, the sense strand sequence comprises the nucleotide sequence of SEQ ID NO: 4966. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4908 and the sense strand sequence comprises at least 15, 16, 17, 18, 19, 20, or 21 contiguous nucleotides differing by 3, 2, 1, or 0 nucleotides from the nucleotide sequence of SEQ ID NO: 4966. In some embodiments, the antisense strand sequence comprises at least 20 or 21 contiguous nucleotides of SEQ ID NO: 4908; and the sense strand sequence comprises at least 20 or 21 contiguous nucleotides of SEQ ID NO: 4966. In some embodiments, the antisense strand sequence comprises the nucleotide sequence of SEQ ID NO: 4908; and the sense strand comprises the nucleotide sequence of SEQ ID NO: 4966.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4907. In some embodiments, the nucleotide sequence encoding the antisense strand comprises at least 20 or 21 contiguous nucleotides of SEQ ID NO: 4907. In some embodiments, the nucleotide sequence encoding the antisense strand comprises the nucleotide sequence of SEQ ID NO: 4907. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4965. In some embodiments, the nucleotide sequence encoding the sense strand sequence comprises at least 20 or 21 contiguous nucleotides of SEQ ID NO: 4965. In some embodiments, the nucleotide sequence encoding the sense strand sequence comprises the nucleotide sequence of SEQ ID NO: 4965. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4907; and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4965. In some embodiments, the nucleotide sequence encoding the antisense strand sequence comprises at least 20 or 21 contiguous nucleotides of SEQ ID NO: 4907; and the nucleotide sequence encoding the sense strand sequence comprises at least 20 or 21 contiguous nucleotides of SEQ ID NO: 4965. In some embodiments, the nucleotide sequence encoding the antisense strand sequence comprises the nucleotide sequence of SEQ ID NO: 4907; and the nucleotide sequence encoding the sense strand comprises the nucleotide sequence of SEQ ID NO: 4965.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4690. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4480-4484. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4690 and the sense strand sequence comprises at least 15, 16, 17, 18, or 19 contiguous nucleotides differing by 3, 2, 1, or 0 nucleotides from the nucleotide sequence of any one of SEQ ID NOs: 4480-4484.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4924. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 4922, 4942, 4962, 4982, 5002, or 5022. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4924 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4922, 4942, 4962, 4982, 5002, or 5022.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4924. In some embodiments, the antisense strand comprises at least 20 or 21 contiguous nucleotides of SEQ ID NO: 4924. In some embodiments, the antisense strand comprises the nucleotide sequence of SEQ ID NO: 4924. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4922. In some embodiments, the sense strand sequence comprises at least 20 or 21 contiguous nucleotides of SEQ ID NO: 4922. In some embodiments, the sense strand sequence comprises the nucleotide sequence of SEQ ID NO: 4922. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4924; and the sense strand sequence comprises at least 15, 16, 17, 18, 19, 20, or 21 contiguous nucleotides differing by 3, 2, 1, or 0 nucleotides from the nucleotide sequence of SEQ ID NO: 4922. In some embodiments, the antisense strand sequence comprises at least 20 or 21 contiguous nucleotides of SEQ ID NO: 4924; and the sense strand sequence comprises at least 20 or 21 contiguous nucleotides of SEQ ID NO: 4922. In some embodiments, the antisense strand sequence comprises the nucleotide sequence of SEQ ID NO: 4924; and the sense strand comprises the nucleotide sequence of SEQ ID NO: 4922.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4923. In some embodiments, the nucleotide sequence encoding the antisense strand comprises at least 20 or 21 contiguous nucleotides of SEQ ID NO: 4923. In some embodiments, the nucleotide sequence encoding the antisense strand comprises the nucleotide sequence of SEQ ID NO: 4923. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4921. In some embodiments, the nucleotide sequence encoding the sense strand sequence comprises at least 20 or 21 contiguous nucleotides of SEQ ID NO: 4921. In some embodiments, the nucleotide sequence encoding the sense strand sequence comprises the nucleotide sequence of SEQ ID NO: 4921. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4923; and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4921. In some embodiments, the nucleotide sequence encoding the antisense strand sequence comprises at least 20 or 21 contiguous nucleotides of SEQ ID NO: 4923; and the nucleotide sequence encoding the sense strand sequence comprises at least 20 or 21 contiguous nucleotides of SEQ ID NO: 4921. In some embodiments, the nucleotide sequence encoding the antisense strand sequence comprises the nucleotide sequence of SEQ ID NO: 4923; and the nucleotide sequence encoding the sense strand comprises the nucleotide sequence of SEQ ID NO: 4921.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4924. In some embodiments, the antisense strand comprises at least 20 or 21 contiguous nucleotides of SEQ ID NO: 4924. In some embodiments, the antisense strand comprises the nucleotide sequence of SEQ ID NO: 4924. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4982. In some embodiments, the sense strand sequence comprises at least 20 or 21 contiguous nucleotides of SEQ ID NO: 4982. In some embodiments, the sense strand sequence comprises the nucleotide sequence of SEQ ID NO: 4982. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4924; and the sense strand sequence comprises at least 15, 16, 17, 18, 19, 20, or 21 contiguous nucleotides differing by 3, 2, 1, or 0 nucleotides from the nucleotide sequence of SEQ ID NO: 4982. In some embodiments, the antisense strand sequence comprises at least 20 or 21 contiguous nucleotides of SEQ ID NO: 4924; and the sense strand sequence comprises at least 20 or 21 contiguous nucleotides of SEQ ID NO: 4982. In some embodiments, the antisense strand sequence comprises the nucleotide sequence of SEQ ID NO: 4924; and the sense strand comprises the nucleotide sequence of SEQ ID NO: 4982.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4923. In some embodiments, the nucleotide sequence encoding the antisense strand comprises at least 20 or 21 contiguous nucleotides of SEQ ID NO: 4923. In some embodiments, the nucleotide sequence encoding the antisense strand comprises the nucleotide sequence of SEQ ID NO: 4923. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4981. In some embodiments, the nucleotide sequence encoding the sense strand sequence comprises at least 20 or 21 contiguous nucleotides of SEQ ID NO: 4981. In some embodiments, the nucleotide sequence encoding the sense strand sequence comprises the nucleotide sequence of SEQ ID NO: 4981. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4923; and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4981. In some embodiments, the nucleotide sequence encoding the antisense strand sequence comprises at least 20 or 21 contiguous nucleotides of SEQ ID NO: 4923; and the nucleotide sequence encoding the sense strand sequence comprises at least 20 or 21 contiguous nucleotides of SEQ ID NO: 4981. In some embodiments, the nucleotide sequence encoding the antisense strand sequence comprises the nucleotide sequence of SEQ ID NO: 4923; and the nucleotide sequence encoding the sense strand comprises the nucleotide sequence of SEQ ID NO: 4981.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4694. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4492, 5052, 5062, 5068, or 5072. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4694 and the sense strand sequence comprises at least 15, 16, 17, 18, or 19 contiguous nucleotides differing by 3, 2, 1, or 0 nucleotides from the nucleotide sequence of any one of SEQ ID NOs: 4492, 5052, 5062, 5068, or 5072.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 5057. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 5054, 5060, 5064, 5066, 5070, or 5074. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 5057; and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 5054, 5060, 5064, 5066, 5070, or 5074.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4691. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4485-4489. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4691 and the sense strand sequence comprises at least 15, 16, 17, 18, or 19 contiguous nucleotides differing by 3, 2, 1, or 0 nucleotides from the nucleotide sequence of any one of SEQ ID NOs: 4485-4489.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4916. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 4914, 4934, 4954, 4974, 4994, or 5014. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4916 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4914, 4934, 4954, 4974, 4994, or 5014.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4701. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4506-4510. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4701 and the sense strand sequence comprises at least 15, 16, 17, 18, or 19 contiguous nucleotides differing by 3, 2, 1, or 0 nucleotides from the nucleotide sequence of any one of SEQ ID NOs: 4506-4510.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4912. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 4910, 4930, 4950, 4970, 4990, or 5010. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4912 and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 4910, 4930, 4950, 4970, 4990, or 5010.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 4912. In some embodiments, the antisense strand comprises at least 20 or 21 contiguous nucleotides of SEQ ID NO: 4912. In some embodiments, the antisense strand comprises the nucleotide sequence of SEQ ID NO: 4912. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of SEQ ID NO: 4970. In some embodiments, the sense strand sequence comprises at least 20 or 21 contiguous nucleotides of SEQ ID NO: 4970. In some embodiments, the sense strand sequence comprises the nucleotide sequence of SEQ ID NO: 4970. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 4912; and the sense strand sequence comprises at least 15, 16, 17, 18, 19, 20, or 21 contiguous nucleotides differing by 3, 2, 1, or 0 nucleotides from the nucleotide sequence of SEQ ID NO: 4970. In some embodiments, the antisense strand sequence comprises at least 20 or 21 contiguous nucleotides of SEQ ID NO: 4912; and the sense strand sequence comprises at least 20 or 21 contiguous nucleotides of SEQ ID NO: 4970. In some embodiments, the antisense strand sequence comprises the nucleotide sequence of SEQ ID NO: 4912; and the sense strand comprises the nucleotide sequence of SEQ ID NO: 4970.

In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4911. In some embodiments, the nucleotide sequence encoding the antisense strand comprises at least 20 or 21 contiguous nucleotides of SEQ ID NO: 4911. In some embodiments, the nucleotide sequence encoding the antisense strand comprises the nucleotide sequence of SEQ ID NO: 4911. In some embodiments, the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4969. In some embodiments, the nucleotide sequence encoding the sense strand sequence comprises at least 20 or 21 contiguous nucleotides of SEQ ID NO: 4969. In some embodiments, the nucleotide sequence encoding the sense strand sequence comprises the nucleotide sequence of SEQ ID NO: 4969. In some embodiments, the nucleotide sequence encoding the antisense strand sequence comprises at least 15, 16, 17, 18, 19, 20, or 21 contiguous nucleotides differing by 3, 2, 1, or 0 nucleotides from the nucleotide sequence of SEQ ID NO: 4911; and the nucleotide sequence encoding the sense strand sequence comprises at least 15, 16, 17, 18, 19, 20, or 21 contiguous nucleotides differing by 3, 2, 1, or 0 nucleotides from the nucleotide sequence of SEQ ID NO: 4969. In some embodiments, the nucleotide sequence encoding the antisense strand sequence comprises at least 20 or 21 contiguous nucleotides of SEQ ID NO: 4911; and the nucleotide sequence encoding the sense strand sequence comprises at least 20 or 21 contiguous nucleotides of SEQ ID NO: 4969. In some embodiments, the nucleotide sequence encoding the antisense strand sequence comprises the nucleotide sequence of SEQ ID NO: 4911; and the nucleotide sequence encoding the sense strand comprises the nucleotide sequence of SEQ ID NO: 4969.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4687. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4477, 5082, 5086, 5090, 5094, or 5086. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4687 and the sense strand sequence comprises at least 15, 16, 17, 18, or 19 contiguous nucleotides differing by 3, 2, 1, or 0 nucleotides from the nucleotide sequence of any one of SEQ ID NOs: 4477, 5082, 5086, 5090, 5094, or 5086.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 5080. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 5077, 5084, 5088, 5092, 5096, or 5098. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 5080; and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 5077, 5084, 5088, 5092, 5096, or 5098.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4712. In some embodiments, the siRNA comprises a sense strand sequence comprising at least 15, 16, 17, 18, or 19 contiguous nucleotides differing by 3, 2, 1, or 0 nucleotides from the nucleotide sequence of SEQ ID NO: 4521. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4712 and the sense strand sequence comprises at least 15, 16, 17, 18, or 19 contiguous nucleotides differing by 3, 2, 1, or 0 nucleotides from the nucleotide sequence of SEQ ID NO: 4521.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4696. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4494, 5106, 5110, 5114, or 5118. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4696 and the sense strand sequence comprises at least 15, 16, 17, 18, or 19 contiguous nucleotides differing by 3, 2, 1, or 0 nucleotides from the nucleotide sequence of any one of SEQ ID NOs: 4494, 5106, 5110, 5114, or 5118.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 5104. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 5101, 5108, 5112, 5116, 5120, or 5122. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 5104; and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 5101, 5108, 5112, 5116, 5120, or 5122.

In some embodiments, the siRNA for targeting MAPT comprises an antisense strand sequence comprising at least 15, 16, 17, 18, or 19 contiguous nucleotides differing by 3, 2, 1, or 0 nucleotides from the nucleotide sequence of SEQ ID NO: 4718. In some embodiments, the siRNA comprises a sense strand sequence comprising at least 15, 16, 17, 18, or 19 contiguous nucleotides differing by 3, 2, 1, or 0 nucleotides from the nucleotide sequence of any one of SEQ ID NOs: 4527, 5130, 5134, 5138, or 5142. In some embodiments, the antisense strand sequence comprises at least 15, 16, 17, 18, or 19 contiguous nucleotides differing by 3, 2, 1, or 0 nucleotides from the nucleotide sequence of SEQ ID NO: 4718 and the sense strand sequence comprises at least 15, 16, 17, 18, or 19 contiguous nucleotides differing by 3, 2, 1, or 0 nucleotides from the nucleotide sequence of any one of SEQ ID NOs: 4527, 5130, 5134, 5138, or 5142.

In some embodiments, the siRNA for targeting MAPT comprises an antisense strand sequence comprising at least 15, 16, 17, 18, 19, 20, or 21 contiguous nucleotides differing by 3, 2, 1, or 0 nucleotides from the nucleotide sequence of SEQ ID NO: 5128. In some embodiments, the siRNA comprises a sense strand sequence comprising at least 15, 16, 17, 18, 19, 20, or 21 contiguous nucleotides differing by 3, 2, 1, or 0 nucleotides from the nucleotide sequence of any one of SEQ ID NOs: 5125, 5132, 5136, 5140, 5144, or 5146. In some embodiments, the antisense strand sequence comprises at least 15, 16, 17, 18, 19, 20, or 21 contiguous nucleotides differing by 3, 2, 1, or 0 nucleotides from the nucleotide sequence of SEQ ID NO: 5128; and the sense strand sequence comprises at least 15, 16, 17, 18, 19, 20, or 21 contiguous nucleotides differing by 3, 2, 1, or 0 nucleotides from the nucleotide sequence of any one of SEQ ID NOs: 5125, 5132, 5136, 5140, 5144, or 5146.

In some embodiments, the siRNA molecule is screened for the ability to inhibit tau (MAPT) expression in a cell. As a non-limiting example, the cell is a HEK293 cell. As another non-limiting example, the cell is a human tau transgenic primary neuron and/or astrocyte. As another non-limiting example, the cell is a frontotemporal lobar degeneration (FTLD) patient fibroblast. As a non-limiting example, other standard cell lines that may be used for initial screening are: naïve neuronal SK-N-AS cells, monkey cell lines (CYNOM-K1, FRhK-4, or LLC-MK2), primary neuron and astrocyte cultures from htau mice, frontotemporal lobar degeneration (FTLD)-tau patient induced pluripotent stem cells (iPSCs), FTLD-tau patient fibroblasts; nervous system cells derived from multiple affected or at-risk donors (with and without MAPT mutations), FTLD-tau patient lymphoblasts, and human neural stem cells bearing homozygous or heterozygous mutations in MAPT (e.g., P301L, R406W, V337M) that may be introduced using CRISPR-Cas9 gene editing technology. The screened siRNA duplex may be selected from any of the siRNA duplexes presented in Table 4 or 5 of the present disclosure.

In some embodiments, the siRNA molecules of the present disclosure can be encoded by a vector, e.g., plasmid vector, a viral genome (e.g., an AAV viral genome), or another nucleic acid expression vector for delivery to a cell. In some embodiments, a siRNA molecule is encoded by a viral genome of an AAV particle.

DNA expression plasmids can be used to stably express the siRNA duplexes or dsRNA of the present disclosure in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

In some embodiments, the sense and antisense strands of a siRNA duplex may be linked by a short spacer sequence, which may optionally be linked to additional flanking sequence, leading to the expression of a flanking arm-stem-loop structure termed primary microRNA (pri-miRNA). The pri-miRNA may be recognized and cleaved by Drosha and Dicer, and thus generate mature siRNA molecules.

The expression of MAPT may be assessed based on the level of expression of MAPT mRNA or MAPT protein. In some embodiments, the expression of MAPT, e.g., MAPT mRNA, is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the siRNA duplexes or encoded dsRNA of the present disclosure suppress (or degrade) the target mRNA in a cell, e.g., a neuron (e.g., a cortical neuron) or a glial cell.

According to the present disclosure, the siRNA molecules are designed and tested for their ability to reduce MAPT transcript levels in cultured cells. Such siRNA molecules may form a duplex such as, but not limited to, any of the sequences listed in Tables 4, 5, 9A, or 9B.

In some embodiments, the siRNA molecules comprise a miRNA seed match for the target (e.g., MAPT transcript) located in the guide strand. In another embodiment, the siRNA molecules comprise a miRNA seed match for the target (e.g., MAPT transcript) located in the passenger strand. In yet another embodiment, the siRNA duplexes or encoded dsRNA targeting MAPT transcript do not comprise a seed match for the target (e.g., MAPT transcript) located in the guide or passenger strand. In some embodiments, the siRNA duplex is designed so there is no miRNA seed match for the sense or antisense sequence to non-MAPT sequences.

The present disclosure also provides a pharmaceutical composition comprising a siRNA duplex targeting a MAPT gene, mRNA, or protein, and a pharmaceutically acceptable carrier. In some aspects, the siRNA duplex is encoded by a viral genome and/or modulatory polynucleotide. In some embodiments, an AAV particle comprises the viral genome and an AAV capsid protein, e.g., an AAV capsid variant described herein.

In some embodiments, the guide to passenger (G: P) (also referred to as the antisense to sense) strand ratio is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. In some embodiments, the guide to passenger ratio refers to the ratio of the guide strands to the passenger strands after the intracellular processing of the pri-microRNA. For example, a 80:20 guide-to-passenger ratio would have 8 guide strands to every 2 passenger strands processed from the precursor.

In some embodiments, the passenger to guide (P: G) (also referred to as the sense to antisense) strand ratio is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. In some embodiments, the passenger to guide ratio refers to the ratio of the passenger strands to the guide strands after the excision of the guide strand. For example, a 80:20 passenger to guide ratio would have 8 passenger strands to every 2 guide strands processed from the precursor.

### Molecular Scaffold

In some embodiments, a viral genome described herein encodes a modulatory polynucleotide comprising a siRNA, e.g., an siRNA described herein for targeting MAPT. In some embodiments, a viral genome described herein comprises a modulatory polynucleotide encoding a siRNA, e.g., an siRNA described herein for targeting MAPT. In some embodiments, the modulatory polynucleotide comprises a molecular scaffold. In some embodiments, a molecular scaffold comprises a framework or starting molecule that forms the sequence or structural basis against which to design or make a subsequent molecule. In some embodiments, the modulatory polynucleotide comprises a 5′ flanking region, a loop region, and a 3′ flanking region. In some embodiments, the modulatory polynucleotide further comprises a passenger strand (e.g., a passenger strand or guide strand sequence of a siRNA for targeting MAPT described herein) and/or a guide strand (e.g., a guide strand or antisense strand sequence of a siRNA for targeting MAPT described herein). In some embodiments, the modulatory polynucleotide is an RNA molecule, e.g., an encoded modulatory polynucleotide. In some embodiments, the modulatory polynucleotide is a DNA molecule, e.g., a DNA molecule encoding the RNA modulatory polynucleotide.

In some embodiments, the encoded modulatory polynucleotide comprises in 5′ to 3′ order: a 5′ flanking region, a passenger strand (e.g., a passenger strand or guide strand sequence of a siRNA for targeting MAPT described herein), a loop region, a guide strand (e.g., a guide strand or antisense strand sequence of a siRNA for targeting MAPT described herein), and a 3′ flanking region. In some embodiments, the encoded modulatory polynucleotide is an RNA molecule.

In some embodiments, the modulatory polynucleotide comprises in 5′ to 3′ order: a 5′ flanking region, a guide strand (e.g., a guide strand or antisense strand sequence of a siRNA for targeting MAPT described herein), a loop region, a passenger strand (e.g., a passenger strand or sense strand sequence of a siRNA for targeting MAPT described herein), and a 3′ flanking region. In some embodiments, the modulatory polynucleotide is a DNA molecule.

In some embodiments, the modulatory polynucleotide comprises a stem-loop structure. In some embodiments, the passenger strand and the guide strand are located, respectively, on a 5′ arm and a 3′ arm of a stem loop structure, wherein the passenger strand is located between the 5′ flanking region and the loop region and the guide strand is located between the loop region and the 3′ flanking region. In some embodiments, the guide strand and the passenger strand are located, respectively, on a 5′ arm and a 3′ arm of a stem loop structure, wherein the guide strand is located between the 5′ flanking region and the loop region and the passenger strand is located between the loop region and the 3′ flanking region.

In some embodiments the 5′ and 3′ flanking sequences comprise the same nucleotide sequence. In some embodiments the nucleotide sequence of the 5′ flanking region differs by at least 2%, 3%, 4%, 5%, relative to the 3′ flanking region, when aligned to each other. In some embodiments, the 5′ flanking region and the 3′ flanking region comprise different nucleotide sequences.

In some embodiments, the loop region comprises a nucleic acid sequence encoding at least one UGUG motif. In some embodiments, the nucleic acid sequence encoding the UGUG motif is located at the 5′ terminus of the loop sequence.

In some embodiments, a molecular scaffold described herein comprises the nucleotide sequence of a 5′ flanking region, a loop region and a 3′ flanking region, provided in Tables 6-8, or a fragment thereof. In some embodiments, the 5′ flanking region, loop region, and 3′ flanking region comprises or is encoded by a nucleotide sequence provided in Tables 6-8, or a fragment thereof.

TABLE 6
Exemplary 5′ Flanking Regions

| | | SEQ ID |
|---|---|---|
| Name | Sequence | NO |
| 5F1 (DNA) | TTTATGCCTCATCCTCTGAGTGCTGAAGGCTTGCTGTA | 4353 |
| | GGCTGTATGCTG | |
| 5F1 (RNA) | UUUAUGCCUCAUCCUCUGAGUGCUGAAGGCUUGCUG | 4878 |
| | UAGGCUGUAUGCUG | |
| 5F2 (DNA) | GTGCTGGGCGGGGGGCGGCGGGCCCTCCCGCAGAAC | 4354 |
| | ACCATGCGCTCTTCGGAA | |
| 5F2 (RNA) | GUGCUGGGCGGGGGGGGCGGGCCCUCCCGCAGAAC | 4879 |
| | ACCAUGCGCUCUUCGGAA | |
| 5F3 (DNA) | GAAGCAAAGAAGGGGCAGAGGGAGCCCGTGAGCTGA | 4355 |
| | GTGGGCCAGGGACTGGGAGAAGGAGTGAGGAGGCAG | |
| | GGCCGGCATGCCTCTGCTGCTGGCCAGA | |
| 5F3 (RNA) | GAAGCAAAGAAGGGGCAGAGGGAGCCCGUGAGCUG | 4880 |
| | AGUGGGCCAGGGACUGGGAGAAGGAGUGAGGAGGC | |
| | AGGGCCGGCAUGCCUCUGCUGCUGGCCAGA | |
| 5F4 (DNA) | GTGCTGGGCGGGGGGCGGCGGGCCCTCCCGCAGAAC | 4356 |
| | ACCATGCGCTCTTCGGGA | |
| 5F4 (RNA) | GUGCUGGGCGGGGGGCGGCGGGCCCUCCCGCAGAAC | 4881 |
| | ACCAUGCGCUCUUCGGGA | |
| 5F5 (DNA) | GTGCTGGGCGGGGGGCGGCGGGCCCTCCCGCAGAAC | 4357 |
| | ACCATGCGCTCCACGGAA | |
| 5F5 (RNA) | GUGCUGGGCGGGGGGCGGCGGGCCCUCCCGCAGAAC | 4882 |
| | ACCAUGCGCUCCACGGAA | |
| 5F6 (DNA) | GGGCCCTCCCGCAGAACACCATGCGCTCCACGGAA | 4358 |
| 5F5 (RNA) | GGGCCCUCCCGCAGAACACCAUGCGCUCCACGGAA | 4883 |
| 5F7 (DNA) | CTCCCGCAGAACACCATGCGCTCCACGGAA | 4359 |
| 5F7 (RNA) | CUCCCGCAGAACACCAUGCGCUCCACGGAA | 4884 |
| 5F8 (DNA) | GTGCTGGGCGGGGGGCGGCGGGCCCTCCCGCAGAAC | 4360 |
| | ACCATGCGCTCCACGGAAG | |
| 5F8 (RNA) | GUGCUGGGCGGGGGGCGGCGGGCCCUCCCGCAGAAC | 4885 |
| | ACCAUGCGCUCCACGGAAG | |
| 5F9 (DNA) | GTGCTGGGCGGGGGGCGGCGGGCCCTCCCGCAGAAC | 4361 |
| | ACCATGCGCTCCTCGGAA | |
| 5F9 (RNA) | GUGCUGGGGGGGGGGGGCGGGCCCUCCCGCAGAAC | 4886 |
| | ACCAUGCGCUCCUCGGAA | |

TABLE 7
Exemplary Loop Regions

| Name | Sequence | SEQ ID NO |
|---|---|---|
| L1 (DNA) | TGTGACCTGG | 4362 |
| L1 (RNA) | UGUGACCUGG | 4887 |
| L2 (DNA) | TGTGATTTGG | 4363 |
| L2 (RNA | UGUGAUUUGG | 4888 |
| L3 (DNA) | TATAATTTGG | 4364 |
| L3 (RNA) | UAUAAUUUGG | 4889 |
| L4 (DNA) | CCTGACCCAGT | 4365 |
| L4 (RNA) | CCUGACCCAGU | 4890 |
| L5 (DNA) | GTCTGCACCTGTCACTAG | 4366 |
| L5 (RNA) | GUCUGCACCUGUCACUAG | 4891 |
| L6 (DNA) | GTGACCCAAG | 4367 |
| L6 (RNA) | GUGACCCAAG | 4892 |
| L7 (DNA) | GTGGCCACTGAGAAG | 4368 |
| L7 (RNA) | GUGGCCACUGAGAAG | 4893 |
| L8 (DNA) | GTGACCCAAT | 4369 |
| L8 (RNA) | GUGACCCAAU | 4894 |
| L9 (DNA) | GTGACCCAAC | 4370 |
| L9 (RNA) | GUGACCCAAC | 4895 |
| L10 (DNA) | GTGGCCACTGAGAAA | 4371 |
| L10 (RNA) | GUGGCCACUGAGAAA | 4896 |

TABLE 8
Exemplary 3′ Flanking Regions

| | | SEQ ID |
|---|---|---|
| Name | Sequence | NO |
| 3F1 (DNA) | AGTGTATGATGCCTGTTACTAGCATTCACATGGAACA | 4372 |
| | AATTGCTGCCGTG | |
| 3F1 (RNA) | AGUGUAUGAUGCCUGUUACUAGCAUUCACAUGGAA | 4897 |
| | CAAAUUGCUGCCGUG | |
| 3F2 (DNA) | CTGAGGAGCGCCTTGACAGCAGCCATGGGAGGGCCG | 4373 |
| | CCCCCTACCTCAGTGA | |
| 3F2 (RNA) | CUGAGGAGCGCCUUGACAGCAGCCAUGGGAGGGCCG | 4898 |
| | CCCCCUACCUCAGUGA | |
| 3F3 (DNA) | CTGTGGAGCGCCTTGACAGCAGCCATGGGAGGGCCG | 4374 |
| | CCCCCTACCTCAGTGA | |
| 3F3 (RNA) | CUGUGGAGCGCCUUGACAGCAGCCAUGGGAGGGCCG | 4899 |
| | CCCCCUACCUCAGUGA | |
| 3F4 (DNA) | TGGCCGTGTAGTGCTACCCAGCGCTGGCTGCCTCCTC | 4375 |
| | AGCATTGCAATTCCTCTCCCATCTGGGCACCAGTCAG | |
| | CTACCCTGGTGGGAATCTGGGTAGCC | |
| 3F4 (RNA) | UGGCCGUGUAGUGCUACCCAGCGCUGGCUGCCUCCU | 4900 |
| | CAGCAUUGCAAUUCCUCUCCCAUCUGGGCACCAGUC | |
| | AGCUACCCUGGUGGGAAUCUGGGUAGCC | |
| 3F5 (DNA) | GGCCGTGTAGTGCTACCCAGCGCTGGCTGCCTCCTCA | 4376 |
| | GCATTGCAATTCCTCTCCCATCTGGGCACCAGTCAGC | |
| | TACCCTGGTGGGAATCTGGGTAGCC | |
| 3F5 (RNA) | GGCCGUGUAGUGCUACCCAGCGCUGGCUGCCUCCUC | 4901 |
| | AGCAUUGCAAUUCCUCUCCCAUCUGGGCACCAGUCA | |
| | GCUACCCUGGUGGGAAUCUGGGUAGCC | |
| 3F6 (DNA) | TCCTGAGGAGCGCCTTGACAGCAGCCATGGGAGGGC | 4377 |
| | CGCCCCCTACCTCAGTGA | |
| 3F6 (RNA) | UCCUGAGGAGCGCCUUGACAGCAGCCAUGGGAGGGC | 4902 |
| | CGCCCCCUACCUCAGUGA | |
| 3F7 (DNA) | CTGAGGAGCGCCTTGACAGCAGCCATGGGAGGGCC | 4378 |
| 3F7 (RNA) | CUGAGGAGCGCCUUGACAGCAGCCAUGGGAGGGCC | 4903 |
| 3F8 (DNA) | CTGCGGAGCGCCTTGACAGCAGCCATGGGAGGGCCG | 4379 |
| | CCCCCTACCTCAGTGA | |
| 3F8 (RNA) | CUGCGGAGCGCCUUGACAGCAGCCAUGGGAGGGCCG | 4904 |
| | CCCCCUACCUCAGUGA | |

Any of the regions described in Table 6, Table 7, and Table 8 or a fragment of variant thereof may be used in the molecular scaffolds described herein, e.g., a molecular scaffold encoding or comprising a siRNA for targeting MAPT described herein.

In some embodiments, the modulatory polynucleotide comprises a 5′ flanking region, fragment or variant thereof, e.g., as listed in Table 6 or as provided in WO2016077689, WO2017201248, WO2018204797, WO2017201258, WO2018204803, or WO2021016505, the contents of which are hereby incorporated by reference in their entirety.

In some embodiments, the encoded 5′ flanking region comprises the nucleotide sequence of any one of SEQ ID NOs: 4878-4886; a nucleotide sequence substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 4878-4886; or a nucleotide sequence comprising one, two, three, four, five, six, or seven, but no more than ten modifications relative to the nucleotide sequence of any of SEQ ID NOs: 4878-4886.

In some embodiments, the nucleotide sequence encoding the 5′ flanking region comprises the nucleotide sequence of any of SEQ ID NOs: 4353-4361, a nucleotide sequence, substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 4353-4361; or a nucleotide sequence comprising one, two, three, four, five, six, or seven, but no more than ten modifications relative to the nucleotide sequence of any of SEQ ID NOs: 4353-4361.

In some embodiments, the 5′ flanking region comprises the nucleotide sequence of SEQ ID NO: 4354; a nucleotide sequence, substantially identical (e.g., having at least 70%, 75%, 80%, 85%, 90%, 95% 96%, 97%, 98%, 99% sequence identity) to SEQ ID NO: 4354; a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354. In some embodiments, the 5′ flanking region comprises the nucleotide sequence of SEQ ID NO: 4355; a nucleotide sequence, substantially identical (e.g., having at least 70%, 75%, 80%, 85%, 90%, 95% 96%, 97%, 98%, 99% sequence identity) to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355.

In some embodiments, the encoded 5′ flanking region comprises the nucleotide sequence of SEQ ID NO: 4879; a nucleotide sequence, substantially identical (e.g., having at least 70%, 75%, 80%, 85%, 90%, 95% 96%, 97%, 98%, 99% sequence identity) to SEQ ID NO: 4879; a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879. In some embodiments, the nucleotide sequence encoding the 5′ flanking region comprises the nucleotide sequence of SEQ ID NO: 4354; a nucleotide sequence, substantially identical (e.g., having at least 70%, 75%, 80%, 85%, 90%, 95% 96%, 97%, 98%, 99% sequence identity) to SEQ ID NO: 4354; a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354.

In some embodiments, the encoded 5′ flanking region comprises the nucleotide sequence of SEQ ID NO: 4880; a nucleotide sequence, substantially identical (e.g., having at least 70%, 75%, 80%, 85%, 90%, 95% 96%, 97%, 98%, 99% sequence identity) to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880. In some embodiments, the nucleotide sequence encoding the 5′ flanking region comprises the nucleotide sequence of SEQ ID NO: 4355; a nucleotide sequence, substantially identical (e.g., having at least 70%, 75%, 80%, 85%, 90%, 95% 96%, 97%, 98%, 99% sequence identity) to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355.

In some embodiments, the modulatory polynucleotide comprises a loop region, fragment or variant thereof, e.g., as provided in Table 7 or as provided in WO2016077689, WO2017201248, WO2018204797, WO2017201258, WO2018204803, or WO2021016505, the contents of which are hereby incorporated by reference in their entirety.

In some embodiments, the encoded loop region comprises the nucleotide sequence of any of SEQ ID NOs: 4887-4896; a nucleotide sequence substantially identical (e.g., having at least 70%, 75%, 80%, 85%, 90%, 95% 96%, 97%, 98%, 99% sequence identity) thereto; a nucleotide sequence comprising one, two, three, or four, but no more than five different nucleotides relative to the nucleotide sequence of any of SEQ ID NOs: 4887-4896; or a nucleotide sequence comprising one, two, three, or four, but no more than five modifications relative to the nucleotide sequence of any of SEQ ID NOs: 4887-4896.

In some embodiments, the nucleotide sequence encoding the loop region comprises the nucleotide sequence of any of SEQ ID NOs: 4362-4371; a nucleotide sequence substantially identical (e.g., having at least 70%, 75%, 80%, 85%, 90%, 95% 96%, 97%, 98%, 99% sequence identity) thereto; a nucleotide sequence comprising one, two, three, or four, but no more than five different nucleotides relative to the nucleotide sequence of any of SEQ ID NOs: 4362-4371; or a nucleotide sequence comprising one, two, three, or four, but no more than five modifications relative to the nucleotide sequence of any of SEQ ID NOs: 4362-4371.

In some embodiments, the loop region comprises the nucleotide sequence of SEQ ID NO: 4362; a nucleotide sequence substantially identical (e.g., having at least 70%, 75%, 80%, 85%, 90%, 95% 96%, 97%, 98%, 99% sequence identity) to SEQ ID NO: 4362; a nucleotide sequence comprising one, two, three, or four, but no more than five different nucleotides relative to the nucleotide sequence of SEQ ID NO: 4362; or a nucleotide sequence comprising one, two, three, or four, but no more than five modifications relative to the nucleotide sequence of SEQ ID NO: 4362. In some embodiments, the loop region comprises the nucleotide sequence of SEQ ID NO: 4363; a nucleotide sequence substantially identical (e.g., having at least 70%, 75%, 80%, 85%, 90%, 95% 96%, 97%, 98%, 99% sequence identity) to SEQ ID NO: 4363; a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363. In some embodiments, the loop region comprises the nucleotide sequence of SEQ ID NO: 4366; a nucleotide sequence substantially identical (e.g., having at least 70%, 75%, 80%, 85%, 90%, 95% 96%, 97%, 98%, 99% sequence identity) to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366.

In some embodiments, the encoded loop region comprises the nucleotide sequence of SEQ ID NO: 4887; a nucleotide sequence substantially identical (e.g., having at least 70%, 75%, 80%, 85%, 90%, 95% 96%, 97%, 98%, 99% sequence identity) to SEQ ID NO: 4887; a nucleotide sequence comprising one, two, three, or four, but no more than five different nucleotides relative to the nucleotide sequence of SEQ ID NO: 4887; or a nucleotide sequence comprising one, two, three, or four, but no more than five modifications relative to the nucleotide sequence of SEQ ID NO: 4887. In some embodiments, the nucleotide sequence encoding the loop region comprises the nucleotide sequence of SEQ ID NO: 4362; a nucleotide sequence substantially identical (e.g., having at least 70%, 75%, 80%, 85%, 90%, 95% 96%, 97%, 98%, 99% sequence identity) to SEQ ID NO: 4362; a nucleotide sequence comprising one, two, three, or four, but no more than five different nucleotides relative to the nucleotide sequence of SEQ ID NO: 4362; or a nucleotide sequence comprising one, two, three, or four, but no more than five modifications relative to the nucleotide sequence of SEQ ID NO: 4362.

In some embodiments, the encoded loop region comprises the nucleotide sequence of SEQ ID NO: 4888; a nucleotide sequence substantially identical (e.g., having at least 70%, 75%, 80%, 85%, 90%, 95% 96%, 97%, 98%, 99% sequence identity) to SEQ ID NO: 4888; a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888. In some embodiments, the nucleotide sequence encoding the loop region comprises the nucleotide sequence of SEQ ID NO: 4363; a nucleotide sequence substantially identical (e.g., having at least 70%, 75%, 80%, 85%, 90%, 95% 96%, 97%, 98%, 99% sequence identity) to SEQ ID NO: 4363; a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363.

In some embodiments, the encoded loop region comprises the nucleotide sequence of SEQ ID NO: 4891; a nucleotide sequence substantially identical (e.g., having at least 70%, 75%, 80%, 85%, 90%, 95% 96%, 97%, 98%, 99% sequence identity) to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891. In some embodiments, the nucleotide sequence encoding the loop region comprises the nucleotide sequence of SEQ ID NO: 4366; a nucleotide sequence substantially identical (e.g., having at least 70%, 75%, 80%, 85%, 90%, 95% 96%, 97%, 98%, 99% sequence identity) to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366.

In some embodiments, the modulatory polynucleotide comprises a 3′ flanking region, fragment or variant thereof, e.g., as listed in Table 8 or as provided in WO2016077689, WO2017201248, WO2018204797, WO2017201258, WO2018204803, or WO2021016505, the contents of which are hereby incorporated by reference in their entirety.

In some embodiments, the encoded 3′ flanking region comprises the nucleotide sequence of any one of SEQ ID NOs: 4897-4904; a nucleotide sequence substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten modifications relative to the nucleotide sequence of any of SEQ ID NOs: 4897-4904; or a nucleotide sequence comprising one, two, three, four, five, six, or seven, but no more than ten modifications relative to the nucleotide sequence of any of SEQ ID NOs: 4897-4904.

In some embodiments, the nucleotide sequence encoding the 3′ flanking region comprises the nucleotide sequence of any of SEQ ID NOs: 4372-4379; a nucleotide sequence, substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 4372-4379; or a nucleotide sequence comprising one, two, three, four, five, six, or seven, but no more than ten modifications relative to the nucleotide sequence of any of SEQ ID NOs: 4372-4379.

In some embodiments, the 3′ flanking region comprises the nucleotide sequence of SEQ ID NO: 4373; a nucleotide sequence, substantially identical (e.g., having at least 70%, 75%, 80%, 85%, 90%, 95% 96%, 97%, 98%, 99% sequence identity) to SEQ ID NO: 4373; a nucleotide sequence comprising one, two, three, four, five, six, or seven, but no more than ten modifications relative to the nucleotide sequence of SEQ ID NO: 4373; or a nucleotide sequence comprising one, two, three, four, five, six, or seven, but no more than ten modifications relative to the nucleotide sequence of SEQ ID NO: 4373. In some embodiments, the 3′ flanking region comprises the nucleotide sequence of SEQ ID NO: 4374; a nucleotide sequence, substantially identical (e.g., having at least 70%, 75%, 80%, 85%, 90%, 95% 96%, 97%, 98%, 99% sequence identity) to SEQ ID NO: 4374; a nucleotide sequence comprising one, two, three, four, five, six, or seven, but no more than ten different nucleotides relative to the nucleotide sequence of SEQ ID NO: 4374; or a nucleotide sequence comprising one, two, three, four, five, six, or seven, but no more than ten modifications relative to the nucleotide sequence of SEQ ID NO: 4374. In some embodiments, the 3′ flanking region comprises the nucleotide sequence of SEQ ID NO: 4375; a nucleotide sequence, substantially identical (e.g., having at least 70%, 75%, 80%, 85%, 90%, 95% 96%, 97%, 98%, 99% sequence identity) to SEQ ID NO: 4375; a nucleotide sequence comprising one, two, three, four, five, six, or seven, but no more than ten modifications relative to the nucleotide sequence of SEQ ID NO: 4375; or a nucleotide sequence comprising one, two, three, four, five, six, or seven, but no more than ten modifications relative to the nucleotide sequence of SEQ ID NO: 4375.

In some embodiments, the encoded 3′ flanking region comprises the nucleotide sequence of SEQ ID NO: 4898; a nucleotide sequence substantially identical (e.g., having at least 70%, 75%, 80%, 85%, 90%, 95% 96%, 97%, 98%, 99% sequence identity) to SEQ ID NO: 4898; a nucleotide sequence comprising one, two, three, four, five, six, or seven, but no more than ten different nucleotides relative to the nucleotide sequence of SEQ ID NO: 4898; or a nucleotide sequence comprising one, two, three, four, five, six, or seven, but no more than ten modifications relative to the nucleotide sequence of SEQ ID NO: 4898. In some embodiments, the nucleotide sequence encoding the 3′ flanking region comprises the nucleotide sequence of SEQ ID NO: 4373; a nucleotide sequence, substantially identical (e.g., having at least 70%, 75%, 80%, 85%, 90%, 95% 96%, 97%, 98%, 99% sequence identity) to SEQ ID NO: 4373; a nucleotide sequence comprising one, two, three, four, five, six, or seven, but no more than ten different nucleotides relative to the nucleotide sequence of SEQ ID NO: 4373; or a nucleotide sequence comprising one, two, three, four, five, six, or seven, but no more than ten modifications relative to the nucleotide sequence of SEQ ID NO: 4373.

In some embodiments, the encoded 3′ flanking region comprises the nucleotide sequence of SEQ ID NO: 4899; a nucleotide sequence substantially identical (e.g., having at least 70%, 75%, 80%, 85%, 90%, 95% 96%, 97%, 98%, 99% sequence identity) to SEQ ID NO: 4899; a nucleotide sequence comprising one, two, three, four, five, six, or seven, but no more than ten different nucleotides relative to the nucleotide sequence of SEQ ID NO: 4898; or a nucleotide sequence comprising one, two, three, four, five, six, or seven, but no more than ten modifications relative to the nucleotide sequence of SEQ ID NO: 4899. In some embodiments, the nucleotide sequence encoding the 3′ flanking region comprises the nucleotide sequence of SEQ ID NO: 4374; a nucleotide sequence, substantially identical (e.g., having at least 70%, 75%, 80%, 85%, 90%, 95% 96%, 97%, 98%, 99% sequence identity) to SEQ ID NO: 4374; a nucleotide sequence comprising one, two, three, four, five, six, or seven, but no more than ten different nucleotides relative to the nucleotide sequence of SEQ ID NO: 4374; or a nucleotide sequence comprising one, two, three, four, five, six, or seven, but no more than ten modifications relative to the nucleotide sequence of SEQ ID NO: 4374.

me embodiments, the encoded 3′ flanking region comprises the nucleotide sequence of SEQ ID NO: 4900; a nucleotide sequence substantially identical (e.g., having at least 70%, 75%, 80%, 85%, 90%, 95% 96%, 97%, 98%, 99% sequence identity) to SEQ ID NO: 4900; a nucleotide sequence comprising one, two, three, four, five, six, or seven, but no more than ten different nucleotides relative to the nucleotide sequence of SEQ ID NO: 4898; or a nucleotide sequence comprising one, two, three, four, five, six, or seven, but no more than ten modifications relative to the nucleotide sequence of SEQ ID NO: 4900. In some embodiments, the nucleotide sequence encoding the 3′ flanking region comprises the nucleotide sequence of SEQ ID NO: 4375; a nucleotide sequence, substantially identical (e.g., having at least 70%, 75%, 80%, 85%, 90%, 95% 96%, 97%, 98%, 99% sequence identity) to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to the nucleotide sequence of SEQ ID NO: 4375.

In some embodiments, the encoded modulatory polynucleotide comprises: (i) a 5′ flanking region comprising the nucleotide sequence of any one of SEQ ID NOs: 4878-4886, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 4878-4886; or a nucleotide sequence comprising one, two, three, four, five, six, or seven, but no more than ten different nucleotides relative to the nucleotide sequence of any one of SEQ ID NOs: 4878-4886; (ii) a loop region comprising the nucleotide sequence of any one of SEQ ID NOs: 4887-4896, a nucleotide sequence substantially identical (e.g., having at least 70%, 75%, 80%, 85%, 90%, 95% 96%, 97%, 98%, 99% sequence identity) thereto, a nucleotide sequence comprising one, two, three, or four, but no more than five different nucleotides relative to the nucleotide sequence of any one of SEQ ID NOs: 4887-4896; or a nucleotide sequence comprising one, two, three, or four, but no more than five different nucleotides relative to the nucleotide sequence of any one of SEQ ID NOs: 4887-4896; and (iii) a 3′ flanking region of any one of SEQ ID NOs: 4897-4904, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 4897-4904, a nucleotide sequence comprising one, two, three, four, five, six, or seven, but no more than ten modifications relative to the nucleotide sequence of any one of SEQ ID NOs: 4897-4904.

In some embodiments, the modulatory polynucleotide comprises: (i) a 5′ flanking region comprising the nucleotide sequence of any one of SEQ ID NOs: 4353-4361, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 4353-4361; or a nucleotide sequence comprising one, two, three, four, five, six, or seven, but no more than ten different nucleotides relative to the nucleotide sequence of any one of SEQ ID NOs: 4353-4361; (ii) a loop region comprising the nucleotide sequence of any one of SEQ ID NOs: 4362-4371, a nucleotide sequence substantially identical (e.g., having at least 70%, 75%, 80%, 85%, 90%, 95% 96%, 97%, 98%, 99% sequence identity) thereto, a nucleotide sequence comprising one, two, three, or four, but no more than five different nucleotides relative to the nucleotide sequence of any one of SEQ ID NOs: 4362-4371; or a nucleotide sequence comprising one, two, three, or four, but no more than five different nucleotides relative to the nucleotide sequence of any one of SEQ ID NOs: 4362-4371; and (iii) a 3′ flanking region of any one of SEQ ID NOs: 4372-4379, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 4372-4379, a nucleotide sequence comprising one, two, three, four, five, six, or seven, but no more than ten modifications relative to the nucleotide sequence of any one of SEQ ID NOs: 4372-4379.

In some embodiments, the encoded modulatory polynucleotide comprises: (i) a 5′ flanking region comprising the nucleotide sequence of SEQ ID NO: 4879, a nucleotide sequence substantially identical (e.g., having at least 70%, 75%, 80%, 85%, 90%, 95% 96%, 97%, 98%, 99% sequence identity) to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten different nucleotides relative to the nucleotide sequence of SEQ ID NO: 4879; (ii) a loop region comprising the nucleotide sequence of SEQ ID NO: 4887, a nucleotide sequence substantially identical (e.g., having at least 70%, 75%, 80%, 85%, 90%, NO: 4887; or a nucleotide sequence comprising one, two, three, or four, but no more than five different nucleotides relative to the nucleotide sequence of SEQ ID NO: 4887; and (iii) a 3′ flanking region of SEQ ID NO: 4898, a nucleotide sequence substantially identical (e.g., having at least 70%, 75%, 80%, 85%, 90%, 95% 96%, 97%, 98%, 99% sequence identity) to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.

In some embodiments, the modulatory polynucleotide comprises: (i) a 5′ flanking region comprising the nucleotide sequence of SEQ ID NO: 4354, a nucleotide sequence substantially identical (e.g., having at least 70%, 75%, 80%, 85%, 90%, 95% 96%, 97%, 98%, 99% sequence identity) to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten different nucleotides relative to the nucleotide sequence of SEQ ID NO: 4354; (ii) a loop region comprising the nucleotide sequence of SEQ ID NO: 4362, a nucleotide sequence substantially identical (e.g., having at least 70%, 75%, 80%, 85%, 90%, 95% 96%, 97%, 98%, 99% sequence identity) to SEQ ID NO: 4362, a nucleotide sequence comprising one, two, three, or four, but no more than five different nucleotides relative to the nucleotide sequence of SEQ ID NO: 4362: or a nucleotide sequence comprising one, two, three, or four, but no more than five different nucleotides relative to the nucleotide sequence of SEQ ID NO: 4362; and (iii) a 3′ flanking region of SEQ ID NO: 4373, a nucleotide sequence substantially identical (e.g., having at least 70%, 75%, 80%, 85%, 90%, 95% 96%, 97%, 98%, 99% sequence identity) to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.

In some embodiments, the encoded modulatory polynucleotide comprises: (i) a 5′ flanking region comprising the nucleotide sequence of SEQ ID NO: 4879, a nucleotide sequence substantially identical (e.g., having at least 70%, 75%, 80%, 85%, 90%, 95% 96%, 97%, 98%, 99% sequence identity) to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879; (ii) a loop region comprising the nucleotide sequence of SEQ ID NO: 4888, a nucleotide sequence substantially identical (e.g., having at least 70%, 75%, 80%, 85%, 90%, 95% 96%, 97%, 98%, 99% sequence identity) to SEQ ID NO: 4888, a nucleotide sequence comprising one, NO: 4888; or a nucleotide sequence comprising one, two, three, or four, but no more than five modifications relative to the nucleotide sequence of SEQ ID NO: 4888; and (iii) a 3′ flanking region of SEQ ID NO: 4898, a nucleotide sequence substantially identical (e.g., having at least 70%, 75%, 80%, 85%, 90%, 95% 96%, 97%, 98%, 99% sequence identity) to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.

In some embodiments, the modulatory polynucleotide comprises: (i) a 5′ flanking region comprising the nucleotide sequence of SEQ ID NO: 4354, a nucleotide sequence substantially identical (e.g., having at least 70%, 75%, 80%, 85%, 90%, 95% 96%, 97%, 98%, 99% sequence identity) to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354; (ii) a loop region comprising the nucleotide sequence of SEQ ID NO: 4363, a nucleotide sequence substantially identical (e.g., having at least 70%, 75%, 80%, 85%, 90%, 95% 96%, 97%, 98%, 99% sequence identity) to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363; and (iii) a 3′ flanking region of SEQ ID NO: 4373, a nucleotide sequence substantially identical (e.g., having at least 70%, 75%, 80%, 85%, 90%, 95% 96%, 97%, 98%, 99% sequence identity) to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.

In some embodiments, the encoded modulatory polynucleotide comprises: (i) a 5′ flanking region comprising the nucleotide sequence of SEQ ID NO: 4879, a nucleotide sequence substantially identical (e.g., having at least 70%, 75%, 80%, 85%, 90%, 95% 96%, 97%, 98%, 99% sequence identity) to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879; (ii) a loop region comprising the nucleotide sequence of SEQ ID NO: 4887, a nucleotide sequence substantially identical (e.g., having at least 70%, 75%, 80%, 85%, 90%, NO: 4887; or a nucleotide sequence comprising one, two, three, or four, but no more than five modifications relative to the nucleotide sequence of SEQ ID NO: 4887; and (iii) a 3′ flanking region of SEQ ID NO: 4899, a nucleotide sequence substantially identical (e.g., having at least 70%, 75%, 80%, 85%, 90%, 95% 96%, 97%, 98%, 99% sequence identity) to SEQ ID NO: 4899, a nucleotide sequence comprising one, two, three, four, five, six, or seven, but no more than ten different nucleotides relative to the nucleotide sequence of SEQ ID NO: 4899, a nucleotide sequence comprising one, two, three, four, five, six, or seven, but no more than ten modifications relative to the nucleotide sequence of SEQ ID NO: 4899.

In some embodiments, the encoded modulatory polynucleotide comprises: (i) a 5′ flanking region comprising the nucleotide sequence of SEQ ID NO: 4354, a nucleotide sequence substantially identical (e.g., having at least 70%, 75%, 80%, 85%, 90%, 95% 96%, 97%, 98%, 99% sequence identity) to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354; (ii) a loop region comprising the nucleotide sequence of SEQ ID NO: 4362, a nucleotide sequence substantially identical (e.g., having at least 70%, 75%, 80%, 85%, 90%, 95% 96%, 97%, 98%, 99% sequence identity) to SEQ ID NO: 4362, a nucleotide sequence comprising one, two, three, or four, but no more than five different nucleotides relative to the nucleotide sequence of SEQ ID NO: 4362, or a nucleotide sequence comprising one, two, three, or four, but no more than five modifications relative to the nucleotide sequence of SEQ ID NO: 4362; and (iii) a 3′ flanking region of SEQ ID NO: 4374, a nucleotide sequence substantially identical (e.g., having at least 70%, 75%, 80%, 85%, 90%, 95% 96%, 97%, 98%, 99% sequence identity) to SEQ ID NO: 4374, a nucleotide sequence comprising one, two, three, four, five, six, or seven, but no more than ten different nucleotides relative to the nucleotide sequence of SEQ ID NO: 4374, a nucleotide sequence comprising one, two, three, four, five, six, or seven, but no more than ten modifications relative to the nucleotide sequence of SEQ ID NO: 4374.

In some embodiments, the encoded modulatory polynucleotide comprises: (i) a 5′ flanking region comprising the nucleotide sequence of SEQ ID NO: 4880, a nucleotide sequence substantially identical (e.g., having at least 70%, 75%, 80%, 85%, 90%, 95% 96%, 97%, 98%, 99% sequence identity) to SEQ ID NO: 4880, a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880; (ii) a loop region comprising the nucleotide sequence of SEQ ID NO: 4891, a nucleotide sequence substantially identical (e.g., having at least 70%, 75%, 80%, 85%, 90%, 95% 96%, 97%, 98%, 99% sequence identity) to SEQ ID NO: 4891, a nucleotide sequence comprising one, NO: 4891; or a nucleotide sequence comprising one, two, three, or four, but no more than five modifications relative to the nucleotide sequence of SEQ ID NO: 4891; and (iii) a 3′ flanking region of SEQ ID NO: 4900, a nucleotide sequence substantially identical (e.g., having at least 70%, 75%, 80%, 85%, 90%, 95% 96%, 97%, 98%, 99% sequence identity) to SEQ ID NO: 4900, a nucleotide sequence comprising one, two, three, four, five, six, or seven, but no more than ten different nucleotides relative to the nucleotide sequence of SEQ ID NO: 4900, or a nucleotide sequence comprising one, two, three, four, five, six, or seven, but no more than ten modifications relative to SEQ ID NO: 4900.

In some embodiments, the encoded modulatory polynucleotide comprises: (i) a 5′ flanking region comprising the nucleotide sequence of SEQ ID NO: 4355, a nucleotide sequence substantially identical (e.g., having at least 70%, 75%, 80%, 85%, 90%, 95% 96%, 97%, 98%, 99% sequence identity) to SEQ ID NO: 4355, a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355; (ii) a loop region comprising the nucleotide sequence of SEQ ID NO: 4366, a nucleotide sequence substantially identical (e.g., having at least 70%, 75%, 80%, 85%, 90%, 95% 96%, 97%, 98%, 99% sequence identity) to SEQ ID NO: 4366, a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366; and (iii) a 3′ flanking region of SEQ ID NO: 4375, a nucleotide sequence substantially identical (e.g., having at least 70%, 75%, 80%, 85%, 90%, 95% 96%, 97%, 98%, 99% sequence identity) to SEQ ID NO: 4375, a nucleotide sequence comprising one, two, three, four, five, six, or seven, but no more than ten different nucleotides relative to the nucleotide sequence of SEQ ID NO: 4375, or a nucleotide sequence comprising one, two, three, four, five, six, or seven, but no more than ten modifications relative to SEQ ID NO: 4375.

In some embodiments, a molecular scaffold may comprise one or more linkers known in the art. The linkers may separate regions or one molecular scaffold from another. As a non-limiting example, the molecular scaffold or modulatory polynucleotide may be polycistronic.

Modulatory Polynucleotide Comprising the Molecular Scaffold and siRNA Molecule

In some embodiments, the modulatory polynucleotide comprises 5′ and 3′ flanking regions, loop region, and nucleic acid sequences encoding the sense strand sequence and the antisense strand sequence as described in Table 9A and Table 9B.

In some embodiments, the modulatory polynucleotide comprises the nucleotide sequence of any one of SEQ ID NOs: 4380-4409 or 5027-5050, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of any one of SEQ ID NOs: 4380-4409 or 5027-5050. In some embodiments, the modulatory polynucleotide comprises a nucleotide sequence comprising at least one, two, three, four, five, six, or seven, but no more than ten modifications relative to the nucleotide sequence of any one of SEQ ID NOs: 4380-4409 or 5027-5050.

In some embodiments, the modulatory polynucleotide comprises the nucleotide sequence of SEQ ID NO: 4405, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4405. In some embodiments, the modulatory polynucleotide comprises a nucleotide sequence comprising at least one, two, three, four, five, six, or seven, but no more than ten modifications relative to the nucleotide sequence of SEQ ID NO: 4405.

In some embodiments, the modulatory polynucleotide comprises the nucleotide sequence of SEQ ID NO: 4408, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4408. In some embodiments, the modulatory polynucleotide comprises a nucleotide sequence comprising at least one, two, three, four, five, six, or seven, but no more than ten modifications relative to the nucleotide sequence of SEQ ID NO: 4408. In some embodiments, the modulatory polynucleotide comprises the nucleotide sequence of SEQ ID NO: 4408.

In some embodiments, the modulatory polynucleotide comprises the nucleotide sequence of SEQ ID NO: 4409, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4409. In some embodiments, the modulatory polynucleotide comprises a nucleotide sequence comprising at least one, two, three, four, five, six, or seven, but no more than ten modifications relative to the nucleotide sequence of SEQ ID NO: 4409. In some embodiments, the modulatory polynucleotide comprises the nucleotide sequence of SEQ ID NO: 4409.

In some embodiments, the modulatory polynucleotide comprises the nucleotide sequence of SEQ ID NO: 4390, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4390. In some embodiments, the modulatory polynucleotide comprises a nucleotide sequence comprising at least one, two, three, four, five, six, or seven, but no more than ten modifications relative to the nucleotide sequence of SEQ ID NO: 4390. In some embodiments, the modulatory polynucleotide comprises the nucleotide sequence of SEQ ID NO: 4390.

In some embodiments, the modulatory polynucleotide comprises the nucleotide sequence of SEQ ID NO: 4391, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4391. In some embodiments, the modulatory polynucleotide comprises a nucleotide sequence comprising at least one, two, three, four, five, six, or seven, but no more than ten modifications relative to the nucleotide sequence of SEQ ID NO: 4391. In some embodiments, the modulatory polynucleotide comprises the nucleotide sequence of SEQ ID NO: 4391.

In some embodiments, the modulatory polynucleotide comprises the nucleotide sequence of SEQ ID NO: 4393, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4393. In some embodiments, the modulatory polynucleotide comprises a nucleotide sequence comprising at least one, two, three, four, five, six, or seven, but no more than ten modifications relative to the nucleotide sequence of SEQ ID NO: 4393. In some embodiments, the modulatory polynucleotide comprises the nucleotide sequence of SEQ ID NO: 4393.

In some embodiments, the modulatory polynucleotide comprises the nucleotide sequence of SEQ ID NO: 4394, or nucleotide sequence at least 70%, 75%, 80%, 85%, 90%, 92%, 95%, 96%, 97%, 987%, or 99% identical thereto. In some embodiments, the modulatory polynucleotide comprises a nucleotide sequence comprising at least one, two, three, four, five, six, or seven, but no more than ten different nucleotides relative to the nucleotide sequence of SEQ ID NO: 4394. In some embodiments, the modulatory polynucleotide comprises a nucleotide sequence comprising at least one, two, three, four, five, six, or seven, but no more than ten modifications relative to the nucleotide sequence of SEQ ID NO: 4394. In some embodiments, the modulatory polynucleotide comprises the nucleotide sequence of SEQ ID NO: 4394.

It is understood that in some embodiments, any of the sequences in Tables 3-8, 9A, and 9B, or herein, can comprise a U nucleotide (uridine) in place of any of or all T nucleotides (thymine) within the sequence (e.g., an RNA molecule). It is also understood that in some embodiments, that any sequence provided herein can comprise a T nucleotide (thymine) in place of any of or all U nucleotides (uracil) within the sequence (e.g., a DNA molecule).

TABLE 9A
Exemplary Modulatory Polynucleotide Sequence Region (5′ to 3′)

| | 5′ Flanking to | | | | | |
|---|---|---|---|---|---|---|
| Modulatory | 3′ Flanking | 5′ Flanking | Passenger | Loop | Guide | 3′ Flanking |
| Polynucleotide | SEQ ID NO | SEQ ID NO | SEQ ID NO | SEQ ID NO | SEQ ID NO | SEQ ID NO |
| VOYTaumiR-102-530 | 4380 | 4354 | 4410 | 4362 | 4434 | 4373 |
| VOYTaumiR-102-556 | 4381 | 4354 | 4411 | 4362 | 4435 | 4373 |
| VOYTaumiR-102-564 | 4382 | 4354 | 4412 | 4362 | 4436 | 4373 |
| VOYTaumiR-102-579 | 4383 | 4354 | 4413 | 4362 | 4437 | 4373 |
| VOYTaumiR-102-586 | 4384 | 4354 | 4414 | 4362 | 4438 | 4373 |
| VOYTaumiR-102-582 | 5027 | 4354 | 5058 | 4362 | 5055 | 4373 |
| VOYTaumiR-102-587 | 5028 | 4354 | 5075 | 4362 | 5078 | 4373 |
| VOYTaumiR-102-552 | 5029 | 4354 | 5099 | 4362 | 5102 | 4373 |
| VOYTaumiR-102-549 | 5030 | 4354 | 5123 | 4362 | 5126 | 4373 |
| VOYTaumiR-104-530 | 4385 | 4354 | 4415 | 4362 | 4434 | 4373 |
| VOYTaumiR-104-556 | 4386 | 4354 | 4416 | 4362 | 4435 | 4373 |
| VOYTaumiR-104-564 | 4387 | 4354 | 4417 | 4362 | 4436 | 4373 |
| VOYTaumiR-104-579 | 4388 | 4354 | 4418 | 4362 | 4437 | 4373 |
| VOYTaumiR-104-586 | 4389 | 4354 | 4419 | 4362 | 4438 | 4373 |
| VOYTaumiR-104-582 | 5031 | 4354 | 5061 | 4362 | 5055 | 4373 |
| VOYTaumiR-104-587 | 5032 | 4354 | 5081 | 4362 | 5078 | 4373 |
| VOYTaumiR-104-552 | 5033 | 4354 | 5105 | 4362 | 5102 | 4373 |
| VOYTaumiR-104-549 | 5034 | 4354 | 5129 | 4362 | 5126 | 4373 |
| VOYTaumiR-109-530 | 4390 | 4354 | 4420 | 4363 | 4434 | 4373 |
| VOYTaumiR-109-556 | 4391 | 4354 | 4421 | 4363 | 4435 | 4373 |
| VOYTaumiR-109-564 | 4392 | 4354 | 4422 | 4363 | 4436 | 4373 |
| VOYTaumiR-109-579 | 4393 | 4354 | 4423 | 4363 | 4437 | 4373 |
| VOYTaumiR-109-586 | 4394 | 4354 | 4424 | 4363 | 4438 | 4373 |
| VOYTaumiR-109-582 | 5035 | 4354 | 5051 | 4363 | 5055 | 4373 |
| VOYTaumiR-109-587 | 5036 | 4354 | 5085 | 4363 | 5078 | 4373 |
| VOYTaumiR-109-552 | 5037 | 4354 | 5109 | 4363 | 5102 | 4373 |
| VOYTaumiR-109-549 | 5038 | 4354 | 5133 | 4363 | 5126 | 4373 |
| VOYTaumiR-114-530 | 4395 | 4354 | 4425 | 4362 | 4434 | 4374 |
| VOYTaumiR-114-556 | 4396 | 4354 | 4426 | 4362 | 4435 | 4374 |
| VOYTaumiR-114-564 | 4397 | 4354 | 4427 | 4362 | 4436 | 4374 |
| VOYTaumiR-114-579 | 4398 | 4354 | 4428 | 4362 | 4437 | 4374 |
| VOYTaumiR-114-586 | 4399 | 4354 | 4429 | 4362 | 4438 | 4374 |
| VOYTaumiR-114-582 | 5039 | 4354 | 5067 | 4362 | 5055 | 4374 |
| VOYTaumiR-114-587 | 5040 | 4354 | 5089 | 4362 | 5078 | 4374 |
| VOYTaumiR-114-552 | 5041 | 4354 | 5113 | 4362 | 5102 | 4374 |
| VOYTaumiR-114-549 | 5042 | 4354 | 5137 | 4362 | 5126 | 4374 |
| VOYTaumiR-116-530 | 4400 | 4354 | 4430 | 4362 | 4434 | 4374 |
| VOYTaumiR-116-556 | 4401 | 4354 | 4431 | 4362 | 4435 | 4374 |
| VOYTaumiR-116-564 | 4402 | 4354 | 4432 | 4362 | 4436 | 4374 |
| VOYTaumiR-116-579 | 4403 | 4354 | 4418 | 4362 | 4437 | 4374 |
| VOYTaumiR-116-586 | 4404 | 4354 | 4433 | 4362 | 4438 | 4374 |
| VOYTaumiR-116-582 | 5043 | 4354 | 5071 | 4362 | 5055 | 4374 |
| VOYTaumiR-116-587 | 5044 | 4354 | 5093 | 4362 | 5078 | 4374 |
| VOYTaumiR-116-552 | 5045 | 4354 | 5117 | 4362 | 5102 | 4374 |
| VOYTaumiR-116-549 | 5046 | 4354 | 5141 | 4362 | 5126 | 4374 |
| VOYTaumiR-127-530 | 4405 | 4355 | 4420 | 4366 | 4434 | 4375 |
| VOYTaumiR-127-556 | 4406 | 4355 | 4421 | 4366 | 4435 | 4375 |
| VOYTaumiR-127-564 | 4407 | 4355 | 4422 | 4366 | 4436 | 4375 |
| VOYTaumiR-127-579 | 4408 | 4355 | 4423 | 4366 | 4437 | 4375 |
| VOYTaumiR-127-586 | 4409 | 4355 | 4424 | 4366 | 4438 | 4375 |
| VOYTaumiR-127-582 | 5047 | 4355 | 5051 | 4366 | 5055 | 4375 |
| VOYTaumiR-127-587 | 5048 | 4355 | 5085 | 4366 | 5078 | 4375 |
| VOYTaumiR-127-552 | 5049 | 4355 | 5109 | 4366 | 5102 | 4375 |
| VOYTaumiR-127-549 | 5050 | 4355 | 5133 | 4366 | 5126 | 4375 |

TABLE 9B
Exemplary Modulatory Polynucleotides

| Name | SEQ ID NO | Description | Sequence |
|---|---|---|---|
| VOYTaumiR- | 4405 | 5′ flanking to 3′ | GAAGCAAAGAAGGGGCAGAGGGAGCCCGTGAGCTGAGTGGGCCAGG |
| 127-530 | | flanking | GACTGGGAGAAGGAGTGAGGAGGCAGGGCCGGCATGCCTCTGCTGC |
| | | | TGGCCAGAGACTGGAAGCGATGACAATCTGTCTGCACCTGTCACTA |
| | | | GTTTGTCATCGCTTCCAGTCTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 4420 | Passenger Strand | GACTGGAAGCGATGACAAT |
| | | (DNA) | |
| | 4497 | Passenger Strand | GACUGGAAGCGAUGACAAU |
| | | (RNA) | |
| | 4905 | Passenger Strand | GACTGGAAGCGATGACAATCT |
| | | (DNA) v2 | |
| | 4906 | Passenger Strand | GACUGGAAGCGAUGACAAUCU |
| | | (RNA) v2 | |
| | 4366 | Loop | GTCTGCACCTGTCACTAG |
| | 4434 | Guide Strand | TTTGTCATCGCTTCCAGTC |
| | | (DNA) | |
| | 4697 | Guide Strand | UUUGUCAUCGCUUCCAGUC |
| | | (RNA) | |
| | 4907 | Guide Strand | TTTGTCATCGCTTCCAGTCTT |
| | | (DNA) v2 | |
| | 4908 | Guide Strand | UUUGUCAUCGCUUCCAGUCUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 4406 | 5′ flanking to 3′ | GAAGCAAAGAAGGGGCAGAGGGAGCCCGTGAGCTGAGTGGGCCAGG |
| 127-556 | | flanking | GACTGGGAGAAGGAGTGAGGAGGCAGGGCCGGCATGCCTCTGCTGC |
| | | | TGGCCAGACTTCCGCGAGAACGCCAATCTGTCTGCACCTGTCACTA |
| | | | GTTTGGCGTTCTCGCGGAAGTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 4421 | Passenger Strand | CTTCCGCGAGAACGCCAAT |
| | | (DNA) | |
| | 4508 | Passenger Strand | CUUCCGCGAGAACGCCAAU |
| | | (RNA) | |
| | 4909 | Passenger Strand | CTTCCGCGAGAACGCCAATCT |
| | | (DNA) v2 | |
| | 4910 | Passenger Strand | CUUCCGCGAGAACGCCAAUCU |
| | | (RNA) v2 | |
| | 4366 | Loop | GTCTGCACCTGTCACTAG |
| | 4435 | Guide Strand | TTTGGCGTTCTCGCGGAAG |
| | | (DNA) | |
| | 4701 | Guide Strand | UUUGGCGUUCUCGCGGAAG |
| | | (RNA) | |
| | 4911 | Guide Strand | TTTGGCGTTCTCGCGGAAGTT |
| | | (DNA) v2 | |
| | 4912 | Guide Strand | UUUGGCGUUCUCGCGGAAGUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 4407 | 5′ flanking to 3′ | GAAGCAAAGAAGGGGCAGAGGGAGCCCGTGAGCTGAGTGGGCCAGG |
| 127-564 | | flanking | GACTGGGAGAAGGAGTGAGGAGGCAGGGCCGGCATGCCTCTGCTGC |
| | | | TGGCCAGAGCATCGACATGGTAGACTTCTGTCTGCACCTGTCACTA |
| | | | GTAGTCTACCATGTCGATGCTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 4422 | Passenger Strand | GCATCGACATGGTAGACTT |
| | | (DNA) | |
| | 4487 | Passenger Strand | GCAUCGACAUGGUAGACUU |
| | | (RNA) | |
| | 4913 | Passenger Strand | GCATCGACATGGTAGACTTCT |
| | | (DNA) v2 | |
| | 4914 | Passenger Strand | GCAUCGACAUGGUAGACUUCU |
| | | (RNA) v2 | |
| | 4366 | Loop | GTCTGCACCTGTCACTAG |
| | 4436 | Guide Strand | TAGTCTACCATGTCGATGC |
| | | (DNA) | |
| | 4691 | Guide Strand | UAGUCUACCAUGUCGAUGC |
| | | (RNA) | |
| | 4915 | Guide Strand | TAGTCTACCATGTCGATGCTT |
| | | (DNA) v2 | |
| | 4916 | Guide Strand | UAGUCUACCAUGUCGAUGCUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 4408 | 5′ flanking to 3′ | GAAGCAAAGAAGGGGCAGAGGGAGCCCGTGAGCTGAGTGGGCCAGG |
| 127-579 | | flanking | GACTGGGAGAAGGAGTGAGGAGGCAGGGCCGGCATGCCTCTGCTGC |
| | | | TGGCCAGAGCTCCTCGCAGTTCGGTTTCTGTCTGCACCTGTCACTA |
| | | | GTAACCGAACTGCGAGGAGCTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 4423 | Passenger Strand | GCTCCTCGCAGTTCGGTTT |
| | | (DNA) | |
| | 4504 | Passenger Strand | GCUCCUCGCAGUUCGGUUU |
| | | (RNA) | |
| | 4917 | Passenger Strand | GCTCCTCGCAGTTCGGTTTCT |
| | | (DNA) v2 | |
| | 4918 | Passenger Strand | GCUCCUCGCAGUUCGGUUUCU |
| | | (RNA) v2 | |
| | 4366 | Loop | GTCTGCACCTGTCACTAG |
| | 4437 | Guide Strand | TAACCGAACTGCGAGGAGC |
| | | (DNA) | |
| | 4700 | Guide Strand | UAACCGAACUGCGAGGAGC |
| | | (RNA) | |
| | 4919 | Guide Strand | TAACCGAACTGCGAGGAGCTT |
| | | (DNA) v2 | |
| | 4920 | Guide Strand | UAACCGAACUGCGAGGAGCUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 4409 | 5′ flanking to 3′ | GAAGCAAAGAAGGGGCAGAGGGAGCCCGTGAGCTGAGTGGGCCAGG |
| 127-586 | | flanking | GACTGGGAGAAGGAGTGAGGAGGCAGGGCCGGCATGCCTCTGCTGC |
| | | | TGGCCAGAGGCTTTGGCTCGGGACTTTCTGTCTGCACCTGTCACTA |
| | | | GTAAGTCCCGAGCCAAAGCCTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 4424 | Passenger Strand | GGCTTTGGCTCGGGACTTT |
| | | (DNA) | |
| | 4482 | Passenger Strand | GGCUUUGGCUCGGGACUUU |
| | | (RNA) | |
| | 4921 | Passenger Strand | GGCTTTGGCTCGGGACTTTCT |
| | | (DNA) v2 | |
| | 4922 | Passenger Strand | GGCUUUGGCUCGGGACUUUCU |
| | | (RNA) v2 | |
| | 4366 | Loop | GTCTGCACCTGTCACTAG |
| | 4438 | Guide Strand | TAAGTCCCGAGCCAAAGCC |
| | | (DNA) | |
| | 4690 | Guide Strand | UAAGUCCCGAGCCAAAGCC |
| | | (RNA) | |
| | 4923 | Guide Strand | TAAGTCCCGAGCCAAAGCCTT |
| | | (DNA) v2 | |
| | 4924 | Guide Strand | UAAGUCCCGAGCCAAAGCCUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 5047 | 5′ flanking to 3′ | GAAGCAAAGAAGGGGCAGAGGGAGCCCGTGAGCTGAGTGGGCCAGG |
| 127-582 | | flanking | GACTGGGAGAAGGAGTGAGGAGGCAGGGCCGGCATGCCTCTGCTGC |
| | | | TGGCCAGACCTCGCAGTTCGGTTAATTCTGTCTGCACCTGTCACTA |
| | | | GTATTAACCGAACTGCGAGGTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 5051 | Passenger Strand | CCTCGCAGTTCGGTTAATT |
| | | (DNA) | |
| | 5052 | Passenger Strand | CCUCGCAGUUCGGUUAAUU |
| | | (RNA) | |
| | 5053 | Passenger Strand | CCTCGCAGTTCGGTTAATTCT |
| | | (DNA) v2 | |
| | 5054 | Passenger Strand | CCUCGCAGUUCGGUUAAUUCU |
| | | (RNA) v2 | |
| | 4366 | Loop | GTCTGCACCTGTCACTAG |
| | 5055 | Guide Strand | TATTAACCGAACTGCGAGG |
| | | (DNA) | |
| | 4694 | Guide Strand | UAUUAACCGAACUGCGAGG |
| | | (RNA) | |
| | 5056 | Guide Strand | TATTAACCGAACTGCGAGGTT |
| | | (DNA) v2 | |
| | 5057 | Guide Strand | UAUUAACCGAACUGCGAGGUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 4380 | 5′ flanking to 3′ | GTGCTGGGCGGGGGGCGGCGGGCCCTCCCGCAGAACACCATGCGCT |
| 102-530 | | flanking | CTTCGGAAGACTGGAAGCGATGACAACCCTGTGACCTGGTTTGTCA |
| | | | TCGCTTCCAGTCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4410 | Passenger Strand | GACTGGAAGCGATGACAAC |
| | | (DNA) | |
| | 4495 | Passenger Strand | GACUGGAAGCGAUGACAAC |
| | | (RNA) | |
| | 4925 | Passenger Strand | GACTGGAAGCGATGACAACCC |
| | | (DNA) v2 | |
| | 4926 | Passenger Strand | GACUGGAAGCGAUGACAACCC |
| | | (RNA) v2 | |
| | 4362 | Loop | TGTGACCTGG |
| | 4434 | Guide Strand | TTTGTCATCGCTTCCAGTC |
| | | (DNA) | |
| | 4697 | Guide Strand | UUUGUCAUCGCUUCCAGUC |
| | | (RNA) | |
| | 4907 | Guide Strand | TTTGTCATCGCTTCCAGTCTT |
| | | (DNA) v2 | |
| | 4908 | Guide Strand | UUUGUCAUCGCUUCCAGUCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4381 | 5′ flanking to 3′ | GTGCTGGGCGGGGGGCGGCGGGCCCTCCCGCAGAACACCATGCGCT |
| 102-556 | | flanking | CTTCGGAACTTCCGCGAGAACGCCAACCCTGTGACCTGGTTTGGCG |
| | | | TTCTCGCGGAAGTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4411 | Passenger Strand | CTTCCGCGAGAACGCCAAC |
| | | (DNA) | |
| | 4506 | Passenger Strand | CUUCCGCGAGAACGCCAAC |
| | | (RNA) | |
| | 4929 | Passenger Strand | CTTCCGCGAGAACGCCAACCC |
| | | (DNA) v2 | |
| | 4930 | Passenger Strand | CUUCCGCGAGAACGCCAACCC |
| | | (RNA) v2 | |
| | 4362 | Loop | TGTGACCTGG |
| | 4435 | Guide Strand | TTTGGCGTTCTCGCGGAAG |
| | | (DNA) | |
| | 4701 | Guide Strand | UUUGGCGUUCUCGCGGAAG |
| | | (RNA) | |
| | 4911 | Guide Strand | TTTGGCGTTCTCGCGGAAGTT |
| | | (DNA) v2 | |
| | 4912 | Guide Strand | UUUGGCGUUCUCGCGGAAGUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4382 | 5′ flanking to 3′ | GTGCTGGGCGGGGGGCGGCGGGCCCTCCCGCAGAACACCATGCGCT |
| 102-564 | | flanking | CTTCGGAAGCATCGACATGGTAGACTCCCTGTGACCTGGTAGTCTA |
| | | | CCATGTCGATGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4412 | Passenger Strand | GCATCGACATGGTAGACTC |
| | | (DNA) | |
| | 4485 | Passenger Strand | GCAUCGACAUGGUAGACUC |
| | | (RNA) | |
| | 4933 | Passenger Strand | GCATCGACATGGTAGACTCCC |
| | | (DNA) v2 | |
| | 4934 | Passenger Strand | GCAUCGACAUGGUAGACUCCC |
| | | (RNA) v2 | |
| | 4362 | Loop | TGTGACCTGG |
| | 4436 | Guide Strand | TAGTCTACCATGTCGATGC |
| | | (DNA) | |
| | 4691 | Guide Strand | UAGUCUACCAUGUCGAUGC |
| | | (RNA) | |
| | 4915 | Guide Strand | TAGTCTACCATGTCGATGCTT |
| | | (DNA) v2 | |
| | 4916 | Guide Strand | UAGUCUACCAUGUCGAUGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4383 | 5′ flanking to 3′ | GTGCTGGGGGGGGGGGGGCGGGCCCTCCCGCAGAACACCATGCGCT |
| 102-579 | | flanking | CTTCGGAAGCTCCTCGCAGTTCGGTTCCCTGTGACCTGGTAACCGA |
| | | | ACTGCGAGGAGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4413 | Passenger Strand | GCTCCTCGCAGTTCGGTTC |
| | | (DNA) | |
| | 4502 | Passenger Strand | GCUCCUCGCAGUUCGGUUC |
| | | (RNA) | |
| | 4937 | Passenger Strand | GCTCCTCGCAGTTCGGTTCCC |
| | | (DNA) v2 | |
| | 4938 | Passenger Strand | GCUCCUCGCAGUUCGGUUCCC |
| | | (RNA) v2 | |
| | 4362 | Loop | TGTGACCTGG |
| | 4437 | Guide Strand | TAACCGAACTGCGAGGAGC |
| | | (DNA) | |
| | 4700 | Guide Strand | UAACCGAACUGCGAGGAGC |
| | | (RNA) | |
| | 4919 | Guide Strand | TAACCGAACTGCGAGGAGCTT |
| | | (DNA) v2 | |
| | 4920 | Guide Strand | UAACCGAACUGCGAGGAGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4384 | 5′ flanking to 3′ | GTGCTGGGCGGGGGGCGGCGGGCCCTCCCGCAGAACACCATGCGCT |
| 102-586 | | flanking | CTTCGGAAGGCTTTGGCTCGGGACTTCCCTGTGACCTGGTAAGTCC |
| | | | CGAGCCAAAGCCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4414 | Passenger Strand | GGCTTTGGCTCGGGACTTC |
| | | (DNA) | |
| | 4480 | Passenger Strand | GGCUUUGGCUCGGGACUUC |
| | | (RNA) | |
| | 4941 | Passenger Strand | GGCTTTGGCTCGGGACTTCCC |
| | | (DNA) v2 | |
| | 4942 | Passenger Strand | GGCUUUGGCUCGGGACUUCCC |
| | | (RNA) v2 | |
| | 4362 | Loop | TGTGACCTGG |
| | 4438 | Guide Strand | TAAGTCCCGAGCCAAAGCC |
| | | (DNA) | |
| | 4690 | Guide Strand | UAAGUCCCGAGCCAAAGCC |
| | | (RNA) | |
| | 4923 | Guide Strand | TAAGTCCCGAGCCAAAGCCTT |
| | | (DNA) v2 | |
| | 4924 | Guide Strand | UAAGUCCCGAGCCAAAGCCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5027 | 5′ flanking to 3′ | GTGCTGGGCGGGGGGCGGCGGGCCCTCCCGCAGAACACCATGCGCT |
| 102-582 | | flanking | CTTCGGAACCTCGCAGTTCGGTTAATCCCTGTGACCTGGTATTAAC |
| | | | CGAACTGCGAGGTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5058 | Passenger Strand | CCTCGCAGTTCGGTTAATC |
| | | (DNA) | |
| | 4492 | Passenger Strand | CCUCGCAGUUCGGUUAAUC |
| | | (RNA) | |
| | 5059 | Passenger Strand | CCTCGCAGTTCGGTTAATCCC |
| | | (DNA) v2 | |
| | 5060 | Passenger Strand | CCUCGCAGUUCGGUUAAUCCC |
| | | (RNA) v2 | |
| | 4362 | Loop | TGTGACCTGG |
| | 5055 | Guide Strand | TATTAACCGAACTGCGAGG |
| | | (DNA) | |
| | 4694 | Guide Strand | UAUUAACCGAACUGCGAGG |
| | | (RNA) | |
| | 5056 | Guide Strand | TATTAACCGAACTGCGAGGTT |
| | | (DNA) v2 | |
| | 5057 | Guide Strand | UAUUAACCGAACUGCGAGGUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4385 | 5′ flanking to 3′ | GTGCTGGGCGGGGGGCGGCGGGCCCTCCCGCAGAACACCATGCGCT |
| 104-530 | | flanking | CTTCGGAAGACTGGAAGCGATGACAAACCTGTGACCTGGTTTGTCA |
| | | | TCGCTTCCAGTCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4415 | Passenger Strand | GACTGGAAGCGATGACAAA |
| | | (DNA) | |
| | 4496 | Passenger Strand | GACUGGAAGCGAUGACAAA |
| | | (RNA) | |
| | 4945 | Passenger Strand | GACTGGAAGCGATGACAAACC |
| | | (DNA) v2 | |
| | 4946 | Passenger Strand | GACUGGAAGCGAUGACAAACC |
| | | (RNA) v2 | |
| | 4362 | Loop | TGTGACCTGG |
| | 4434 | Guide Strand | TTTGTCATCGCTTCCAGTC |
| | | (DNA) | |
| | 4697 | Guide Strand | UUUGUCAUCGCUUCCAGUC |
| | | (RNA) | |
| | 4907 | Guide Strand | TTTGTCATCGCTTCCAGTCTT |
| | | (DNA) v2 | |
| | 4908 | Guide Strand | UUUGUCAUCGCUUCCAGUCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4386 | 5′ flanking to 3′ | GTGCTGGGCGGGGGGCGGCGGGCCCTCCCGCAGAACACCATGCGCT |
| 104-556 | | flanking | CTTCGGAACTTCCGCGAGAACGCCAAACCTGTGACCTGGTTTGGCG |
| | | | TTCTCGCGGAAGTTCTGAGGAGCGCCTTGACAGCAGCCATGGGAGG |
| | | | GCCGCCCCCTACCTCAGTGA |
| | 4354 | 5′ flanking | GTGCTGGGGGGGGGGGGGCGGGCCCTCCCGCAGAACACCATGCGCT |
| | | | CTTCGGAA |
| | 4416 | Passenger Strand | CTTCCGCGAGAACGCCAAA |
| | | (DNA) | |
| | 4507 | Passenger Strand | CUUCCGCGAGAACGCCAAA |
| | | (RNA) | |
| | 4949 | Passenger Strand | CTTCCGCGAGAACGCCAAACC |
| | | (DNA) v2 | |
| | 4950 | Passenger Strand | CUUCCGCGAGAACGCCAAACC |
| | | (RNA) v2 | |
| | 4362 | Loop | TGTGACCTGG |
| | 4435 | Guide Strand | TTTGGCGTTCTCGCGGAAG |
| | | (DNA) | |
| | 4701 | Guide Strand | UUUGGCGUUCUCGCGGAAG |
| | | (RNA) | |
| | 4911 | Guide Strand | TTTGGCGTTCTCGCGGAAGTT |
| | | (DNA) v2 | |
| | 4912 | Guide Strand | UUUGGCGUUCUCGCGGAAGUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4387 | 5′ flanking to 3′ | GTGCTGGGCGGGGGGCGGCGGGCCCTCCCGCAGAACACCATGCGCT |
| 104-564 | | flanking | CTTCGGAAGCATCGACATGGTAGACTACCTGTGACCTGGTAGTCTA |
| | | | CCATGTCGATGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4417 | Passenger Strand | GCATCGACATGGTAGACTA |
| | | (DNA) | |
| | 4486 | Passenger Strand | GCAUCGACAUGGUAGACUA |
| | | (RNA) | |
| | 4953 | Passenger Strand | GCATCGACATGGTAGACTACC |
| | | (DNA) v2 | |
| | 4954 | Passenger Strand | GCAUCGACAUGGUAGACUACC |
| | | (RNA) v2 | |
| | 4362 | Loop | TGTGACCTGG |
| | 4436 | Guide Strand | TAGTCTACCATGTCGATGC |
| | | (DNA) | |
| | 4691 | Guide Strand | UAGUCUACCAUGUCGAUGC |
| | | (RNA) | |
| | 4915 | Guide Strand | TAGTCTACCATGTCGATGCTT |
| | | (DNA) v2 | |
| | 4916 | Guide Strand | UAGUCUACCAUGUCGAUGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4388 | 5′ flanking to 3′ | GCGCTGGGCGGGGGGCGGCGGGCCCTCCCGCAGAACACCATGCGCT |
| 104-579 | | flanking | CTTCGGAAGCTCCTCGCAGTTCGGTTGCCTGTGACCTGGTAACCGA |
| | | | ACTGCGAGGAGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4418 | Passenger Strand | GCTCCTCGCAGTTCGGTTG |
| | | (DNA) | |
| | 4503 | Passenger Strand | GCUCCUCGCAGUUCGGUUG |
| | | (RNA) | |
| | 4957 | Passenger Strand | GCTCCTCGCAGTTCGGTTGCC |
| | | (DNA) v2 | |
| | 4958 | Passenger Strand | GCUCCUCGCAGUUCGGUUGCC |
| | | (RNA) v2 | |
| | 4362 | Loop | TGTGACCTGG |
| | 4437 | Guide Strand | TAACCGAACTGCGAGGAGC |
| | | (DNA) | |
| | 4700 | Guide Strand | UAACCGAACUGCGAGGAGC |
| | | (RNA) | |
| | 4919 | Guide Strand | TAACCGAACTGCGAGGAGCTT |
| | | (DNA) v2 | |
| | 4920 | Guide Strand | UAACCGAACUGCGAGGAGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4389 | 5′ flanking to 3′ | GTGCTGGGCGGGGGGCGGCGGGCCCTCCCGCAGAACACCATGCGCT |
| 104-586 | | flanking | CTTCGGAAGGCTTTGGCTCGGGACTTACCTGTGACCTGGTAAGTCC |
| | | | CGAGCCAAAGCCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4419 | Passenger Strand | GGCTTTGGCTCGGGACTTA |
| | | (DNA) | |
| | 4481 | Passenger Strand | GGCUUUGGCUCGGGACUUA |
| | | (RNA) | |
| | 4961 | Passenger Strand | GGCTTTGGCTCGGGACTTACC |
| | | (DNA) v2 | |
| | 4962 | Passenger Strand | GGCUUUGGCUCGGGACUUACC |
| | | (RNA) v2 | |
| | 4362 | Loop | TGTGACCTGG |
| | 4438 | Guide Strand | TAAGTCCCGAGCCAAAGCC |
| | | (DNA) | |
| | 4690 | Guide Strand | UAAGUCCCGAGCCAAAGCC |
| | | (RNA) | |
| | 4923 | Guide Strand | TAAGTCCCGAGCCAAAGCCTT |
| | | (DNA) v2 | |
| | 4924 | Guide Strand | UAAGUCCCGAGCCAAAGCCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5031 | 5′ flanking to 3′ | GTGCTGGGCGGGGGGCGGCGGGCCCTCCCGCAGAACACCATGCGCT |
| 104-582 | | flanking | CTTCGGAACCTCGCAGTTCGGTTAATACCTGTGACCTGGTATTAAC |
| | | | CGAACTGCGAGGTTCTGAGGAGCGCCTTGACAGCAGCCATGGGAGG |
| | | | GCCGCCCCCTACCTCAGTGA |
| | 4354 | 5′ flanking | GTGCTGGGCGGGGGGGGGGGGGCCCTCCCGCAGAACACCATGCGCT |
| | | | CTTCGGAA |
| | 5061 | Passenger Strand | CCTCGCAGTTCGGTTAATA |
| | | (DNA) | |
| | 5062 | Passenger Strand | CCUCGCAGUUCGGUUAAUA |
| | | (RNA) | |
| | 5063 | Passenger Strand | CCTCGCAGTTCGGTTAATACC |
| | | (DNA) v2 | |
| | 5064 | Passenger Strand | CCUCGCAGUUCGGUUAAUACC |
| | | (RNA) v2 | |
| | 4362 | Loop | TGTGACCTGG |
| | 5055 | Guide Strand | TATTAACCGAACTGCGAGG |
| | | (DNA) | |
| | 4694 | Guide Strand | UAUUAACCGAACUGCGAGG |
| | | (RNA) | |
| | 5056 | Guide Strand | TATTAACCGAACTGCGAGGTT |
| | | (DNA) v2 | |
| | 5057 | Guide Strand | UAUUAACCGAACUGCGAGGUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4390 | 5′ flanking to 3′ | GTGCTGGGGGGGGGGGGGCGGGCCCTCCCGCAGAACACCATGCGCT |
| 109-530 | | flanking | CTTCGGAAGACTGGAAGCGATGACAATCCTGTGATTTGGTTTGTCA |
| | | | TCGCTTCCAGTCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4420 | Passenger Strand | GACTGGAAGCGATGACAAT |
| | | (DNA) | |
| | 4497 | Passenger Strand | GACUGGAAGCGAUGACAAU |
| | | (RNA) | |
| | 4965 | Passenger Strand | GACTGGAAGCGATGACAATCC |
| | | (DNA) v2 | |
| | 4966 | Passenger Strand | GACUGGAAGCGAUGACAAUCC |
| | | (RNA) v2 | |
| | 4363 | Loop | TGTGATTTGG |
| | 4434 | Guide Strand | TTTGTCATCGCTTCCAGTC |
| | | (DNA) | |
| | 4697 | Guide Strand | UUUGUCAUCGCUUCCAGUC |
| | | (RNA) | |
| | 4907 | Guide Strand | TTTGTCATCGCTTCCAGTCTT |
| | | (DNA) v2 | |
| | 4908 | Guide Strand | UUUGUCAUCGCUUCCAGUCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4391 | 5′ flanking to 3′ | GTGCTGGGCGGGGGGCGGCGGGCCCTCCCGCAGAACACCATGCGCT |
| 109-556 | | flanking | CTTCGGAACTTCCGCGAGAACGCCAATCCTGTGATTTGGTTTGGCG |
| | | | TTCTCGCGGAAGTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4421 | Passenger Strand | CTTCCGCGAGAACGCCAAT |
| | | (DNA) | |
| | 4508 | Passenger Strand | CUUCCGCGAGAACGCCAAU |
| | | (RNA) | |
| | 4969 | Passenger Strand | CTTCCGCGAGAACGCCAATCC |
| | | (DNA) v2 | |
| | 4970 | Passenger Strand | CUUCCGCGAGAACGCCAAUCC |
| | | (RNA) v2 | |
| | 4363 | Loop | TGTGATTTGG |
| | 4435 | Guide Strand | TTTGGCGTTCTCGCGGAAG |
| | | (DNA) | |
| | 4701 | Guide Strand | UUUGGCGUUCUCGCGGAAG |
| | | (RNA) | |
| | 4911 | Guide Strand | TTTGGCGTTCTCGCGGAAGTT |
| | | (DNA) v2 | |
| | 4912 | Guide Strand | UUUGGCGUUCUCGCGGAAGUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4392 | 5′ flanking to 3′ | GTGCTGGGCGGGGGGCGGCGGGCCCTCCCGCAGAACACCATGCGCT |
| 109-564 | | flanking | CTTCGGAAGCATCGACATGGTAGACTTCCTGTGATTTGGTAGTCTA |
| | | | CCATGTCGATGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4422 | Passenger Strand | GCATCGACATGGTAGACTT |
| | | (DNA) | |
| | 4487 | Passenger Strand | GCAUCGACAUGGUAGACUU |
| | | (RNA) | |
| | 4973 | Passenger Strand | GCATCGACATGGTAGACTTCC |
| | | (DNA) v2 | |
| | 4974 | Passenger Strand | GCAUCGACAUGGUAGACUUCC |
| | | (RNA) v2 | |
| | 4363 | Loop | TGTGATTTGG |
| | 4436 | Guide Strand | TAGTCTACCATGTCGATGC |
| | | (DNA) | |
| | 4691 | Guide Strand | UAGUCUACCAUGUCGAUGC |
| | | (RNA) | |
| | 4915 | Guide Strand | TAGTCTACCATGTCGATGCTT |
| | | (DNA) v2 | |
| | 4916 | Guide Strand | UAGUCUACCAUGUCGAUGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4393 | 5′ flanking to 3′ | GTGCTGGGCGGGGGGCGGCGGGCCCTCCCGCAGAACACCATGCGCT |
| 109-579 | | flanking | CTTCGGAAGCTCCTCGCAGTTCGGTTTCCTGTGATTTGGTAACCGA |
| | | | ACTGCGAGGAGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4423 | Passenger Strand | GCTCCTCGCAGTTCGGTTT |
| | | (DNA) | |
| | 4504 | Passenger Strand | GCUCCUCGCAGUUCGGUUU |
| | | (RNA) | |
| | 4977 | Passenger Strand | GCTCCTCGCAGTTCGGTTTCC |
| | | (DNA) v2 | |
| | 4978 | Passenger Strand | GCUCCUCGCAGUUCGGUUUCC |
| | | (RNA) v2 | |
| | 4363 | Loop | TGTGATTTGG |
| | 4437 | Guide Strand | TAACCGAACTGCGAGGAGC |
| | | (DNA) | |
| | 4700 | Guide Strand | UAACCGAACUGCGAGGAGC |
| | | (RNA) | |
| | 4919 | Guide Strand | TAACCGAACTGCGAGGAGCTT |
| | | (DNA) v2 | |
| | 4920 | Guide Strand | UAACCGAACUGCGAGGAGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4394 | 5′ flanking to 3′ | GTGCTGGGCGGGGGGCGGCGGGCCCTCCCGCAGAACACCATGCGCT |
| 109-586 | | flanking | CTTCGGAAGGCTTTGGCTCGGGACTTTCCTGTGATTTGGTAAGTCC |
| | | | CGAGCCAAAGCCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4424 | Passenger Strand | GGCTTTGGCTCGGGACTTT |
| | | (DNA) | |
| | 4482 | Passenger Strand | GGCUUUGGCUCGGGACUUU |
| | | (RNA) | |
| | 4981 | Passenger Strand | GGCTTTGGCTCGGGACTTTCC |
| | | (DNA) v2 | |
| | 4982 | Passenger Strand | GGCUUUGGCUCGGGACUUUCC |
| | | (RNA) v2 | |
| | 4363 | Loop | TGTGATTTGG |
| | 4438 | Guide Strand | TAAGTCCCGAGCCAAAGCC |
| | | (DNA) | |
| | 4690 | Guide Strand | UAAGUCCCGAGCCAAAGCC |
| | | (RNA) | |
| | 4923 | Guide Strand | TAAGTCCCGAGCCAAAGCCTT |
| | | (DNA) v2 | |
| | 4924 | Guide Strand | UAAGUCCCGAGCCAAAGCCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5035 | 5′ flanking to 3′ | GTGCTGGGCGGGGGGGGGGGGGCCCTCCCGCAGAACACCATGCGCT |
| 109-582 | | flanking | CTTCGGAACCTCGCAGTTCGGTTAATTCCTGTGATTTGGTATTAAC |
| | | | CGAACTGCGAGGTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5051 | Passenger Strand | CCTCGCAGTTCGGTTAATT |
| | | (DNA) | |
| | 5052 | Passenger Strand | CCUCGCAGUUCGGUUAAUU |
| | | (RNA) | |
| | 5065 | Passenger Strand | CCTCGCAGTTCGGTTAATTCC |
| | | (DNA) v2 | |
| | 5066 | Passenger Strand | CCUCGCAGUUCGGUUAAUUCC |
| | | (RNA) v2 | |
| | 4363 | Loop | TGTGATTTGG |
| | 5055 | Guide Strand | TATTAACCGAACTGCGAGG |
| | | (DNA) | |
| | 4694 | Guide Strand | UAUUAACCGAACUGCGAGG |
| | | (RNA) | |
| | 5056 | Guide Strand | TATTAACCGAACTGCGAGGTT |
| | | (DNA) v2 | |
| | 5057 | Guide Strand | UAUUAACCGAACUGCGAGGUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 4395 | 5′ flanking to 3′ | GTGCTGGGCGGGGGGCGGCGGGCCCTCCCGCAGAACACCATGCGCT |
| 114-530 | | flanking | CTTCGGAAGACTGGAAGGGATGACAACCCTGTGACCTGGTTTGTCA |
| | | | TCGCTTCCAGTCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4425 | Passenger Strand | GACTGGAAGGGATGACAAC |
| | | (DNA) | |
| | 4498 | Passenger Strand | GACUGGAAGGGAUGACAAC |
| | | (RNA) | |
| | 4985 | Passenger Strand | GACTGGAAGGGATGACAACCC |
| | | (DNA) v2 | |
| | 4986 | Passenger Strand | GACUGGAAGGGAUGACAACCC |
| | | (RNA) v2 | |
| | 4362 | Loop | TGTGACCTGG |
| | 4434 | Guide Strand | TTTGTCATCGCTTCCAGTC |
| | | (DNA) | |
| | 4697 | Guide Strand | UUUGUCAUCGCUUCCAGUC |
| | | (RNA) | |
| | 4907 | Guide Strand | TTTGTCATCGCTTCCAGTCTT |
| | | (DNA) v2 | |
| | 4908 | Guide Strand | UUUGUCAUCGCUUCCAGUCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4396 | 5′ flanking to 3′ | GTGCTGGGCGGGGGGCGGCGGGCCCTCCCGCAGAACACCATGCGCT |
| 114-556 | | flanking | CTTCGGAACTTCCGCGACAACGCCAACCCTGTGACCTGGTTTGGCG |
| | | | TTCTCGCGGAAGTTCTGTGGAGCGCCTTGACAGCAGCCATGGGAGG |
| | | | GCCGCCCCCTACCTCAGTGA |
| | 4354 | 5′ flanking | GTGCTGGGGGGGGGGGGCGGGCCCTCCCGCAGAACACCATGCGCT |
| | | | CTTCGGAA |
| | 4426 | Passenger Strand | CTTCCGCGACAACGCCAAC |
| | | (DNA) | |
| | 4509 | Passenger Strand | CUUCCGCGACAACGCCAAC |
| | | (RNA) | |
| | 4989 | Passenger Strand | CTTCCGCGACAACGCCAACCC |
| | | (DNA) v2 | |
| | 4990 | Passenger Strand | CUUCCGCGACAACGCCAACCC |
| | | (RNA) v2 | |
| | 4362 | Loop | TGTGACCTGG |
| | 4435 | Guide Strand | TTTGGCGTTCTCGCGGAAG |
| | | (DNA) | |
| | 4701 | Guide Strand | UUUGGCGUUCUCGCGGAAG |
| | | (RNA) | |
| | 4911 | Guide Strand | TTTGGCGTTCTCGCGGAAGTT |
| | | (DNA) v2 | |
| | 4912 | Guide Strand | UUUGGCGUUCUCGCGGAAGUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4397 | 5′ flanking to 3′ | GTGCTGGGCGGGGGGCGGCGGGCCCTCCCGCAGAACACCATGCGCT |
| 114-564 | | flanking | CTTCGGAAGCATCGACAAGGTAGACTCCCTGTGACCTGGTAGTCTA |
| | | | CCATGTCGATGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4427 | Passenger Strand | GCATCGACAAGGTAGACTC |
| | | (DNA) | |
| | 4488 | Passenger Strand | GCAUCGACAAGGUAGACUC |
| | | (RNA) | |
| | 4993 | Passenger Strand | GCATCGACAAGGTAGACTCCC |
| | | (DNA) v2 | |
| | 4994 | Passenger Strand | GCAUCGACAAGGUAGACUCCC |
| | | (RNA) v2 | |
| | 4362 | Loop | TGTGACCTGG |
| | 4436 | Guide Strand | TAGTCTACCATGTCGATGC |
| | | (DNA) | |
| | 4691 | Guide Strand | UAGUCUACCAUGUCGAUGC |
| | | (RNA) | |
| | 4915 | Guide Strand | TAGTCTACCATGTCGATGCTT |
| | | (DNA) v2 | |
| | 4916 | Guide Strand | UAGUCUACCAUGUCGAUGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4398 | 5′ flanking to 3′ | GTGCTGGGCGGGGGGCGGCGGGCCCTCCCGCAGAACACCATGCGCT |
| 114-579 | | flanking | CTTCGGAAGCTCCTCGCTGTTCGGTTCCCTGTGACCTGGTAACCGA |
| | | | ACTGCGAGGAGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4428 | Passenger Strand | GCTCCTCGCTGTTCGGTTC |
| | | (DNA) | |
| | 4505 | Passenger Strand | GCUCCUCGCUGUUCGGUUC |
| | | (RNA) | |
| | 4997 | Passenger Strand | GCTCCTCGCTGTTCGGTTCCC |
| | | (DNA) v2 | |
| | 4998 | Passenger Strand | GCUCCUCGCUGUUCGGUUCCC |
| | | (RNA) v2 | |
| | 4362 | Loop | TGTGACCTGG |
| | 4437 | Guide Strand | TAACCGAACTGCGAGGAGC |
| | | (DNA) | |
| | 4700 | Guide Strand | UAACCGAACUGCGAGGAGC |
| | | (RNA) | |
| | 4919 | Guide Strand | TAACCGAACTGCGAGGAGCTT |
| | | (DNA) v2 | |
| | 4920 | Guide Strand | UAACCGAACUGCGAGGAGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4399 | 5′ flanking to 3′ | GTGCTGGGCGGGGGGCGGCGGGCCCTCCCGCAGAACACCATGCGCT |
| 114-586 | | flanking | CTTCGGAAGGCTTTGGCACGGGACTTCCCTGTGACCTGGTAAGTCC |
| | | | CGAGCCAAAGCCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4429 | Passenger Strand | GGCTTTGGCACGGGACTTC |
| | | (DNA) | |
| | 4483 | Passenger Strand | GGCUUUGGCACGGGACUUC |
| | | (RNA) | |
| | 5001 | Passenger Strand | GGCTTTGGCACGGGACTTCCC |
| | | (DNA) v2 | |
| | 5002 | Passenger Strand | GGCUUUGGCACGGGACUUCCC |
| | | (RNA) v2 | |
| | 4362 | Loop | TGTGACCTGG |
| | 4438 | Guide Strand | TAAGTCCCGAGCCAAAGCC |
| | | (DNA) | |
| | 4690 | Guide Strand | UAAGUCCCGAGCCAAAGCC |
| | | (RNA) | |
| | 4923 | Guide Strand | TAAGTCCCGAGCCAAAGCCTT |
| | | (DNA) v2 | |
| | 4924 | Guide Strand | UAAGUCCCGAGCCAAAGCCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5039 | 5′ flanking to 3′ | GTGCTGGGCGGGGGGCGGCGGGCCCTCCCGCAGAACACCATGCGCT |
| 114-582 | | flanking | CTTCGGAACCTCGCAGTACGGTTAATCCCTGTGACCTGGTATTAAC |
| | | | CGAACTGCGAGGTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5067 | Passenger Strand | CCTCGCAGTACGGTTAATC |
| | | (DNA) | |
| | 5068 | Passenger Strand | CCUCGCAGUACGGUUAAUC |
| | | (RNA) | |
| | 5069 | Passenger Strand | CCTCGCAGTACGGTTAATCCC |
| | | (DNA) v2 | |
| | 5070 | Passenger Strand | CCUCGCAGUACGGUUAAUCCC |
| | | (RNA) v2 | |
| | 4362 | Loop | TGTGACCTGG |
| | 5055 | Guide Strand | TATTAACCGAACTGCGAGG |
| | | (DNA) | |
| | 4694 | Guide Strand | UAUUAACCGAACUGCGAGG |
| | | (RNA) | |
| | 5056 | Guide Strand | TATTAACCGAACTGCGAGGTT |
| | | (DNA) v2 | |
| | 5057 | Guide Strand | UAUUAACCGAACUGCGAGGUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4400 | 5′ flanking to 3′ | GTGCTGGGCGGGGGGCGGCGGGCCCTCCCGCAGAACACCATGCGCT |
| 116-530 | | flanking | CTTCGGAAGACTGGAAGCGATGACAAGCCTGTGACCTGGTTTGTCA |
| | | | TCGCTTCCAGTCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4430 | Passenger Strand | GACTGGAAGCGATGACAAG |
| | | (DNA) | |
| | 4499 | Passenger Strand | GACUGGAAGCGAUGACAAG |
| | | (RNA) | |
| | 5005 | Passenger Strand | GACTGGAAGCGATGACAAGCC |
| | | (DNA) v2 | |
| | 5006 | Passenger Strand | GACUGGAAGCGAUGACAAGCC |
| | | (RNA) v2 | |
| | 4362 | Loop | TGTGACCTGG |
| | 4434 | Guide Strand | TTTGTCATCGCTTCCAGTC |
| | | (DNA) | |
| | 4697 | Guide Strand | UUUGUCAUCGCUUCCAGUC |
| | | (RNA) | |
| | 4907 | Guide Strand | TTTGTCATCGCTTCCAGTCTT |
| | | (DNA) v2 | |
| | 4908 | Guide Strand | UUUGUCAUCGCUUCCAGUCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4401 | 5′ flanking to 3′ | GTGCTGGGCGGGGGGCGGCGGGCCCTCCCGCAGAACACCATGCGCT |
| 116-556 | | flanking | CTTCGGAACTTCCGCGAGAACGCCAAGCCTGTGACCTGGTTTGGCG |
| | | | TTCTCGCGGAAGTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4431 | Passenger Strand | CTTCCGCGAGAACGCCAAG |
| | | (DNA) | |
| | 4510 | Passenger Strand | CUUCCGCGAGAACGCCAAG |
| | | (RNA) | |
| | 5009 | Passenger Strand | CTTCCGCGAGAACGCCAAGCC |
| | | (DNA) v2 | |
| | 5010 | Passenger Strand | CUUCCGCGAGAACGCCAAGCC |
| | | (RNA) v2 | |
| | 4362 | Loop | TGTGACCTGG |
| | 4435 | Guide Strand | TTTGGCGTTCTCGCGGAAG |
| | | (DNA) | |
| | 4701 | Guide Strand | UUUGGCGUUCUCGCGGAAG |
| | | (RNA) | |
| | 4911 | Guide Strand | TTTGGCGTTCTCGCGGAAGTT |
| | | (DNA) v2 | |
| | 4912 | Guide Strand | UUUGGCGUUCUCGCGGAAGUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4402 | 5′ flanking to 3′ | GTGCTGGGCGGGGGGCGGCGGGCCCTCCCGCAGAACACCATGCGCT |
| 116-564 | | flanking | CTTCGGAAGCATCGACATGGTAGACTGCCTGTGACCTGGTAGTCTA |
| | | | CCATGTCGATGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 4432 | Passenger Strand | GCATCGACATGGTAGACTG |
| | | (DNA) | |
| | 4489 | Passenger Strand | GCAUCGACAUGGUAGACUG |
| | | (RNA) | |
| | 5013 | Passenger Strand | GCATCGACATGGTAGACTGCC |
| | | (DNA) v2 | |
| | 5014 | Passenger Strand | GCAUCGACAUGGUAGACUGCC |
| | | (RNA) v2 | |
| | 4362 | Loop | TGTGACCTGG |
| | 4436 | Guide Strand | TAGTCTACCATGTCGATGC |
| | | (DNA) | |
| | 4691 | Guide Strand | UAGUCUACCAUGUCGAUGC |
| | | (RNA) | |
| | 4915 | Guide Strand | TAGTCTACCATGTCGATGCTT |
| | | (DNA) v2 | |
| | 4916 | Guide Strand | UAGUCUACCAUGUCGAUGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4403 | 5′ flanking to 3′ | GTGCTGGGCGGGGGGCGGCGGGCCCTCCCGCAGAACACCATGCGCT |
| 116-579 | | flanking | CTTCGGAAGCTCCTCGCAGTTCGGTTGCCTGTGACCTGGTAACCGA |
| | | | ACTGCGAGGAGCTTCTGTGGAGCGCCTTGACAGCAGCCATGGGAGG |
| | | | GCCGCCCCCTACCTCAGTGA |
| | 4354 | 5′ flanking | GTGCTGGGGGGGGGGGGGCGGGCCCTCCCGCAGAACACCATGCGCT |
| | | | CTTCGGAA |
| | 4418 | Passenger Strand | GCTCCTCGCAGTTCGGTTG |
| | | (DNA) | |
| | 4503 | Passenger Strand | GCUCCUCGCAGUUCGGUUG |
| | | (RNA) | |
| | 4957 | Passenger Strand | GCTCCTCGCAGTTCGGTTGCC |
| | | (DNA) v2 | |
| | 4958 | Passenger Strand | GCUCCUCGCAGUUCGGUUGCC |
| | | (RNA) v2 | |
| | 4362 | Loop | TGTGACCTGG |
| | 4437 | Guide Strand | TAACCGAACTGCGAGGAGC |
| | | (DNA) | |
| | 4700 | Guide Strand | UAACCGAACUGCGAGGAGC |
| | | (RNA) | |
| | 4919 | Guide Strand | TAACCGAACTGCGAGGAGCTT |
| | | (DNA) v2 | |
| | 4920 | Guide Strand | UAACCGAACUGCGAGGAGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 4404 | 5′ flanking to 3′ | GTGCTGGGCGGGGGGCGGCGGGCCCTCCCGCAGAACACCATGCGCT |
| | | flanking | CTTCGGAAGGCTTTGGCTCGGGACTTGCCTGTGACCTGGTAAGTCC |
| | | | CGAGCCAAAGCCTTCTGTGGAGCGCCTTGACAGCAGCCATGGGAGG |
| | | | GCCGCCCCCTACCTCAGTGA |
| 116-586 | 4354 | 5′ flanking | GTGCTGGGCGGGGGGCGGCGGGCCCTCCCGCAGAACACCATGCGCT |
| | | | CTTCGGAA |
| | 4433 | Passenger Strand | GGCTTTGGCTCGGGACTTG |
| | | (DNA) | |
| | 4484 | Passenger Strand | GGCUUUGGCUCGGGACUUG |
| | | (RNA) | |
| | 5021 | Passenger Strand | GGCTTTGGCTCGGGACTTGCC |
| | | (DNA) v2 | |
| | 5022 | Passenger Strand | GGCUUUGGCUCGGGACUUGCC |
| | | (RNA) v2 | |
| | 4362 | Loop | TGTGACCTGG |
| | 4438 | Guide Strand | TAAGTCCCGAGCCAAAGCC |
| | | (DNA) | |
| | 4690 | Guide Strand | UAAGUCCCGAGCCAAAGCC |
| | | (RNA) | |
| | 4923 | Guide Strand | TAAGTCCCGAGCCAAAGCCTT |
| | | (DNA) v2 | |
| | 4924 | Guide Strand | UAAGUCCCGAGCCAAAGCCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5043 | 5′ flanking to 3′ | GTGCTGGGCGGGGGGCGGCGGGCCCTCCCGCAGAACACCATGCGCT |
| 116-582 | | flanking | CTTCGGAACCTCGCAGTTCGGTTAATGCCTGTGACCTGGTATTAAC |
| | | | CGAACTGCGAGGTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5071 | Passenger Strand | CCTCGCAGTTCGGTTAATG |
| | | (DNA) | |
| | 5072 | Passenger Strand | CCUCGCAGUUCGGUUAAUG |
| | | (RNA) | |
| | 5073 | Passenger Strand | CCTCGCAGTTCGGTTAATGCC |
| | | (DNA) v2 | |
| | 5074 | Passenger Strand | CCUCGCAGUUCGGUUAAUGCC |
| | | (RNA) v2 | |
| | 4362 | Loop | TGTGACCTGG |
| | 5055 | Guide Strand | TATTAACCGAACTGCGAGG |
| | | (DNA) | |
| | 4694 | Guide Strand | UAUUAACCGAACUGCGAGG |
| | | (RNA) | |
| | 5056 | Guide Strand | TATTAACCGAACTGCGAGGTT |
| | | (DNA) v2 | |
| | 5057 | Guide Strand | UAUUAACCGAACUGCGAGGUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5028 | 5′ flanking to 3′ | GTGCTGGGCGGGGGGCGGCGGGCCCTCCCGCAGAACACCATGCGCT |
| 102-587 | | flanking | CTTCGGAAGCTTTGGCTCGGGACTTCCCCTGTGACCTGGTGAAGTC |
| | | | CCGAGCCAAAGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5075 | Passenger Strand | GCTTTGGCTCGGGACTTCC |
| | | (DNA) | |
| | 4477 | Passenger Strand | GCUUUGGCUCGGGACUUCC |
| | | (RNA) | |
| | 5076 | Passenger Strand | GCTTTGGCTCGGGACTTCCCC |
| | | (DNA) v2 | |
| | 5077 | Passenger Strand | GCUUUGGCUCGGGACUUCCCC |
| | | (RNA) v2 | |
| | 4362 | Loop | TGTGACCTGG |
| | 5078 | Guide Strand | TGAAGTCCCGAGCCAAAGC |
| | | (DNA) | |
| | 4687 | Guide Strand | UGAAGUCCCGAGCCAAAGC |
| | | (RNA) | |
| | 5079 | Guide Strand | TGAAGTCCCGAGCCAAAGCTT |
| | | (DNA) v2 | |
| | 5080 | Guide Strand | UGAAGUCCCGAGCCAAAGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5032 | 5′ flanking to 3′ | GTGCTGGGCGGGGGGCGGCGGGCCCTCCCGCAGAACACCATGCGCT |
| 104-587 | | flanking | CTTCGGAAGCTTTGGCTCGGGACTTCACCTGTGACCTGGTGAAGTC |
| | | | CCGAGCCAAAGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5081 | Passenger Strand | GCTTTGGCTCGGGACTTCA |
| | | (DNA) | |
| | 5082 | Passenger Strand | GCUUUGGCUCGGGACUUCA |
| | | (RNA) | |
| | 5083 | Passenger Strand | GCTTTGGCTCGGGACTTCACC |
| | | (DNA) v2 | |
| | 5084 | Passenger Strand | GCUUUGGCUCGGGACUUCACC |
| | | (RNA) v2 | |
| | 4362 | Loop | TGTGACCTGG |
| | 5078 | Guide Strand | TGAAGTCCCGAGCCAAAGC |
| | | (DNA) | |
| | 4687 | Guide Strand | UGAAGUCCCGAGCCAAAGC |
| | | (RNA) | |
| | 5079 | Guide Strand | TGAAGTCCCGAGCCAAAGCTT |
| | | (DNA) v2 | |
| | 5080 | Guide Strand | UGAAGUCCCGAGCCAAAGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5036 | 5′ flanking to 3′ | GTGCTGGGCGGGGGGCGGCGGGCCCTCCCGCAGAACACCATGCGCT |
| 109-587 | | flanking | CTTCGGAAGCTTTGGCTCGGGACTTCTCCTGTGATTTGGTGAAGTC |
| | | | CCGAGCCAAAGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5085 | Passenger Strand | GCTTTGGCTCGGGACTTCT |
| | | (DNA) | |
| | 5086 | Passenger Strand | GCUUUGGCUCGGGACUUCU |
| | | (RNA) | |
| | 5087 | Passenger Strand | GCTTTGGCTCGGGACTTCTCC |
| | | (DNA) v2 | |
| | 5088 | Passenger Strand | GCUUUGGCUCGGGACUUCUCC |
| | | (RNA) v2 | |
| | 4363 | Loop | TGTGATTTGG |
| | 5078 | Guide Strand | TGAAGTCCCGAGCCAAAGC |
| | | (DNA) | |
| | 4687 | Guide Strand | UGAAGUCCCGAGCCAAAGC |
| | | (RNA) | |
| | 5079 | Guide Strand | TGAAGTCCCGAGCCAAAGCTT |
| | | (DNA) v2 | |
| | 5080 | Guide Strand | UGAAGUCCCGAGCCAAAGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5040 | 5′ flanking to 3′ | GTGCTGGGCGGGGGGCGGCGGGCCCTCCCGCAGAACACCATGCGCT |
| 114-587 | | flanking | CTTCGGAAGCTTTGGCTGGGGACTTCCCCTGTGACCTGGTGAAGTC |
| | | | CCGAGCCAAAGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5089 | Passenger Strand | GCTTTGGCTGGGGACTTCC |
| | | (DNA) | |
| | 5090 | Passenger Strand | GCUUUGGCUGGGGACUUCC |
| | | (RNA) | |
| | 5091 | Passenger Strand | GCTTTGGCTGGGGACTTCCCC |
| | | (DNA) v2 | |
| | 5092 | Passenger Strand | GCUUUGGCUGGGGACUUCCCC |
| | | (RNA) v2 | |
| | 4362 | Loop | TGTGACCTGG |
| | 5078 | Guide Strand | TGAAGTCCCGAGCCAAAGC |
| | | (DNA) | |
| | 4687 | Guide Strand | UGAAGUCCCGAGCCAAAGC |
| | | (RNA) | |
| | 5079 | Guide Strand | TGAAGTCCCGAGCCAAAGCTT |
| | | (DNA) v2 | |
| | 5080 | Guide Strand | UGAAGUCCCGAGCCAAAGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5044 | 5′ flanking to 3′ | GTGCTGGGCGGGGGGCGGCGGGCCCTCCCGCAGAACACCATGCGCT |
| 116-587 | | flanking | CTTCGGAAGCTTTGGCTCGGGACTTCGCCTGTGACCTGGTGAAGTC |
| | | | CCGAGCCAAAGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5093 | Passenger Strand | GCTTTGGCTCGGGACTTCG |
| | | (DNA) | |
| | 5094 | Passenger Strand | GCUUUGGCUCGGGACUUCG |
| | | (RNA) | |
| | 5095 | Passenger Strand | GCTTTGGCTCGGGACTTCGCC |
| | | (DNA) v2 | |
| | 5096 | Passenger Strand | GCUUUGGCUCGGGACUUCGCC |
| | | (RNA) v2 | |
| | 4362 | Loop | TGTGACCTGG |
| | 5078 | Guide Strand | TGAAGTCCCGAGCCAAAGC |
| | | (DNA) | |
| | 4687 | Guide Strand | UGAAGUCCCGAGCCAAAGC |
| | | (RNA) | |
| | 5079 | Guide Strand | TGAAGTCCCGAGCCAAAGCTT |
| | | (DNA) v2 | |
| | 5080 | Guide Strand | UGAAGUCCCGAGCCAAAGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5048 | 5′ flanking to 3′ | GAAGCAAAGAAGGGGCAGAGGGAGCCCGTGAGCTGAGTGGGCCAGG |
| 127-587 | | flanking | GACTGGGAGAAGGAGTGAGGAGGCAGGGCCGGCATGCCTCTGCTGC |
| | | | TGGCCAGAGCTTTGGCTCGGGACTTCTCTGTCTGCACCTGTCACTA |
| | | | GTGAAGTCCCGAGCCAAAGCTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 5085 | Passenger Strand | GCTTTGGCTCGGGACTTCT |
| | | (DNA) | |
| | 5086 | Passenger Strand | GCUUUGGCUCGGGACUUCU |
| | | (RNA) | |
| | 5097 | Passenger Strand | GCTTTGGCTCGGGACTTCTCT |
| | | (DNA) v2 | |
| | 5098 | Passenger Strand | GCUUUGGCUCGGGACUUCUCU |
| | | (RNA) v2 | |
| | 4366 | Loop | GTCTGCACCTGTCACTAG |
| | 5078 | Guide Strand | TGAAGTCCCGAGCCAAAGC |
| | | (DNA) | |
| | 4687 | Guide Strand | UGAAGUCCCGAGCCAAAGC |
| | | (RNA) | |
| | 5079 | Guide Strand | TGAAGTCCCGAGCCAAAGCTT |
| | | (DNA) v2 | |
| | 5080 | Guide Strand | UGAAGUCCCGAGCCAAAGCUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 5029 | 5′ flanking to 3′ | GTGCTGGGCGGGGGGCGGCGGGCCCTCCCGCAGAACACCATGCGCT |
| 102-552 | | flanking | CTTCGGAAAGCTGACCTTCCGCGAGACCCTGTGACCTGGTTCTCGC |
| | | | GGAAGGTCAGCTTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5099 | Passenger Strand | AGCTGACCTTCCGCGAGAC |
| | | (DNA) | |
| | 4494 | Passenger Strand | AGCUGACCUUCCGCGAGAC |
| | | (RNA) | |
| | 5100 | Passenger Strand | AGCTGACCTTCCGCGAGACCC |
| | | (DNA) v2 | |
| | 5101 | Passenger Strand | AGCUGACCUUCCGCGAGACCC |
| | | (RNA) v2 | |
| | 4362 | Loop | TGTGACCTGG |
| | 5102 | Guide Strand | TTCTCGCGGAAGGTCAGCT |
| | | (DNA) | |
| | 4696 | Guide Strand | UUCUCGCGGAAGGUCAGCU |
| | | (RNA) | |
| | 5103 | Guide Strand | TTCTCGCGGAAGGTCAGCTTT |
| | | (DNA) v2 | |
| | 5104 | Guide Strand | UUCUCGCGGAAGGUCAGCUUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5033 | 5′ flanking to 3′ | GTGCTGGGCGGGGGGGGGGGGGCCCTCCCGCAGAACACCATGCGCT |
| 104-552 | | flanking | CTTCGGAAAGCTGACCTTCCGCGAGAACCTGTGACCTGGTTCTCGC |
| | | | GGAAGGTCAGCTTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5105 | Passenger Strand | AGCTGACCTTCCGCGAGAA |
| | | (DNA) | |
| | 5106 | Passenger Strand | AGCUGACCUUCCGCGAGAA |
| | | (RNA) | |
| | 5107 | Passenger Strand | AGCTGACCTTCCGCGAGAACC |
| | | (DNA) v2 | |
| | 5108 | Passenger Strand | AGCUGACCUUCCGCGAGAACC |
| | | (RNA) v2 | |
| | 4362 | Loop | TGTGACCTGG |
| | 5102 | Guide Strand | TTCTCGCGGAAGGTCAGCT |
| | | (DNA) | |
| | 4696 | Guide Strand | UUCUCGCGGAAGGUCAGCU |
| | | (RNA) | |
| | 5103 | Guide Strand | TTCTCGCGGAAGGTCAGCTTT |
| | | (DNA) v2 | |
| | 5104 | Guide Strand | UUCUCGCGGAAGGUCAGCUUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5037 | 5′ flanking to 3′ | GTGCTGGGCGGGGGGGGGGGGGCCCTCCCGCAGAACACCATGCGCT |
| 109-552 | | flanking | CTTCGGAAAGCTGACCTTCCGCGAGATCCTGTGATTTGGTTCTCGC |
| | | | GGAAGGTCAGCTTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5109 | Passenger Strand | AGCTGACCTTCCGCGAGAT |
| | | (DNA) | |
| | 5110 | Passenger Strand | AGCUGACCUUCCGCGAGAU |
| | | (RNA) | |
| | 5111 | Passenger Strand | AGCTGACCTTCCGCGAGATCC |
| | | (DNA) v2 | |
| | 5112 | Passenger Strand | AGCUGACCUUCCGCGAGAUCC |
| | | (RNA) v2 | |
| | 4363 | Loop | TGTGATTTGG |
| | 5102 | Guide Strand | TTCTCGCGGAAGGTCAGCT |
| | | (DNA) | |
| | 4696 | Guide Strand | UUCUCGCGGAAGGUCAGCU |
| | | (RNA) | |
| | 5103 | Guide Strand | TTCTCGCGGAAGGTCAGCTTT |
| | | (DNA) v2 | |
| | 5104 | Guide Strand | UUCUCGCGGAAGGUCAGCUUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5041 | 5′ flanking to 3′ | GTGCTGGGCGGGGGGCGGCGGGCCCTCCCGCAGAACACCATGCGCT |
| 114-552 | | flanking | CTTCGGAAAGCTGACCTACCGCGAGACCCTGTGACCTGGTTCTCGC |
| | | | GGAAGGTCAGCTTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5113 | Passenger Strand | AGCTGACCTACCGCGAGAC |
| | | (DNA) | |
| | 5114 | Passenger Strand | AGCUGACCUACCGCGAGAC |
| | | (RNA) | |
| | 5115 | Passenger Strand | AGCTGACCTACCGCGAGACCC |
| | | (DNA) v2 | |
| | 5116 | Passenger Strand | AGCUGACCUACCGCGAGACCC |
| | | (RNA) v2 | |
| | 4362 | Loop | TGTGACCTGG |
| | 5102 | Guide Strand | TTCTCGCGGAAGGTCAGCT |
| | | (DNA) | |
| | 4696 | Guide Strand | UUCUCGCGGAAGGUCAGCU |
| | | (RNA) | |
| | 5103 | Guide Strand | TTCTCGCGGAAGGTCAGCTTT |
| | | (DNA) v2 | |
| | 5104 | Guide Strand | UUCUCGCGGAAGGUCAGCUUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5045 | 5′ flanking to 3′ | GTGCTGGGCGGGGGGCGGCGGGCCCTCCCGCAGAACACCATGCGCT |
| 116-552 | | flanking | CTTCGGAAAGCTGACCTTCCGCGAGAGCCTGTGACCTGGTTCTCGC |
| | | | GGAAGGTCAGCTTTCTGTGGAGCGCCTTGACAGCAGCCATGGGAGG |
| | | | GCCGCCCCCTACCTCAGTGA |
| | 4354 | 5′ flanking | GTGCTGGGGGGGGGGGGGCGGGCCCTCCCGCAGAACACCATGCGCT |
| | | | CTTCGGAA |
| | 5117 | Passenger Strand | AGCTGACCTTCCGCGAGAG |
| | | (DNA) | |
| | 5118 | Passenger Strand | AGCUGACCUUCCGCGAGAG |
| | | (RNA) | |
| | 5119 | Passenger Strand | AGCTGACCTTCCGCGAGAGCC |
| | | (DNA) v2 | |
| | 5120 | Passenger Strand | AGCUGACCUUCCGCGAGAGCC |
| | | (RNA) v2 | |
| | 4362 | Loop | TGTGACCTGG |
| | 5102 | Guide Strand | TTCTCGCGGAAGGTCAGCT |
| | | (DNA) | |
| | 4696 | Guide Strand | UUCUCGCGGAAGGUCAGCU |
| | | (RNA) | |
| | 5103 | Guide Strand | TTCTCGCGGAAGGTCAGCTTT |
| | | (DNA) v2 | |
| | 5104 | Guide Strand | UUCUCGCGGAAGGUCAGCUUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5049 | 5′ flanking to 3′ | GAAGCAAAGAAGGGGCAGAGGGAGCCCGTGAGCTGAGTGGGCCAGG |
| 127-552 | | flanking | GACTGGGAGAAGGAGTGAGGAGGCAGGGCCGGCATGCCTCTGCTGC |
| | | | TGGCCAGAAGCTGACCTTCCGCGAGATCTGTCTGCACCTGTCACTA |
| | | | GTTCTCGCGGAAGGTCAGCTTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 5109 | Passenger Strand | AGCTGACCTTCCGCGAGAT |
| | | (DNA) | |
| | 5110 | Passenger Strand | AGCUGACCUUCCGCGAGAU |
| | | (RNA) | |
| | 5121 | Passenger Strand | AGCTGACCTTCCGCGAGATCT |
| | | (DNA) v2 | |
| | 5122 | Passenger Strand | AGCUGACCUUCCGCGAGAUCU |
| | | (RNA) v2 | |
| | 4366 | Loop | GTCTGCACCTGTCACTAG |
| | 5102 | Guide Strand | TTCTCGCGGAAGGTCAGCT |
| | | (DNA) | |
| | 4696 | Guide Strand | UUCUCGCGGAAGGUCAGCU |
| | | (RNA) | |
| | 5103 | Guide Strand | TTCTCGCGGAAGGTCAGCTTT |
| | | (DNA) v2 | |
| | 5104 | Guide Strand | UUCUCGCGGAAGGUCAGCUUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |
| VOYTaumiR- | 5030 | 5′ flanking to 3′ | GTGCTGGGCGGGGGGCGGCGGGCCCTCCCGCAGAACACCATGCGCT |
| 102-549 | | flanking | CTTCGGAAGCCGTCTTCCGCCAAGAGCCCTGTGACCTGGTCTCTTG |
| | | | GCGGAAGACGGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5123 | Passenger Strand | GCCGTCTTCCGCCAAGAGC |
| | | (DNA) | |
| | 4527 | Passenger Strand | GCCGUCUUCCGCCAAGAGC |
| | | (RNA) | |
| | 5124 | Passenger Strand | GCCGTCTTCCGCCAAGAGCCC |
| | | (DNA) v2 | |
| | 5125 | Passenger Strand | GCCGUCUUCCGCCAAGAGCCC |
| | | (RNA) v2 | |
| | 4362 | Loop | TGTGACCTGG |
| | 5126 | Guide Strand | TCTCTTGGCGGAAGACGGC |
| | | (DNA) | |
| | 4718 | Guide Strand | UCUCUUGGCGGAAGACGGC |
| | | (RNA) | |
| | 5127 | Guide Strand | TCTCTTGGCGGAAGACGGCTT |
| | | (DNA) v2 | |
| | 5128 | Guide Strand | UCUCUUGGCGGAAGACGGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5034 | 5′ flanking to 3′ | GTGCTGGGCGGGGGGCGGCGGGCCCTCCCGCAGAACACCATGCGCT |
| 104-549 | | flanking | CTTCGGAAGCCGTCTTCCGCCAAGAGACCTGTGACCTGGTCTCTTG |
| | | | GCGGAAGACGGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5129 | Passenger Strand | GCCGTCTTCCGCCAAGAGA |
| | | (DNA) | |
| | 5130 | Passenger Strand | GCCGUCUUCCGCCAAGAGA |
| | | (RNA) | |
| | 5131 | Passenger Strand | GCCGTCTTCCGCCAAGAGACC |
| | | (DNA) v2 | |
| | 5132 | Passenger Strand | GCCGUCUUCCGCCAAGAGACC |
| | | (RNA) v2 | |
| | 4362 | Loop | TGTGACCTGG |
| | 5126 | Guide Strand | TCTCTTGGCGGAAGACGGC |
| | | (DNA) | |
| | 4718 | Guide Strand | UCUCUUGGCGGAAGACGGC |
| | | (RNA) | |
| | 5127 | Guide Strand | TCTCTTGGCGGAAGACGGCTT |
| | | (DNA) v2 | |
| | 5128 | Guide Strand | UCUCUUGGCGGAAGACGGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5038 | 5′ flanking to 3′ | GTGCTGGGCGGGGGGCGGCGGGCCCTCCCGCAGAACACCATGCGCT |
| 109-549 | | flanking | CTTCGGAAGCCGTCTTCCGCCAAGAGTCCTGTGATTTGGTCTCTTG |
| | | | GCGGAAGACGGCTTCTGAGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5133 | Passenger Strand | GCCGTCTTCCGCCAAGAGT |
| | | (DNA) | |
| | 5134 | Passenger Strand | GCCGUCUUCCGCCAAGAGU |
| | | (RNA) | |
| | 5135 | Passenger Strand | GCCGTCTTCCGCCAAGAGTCC |
| | | (DNA) v2 | |
| | 5136 | Passenger Strand | GCCGUCUUCCGCCAAGAGUCC |
| | | (RNA) v2 | |
| | 4363 | Loop | TGTGATTTGG |
| | 5126 | Guide Strand | TCTCTTGGCGGAAGACGGC |
| | | (DNA) | |
| | 4718 | Guide Strand | UCUCUUGGCGGAAGACGGC |
| | | (RNA) | |
| | 5127 | Guide Strand | TCTCTTGGCGGAAGACGGCTT |
| | | (DNA) v2 | |
| | 5128 | Guide Strand | UCUCUUGGCGGAAGACGGCUU |
| | | (RNA) v2 | |
| | 4373 | 3′ Flanking | CTGAGGAGCGCCTTGACAGCAGCCATGGGAGGGCCGCCCCCTACCT |
| | | | CAGTGA |
| VOYTaumiR- | 5042 | 5′ flanking to 3′ | GTGCTGGGCGGGGGGCGGCGGGCCCTCCCGCAGAACACCATGCGCT |
| 114-549 | | flanking | CTTCGGAAGCCGTCTTCGGCCAAGAGCCCTGTGACCTGGTCTCTTG |
| | | | GCGGAAGACGGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5137 | Passenger Strand | GCCGTCTTCGGCCAAGAGC |
| | | (DNA) | |
| | 5138 | Passenger Strand | GCCGUCUUCGGCCAAGAGC |
| | | (RNA) | |
| | 5139 | Passenger Strand | GCCGTCTTCGGCCAAGAGCCC |
| | | (DNA) v2 | |
| | 5140 | Passenger Strand | GCCGUCUUCGGCCAAGAGCCC |
| | | (RNA) v2 | |
| | 4362 | Loop | TGTGACCTGG |
| | 5126 | Guide Strand | TCTCTTGGCGGAAGACGGC |
| | | (DNA) | |
| | 4718 | Guide Strand | UCUCUUGGCGGAAGACGGC |
| | | (RNA) | |
| | 5127 | Guide Strand | TCTCTTGGCGGAAGACGGCTT |
| | | (DNA) v2 | |
| | 5128 | Guide Strand | UCUCUUGGCGGAAGACGGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5046 | 5′ flanking to 3′ | GTGCTGGGGGGGGGGGGGCGGGCCCTCCCGCAGAACACCATGCGCT |
| 116-549 | | flanking | CTTCGGAAGCCGTCTTCCGCCAAGAGGCCTGTGACCTGGTCTCTTG |
| | | | GCGGAAGACGGCTTCTGTGGAGCGCCTTGACAGCAGCCATGGGAGG |
| | | | GCCGCCCCCTACCTCAGTGA |
| | 4354 | 5′ flanking | GTGCTGGGCGGGGGGCGGCGGGCCCTCCCGCAGAACACCATGCGCT |
| | | | CTTCGGAA |
| | 5141 | Passenger Strand | GCCGTCTTCCGCCAAGAGG |
| | | (DNA) | |
| | 5142 | Passenger Strand | GCCGUCUUCCGCCAAGAGG |
| | | (RNA) | |
| | 5143 | Passenger Strand | GCCGTCTTCCGCCAAGAGGCC |
| | | (DNA) v2 | |
| | 5144 | Passenger Strand | GCCGUCUUCCGCCAAGAGGCC |
| | | (RNA) v2 | |
| | 4362 | Loop | TGTGACCTGG |
| | 5126 | Guide Strand | TCTCTTGGCGGAAGACGGC |
| | | (DNA) | |
| | 4718 | Guide Strand | UCUCUUGGCGGAAGACGGC |
| | | (RNA) | |
| | 5127 | Guide Strand | TCTCTTGGCGGAAGACGGCTT |
| | | (DNA) v2 | |
| | 5128 | Guide Strand | UCUCUUGGCGGAAGACGGCUU |
| | | (RNA) v2 | |
| | 4374 | 3′ Flanking | CTGTGGAGCGCCTTGACAGCAGCCATGGGAGGGCCGCCCCCTACCT |
| | | | CAGTGA |
| VOYTaumiR- | 5050 | 5′ flanking to 3′ | GAAGCAAAGAAGGGGCAGAGGGAGCCCGTGAGCTGAGTGGGCCAGG |
| 127-549 | | flanking | GACTGGGAGAAGGAGTGAGGAGGCAGGGCCGGCATGCCTCTGCTGC |
| | | | TGGCCAGAGCCGTCTTCCGCCAAGAGTCTGTCTGCACCTGTCACTA |
| | | | GTCTCTTGGCGGAAGACGGCTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |
| | 4355 | 5′ flanking | GAAGCAAAGAAGGGGCAGAGGGAGCCCGTGAGCTGAGTGGGCCAGG |
| | | | GACTGGGAGAAGGAGTGAGGAGGCAGGGCCGGCATGCCTCTGCTGC |
| | | | TGGCCAGA |
| | 5133 | Passenger Strand | GCCGTCTTCCGCCAAGAGT |
| | | (DNA) | |
| | 5134 | Passenger Strand | GCCGUCUUCCGCCAAGAGU |
| | | (RNA) | |
| | 5145 | Passenger Strand | GCCGTCTTCCGCCAAGAGTCT |
| | | (DNA) v2 | |
| | 5146 | Passenger Strand | GCCGUCUUCCGCCAAGAGUCU |
| | | (RNA) v2 | |
| | 4366 | Loop | GTCTGCACCTGTCACTAG |
| | 5126 | Guide Strand | TCTCTTGGCGGAAGACGGC |
| | | (DNA) | |
| | 4718 | Guide Strand | UCUCUUGGCGGAAGACGGC |
| | | (RNA) | |
| | 5127 | Guide Strand | TCTCTTGGCGGAAGACGGCTT |
| | | (DNA) v2 | |
| | 5128 | Guide Strand | UCUCUUGGCGGAAGACGGCUU |
| | | (RNA) v2 | |
| | 4375 | 3′ Flanking | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCC |

### AAV Particles Comprising Modulatory Polynucleotides

In some embodiments, the AAV particle comprises a viral genome comprising or encoding a modulatory polynucleotide. In such embodiments, a viral genome comprises or encodes a modulatory polynucleotide, e.g., an encoded modulatory polynucleotide comprising a passenger and guide strand for targeting a MAPT gene, mRNA, or protein described herein, is replicated and packaged into a viral particle. A target cell transduced with a viral particle comprising a modulatory polynucleotide may express the encoded sense and/or antisense sequences in a single cell.

Non-limiting examples of ITR to ITR sequences of AAV particles comprising a viral genome comprising a modulatory polynucleotide sequence are described, e.g., in Tables 10A, 10B, 11-18, and 26.

TABLE 10A
Exemplary Sequences of AAV Viral Genomes

| ITR to ITR | | Modulatory |
|---|---|---|
| Construct | ITR to ITR | Polynucleotide |
| Name | SEQ ID NO | SEQ ID NO |
| VOYTAU1 | 4439 | 4380 |
| VOYTAU2 | 4440 | 4381 |
| VOYTAU3 | 4441 | 4382 |
| VOYTAU4 | 4442 | 4383 |
| VOYTAU5 | 4443 | 4384 |
| VOYTAU6 | 4444 | 4385 |
| VOYTAU7 | 4445 | 4386 |
| VOYTAU8 | 4446 | 4387 |
| VOYTAU9 | 4447 | 4388 |
| VOYTAU10 | 4448 | 4389 |
| VOYTAU11 | 4449 | 4390 |
| VOYTAU12 | 4450 | 4391 |
| VOYTAU13 | 4451 | 4392 |
| VOYTAU14 | 4452 | 4393 |
| VOYTAU15 | 4453 | 4394 |
| VOYTAU16 | 4454 | 4395 |
| VOYTAU17 | 4455 | 4396 |
| VOYTAU18 | 4456 | 4397 |
| VOYTAU19 | 4457 | 4398 |
| VOYTAU20 | 4458 | 4399 |
| VOYTAU21 | 4459 | 4400 |
| VOYTAU22 | 4460 | 4401 |
| VOYTAU23 | 4461 | 4402 |
| VOYTAU24 | 4462 | 4403 |
| VOYTAU25 | 4463 | 4404 |
| VOYTAU26 | 4464 | 4405 |
| VOYTAU27 | 4465 | 4406 |
| VOYTAU28 | 4466 | 4407 |
| VOYTAU29 | 4467 | 4408 |
| VOYTAU30 | 4468 | 4409 |
| VOYTAU31 | 5149 | 5047 |
| VOYTAU32 | 5150 | 5048 |
| VOYTAU33 | 5151 | 5049 |
| VOYTAU34 | 5152 | 5050 |
| VOYTAU35 | 5153 | 5043 |
| VOYTAU36 | 5154 | 5044 |
| VOYTAU37 | 5155 | 5045 |
| VOYTAU38 | 5156 | 5046 |
| VOYTAU39 | 5157 | 5039 |
| VOYTAU40 | 5158 | 5040 |
| VOYTAU41 | 5159 | 5041 |
| VOYTAU42 | 5160 | 5042 |
| VOYTAU43 | 5161 | 5035 |
| VOYTAU44 | 5162 | 5036 |
| VOYTAU45 | 5163 | 5037 |
| VOYTAU46 | 5164 | 5038 |
| VOYTAU47 | 5165 | 5031 |
| VOYTAU48 | 5166 | 5032 |
| VOYTAU49 | 5167 | 5033 |
| VOYTAU50 | 5168 | 5034 |
| VOYTAU51 | 5169 | 5027 |
| VOYTAU52 | 5170 | 5028 |
| VOYTAU53 | 5171 | 5029 |
| VOYTAU54 | 5172 | 5030 |
| VOYTAU55 | 5173 | 4405 |
| VOYTAU56 | 5174 | 4380 |
| VOYTAU57 | 5175 | 4381 |
| VOYTAU58 | 5176 | 4383 |
| VOYTAU59 | 5177 | 4384 |
| VOYTAU60 | 5178 | 4385 |
| VOYTAU61 | 5179 | 4386 |
| VOYTAU62 | 5180 | 4388 |
| VOYTAU63 | 5181 | 4389 |
| VOYTAU64 | 5182 | 4390 |
| VOYTAU65 | 5183 | 4391 |
| VOYTAU66 | 5184 | 4393 |
| VOYTAU67 | 5185 | 4394 |
| VOYTAU68 | 5186 | 4395 |
| VOYTAU69 | 5187 | 4396 |
| VOYTAU70 | 5188 | 4398 |
| VOYTAU71 | 5189 | 4399 |
| VOYTAU72 | 5190 | 4400 |
| VOYTAU73 | 5191 | 4401 |
| VOYTAU74 | 5192 | 4403 |
| VOYTAU75 | 5193 | 4404 |
| VOYTAU76 | 5194 | 4406 |
| VOYTAU77 | 5195 | 4408 |
| VOYTAU78 | 5196 | 4409 |

TABLE 10B
Exemplary Sequences of AAV Viral Genomes

| Name | SEQ ID NO | Sequence |
|---|---|---|
| VOYTAU55 | 5173 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGAGACTGGAAGCGATGACAATCTGTCTGCACCTGTCACTAGTTTGTCATCGCTTC |
| | | CAGTCTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU56 | 5174 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGACTGGAAGCGATGACAACCCTGTGACCTGGTTTGTC |
| | | ATCGCTTCCAGTCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU57 | 5175 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGGGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACTTCCGCGAGAACGCCAACCCTGTGACCTGGTTTGGC |
| | | GTTCTCGCGGAAGTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU58 | 5176 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGGGGGGGGGGGCGAGGGGCGGGGGGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTCCTCGCAGTTCGGTTCCCTGTGACCTGGTAACCG |
| | | AACTGCGAGGAGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU59 | 5177 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAGGCTTTGGCTCGGGACTTCCCTGTGACCTGGTAAGTC |
| | | CCGAGCCAAAGCCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU60 | 5178 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGGGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAGACTGGAAGCGATGACAAACCTGTGACCTGGTTTGTC |
| | | ATCGCTTCCAGTCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU61 | 5179 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACTTCCGCGAGAACGCCAAACCTGTGACCTGGTTTGGC |
| | | GTTCTCGCGGAAGTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU62 | 5180 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTCCTCGCAGTTCGGTTGCCTGTGACCTGGTAACCG |
| | | AACTGCGAGGAGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU63 | 5181 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGGCTTTGGCTCGGGACTTACCTGTGACCTGGTAAGTC |
| | | CCGAGCCAAAGCCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU64 | 5182 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAGACTGGAAGCGATGACAATCCTGTGATTTGGTTTGTC |
| | | ATCGCTTCCAGTCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU65 | 5183 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAACTTCCGCGAGAACGCCAATCCTGTGATTTGGTTTGGC |
| | | GTTCTCGCGGAAGTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU66 | 5184 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTCCTCGCAGTTCGGTTTCCTGTGATTTGGTAACCG |
| | | AACTGCGAGGAGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU67 | 5185 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGGCTTTGGCTCGGGACTTTCCTGTGATTTGGTAAGTC |
| | | CCGAGCCAAAGCCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU68 | 5186 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGACTGGAAGGGATGACAACCCTGTGACCTGGTTTGTC |
| | | ATCGCTTCCAGTCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU69 | 5187 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGGGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACTTCCGCGACAACGCCAACCCTGTGACCTGGTTTGGC |
| | | GTTCTCGCGGAAGTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU70 | 5188 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTCCTCGCTGTTCGGTTCCCTGTGACCTGGTAACCG |
| | | AACTGCGAGGAGCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU71 | 5189 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAGGCTTTGGCACGGGACTTCCCTGTGACCTGGTAAGTC |
| | | CCGAGCCAAAGCCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU72 | 5190 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGACTGGAAGCGATGACAAGCCTGTGACCTGGTTTGTC |
| | | ATCGCTTCCAGTCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU73 | 5191 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACTTCCGCGAGAACGCCAAGCCTGTGACCTGGTTTGGC |
| | | GTTCTCGCGGAAGTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU74 | 5192 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTCCTCGCAGTTCGGTTGCCTGTGACCTGGTAACCG |
| | | AACTGCGAGGAGCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU75 | 5193 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAGGCTTTGGCTCGGGACTTGCCTGTGACCTGGTAAGTC |
| | | CCGAGCCAAAGCCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU76 | 5194 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGACTTCCGCGAGAACGCCAATCTGTCTGCACCTGTCACTAGTTTGGCGTTCTCGC |
| | | GGAAGTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU77 | 5195 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGAGCTCCTCGCAGTTCGGTTTCTGTCTGCACCTGTCACTAGTAACCGAACTGCGA |
| | | GGAGCTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU78 | 5196 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGGGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGAGGCTTTGGCTCGGGACTTTCTGTCTGCACCTGTCACTAGTAAGTCCCGAGCCA |
| | | AAGCCTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU31 | 5149 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGACCTCGCAGTTCGGTTAATTCTGTCTGCACCTGTCACTAGTATTAACCGAACTG |
| | | CGAGGTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU32 | 5150 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGGGGGGGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGAGCTTTGGCTCGGGACTTCTCTGTCTGCACCTGTCACTAGTGAAGTCCCGAGCC |
| | | AAAGCTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU33 | 5151 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGAAGCTGACCTTCCGCGAGATCTGTCTGCACCTGTCACTAGTTCTCGCGGAAGGT |
| | | CAGCTTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU34 | 5152 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGGGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGAAGCAAAGAAGGGGCAGAGGGAGCCCGT |
| | | GAGCTGAGTGGGCCAGGGACTGGGAGAAGGAGTGAGGAGGCAGGGCCGGCATGCCTCTGCTG |
| | | CTGGCCAGAGCCGTCTTCCGCCAAGAGTCTGTCTGCACCTGTCACTAGTCTCTTGGCGGAAG |
| | | ACGGCTTTGGCCGTGTAGTGCTACCCAGCGCTGGCTGCCTCCTCAGCATTGCAATTCCTCTC |
| | | CCATCTGGGCACCAGTCAGCTACCCTGGTGGGAATCTGGGTAGCCCTCGAGGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGCGG |
| | | GGTCTATTGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGCATGCATG |
| | | ACCAGGCTCAGCTAATTTTTGTTTTTTTGGTAGAGACGGGGTTTCACCATATTGGCCAGGCT |
| | | GGTCTCCAACTCCTAATCTCAGGTGATCTACCCACCTTGGCCTCCCAAATTGCTGGGATTAC |
| | | AGGCGTGAACCACTGCTCCCTTCCCTGTCCTTATCGATGCCTAGGGTTAATCATTAACTACA |
| | | AGGAACCCCTAGTGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAGTGAGCGAGCGAGC |
| | | GCGCAG |
| VOYTAU35 | 5153 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGGGGGGGGGGGCGAGGGGGGGGGGGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACCTCGCAGTTCGGTTAATGCCTGTGACCTGGTATTAA |
| | | CCGAACTGCGAGGTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU36 | 5154 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGGGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTTTGGCTCGGGACTTCGCCTGTGACCTGGTGAAGT |
| | | CCCGAGCCAAAGCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU37 | 5155 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAAGCTGACCTTCCGCGAGAGCCTGTGACCTGGTTCTCG |
| | | CGGAAGGTCAGCTTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU38 | 5156 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCCGTCTTCCGCCAAGAGGCCTGTGACCTGGTCTCTT |
| | | GGCGGAAGACGGCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU39 | 5157 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACCTCGCAGTACGGTTAATCCCTGTGACCTGGTATTAA |
| | | CCGAACTGCGAGGTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU40 | 5158 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTTTGGCTGGGGACTTCCCCTGTGACCTGGTGAAGT |
| | | CCCGAGCCAAAGCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU41 | 5159 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGGGGGGGGCCCTCCC |
| | | GCAGAACACCATGCGCTCTTCGGAAAGCTGACCTACCGCGAGACCCTGTGACCTGGTTCTCG |
| | | CGGAAGGTCAGCTTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU42 | 5160 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGGGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCCGTCTTCGGCCAAGAGCCCTGTGACCTGGTCTCTT |
| | | GGCGGAAGACGGCTTCTGTGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU43 | 5161 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACCTCGCAGTTCGGTTAATTCCTGTGATTTGGTATTAA |
| | | CCGAACTGCGAGGTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU44 | 5162 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTTTGGCTCGGGACTTCTCCTGTGATTTGGTGAAGT |
| | | CCCGAGCCAAAGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU45 | 5163 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAAGCTGACCTTCCGCGAGATCCTGTGATTTGGTTCTCG |
| | | CGGAAGGTCAGCTTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU46 | 5164 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCCGTCTTCCGCCAAGAGTCCTGTGATTTGGTCTCTT |
| | | GGCGGAAGACGGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU47 | 5165 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACCTCGCAGTTCGGTTAATACCTGTGACCTGGTATTAA |
| | | CCGAACTGCGAGGTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU48 | 5166 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTTTGGCTCGGGACTTCACCTGTGACCTGGTGAAGT |
| | | CCCGAGCCAAAGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU49 | 5167 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAAGCTGACCTTCCGCGAGAACCTGTGACCTGGTTCTCG |
| | | CGGAAGGTCAGCTTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU50 | 5168 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCCGTCTTCCGCCAAGAGACCTGTGACCTGGTCTCTT |
| | | GGCGGAAGACGGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU51 | 5169 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAACCTCGCAGTTCGGTTAATCCCTGTGACCTGGTATTAA |
| | | CCGAACTGCGAGGTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU52 | 5170 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCTTTGGCTCGGGACTTCCCCTGTGACCTGGTGAAGT |
| | | CCCGAGCCAAAGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU53 | 5171 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGGGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGGGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAAGCTGACCTTCCGCGAGACCCTGTGACCTGGTTCTCG |
| | | CGGAAGGTCAGCTTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAG |
| VOYTAU54 | 5172 | CTGCGCGCTCGCTCGCTCACTGAGGCCGCCCGGGCAAAGCCCGGGCGTCGGGCGACCTTTGG |
| | | TCGCCCGGCCTCAGTGAGCGAGCGAGCGCGCAGAGAGGGAGTGTAGCCATGCTCTAGGAAGA |
| | | TCAATTCAATTCACGCGTCGACATTGATTATTGACTAGTTATTAATAGTAATCAATTACGGG |
| | | GTCATTAGTTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTT |
| | | GGCAGTACATCAAGTGTATCATATGCCAAGTACGCCCCCTATTGACGTCAATGACGGTAAAT |
| | | GGCCCGCCTGGCATTATGCCCAGTACATGACCTTATGGGACTTTCCTACTTGGCAGTACATC |
| | | TACGTATTAGTCATCGCTATTACCATGTCGAGGCCACGTTCTGCTTCACTCTCCCCATCTCC |
| | | CCCCCCTCCCCACCCCCAATTTTGTATTTATTTATTTTTTAATTATTTTGTGCAGCGATGGG |
| | | GGCGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGCAAGCTTCGTT |
| | | TAGTGAACCGTCAGATCGCCTGGAGACGCCATCCACGCTGTTTTGACCTCCATAGAAGACAC |
| | | CGGGACCGATCCAGCCTCCGCGGATTCGAATCCCGGCCGGGAACGGTGCATTGGAACGCGGA |
| | | TTCCCCGTGCCAAGAGTGACGTAAGTACCGCCTATAGAGTCTATAGGCCCACAAAAAATGCT |
| | | TTCTTCTTTTAATATACTTTTTTGTTTATCTTATTTCTAATACTTTCCCTAATCTCTTTCTT |
| | | TCAGGGCAATAATGATACAATGTATCATGCCTCTTTGCACCATTCTAAAGAATAACAGTGAT |
| | | AATTTCTGGGTTAAGGCAATAGCAATATTTCTGCATATAAATATTTCTGCATATAAATTGTA |
| | | ACTGATGTAAGAGGTTTCATATTGCTAATAGCAGCTACAATCCAGCTACCATTCTGCTTTTA |
| | | TTTTATGGTTGGGATAAGGCTGGATTATTCTGAGTCCAAGCTAGGCCCTTTTGCTAATCATG |
| | | TTCATACCTCTTATCTTCCTCCCACAGCTCCTGGGCAACGTGCTGGTCTGTGTGCTGGCCCA |
| | | TCACTTTGGCAAAGAATTGGGATTCGAACCGGTGTGCTGGGCGGGGGGCGGCGGGCCCTCCC |
| | | GCAGAACACCATGCGCTCTTCGGAAGCCGTCTTCCGCCAAGAGCCCTGTGACCTGGTCTCTT |
| | | GGCGGAAGACGGCTTCTGAGGAGCGCCTTGACAGCAGCCATGGGAGGGCCGCCCCCTACCTC |
| | | AGTGACTCGAGGGGTGGCATCCCTGTGACCCCTCCCCAGTGCCTCTCCTGGCCCTGGAAGTT |
| | | GCCACTCCAGTGCCCACCAGCCTTGTCCTAATAAAATTAAGTTGCATCATTTTGTCTGACTA |
| | | GGTGTCCTTCTATAATATTATGGGGTGGAGGGGGGTGGTATGGAGCAAGGGGCAAGTTGGGA |
| | | AGACAACCTGTAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGCCTCCCGAGTT |
| | | GTTGGGATTCCAGGCATGCATGACCAGGCTCAGCTAATTTTTGTTTTTTTGGTAGAGACGGG |
| | | GTTTCACCATATTGGCCAGGCTGGTCTCCAACTCCTAATCTCAGGTGATCTACCCACCTTGG |
| | | CCTCCCAAATTGCTGGGATTACAGGCGTGAACCACTGCTCCCTTCCCTGTCCTTATCGATGC |
| | | CTAGGGTTAATCATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCGGGCTTTGCCCGGG |
| | | CGGCCTCAGTGAGCAGCGAGCGCGCAG |

TABLE 10C
Exemplary Sequences of AAV Viral Genomes Components

| | SEQ | |
|---|---|---|
| | ID | |
| Name | NO | Sequence |
| ITR | 5197 | CTGCGCGCTCGCTCGCTCACTGAGGCCGCCCGGGCAAAGCCCGGGCGTCGGGCGACCTTTG |
| | | GTCGCCCGGCCTCAGTGAGCGAGCGAGCGCGCAGAGAGGGAGTG |
| ITR | 5200 | AGGAACCCCTAGTGATGGAGTTGGCCACTCCCTCTCTGCGCGCTCGCTCGCTCACTGAGGC |
| | | CGGGCGACCAAAGGTCGCCCGACGCCCGGGCTTTGCCCGGGCGGCCTCAGTGAGCGAGCGA |
| | | GCGCGCAG |
| ITR | 4469 | CCTGCAGGCAGCTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAGGGAGTGGCCAAC |
| | | TCCATCACTAGGGGTTCCT |
| ITR | 4470 | AGGAACCCCTAGTGATGGAGTTGGCCACTCCCTCTCTGCGCGCTCGCTCGCTCACTGAGGC |
| | | CGGGCGACCAAAGGTCGCCCGACGCCCGGGCTTTGCCCGGGCGGCCTCAGTGAGCGAGCGA |
| | | GCGCGCAGCTGCCTGCAGG |
| CMVie (v1) | 4472 | CGCGTCGACATTGATTATTGACTAGTTATTAATAGTAATCAATTACGGGGTCATTAGTTCA |
| | | TAGCCCATATATGGAGTTCCGCGTTACATAACTTACGGTAAATGGCCCGCCTGGCTGACCG |
| | | CCCAACGACCCCCGCCCATTGACGTCAATAATGACGTATGTTCCCATAGTAACGCCAATAG |
| | | GGACTTTCCATTGACGTCAATGGGTGGAGTATTTACGGTAAACTGCCCACTTGGCAGTACA |
| | | TCAAGTGTATCATATGCCAAGTACGCCCCCTATTGACGTCAATGACGGTAAATGGCCCGCC |
| | | TGGCATTATGCCCAGTACATGACCTTATGGGACTTTCCTACTTGGCAGTACATCTACGTAT |
| | | TAGTCATCGCTATTAC |
| CMVie (v2) | 4471 | GACATTGATTATTGACTAGTTATTAATAGTAATCAATTACGGGGTCATTAGTTCATAGCCC |
| | | ATATATGGAGTTCCGCGTTACATAACTTACGGTAAATGGCCCGCCTGGCTGACCGCCCAAC |
| | | GACCCCCGCCCATTGACGTCAATAATGACGTATGTTCCCATAGTAACGCCAATAGGGACTT |
| | | TCCATTGACGTCAATGGGTGGAGTATTTACGGTAAACTGCCCACTTGGCAGTACATCAAGT |
| | | GTATCATATGCCAAGTACGCCCCCTATTGACGTCAATGACGGTAAATGGCCCGCCTGGCAT |
| | | TATGCCCAGTACATGACCTTATGGGACTTTCCTACTTGGCAGTACATCTACGTATTAGTCA |
| | | TCGCTATTACCATG |
| CMVie | 4473 | CGACATTGATTATTGACTAGTTATTAATAGTAATCAATTACGGGGTCATTAGTTCATAGCC |
| Enhancer- | | CATATATGGAGTTCCGCGTTACATAACTTACGGTAAATGGCCCGCCTGGCTGACCGCCCAA |
| CBA | | CGACCCCCGCCCATTGACGTCAATAATGACGTATGTTCCCATAGTAACGCCAATAGGGACT |
| Promoter (v1) | | TTCCATTGACGTCAATGGGTGGAGTATTTACGGTAAACTGCCCACTTGGCAGTACATCAAG |
| | | TGTATCATATGCCAAGTACGCCCCCTATTGACGTCAATGACGGTAAATGGCCCGCCTGGCA |
| | | TTATGCCCAGTACATGACCTTATGGGACTTTCCTACTTGGCAGTACATCTACGTATTAGTC |
| | | ATCGCTATTACCATGTCGAGGCCACGTTCTGCTTCACTCTCCCCATCTCCCCCCCCTCCCC |
| | | ACCCCCAATTTTGTATTTATTTATTTTTTAATTATTTTGTGCAGCGATGGGGGGGGGGGGG |
| | | GGGGGCGCGCGCCAGGCGGGGCGGGGCGGGGCGAGGGGCGGGGCGGGGCGAGGCGGAGAGG |
| | | TGCGGCGGCAGCCAATCAGAGCGGCGCGCTCCGAAAGTTTCCTTTTATGGCGAGGCGGCGG |
| | | CGGCGGCGGCCCTATAAAAAGCGAAGCGCGCGGCGGGCGGGAGC |
| CBA | 5199 | CCACGTTCTGCTTCACTCTCCCCATCTCCCCCCCCTCCCCACCCCCAATTTTGTATTTATT |
| Promoter | | TATTTTTTAATTATTTTGTGCAGCGATGGGGGCGGGGGGGGGGGGCGCGCGCCAGGCGGGG |
| | | CGGGGCGGGGCGAGGGGCGGGGCGGGGCGAGGCGGAGAGGTGCGGCGGCAGCCAATCAGAG |
| | | CGGCGCGCTCCGAAAGTTTCCTTTTATGGCGAGGGGGGGGCGGCGGCGGCCCTATAAAAAG |
| | | CGAAGCGCGCGGCGGGCGGGAGC |
| CMVie | 4474 | CGCGTCGACATTGATTATTGACTAGTTATTAATAGTAATCAATTACGGGGTCATTAGTTCA |
| Enhancer- | | TAGCCCATATATGGAGTTCCGCGTTACATAACTTACGGTAAATGGCCCGCCTGGCTGACCG |
| CBA | | CCCAACGACCCCCGCCCATTGACGTCAATAATGACGTATGTTCCCATAGTAACGCCAATAG |
| Promoter (v2) | | GGACTTTCCATTGACGTCAATGGGTGGAGTATTTACGGTAAACTGCCCACTTGGCAGTACA |
| | | TCAAGTGTATCATATGCCAAGTACGCCCCCTATTGACGTCAATGACGGTAAATGGCCCGCC |
| | | TGGCATTATGCCCAGTACATGACCTTATGGGACTTTCCTACTTGGCAGTACATCTACGTAT |
| | | TAGTCATCGCTATTACCATGTCGAGGCCACGTTCTGCTTCACTCTCCCCATCTCCCCCCCC |
| | | TCCCCACCCCCAATTTTGTATTTATTTATTTTTTAATTATTTTGTGCAGCGATGGGGGGGG |
| | | GGGGGGGGGGGGCGCGCCAGGCGGGGCGGGGGGGGGCGAGGGGGGGGGGGGGGCGAGGCGG |
| | | AGAGGTGCGGCGGCAGCCAATCAGAGCGGCGCGCTCCGAAAGTTTCCTTTTATGGCGAGGC |
| | | GGCGGCGGCGGCGGCCCTATAAAAAGCGAAGCGCGCGGCGGGGGGAGC |
| hBG Intron | 4475 | TCAGATCGCCTGGAGACGCCATCCACGCTGTTTTGACCTCCATAGAAGACACCGGGACCGA |
| | | TCCAGCCTCCGCGGATTCGAATCCCGGCCGGGAACGGTGCATTGGAACGCGGATTCCCCGT |
| | | GCCAAGAGTGACGTAAGTACCGCCTATAGAGTCTATAGGCCCACAAAAAATGCTTTCTTCT |
| | | TTTAATATACTTTTTTGTTTATCTTATTTCTAATACTTTCCCTAATCTCTTTCTTTCAGGG |
| | | CAATAATGATACAATGTATCATGCCTCTTTGCACCATTCTAAAGAATAACAGTGATAATTT |
| | | CTGGGTTAAGGCAATAGCAATATTTCTGCATATAAATATTTCTGCATATAAATTGTAACTG |
| | | ATGTAAGAGGTTTCATATTGCTAATAGCAGCTACAATCCAGCTACCATTCTGCTTTTATTT |
| | | TATGGTTGGGATAAGGCTGGATTATTCTGAGTCCAAGCTAGGCCCTTTTGCTAATCATGTT |
| | | CATACCTCTTATCTTCCTCCCACAGCTCCTGGGCAACGTGCTGGTCTGTGTGCTGGCCCAT |
| | | CACTTTGGCAAAGAATT |
| hGH PolyA | 4476 | GGGTGGCATCCCTGTGACCCCTCCCCAGTGCCTCTCCTGGCCCTGGAAGTTGCCACTCCAG |
| | | TGCCCACCAGCCTTGTCCTAATAAAATTAAGTTGCATCATTTTGTCTGACTAGGTGTCCTT |
| | | CTATAATATTATGGGGTGGAGGGGGGTGGTATGGAGCAAGGGGCAAGTTGGGAAGACAACC |
| | | TGTAGGGCCTGCGGGGTCTATTGGGAACCAAGCTGGAGTGCAGTGGCACAATCTTGGCTCA |
| | | CTGCAATCTCCGCCTCCTGGGTTCAAGCGATTCTCCTGCCTCAGCCTCCCGAGTTGTTGGG |
| | | ATTCCAGGCATGCATGACCAGGCTCAGCTAATTTTTGTTTTTTTGGTAGAGACGGGGTTTC |
| | | ACCATATTGGCCAGGCTGGTCTCCAACTCCTAATCTCAGGTGATCTACCCACCTTGGCCTC |
| | | CCAAATTGCTGGGATTACAGGCGTGAACCACTGCTCCCTTCCCTGTCCTT |

In some embodiments, the a viral genome described herein comprises the nucleotide sequence of any one of SEQ ID NOs: 4439-4468 or 5149-5196, or a nucleotide sequence at least 70%, 75%, 80%, 85%, 90%, 95%, 99% or 100% identical to any one of SEQ ID NOs: 4439-4468 or 5149-5196. In some embodiments, the viral genome comprises the nucleotide sequence of any one of SEQ ID NOs: 4439-4468 or 5149-5196, or a nucleotide sequence at least 80% identical to any one of SEQ ID NOs: 4439-4468 or 5149-5196. In some embodiments, the viral genome comprises the nucleotide sequence of any one of SEQ ID NOs: 4439-4468 or 5149-5196, or a nucleotide sequence at least 85% identical to any one of SEQ ID NOs: 4439-4468 or 5149-5196. In some embodiments, the viral genome comprises the nucleotide sequence of any one of SEQ ID NOs: 4439-4468 or 5149-5196, or a nucleotide sequence at least 90% identical to any one of SEQ ID NOs: 4439-4468 or 5149-5196. In some embodiments, the viral genome comprises the nucleotide sequence of any one of SEQ ID NOs: 4439-4468 or 5149-5196, or a nucleotide sequence at least 95% identical to any one of SEQ ID NOs: 4439-4468 or 5149-5196. In some embodiments, the viral genome comprises the nucleotide sequence of any one of SEQ ID NOs: 4439-4468 or 5149-5196, or a nucleotide sequence at least 99% identical to any one of SEQ ID NOs: 4439-4468 or 5149-5196.

In some embodiments, the AAV viral genome comprises the nucleotide sequence of SEQ ID NO: 5173, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5173, or a nucleotide sequence having at least one, two or three, but not more than 30, 20 or 10 different nucleotides relative to the nucleotide sequence of SEQ ID NO: 5173. In some embodiments, the AAV viral genome comprises the nucleotide sequence of SEQ ID NO: 5173.

In some embodiments, the AAV viral genome comprises the nucleotide sequence of SEQ ID NO: 5195, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5195, or a nucleotide sequence having at least one, two or three, but not more than 30, 20 or 10 different nucleotides relative to the nucleotide sequence of SEQ ID NO: 5195. In some embodiments, the AAV viral genome comprises the nucleotide sequence of SEQ ID NO: 5195.

In some embodiments, the AAV viral genome comprises the nucleotide sequence of SEQ ID NO: 5195, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5196, or a nucleotide sequence having at least one, two or three, but not more than 30, 20 or 10 different nucleotides relative to the nucleotide sequence of SEQ ID NO: 5196. In some embodiments, the AAV viral genome comprises the nucleotide sequence of SEQ ID NO: 5196.

In some embodiments, the AAV viral genome comprises the nucleotide sequence of SEQ ID NO: 5182, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5182, or a nucleotide sequence having at least one, two or three, but not more than 30, 20 or 10 different nucleotides relative to the nucleotide sequence of SEQ ID NO: 5182. In some embodiments, the AAV viral genome comprises the nucleotide sequence of SEQ ID NO: 5182.

In some embodiments, the AAV viral genome comprises the nucleotide sequence of SEQ ID NO: 5183, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5183, or a nucleotide sequence having at least one, two or three, but not more than 30, 20 or 10 different nucleotides relative to the nucleotide sequence of SEQ ID NO: 5183. In some embodiments, the AAV viral genome comprises the nucleotide sequence of SEQ ID NO: 5183.

In some embodiments, the AAV viral genome comprises the nucleotide sequence of SEQ ID NO: 5184, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5184, or a nucleotide sequence having at least one, two or three, but not more than 30, 20 or 10 different nucleotides relative to the nucleotide sequence of SEQ ID NO: 5184. In some embodiments, the AAV viral genome comprises the nucleotide sequence of SEQ ID NO: 5184.

In some embodiments, the AAV viral genome comprises the nucleotide sequence of SEQ ID NO: 5185, or a nucleotide sequence at least 70%, 75%, 80%, 85%, 90%, 92%, 95%, 96%, 97%, 98%, or 99% identical thereto. In some embodiments, the AAV viral genome comprises the nucleotide sequence of SEQ ID NO: 5185, or a nucleotide sequence having at least one, two or three, but not more than 30, 20 or 10 different nucleotides relative to the nucleotide sequence of SEQ ID NO: 5185. In some embodiments, the AAV viral genome comprises the nucleotide sequence of SEQ ID NO: 5185.

In some embodiments, the viral genome may comprise at least one inverted terminal repeat (ITR) region. In some embodiments, the AAV particle viral genome may comprise two inverted terminal repeat (ITR) regions, e.g., a 5′ ITR region (e.g., an ITR region present 5′ relative to the encoded modulatory polynucleotide) and a 3′ ITR region (e.g., an ITR region present 3′ relative to the encoded modulatory polynucleotide). In some embodiments, the ITR region comprises the nucleotide sequence of SEQ ID NO: 4469, 4470, 5197, or 5200; a nucleotide sequence at least 70%, 75%, 80%, 85%, 90%, 95%, or 99% identical to the nucleotide sequence of SEQ ID NO: 4469, 4470, 5197, or 5200; a nucleotide sequence comprising one, two, three, but no more than four different nucleotides relative to SEQ ID NO: 4469, 4470, 5197, or 5200; or a nucleotide sequence comprising one, two, three, but no more than four modifications relative to SEQ ID NO: 4469, 4470, 5197, or 5200. In some embodiments, the 5′ ITR region comprises the nucleotide sequence of SEQ ID NO: 4469; a nucleotide sequence at least 70%, 75%, 80%, 85%, 90%, 95%, or 99% identical to the nucleotide sequence of SEQ ID NO: 4469; a nucleotide sequence comprising one, two, three, but no more than four different nucleotides relative to SEQ ID NO: 4469; or a nucleotide sequence comprising one, two, three, but no more than four modifications relative to SEQ ID NO: 4469. In some embodiments, the 3′ ITR comprises the nucleotide sequence of SEQ ID NO: 4470; a nucleotide sequence at least 70%, 75%, 80%, 85%, 90%, 95%, or 99% identical to the nucleotide sequence of SEQ ID NO: 4470; a nucleotide sequence comprising one, two, three, but no more than four different nucleotides relative to SEQ ID NO: 4470; or a nucleotide sequence comprising one, two, three, but no more than four modifications relative to SEQ ID NO: 4470. In some embodiments, the 5′ ITR region comprises the nucleotide sequence of SEQ ID NO: 5197; a nucleotide sequence at least 70%, 75%, 80%, 85%, 90%, 95%, or 99% identical to the nucleotide sequence of SEQ ID NO: 5197; a nucleotide sequence comprising one, two, three, but no more than four different nucleotides relative to SEQ ID NO: 5197; or a nucleotide sequence comprising one, two, three, but no more than four modifications relative to SEQ ID NO: 5197. In some embodiments, the 3′ ITR comprises the nucleotide sequence of SEQ ID NO: 5200; a nucleotide sequence at least 70%, 75%, 80%, 85%, 90%, 95%, or 99% identical to the nucleotide sequence of SEQ ID NO: 5200; a nucleotide sequence comprising one, two, three, but no more than four different nucleotides relative to SEQ ID NO: 5200; or a nucleotide sequence comprising one, two, three, but no more than four modifications relative to SEQ ID NO: 5200.

In some embodiments, the viral genome comprises an enhancer. In some embodiments, the enhancer region is a cytomegalovirus major immediate-early enhancer region (CMVie) enhancer. In some embodiments, the enhancer comprises the nucleotide sequence of SEQ ID NO: 4471; a nucleotide sequence at least 70%, 75%, 80%, 85%, 90%, 95%, or 99% identical to the nucleotide sequence of SEQ ID NO: 4471; a nucleotide sequence comprising one, two, three, but no more than four different nucleotides relative to SEQ ID NO: 4471; or a nucleotide sequence comprising one, two, three, but no more than four modifications relative to SEQ ID NO: 4471. In some embodiments, the enhancer comprises the nucleotide sequence of SEQ ID NO: 4472; a nucleotide sequence at least 70%, 75%, 80%, 85%, 90%, 95%, or 99% identical to the nucleotide sequence of SEQ ID NO: 4472; a nucleotide sequence comprising one, two, three, but no more than four different nucleotides relative to SEQ ID NO: 4472; or a nucleotide sequence comprising one, two, three, but no more than four modifications relative to SEQ ID NO: 4472.

In some embodiments, the viral genome comprises a promoter. In some embodiments, the promoter is a tissue specific promoter. In some embodiments, the promoter is a ubiquitous promoter. In some embodiments, the promoter comprises the nucleotide sequence of SEQ ID NO: 5199; a nucleotide sequence at least 70%, 75%, 80%, 85%, 90%, 95%, or 99% identical to the nucleotide sequence of SEQ ID NO: 5199; a nucleotide sequence comprising one, two, three, but no more than four different nucleotides relative to SEQ ID NO: 5199; or a nucleotide sequence comprising one, two, three, but no more than four modifications relative to SEQ ID NO: 5199.

In some embodiments, the viral genome comprises a promoter and enhancer, wherein the promoter and enhancer region comprises the nucleotide sequence of SEQ ID NO: 4474 or 4473; a nucleotide sequence at least 70%, 75%, 80%, 85%, 90%, 95%, or 99% identical to the nucleotide sequence of SEQ ID NO: 4474 or 4473; a nucleotide sequence comprising one, two, three, but no more than four different nucleotides relative to SEQ ID NO: 4474 or 4473; or a nucleotide sequence comprising one, two, three, but no more than four modifications relative to SEQ ID NO: 4474 or 4473.

In some embodiments, the viral genome comprises an intron. In some embodiments, the intron comprises the nucleotide sequence of SEQ ID NO: 4475; a nucleotide sequence at least 70%, 75%, 80%, 85%, 90%, 95%, or 99% identical to the nucleotide sequence of SEQ ID NO: 4475; a nucleotide sequence comprising one, two, three, but no more than four different nucleotides relative to SEQ ID NO: 4475; or a nucleotide sequence comprising one, two, three, but no more than four modifications relative to SEQ ID NO: 4475.

In some embodiments, the viral genome comprises a polyadenylation (polyA) signal sequence region. In some embodiments, the polyA signal sequence region comprises the nucleotide sequence of SEQ ID NO: 4476; a nucleotide sequence at least 70%, 75%, 80%, 85%, 90%, 95%, or 99% identical to the nucleotide sequence of SEQ ID NO: 4476; a nucleotide sequence comprising one, two, three, but no more than four different nucleotides relative to SEQ ID NO: 4476; or a nucleotide sequence comprising one, two, three, but no more than four modifications relative to SEQ ID NO: 4476.

In some embodiments, the viral genome comprises at least one ITR region (e.g., two ITR regions), an enhancer, a promoter sequence region, an intron, a modulatory polynucleotide, and a polyadenylation signal sequence region, e.g., as provided in Table 10C.

In some embodiments, the AAV particle viral genome comprises a 5′ ITR region, a 3′ ITR region, a CMV enhancer sequence, a CBA promoter sequence, an hBG intron, a modulatory polynucleotide, and a human growth hormone (hGH) polyA signal sequence region. In some embodiments, the viral genome comprises in 5′ to 3′ order: a 5′ ITR, an enhancer (e.g., a CMVie enhancer or variant thereof), a promoter (e.g., a ubiquitous promoter, e.g., a CBA promoter or variant thereof), an intron (e.g., an hBG intron or variant thereof), a modulatory polynucleotide (e.g., a modulatory polynucleotide encoding an siRNA for targeting MAPT described herein, e.g., a modulatory polynucleotide as provided in Tables 9A and 9B or a variant thereof), a polyA signal sequence region (e.g., a hGH polyA signal sequence region or variant thereof), and a 3′ ITR region. In some embodiments, the viral genome comprises one or more of the sequences provided in any one of Tables 10A, 10B, 11-18, or 26, or a variant thereof, e.g., a sequence at least 70%, 75%, 80%, 85%, 90%, 95%, or 99% identical to any one of the sequences provided in Tables 10A, 10B, 11-18, or 26; a nucleotide sequence comprising one, two, three, but no more than four different nucleotides relative to any one of the sequences provided in Tables 10A, 10B, 11-18, or 26; or a nucleotide sequence comprising one, two, three, but no more than four modifications relative to any one of the sequences provided in Tables 10A, 10B, 11-18, or 26.

TABLE 11
Sequence Regions in the ITR to ITR Sequence

| | VOYTAU1 (SEQ | | VOYTAU2 (SEQ | | VOYTAU3 (SEQ | | VOYTAU4 (SEQ | |
|---|---|---|---|---|---|---|---|---|
| | ID NO: 4439) | | ID NO: 4440) | | ID NO: 4441) | | ID NO: 4442) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie Enhancer | 4472 | 382 | 4472 | 382 | 4472 | 382 | 4472 | 382 |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4380 | 158 | 4381 | 158 | 4382 | 158 | 4383 | 158 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 12
Sequence Regions in the ITR to ITR Sequence

| | VOYTAU5 (SEQ | | VOYTAU6 (SEQ | | VOYTAU7 (SEQ | | VOYTAU8 (SEQ | |
|---|---|---|---|---|---|---|---|---|
| | ID NO: 4443) | | ID NO: 4444) | | ID NO: 4445) | | ID NO: 4446) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie Enhancer | 4472 | 382 | 4472 | 382 | 4472 | 382 | 4472 | 382 |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4384 | 158 | 4385 | 158 | 4386 | 158 | 4387 | 158 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 13
Sequence Regions in the ITR to ITR Sequence

| | VOYTAU9 (SEQ | | VOYTAU10 (SEQ | | VOYTAU11 (SEQ | | VOYTAU12 (SEQ | |
|---|---|---|---|---|---|---|---|---|
| | ID NO: 4447) | | ID NO: 4448) | | ID NO: 4449) | | ID NO: 4450) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie Enhancer | 4472 | 382 | 4472 | 382 | 4472 | 382 | 4472 | 382 |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4388 | 158 | 4389 | 158 | 4390 | 158 | 4391 | 158 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 14
Sequence Regions in the ITR to ITR Sequence

| | VOYTAU13 (SEQ | | VOYTAU14 (SEQ | | VOYTAU15 (SEQ | | VOYTAU16 (SEQ | |
|---|---|---|---|---|---|---|---|---|
| | ID NO: 4451) | | ID NO: 4452) | | ID NO: 4453) | | ID NO: 4454) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie Enhancer | 4472 | 382 | 4472 | 382 | 4472 | 382 | 4472 | 382 |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4392 | 158 | 4393 | 158 | 4394 | 158 | 4395 | 158 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 15
Sequence Regions in the ITR to ITR Sequence

| | VOYTAU17 (SEQ | | VOYTAU18 (SEQ | | VOYTAU19 (SEQ | | VOYTAU20 (SEQ | |
|---|---|---|---|---|---|---|---|---|
| | ID NO: 4455) | | ID NO: 4456) | | ID NO: 4457) | | ID NO: 4458) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie Enhancer | 4472 | 382 | 4472 | 382 | 4472 | 382 | 4472 | 382 |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4396 | 158 | 4397 | 158 | 4398 | 158 | 4399 | 158 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 16
Sequence Regions in the ITR to ITR Sequence

| | VOYTAU21 (SEQ | | VOYTAU22 (SEQ | | VOYTAU23 (SEQ | | VOYTAU24 (SEQ | |
|---|---|---|---|---|---|---|---|---|
| | ID NO: 4459) | | ID NO: 4460) | | ID NO: 4461) | | ID NO: 4462) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie Enhancer | 4472 | 382 | 4472 | 382 | 4472 | 382 | 4472 | 382 |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4400 | 158 | 4401 | 158 | 4402 | 158 | 4403 | 158 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 17
Sequence Regions in the ITR to ITR Sequence

| | VOYTAU25 (SEQ | | VOYTAU26 (SEQ | | VOYTAU27 (SEQ | | VOYTAU28 (SEQ | |
|---|---|---|---|---|---|---|---|---|
| | ID NO: 4463) | | ID NO: 4464) | | ID NO: 4465) | | ID NO: 4466) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 4469 | 141 | 4469 | 141 | 4469 | 141 | 4469 | 141 |
| CMVie | 4472 | 382 | 4472 | 382 | 4472 | 382 | 4472 | 382 |
| Enhancer | | | | | | | | |
| CBA Promoter | 5199 | 267 | 5199 | 267 | 5199 | 267 | 5199 | 267 |
| hBG Intron | 4475 | 566 | 4475 | 566 | 4475 | 566 | 4475 | 566 |
| Modulatory | 4404 | 158 | 4405 | 260 | 4406 | 260 | 4407 | 260 |
| Polynucleotide | | | | | | | | |
| hGH PolyA | 4476 | 477 | 4476 | 477 | 4476 | 477 | 4476 | 477 |
| 3′ ITR | 4470 | 141 | 4470 | 141 | 4470 | 141 | 4470 | 141 |

TABLE 18
Sequence Regions in the ITR to ITR Sequence

| | VOYTAU29 (SEQ | | VOYTAU30 (SEQ | | |
|---|---|---|---|---|---|
| | ID NO: 4467) | | ID NO: 4468) | | VOYTAU77 (SEQ |
| | Region | | Region | | ID NO: 5195) |
| | SEQ | Region | SEQ | Region | Region |
| Sequence Regions | ID NO | length | ID NO | length | SEQ ID NO |
| 5′ ITR | 4469 | 141 | 4469 | 141 | 5197 |
| CMVie Enhancer | 4472 | 382 | 4472 | 382 | 4471 |
| CBA Promoter | 5199 | 267 | 5199 | 267 | 5199 |
| hBG Intron | 4475 | 566 | 4475 | 566 | 4475 |
| Modulatory | 4408 | 260 | 4409 | 260 | 4408 |
| Polynucleotide | | | | | |
| hGH Poly A | 4476 | 477 | 4476 | 477 | 4476 |
| 3′ ITR | 4470 | 141 | 4470 | 141 | 5200 |

TABLE 26
Sequence Regions in the ITR to ITR Sequences

| | VOYTAU55 | VOYTAU78 | VOYTAU64 | VOYTAU65 | VOYTAU66 | VOYTAU67 |
|---|---|---|---|---|---|---|
| | (SEQ ID NO: | (SEQ ID NO: | (SEQ ID NO: | (SEQ ID NO: | (SEQ ID NO: | (SEQ ID NO: |
| | 5173) | 5196) | 5182) | 5183) | 5184) | 5185) |
| Sequence | Region SEQ | Region SEQ | Region SEQ | Region SEQ | Region SEQ | Region SEQ |
| Regions | ID NO: | ID NO: | ID NO: | ID NO: | ID NO: | ID NO: |
| 5′ ITR | 5197 | 5197 | 5197 | 5197 | 5197 | 5197 |
| CMVie | 4471 | 4471 | 4471 | 4471 | 4471 | 4471 |
| Enhancer | | | | | | |
| CBA | 5199 | 5199 | 5199 | 5199 | 5199 | 5199 |
| Promoter | | | | | | |
| hBG Intron | 4475 | 4475 | 4475 | 4475 | 4475 | 4475 |
| Modulatory | 4405 | 4409 | 4390 | 4391 | 4393 | 4394 |
| Polynucleotide | | | | | | |
| hGH PolyA | 4476 | 4476 | 4476 | 4476 | 4476 | 4476 |
| 3′ ITR | 5200 | 5200 | 5200 | 5200 | 5200 | 5200 |

In some embodiments, the present disclosure provides AAV particles comprising a viral genome described herein, and an AAV capsid protein, e.g., an AAV capsid variant described herein. In some embodiments, the present disclosure provides a vector comprising a viral genome described herein.

In other embodiments, an AAV particle comprising a nucleic acid sequence encoding a siRNA molecule is to deliver siRNA molecules to the central nervous system, e.g., as described in U.S. Pat. No. 6,180,613; the contents of which is herein incorporated by reference in its entirety.

### Viral Production

Cells for the production of AAV, e.g., rAAV, particles may comprise, in some embodiments, mammalian cells (such as HEK293 cells) and/or insect cells (such as Sf9 cells).

In some embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome described herein. In some embodiments, disclosed herein is a vector comprising a nucleotide sequence encoding a modulatory polynucleotide, e.g., a modulatory polynucleotide described herein. In some embodiments, the present disclosure provides a vector comprising a nucleotide sequence encoding an siRNA, e.g., an siRNA for targeting a MAPT gene, mRNA, and/or protein as described herein.

In some embodiments, disclosed herein is a cell comprising a viral genome, modulatory polynucleotide, siRNA, and/or AAV particle of the present disclosure. In some embodiments, the cell is a bacterial cell (e.g. an E. coli cell), a mammalian cell (e.g., a HEK293 cell or a CNS cell), or an insect cell (e.g., an Sf9 cell or an Sf21 cell).

In some embodiments, disclosed herein is a method of making a recombinant AAV particle of the present disclosure, the method comprising (i) providing a host cell comprising a viral genome described herein and incubating the host cell under conditions suitable to enclose the viral genome in an AAV capsid polypeptide, e.g., an AAV capsid variant, as described herein (e.g., an AAV capsid variant as provided in Table 1 or functional variant thereof), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In some embodiments, disclosed herein is a method of making a viral genome. The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral genome from the backbone region, e.g., by cleaving the nucleic acid molecule at upstream and downstream of the viral genome. In some embodiments, the viral genome comprising a promoter operably linked to nucleic acid comprising a transgene encoding a polynucleotide (e.g., a MAPT targeting siRNA described herein), will be incorporated into an AAV particle produced in the cell. In some embodiments, the cell is a bacterial cell, a mammalian cell (e.g., a HEK293 cell), or an insect cell (e.g., an Sf9 cell).

In some embodiments, a vector used for AAV production comprises a nucleotide sequence encoding an AAV capsid protein, e.g., one, two, or all of a VP1 capsid protein, a VP2 capsid protein, or a VP3 capsid protein. In some embodiments, the nucleotide sequence encoding the capsid protein, e.g., the VP1 capsid protein, comprises a canonical start codon (e.g., an ATG). In some embodiments, the nucleotide sequence encoding the AAV capsid protein, e.g., an AAV VP1 capsid protein, is a non-ATG start codon, e.g., a non-canonical start codon (e.g., ACG, TTG, or GTG), e.g., a start codon capable of altering the ratio of the viral capsid proteins in the production system.

In some embodiments, the vector(s) used for AAV production comprises a nucleotide sequence encoding an AAV rep protein, e.g., a Rep78 protein, a Rep52 protein, a Rep40 protein, and/or a Rep68 protein (e.g., a Rep78 and Rep52 protein). In some embodiments, the nucleotide sequence encoding the Rep protein comprises an ATG start codon. In some embodiments, the nucleotide sequence comprises a non-ATG start codon, e.g., a non-canonical stop codon, e.g., ACG, TTG, CTG or GTG.

In some embodiments, an AAV particle described herein is generated using a method described in WO 2022/187473, WO 2022/187548, WO 2021/041485, WO 2020/150556, WO 2020/081490, WO 2020/072844, WO 2020/023612, WO 2021/030125, WO 2020/223274, WO 2019/241486, WO2022032153, the contents of each of which are hereby incorporated by reference it their entirety.

### Cells

In some embodiments, the present disclosure provides a cell comprising an AAV particle, a viral genome, a modulatory polynucleotide, and/or an siRNA targeting a MAPT gene, mRNA, and/or protein described herein.

Viral production disclosed herein describes processes and methods for producing an AAV particle, which comprise contacting a target cell to deliver a payload, e.g. a siRNA for targeting MAPT or an modulatory polynucleotide comprising a siRNA for targeting MAPT.

In some embodiments, the AAV particle of the present disclosure may be produced in insect cells (e.g., Sf9 cells). In some embodiments, the AAV particle of the present disclosure may be produced using triple transfection. In some embodiments, the AAV particle of the present disclosure may be produced in mammalian cells. In some embodiments, the AAV particle of the present disclosure may be produced by triple transfection in mammalian cells. In some embodiments, the AAV particle of the present disclosure may be produced by triple transfection in HEK293 cells.

In some embodiments, the AAV particles may be produced in a viral replication cell that comprises an insect cell. Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which is herein incorporated by reference in its entirety.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

### Small Scale Production of AAV Particles

In some embodiments, viral production comprises processes and methods for producing an AAV particle, e.g., an AAV particle for contacting a target cell to deliver a payload, e.g. a siRNA for targeting MAPT or an modulatory polynucleotide comprising a siRNA for targeting MAPT.

In some embodiments, the AAV particles may be produced in a viral replication cell that comprises a mammalian cell. In some embodiments, the viral replication cell is a HEK293 cell, a COS cell, a HeLa cell, a KB cell, or a mammalian cell line as described in U.S. Pat. Nos. 6,156,303, 5,387,484, 5,741,683, 5,691,176, and 5,688,676; U.S. patent application 2002/0081721, and International Patent Applications WO00/47757, WO 00/24916, and WO 96/17947, the contents of each of which are herein incorporated by reference in their entireties.

In some embodiments, an AAV particle is produced in a mammalian cell wherein all three VP proteins are expressed at a stoichiometry of about 1:1:10 (VP1:VP2:VP3). In some embodiments, the regulatory mechanisms that allow this controlled level of expression include the production of two mRNAs, one for VP1, and the other for VP2 and VP3, produced by differential splicing.

In some embodiments, an AAV particle is produced in a mammalian cells using a triple transfection method. In some embodiments, the method comprises providing a payload construct, parvoviral Rep and parvoviral Cap and a helper construct are comprised.

### Baculovirus Production

In certain embodiments, processes of the present disclosure can include production of AAV particles or viral vectors in a baculoviral system, e.g., an AAV viral production system. In certain embodiments, the AAV production system comprises an AAV expression construct. In some embodiments, the AAV expression construct comprises a baculovirus genome, e.g., a variant baculovirus genome, e.g., a variant baculovirus genome comprising a modification in at least one (e.g., at least two, three, four or more) non-essential genes. In some, embodiments, the AAV expression construct comprises at least one or two Rep-coding region and one, two, or three VP-coding regions. In some embodiments, the AAV production system further comprises an AAV payload construct. In some embodiments, the AAV payload construct encodes a payload described herein e.g., a modulatory polynucleotide comprising a siRNA for targeting MAPT or an siRNA for targeting MAPT. In some embodiments, the AAV expression construct, AAV payload construct, and/or AAV production system is as described and provided in WO 2022/187473, WO 2022/187548, WO 2021/041485, WO 2020/150556, WO 2020/081490, WO 2020/072844, WO 2020/023612, WO 2021/030125, WO 2020/223274, WO 2019/241486, WO2022032153, the contents of each of which are hereby incorporated by reference it their entirety.

In certain embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

In some embodiments, the AAV expression constructs are capable of producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety as related to the production and use of BEVs and viral particles.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

### Large-Scale Production

In some embodiments, AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CellSTACK®, CellCube® (Corning Corp., Corning, NY) and Nunc™ Cell Factory™ (Thermo Scientific, Waltham, MA.) In some cases, large-scale adherent cell surfaces may comprise from about 1,000 cm² to about 100,000 cm². In some cases, large-scale adherent cell cultures may comprise from about 107 to about 109 cells, from about 108 to about 1010 cells, from about 109 to about 1012 cells or at least 1012 cells. In some cases, large-scale adherent cultures may produce from about 109 to about 1012, from about 1010 to about 1013, from about 1011 to about 1014, from about 1012 to about 1015 or at least 1015 viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm3 volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds [e.g. polyethyleneimine (PEI)] or the use of non-chemical methods (e.g. electroporation.) With cells grown in suspension, transfection methods may include, but are not limited to the use of calcium phosphate and the use of PEI. In some cases, transfection of large-scale suspension cultures may be carried out according to the section entitled “Transfection Procedure” described in Feng, L. et al., 2008. Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids from one or more AAV payload constructs. Such methods may enhance the production of viral particles by reducing cellular resources wasted on expressing payload constructs. In some cases, such methods may be carried according to those taught in US Publication No. US2014/0099666, the contents of which are herein incorporated by reference in their entirety.

AAV particles of the present disclosure may be prepared by any method known in the art, such as, but not limited to, mammalian cell production methods or insect cell production methods.

### II. FORMULATION AND DELIVERY

### Pharmaceutical Compositions and Formulation

The AAV particles of the present disclosure may be used to prepare a pharmaceutical composition.

It will be understood by the skilled artisan that the pharmaceutical compositions of the present disclosure are suitable for administration to humans, but that such compositions are also generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates.

In some embodiments, compositions are administered to humans, human patients or subjects. For the purposes of the present disclosure, the phrase “active ingredient” generally refers either to the synthetic siRNA duplexes, the vector, e.g., AAV vector, encoding the siRNA duplexes, or to the siRNA molecule delivered by a vector as described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

The vector genomes comprising the nucleic acid sequence encoding the siRNA molecules of the present disclosure can be formulated using one or more excipients to: (1) increase stability; (2) increase cell transfection or transduction; (3) permit the sustained or delayed release; or (4) alter the biodistribution (e.g., target the AAV particles and/or vector genome to specific tissues or cell types such as brain and neurons).

Formulations of the present disclosure can include, without limitation, saline, lipidoids, liposomes, lipid nanoparticles, polymers, lipoplexes, core-shell nanoparticles, peptides, proteins, cells transfected with viral vectors such as, but not limited to, AAV particles (e.g., for transplantation into a subject), nanoparticle mimics and combinations thereof. Further, the viral vectors of the present disclosure (e.g., AAV particles) may be formulated using self-assembled nucleic acid nanoparticles.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by the United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of vectors comprising the nucleic acid sequence for the siRNA molecules of the present disclosure may include cations or anions. In some embodiments, the formulations include metal cations such as, but not limited to, Zn2+, Ca2+, Cu2+, Mg+ and combinations thereof.

As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in its entirety.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

According to the present disclosure, the vector, e.g., AAV vector, comprising the nucleic acid sequence for the siRNA molecules of the present disclosure may be formulated for CNS delivery. Agents that cross the brain blood barrier (BBB) may be used. For example, some cell-penetrating peptides that can target the BBB endothelium may be used.

### Inactive Ingredients

In some embodiments, formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of AAV particles and viral vectors carrying compositions described herein may include cations or anions. In some embodiments, the formulations include metal cations such as, but not limited to, Zn2+, Ca2+, Cu2+, Mg+ and combinations thereof. As a non-limiting example, formulations may include polymers and compositions described herein complexed with a metal cation (See e.g., U.S. Pat. Nos. 6,265,389 and 6,555,525, each of which is herein incorporated by reference in its entirety).

### Delivery

In some embodiments, the AAV particles and viral vector comprising compositions described herein may be administered or delivered using the methods for the delivery of AAV virions described in European Patent Application No. EP1857552, the contents of which are herein incorporated by reference in their entirety.

In some embodiments, the AAV particles and viral vector comprising compositions described herein may be administered or delivered using the methods for delivering proteins using AAV vectors described in European Patent Application No. EP2678433, the contents of which are herein incorporated by reference in their entirety.

In some embodiments, the AAV particles and viral vector comprising compositions described herein may be administered or delivered using the methods for delivering DNA molecules using AAV vectors described in U.S. Pat. No. 5,858,351, the contents of which are herein incorporated by reference in their entirety.

In some embodiments, the AAV particles and viral vector comprising compositions described herein may be administered or delivered using the methods for delivering DNA to the bloodstream described in U.S. Pat. No. 6,211,163, the contents of which are herein incorporated by reference in their entirety.

In some embodiments, the AAV particles and viral vector comprising compositions described herein may be administered or delivered using the methods for delivering AAV virions described in U.S. Pat. No. 6,325,998, the contents of which are herein incorporated by reference in their entirety.

In some embodiments, the AAV particles and viral vector comprising compositions described herein may be administered or delivered using the methods for delivering a payload to the central nervous system described in U.S. Pat. No. 7,588,757, the contents of which are herein incorporated by reference in their entirety.

In some embodiments, the AAV particles and viral vector comprising compositions described herein may be administered or delivered using the methods for delivering a payload described in U.S. Pat. No. 8,283,151, the contents of which are herein incorporated by reference in their entirety.

In some embodiments, the AAV particles and viral vector comprising compositions described herein may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particles and viral vector comprising compositions described herein may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described AAV polynucleotides or AAV genomes, comprising contacting the cell or tissue with said AAV polynucleotide or AAV genomes or contacting the cell or tissue with a particle comprising said AAV polynucleotide or AAV genome, or contacting the cell or tissue with any of the described compositions, including pharmaceutical compositions. The method of delivering the AAV polynucleotide or AAV genome to a cell or tissue can be accomplished in vitro, ex vivo, or in vivo.

Introduction into Cells—AAV Vectors

The siRNA molecules (e.g., siRNA duplexes) of the present disclosure may be introduced into cells using any of a variety of approaches such as, but not limited to, viral vectors (e.g., AAV vectors or AAV particles). These viral vectors are engineered and optimized to facilitate the entry of siRNA molecule into cells that are not readily amendable to transfection. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In some embodiments, the siRNA molecules of the present disclosure are introduced into a cell by contacting the cell with a composition comprising a lipophilic carrier and a vector, e.g., an AAV vector, comprising a nucleic acid sequence encoding the siRNA molecules of the present disclosure. In other embodiments, the siRNA molecule is introduced into a cell by transfecting or infecting the cell with a vector, e.g., an AAV vector, comprising nucleic acid sequences capable of producing the siRNA molecule when transcribed in the cell. In some embodiments, the siRNA molecule is introduced into a cell by injecting into the cell a vector, e.g., an AAV vector, comprising a nucleic acid sequence capable of producing the siRNA molecule when transcribed in the cell.

In some embodiments, prior to transfection, a vector, e.g., an AAV vector, comprising a nucleic acid sequence encoding the siRNA molecules of the present disclosure may be transfected into cells.

In other embodiments, the vectors, e.g., AAV vectors, comprising the nucleic acid sequence encoding the siRNA molecules of the present disclosure may be delivered into cells by electroporation (e.g. U.S. Patent Publication No. 20050014264; the content of which is herein incorporated by reference in its entirety).

Other methods for introducing vectors, e.g., AAV vectors, comprising the nucleic acid sequence for the siRNA molecules described herein may include photochemical internalization as described in U.S. Patent publication No. 20120264807; the content of which is herein incorporated by reference in its entirety.

In some embodiments, the formulations described herein may contain at least one vector, e.g., AAV vectors, comprising the nucleic acid sequence encoding the siRNA molecules described herein. In some embodiments, the siRNA molecules may target the MAPT transcript at one target site. In another embodiment, the formulation comprises a plurality of vectors, e.g., AAV vectors, each vector comprising a nucleic acid sequence encoding a siRNA molecule targeting the MAPT transcript at a different target site. The MAPT transcript may be targeted at 2, 3, 4, 5 or more than 5 sites.

In some embodiments, the vectors, e.g., AAV vectors, from any relevant species, such as, but not limited to, human, dog, mouse, rat or monkey may be introduced into cells.

In some embodiments, the vectors, e.g., AAV vectors, may be introduced into cells which are relevant to the disease to be treated. As a non-limiting example, the disease is Alzheimer's disease (AD), frontotemporal dementia (FTD), and/or Dravet syndrome (DS), and the target cells are neurons, e.g., interneurons, and astrocytes.

In some embodiments, the vectors, e.g., AAV vectors, may be introduced into cells which have a high level of endogenous expression of the target sequence.

In another embodiment, the vectors, e.g., AAV vectors, may be introduced into cells which have a low level of endogenous expression of the target sequence.

In some embodiments, the cells may be those which have a high efficiency of AAV transduction.

### Delivery to Subjects

The present disclosure additionally provides a method of delivering to a subject, including a mammalian subject, any of the above-described AAV particles which comprises a vector genome encoding the sense, antisense and/or siRNA duplex described herein. As a non-limiting example, the mammalian subject may be a rodent, such as but not limited to, a transgenic mouse (e.g., htau, P301S, rTg4510, rTgTauEC, SCN1a-A1783V) or a non-transgenic (wild type) mouse. As another non-limiting example, the subject may be a non-human primate, such as but not limited to a rhesus macaque (Macaca mulatta). As yet another non-limiting example, the subject may be a human. In some embodiments, the human is a healthy human. In some embodiments, the human is a patient who has been diagnosed with a neurological disorder. In some embodiments, the human is a patient who has been diagnosed with or is suspected of having or is susceptible to a tauopathy. In some embodiments, the human is a patient who has been diagnosed with or is suspected of having or is susceptible to a epilepsy. Administration of any of the here within described AAV particles comprising a vector genome encoding a modulatory polynucleotide may be used to manage, prevent, slow the progression of, treat the symptoms associated with, and/or cure a disease afflicting the mammalian subject. The subject may be administered any of the described compositions, including pharmaceutical compositions.

In some embodiments, an AAV particle described herein, e.g., an AAV particle encoding an siRNA for targeting MAPT described herein, is administered via intravenous administration. In some embodiments, an AAV particle described herein, e.g., an AAV particle encoding an siRNA for targeting MAPT described herein, is administered via intracisternal magna administration.

The pharmaceutical compositions of AAV particles described herein may be characterized by one or more of bioavailability, therapeutic window and/or volume of distribution.

### III. ADMINISTRATION AND DOSING

### Administration

The AAV particles comprising a nucleic acid sequence encoding the siRNA molecules of the present disclosure may be administered by any route which results in a therapeutically effective outcome. These include, but are not limited to, within the parenchyma of an organ such as, but not limited to, a brain (e.g., intraparenchymal), spinal cord (intraparenchymal), corpus striatum (intrastriatal), enteral (into the intestine), gastroenteral, epidural, oral (by way of the mouth), transdermal, peridural, intracerebral (into the cerebrum), intracerebroventricular (into the cerebral ventricles), subpial (under the pia), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraganglionic (into the ganglion), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In specific embodiments, compositions of AAV particles comprising a nucleic acid sequence encoding the siRNA molecules of the present disclosure may be administered in a way which facilitates entry to cells of the central nervous system (e.g., cells of the brain and spinal cord). The cells of the brain may be localized within one or specific brain areas. Non-limiting examples of brain areas may be the frontal, temporal, or parietal lobes, basal ganglia, thalamus, cerebellum and brainstem.

In specific embodiments, compositions of AAV particles comprising a nucleic acid sequence encoding the siRNA molecules of the present disclosure may be administered in a way which facilitates the vectors or siRNA molecule to enter the central nervous system and penetrate into cortical neurons, hippocampal neurons, entorhinal neurons, cholinergic neurons, interneurons, dentate gyrus granule cells, glial cells, and oligodendrocytes.

In some embodiments, the AAV particles comprising a nucleic acid sequence encoding the siRNA molecules of the present disclosure may be administered by intramuscular (IM) injection. In some embodiments, the AAV particles comprising a nucleic acid sequence encoding the siRNA molecules of the present disclosure may be administered by intraperitoneal (IP) injection. In some embodiments, the AAV particles comprising a nucleic acid sequence encoding the siRNA molecules of the present disclosure may be administered by intracerebroventricular (ICV) injection. In some embodiments, the AAV particles comprising a nucleic acid sequence encoding the siRNA molecules of the present disclosure may be administered via intraparenchymal (IPa) injection into the spinal cord and/or brain. In some embodiments, the AAV particles comprising a nucleic acid sequence encoding the siRNA molecules of the present disclosure may be administered via intra-cisterna magna (ICM) injection. In some embodiments, the AAV particles comprising a nucleic acid sequence encoding the siRNA molecules of the present disclosure may be administered by via intrathecal (IT) injection. In some embodiments, the AAV particles comprising a nucleic acid sequence encoding the siRNA molecules of the present disclosure may be administered via subpial injection. In some embodiments, the AAV particles comprising a nucleic acid sequence encoding the siRNA molecules of the present disclosure may be administered via ICM injection and any other route of administration described herein. As a non-limiting example, the AAV particles comprising a nucleic acid sequence encoding the siRNA molecules of the present disclosure may be administered via combined ICM and IPa, e.g. intrathalamic, injection.

In some embodiments, an AAV particle described herein (e.g., an AAV particle comprising an AAV capsid polypeptide, e.g., an AAV capsid variant) may be administered to a subject by systemic administration. In some embodiments, the systemic administration is intravenous administration. In another embodiment, the systemic administration is intraarterial administration. In some embodiments, an AAV particle of the present disclosure may be administered to a subject by intravenous administration. In some embodiments, the intravenous administration may be achieved by subcutaneous delivery. In some embodiments, the AAV particle is administered to the subject via focused ultrasound (FUS), e.g., coupled with the intravenous administration of microbubbles (FUS-MB) or MRI-guided FUS coupled with intravenous administration, e.g., as described in Terstappen et al. (Nat Rov Drug Discovery, doi.org/10.1038/s41573-021-00139-y (2021)), the contents of which are incorporated herein by reference in its entirety. In some embodiments, the AAV particle, e.g., an AAV particle described herein, is administered to the subject intravenously. In some embodiments, the subject is a human.

In some embodiments, AAV particles that express siRNA duplexes of the present disclosure may be administered to a subject by peripheral injections (e.g., intravenous) and/or intranasal delivery. It was disclosed in the art that the peripheral administration of AAV particles for siRNA duplexes can be transported to the central nervous system, for example, to the neurons (e.g., U.S. Patent Publication Nos. 20100240739; and 20100130594; the content of each of which is incorporated herein by reference in their entirety).

In other embodiments, compositions comprising at least one AAV particle comprising a nucleic acid sequence encoding the siRNA molecules of the present disclosure may be administered to a subject by intracranial delivery (See, e.g., U.S. Pat. No. 8,119,611; the content of which is incorporated herein by reference in its entirety).

The AAV particle comprising a nucleic acid sequence encoding the siRNA molecules of the present disclosure may be administered in any suitable form, either as a liquid solution or suspension, as a solid form suitable for liquid solution or suspension in a liquid solution. The siRNA duplexes may be formulated with any appropriate and pharmaceutically acceptable excipient.

The AAV particle comprising a nucleic acid sequence encoding the siRNA molecules of the present disclosure may be administered in a therapeutically effective amount, e.g., an amount that is sufficient to alleviate and/or prevent at least one symptom associated with the disease, or provide improvement in the condition of the subject.

In some embodiments, the AAV particle may be administered to the CNS in a therapeutically effective amount to improve function and/or survival for a subject with AD, FTD, and/or DS.

In some embodiments, an siRNA for targeting MAPT; or AAV particle or pharmaceutical composition described herein comprising or encoding a siRNA for inhibiting MAPT can be administered or used to treat mild cognitive impairment (MCI), neurodegenerative disease, Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive supranuclear palsy (PSP), Down's syndrome, Pick's disease, corticobasal degeneration (CBD), corticobasal syndrome, amyotrophic lateral sclerosis (ALS), a prion disease, Creutzfeldt-Jakob disease (CJD), multiple system atrophy, tangle-only dementia, stroke, and progressive subcortical gliosis.

In some embodiments, an siRNA that targets MAPT described herein or an AAV particle comprising a payload, e.g., a siRNA that binds to MAPT described herein, can be used to treat a traumatic brain injury (TBI), e.g., as described in Edwards et al. “Traumatic Brain Injury Induces Tau Aggregation and Spreading,” J. Neurotrauma, 2020, 37(1):80-92, the contents of which are hereby incorporated by reference in their entirety.

In some embodiments, the AAV particle may be administered to a subject in a therapeutically effective amount for the siRNA duplexes or dsRNA to target cortical neurons. In another embodiment, the AAV particle may be administered to a subject in a therapeutically effective amount for the siRNA duplexes or dsRNA to target cholinergic neurons. In yet another embodiment, the AAV particle may be administered to a subject in a therapeutically effective amount for the siRNA duplexes or dsRNA to target interneurons. In yet another embodiment, the AAV particle may be administered to a subject in a therapeutically effective amount for the siRNA duplexes or dsRNA to target glial cells.

In some embodiments, the siRNA duplexes or dsRNA may target MAPT transcript and reduce the levels of RNA transcript and/or resultant protein product(s). As another non-limiting example, the siRNA duplexes or dsRNA target MAPT transcript and can suppress or inhibit or silence MAPT transcript and reduce tau mediated toxicity.

In some embodiments, the AAV particle may be administered via intracerebroventricular (ICV) injection in a therapeutically effective amount to transduce cortical neurons, interneurons, hippocampal neurons, entorhinal neurons, cholinergic neurons, and/or glial cells such as, but not limited to, oligodendrocytes and/or astrocytes. As a non-limiting example, the vector may be administered via intraventricular injection into the lateral ventricle.

In some embodiments, the AAV particle may be administered via intracisternal injection in a therapeutically effective amount to transduce cortical neurons, interneurons, hippocampal neurons, entorhinal neurons, cholinergic neurons, and/or glial cells such as, but not limited to, oligodendrocytes and/or astrocytes. As a non-limiting example, the vector may be administered via intraventricular injection into the cisterna magna. In some embodiments, the AAV particle may be administered using intrathecal (IT) infusion in a therapeutically effective amount to transduce cortical neurons, interneurons, hippocampal neurons, entorhinal neurons, cholinergic neurons, and/or glial cells such as, but not limited to, oligodendrocytes and/or astrocytes. As a non-limiting example, the vector may be administered into the lumbar spinal cord. In some embodiments, the AAV particle may be administered using intramuscular (IM) infusion in a therapeutically effective amount to transduce cortical neurons, interneurons, hippocampal neurons, entorhinal neurons, cholinergic neurons, and/or glial cells such as, but not limited to, oligodendrocytes and/or astrocytes. As a non-limiting example, the vector may be administered into the deltoid. In some embodiments, the AAV particle may be administered to the CNS in a therapeutically effective amount for the siRNA duplexes or dsRNA to target cells in various brain areas. As a non-limiting example, the brain areas may be the hippocampus, cortex, thalamus, brainstem, striatum, and cerebellum. In some embodiments, the AAV particle may be administered to the CNS in a therapeutically effective amount for the siRNA duplexes or dsRNA to target cells in the olfactory bulb. In some embodiments, the AAV particle may be administered to the CNS in a therapeutically effective amount for the siRNA duplexes or dsRNA to target cells in the liver.

In some embodiments, the AAV particle comprising a modulatory polynucleotide may be formulated. As a non-limiting example, the baricity and/or osmolality of the formulation may be optimized to ensure optimal drug distribution in the central nervous system or a region or component of the central nervous system.

In some embodiments, the AAV particle comprising a modulatory polynucleotide may be delivered to a subject via a single route administration. In some embodiments, the AAV particle comprising a modulatory polynucleotide may be delivered to a subject via a multi-site route of administration. A subject may be administered the AAV particle comprising a modulatory polynucleotide at 2, 3, 4, 5 or more than 5 sites. In some embodiments, a subject may be administered the AAV particle comprising a modulatory polynucleotide described herein using a bolus injection. In some embodiments, the efficacy of administration of the AAV particle comprising a modulatory polynucleotide using a bolus injection may be measured by monitoring the gene transfer to the spinal cord, brain stem, or cortex. The biodistribution and cellular tropism may be monitored by any methods known in the art, such as, but not limited to, immunostaining, and the vector genome levels may be measured by digital PCR.

In some embodiments, a subject may be administered the AAV particle comprising a modulatory polynucleotide described herein using sustained delivery over a period of minutes, hours or days. The infusion rate may be changed depending on the subject, distribution, formulation or another delivery parameter.

In some embodiments, the AAV particle described herein is administered via putamen and caudate infusion. As a non-limiting example, the dual infusion provides a broad striatal distribution as well as a frontal and temporal cortical distribution.

In some embodiments, an AAV particle or pharmaceutical composition described herein is administered intravenously.

In some embodiments, the selection of subjects for administration of the AAV particle described herein and/or the effectiveness of the dose, route of administration and/or volume of administration may be evaluated using imaging of the perivascular spaces (PVS) which are also known as Virchow-Robin spaces. PVS surround the arterioles and venules as they perforate brain parenchyma and are filled with cerebrospinal fluid (CSF)/interstitial fluid. PVS are common in the midbrain, basal ganglia, and centrum semiovale. While not wishing to be bound by theory, PVS may play a role in the normal clearance of metabolites and have been associated with worse cognition and several disease states including Parkinson's disease. PVS are usually normal in size but they can increase in size in a number of disease states. Potter et al. (Cerebrovasc Dis. 2015 January; 39(4):224-231; the contents of which are herein incorporated by reference in its entirety) developed a grading method where they studied a full range of PVS and rated basal ganglia, centrum semiovale and midbrain PVS. They used the frequency and range of PVS used by Mac and Lullich et al. (J Neurol Neurosurg Psychiatry. 2004 November; 75(11):1519-23; the contents of which are herein incorporated by reference in its entirety) and Potter et al. gave 5 ratings to basal ganglia and centrum semiovale PVS: 0 (none), 1 (1-10), 2 (11-20), 3 (21-40) and 4 (>40) and 2 ratings to midbrain PVS: 0 (non-visible) or 1 (visible). The user guide for the rating system by Potter et al. can be found at: sbirc.ed.ac.uk/documents/epvs-rating-scale-user-guide.pdf, the contents of which are hereby incorporated by reference in their entirety.

In some embodiments, administration of an siRNA or modulatory polynucleotide for targeting MAPT or an AAV viral genome encoding an siRNA or modulatory polynucleotide for targeting MAPT described herein results in a reduction of tau pathology, e.g., a decrease in a biomarker of tau pathology (e.g., 18F-flortaucipir, plasma ptau 181, or 18F-PM-PBB3), e.g., as measured by a PET scan or PET scan in combination with Braak neuropathological staging and/or serum biomarker staining.

### Dosing

The pharmaceutical compositions of the present disclosure may be administered to a subject using any amount effective for reducing, preventing and/or treating a disease and/or disorder. The exact amount required will vary from subject to subject, depending on the species, age, and general condition of the subject, the severity of the disease, the particular composition, its mode of administration, its mode of activity, and the like.

The compositions of the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutic effectiveness for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the siRNA duplexes employed; the duration of the treatment; drugs used in combination or coincidental with the specific compound employed; and like factors well known in the medical arts.

As a non-limiting example, the dose may be determined based on the total volume of CSF of a subject. For example, cynomolgus monkeys have a total estimated cerebrospinal fluid (CSF) volume of approximately 6 to 12 mL and humans have a total estimated CSF of approximately 120 to 150 mL, a factor of at least 10 to 12-fold. Therefore, a factor of 10× to 12× may be used to determine a human dose based on the dose to a cynomolgus monkey. In some embodiments, the factor is 10×. In another embodiment the factor is 11×. In yet another embodiment, the factor is 12×. In yet another embodiment, the factor may be, but is not limited to, 10×, 10.1×, 10.2×, 10.3×, 10.4×, 10.5×, 10.6×, 10.7×, 10.8×, 10.9×, 11×, 11.1×, 11.2×, 11.3×, 11.4×, 11.5×, 11.6×, 11.7×, 11.8×, 11.9×, 12×, 12.1×, 12.2×, 12.3×, 12.4×, and 12.5×.

In some embodiments, the age and sex of a subject may be used to determine the dose of the compositions of the present disclosure. As a non-limiting example, a subject who is older may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a female subject

In some specific embodiments, the doses of AAV particles for delivering siRNA duplexes of the present disclosure may be adapted depending on the disease condition, the subject and the treatment strategy.

In some embodiments, delivery of the compositions in accordance with the present disclosure to cells comprises a rate of delivery defined by [VG/hour=mL/hour*VG/mL] wherein VG is vector genomes, VG/mL is composition concentration, and mL/hour is rate of prolonged delivery.

In some embodiments, delivery of compositions in accordance with the present disclosure to cells may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹. 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹² 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹². 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In some embodiments, delivery of compositions in accordance with the present disclosure to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 3×10¹³, 4×10¹³, 5×10¹³, 6×10¹³, 6.7×10¹³. 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg.

In some embodiments, about 10⁵ to 10⁶ vector genome (unit) may be administered per dose.

In some embodiments, delivery of the compositions in accordance with the present disclosure to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴ 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In certain embodiments, the desired siRNA duplex dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in a 24-hour period. It may be administered as a single unit dose. In some embodiments, the AAV particles comprising the modulatory polynucleotides of the present disclosure are administered to a subject in split doses. They may be formulated in buffer only or in a formulation described herein.

In some embodiments, the dose, concentration and/or volume of the composition described herein may be adjusted depending on the contribution of the thalamus to cortical and subcortical distribution after administration. The administration may be intracerebroventricular (ICV), intrathalamic, intraparenchymal into the spinal cord, subpial, and/or intrathecal administration.

In some embodiments, the dose, concentration and/or volume of the composition described herein may be adjusted depending on the cortical and neuraxial distribution following administration by intravenous, intracerebroventricular, intrathalamic, intraparenchymal into the spinal cord, subpial, and/or intrathecal delivery.

### IV. METHODS AND USES OF THE COMPOSITIONS OF THE DISCLOSURE

The present disclosure relates to modulatory polynucleotides, e.g., RNA or DNA molecules, which may be used as therapeutic agents as RNA interference mediated gene silencing can specifically inhibit targeted gene expression.

Described herein are double stranded RNA (dsRNA) molecules (e.g., small interfering RNA, siRNA) targeting MAPT mRNA, pharmaceutical compositions comprising such dsRNA molecules, as well as processes of their design. The present disclosure also provides methods of their use for inhibiting MAPT gene expression, MAPT transcript expression and/or MAPT protein production, which may be used for treating neurodegenerative disease, such as, but not limited to tauopathies, and other indications described here. Such indications may include, but are not limited to mild cognitive impairment (MCI), neurodegenerative disease, Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive supranuclear palsy (PSP), Down's syndrome, Pick's disease, corticobasal degeneration (CBD), corticobasal syndrome, amyotrophic lateral sclerosis (ALS), a prion disease, Creutzfeldt-Jakob disease (CJD), multiple system atrophy, tangle-only dementia, stroke, and progressive subcortical gliosis. As a non-limiting example, the tauopathy is Alzheimer's disease (AD). As a non-limiting example, the tauopathy is frontotemporal dementia (FTD). As a non-limiting example, the tauopathy is Frontotemporal dementia and parkinsonism linked to chromosome 17 (FTDP-17). As a non-limiting example, the tauopathy is progressive supranuclear palsy (PSP). As a non-limiting example, the tauopathy is central nervous system trauma. As a non-limiting example, the tauopathy is chronic traumatic encephalopathy (CTE). As a non-limiting example, the tauopathy is traumatic brain injury (TBI). As a non-limiting example, the tauopathy is prion disease. As a non-limiting example, the tauopathy is neurodegeneration. As a non-limiting example, the tauopathy is stroke. As a non-limiting example, the tauopathy is an epilepsy or a variant thereof, such as, but not limited to, Dravet syndrome (DS).

In particular, the AAV particles comprising modulatory polynucleotides comprising a nucleic acid sequence encoding the siRNA molecules of the present disclosure may be delivered into specific types of targeted cells, including neurons such as cortical neurons, hippocampal neurons (e.g., dentate gyrus granule cell neurons), entorhinal neurons, cholinergic neurons, and interneurons (e.g., inhibitory interneurons), or glial cells such as oligodendrocytes, astrocytes and microglia.

In some embodiments, the present disclosure provides methods for treating, or ameliorating Alzheimer's disease (AD), frontotemporal dementia (FTD), and/or Dravet syndrome (DS) associated with abnormal tau RNA and/or protein products in a subject in need of treatment, the method comprising administering to the subject a pharmaceutically effective amount of at least one siRNA duplex or a nucleic acid encoding a siRNA duplex targeting the MAPT transcript, delivering said siRNA duplex (or encoded duplex) into targeted cells, inhibiting MAPT transcript expression and production of toxic proteins, and ameliorating symptoms of AD and/or FTD in the subject.

In some embodiments, the siRNA molecules or the AAV particles encoding such siRNA molecules may be introduced directly into the central nervous system of the subject, for example, by intraparenchymal (IPa), subpial, intrathecal, intracranial injection or intravenous (IV) administration.

In some embodiments, the pharmaceutical composition of the present disclosure is used as a solo therapy. In other embodiments, the pharmaceutical composition of the present disclosure is used in combination therapy. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

The combination therapy may be in combination with one or more neuroprotective agents such as small molecule compounds, growth factors and hormones which have been tested for their neuroprotective effect on neuron degeneration.

In some embodiments, the present disclosure provides methods for treating, or ameliorating Alzheimer's disease (AD), frontotemporal dementia (FTD), and/or Dravet syndrome (DS) by administering to a subject in need thereof a therapeutically effective amount of an siRNA or AAV particle described herein.

### Alzheimer's Disease

Alzheimer Disease (AD) is a debilitating neurodegenerative disease currently afflicting more than 35 million people worldwide, with that number expected to double in coming decades. Symptomatic treatments have been available for many years, but these treatments do not address the underlying pathophysiology. Recent clinical trials using these and other treatments have largely failed and, to date, no known cure has been identified.

The AD brain is characterized by the presence of two forms of pathological aggregates, the extracellular plaques composed of β-amyloid (Aβ) and the intracellular neurofibrillary tangles (NFT) comprised of hyperphosphorylated microtubule associated protein tau. Based on early genetic findings, β-amyloid alterations were thought to initiate disease, with changes in tau considered downstream. Thus, most clinical trials have been Aβ-centric. Although no mutations of the MAPT gene have been linked to AD, such alterations have been shown to result in a family of dementias known as tauopathies, demonstrating that changes in tau can contribute to neurodegenerative processes. Tau is normally a very soluble protein known to associate with microtubules based on the extent of its phosphorylation. Hyperphosphorylation of tau depresses its binding to microtubules and microtubule assembly activity. In tauopathies, the tau becomes hyperphosphorylated, misfolds and aggregates as NFT of paired helical filaments (PHF), twisted ribbons or straight filaments. In AD, NFT pathology, rather than plaque pathology, correlates more strongly with neuropathological markers such as neuronal loss, synaptic deficits, severity of disease and cognitive decline. NFT pathology marches through the brain in a stereotyped manner and animal studies suggest a trans-cellular propagation mechanism along neuronal connections.

Several approaches have been proposed for therapeutically interfering with the progression of tau pathology to prevent or hasten downstream molecular, cellular, and/or cognitive consequences. Given that NFT are composed of hyperphosphorylated, misfolded and aggregated tau, each pathological form has yielded a set of avidly pursued targets. Tau reduction has also been a goal of many treatment strategies as continued- or over-production of tau, and specifically mutant tau, may exacerbate disease progression, e.g., by contributing to already impaired neuronal trafficking processes, or accelerating NFT formation. Introducing agents that limit phosphorylation, block misfolding, prevent aggregation, or reduce tau expression have all generated promising results, however the success of these strategies has been limited. As such, there is presently no known effective treatment for tauopathies. Thus, there remains a need in the art for effectively reducing, preventing and/or treating tauopathies.

The delivery of vectored RNAi targeting MAPT of present disclosure may be used to treat subjects suffering from AD and other tauopathies. In some cases, methods of the present disclosure may be used to treat subjects suspected of developing AD or other tauopathies.

### Frontotemporal Dementia

Frontotemporal dementia (FTD), also referred to as frontotemporal lobar degeneration (FTLD), is a neurological condition often associated with AD. FTD represents a group of brain disorders that result from the progressive degeneration of the temporal and frontal lobes of the brain. FTD is the second most common form of early-onset (<65 years) dementia after Alzheimer's disease (AD). Common signs and symptoms of FTD include significant changes in behavior and personality (such as lack of judgment and inhibition, apathy, and repetitive compulsive behavior), impairment or loss of speech and language function, and movement disorders (such as rigidity, muscle spasms and muscle weakness).

The prevalence of FTD in the United States was estimated to around 60,000 cases (Knopman and Roberts. J Mol Neurosci. 2011; 45(3):330-5). Signs and symptoms typically manifest in late adulthood, more commonly between the ages of 45 and 65, approximately equally affecting men and women. The course of the disease ranges from 2 to over 20 years, with a mean course of 8 years from the onset of symptoms.

Clinicopathological and genetic data have long supported the concept that AD and FTD may represent an overlapping continuum of disease with a shared underlying pathogenesis (Lillo and Hodges, J Clin Neurosci. 2009; 16(9):1131-5; Neumann et al., Science. 2006; 314(5796):130-3). They often co-occur in a family, and the prevalence of frontal lobe impairment in AD populations may approach 50%. Similarly, as many as half of FTD patients develop clinical symptoms of motor neuron dysfunction (Blitterswijk et al., Curr Opin Neurol. 2012; 25(6):689-700). Both diseases commonly show accumulations of abnormal proteins including the DNA/RNA binding protein TDP-43 (Neumann et al., Science. 2006; 314(5796):130-3).

### Frontotemporal Dementia and Parkinsonism Linked to Chromosome 17

Although Alzheimer's disease is, in part, characterized by the presence of tau pathology, no known mutations in the MAPT gene have been causally linked to the disease. Mutations in the MAPT gene have been shown to lead to an autosomal dominantly inherited tauopathy known as frontotemporal dementia and parkinsonism linked to chromosome 17 (FTDP-17) and demonstrate that alterations in tau can lead to neurodegenerative changes in the brain. Mutations in the MAPT gene that lead to FTDP-17 are thought to influence splicing patterns, thereby leading to an elevated proportion of tau with four microtubule binding domains (rather than three). These molecules are considered to be more amyloidogenic, meaning they are more likely to become hyperphosphorylated and more likely to aggregate into NFT (Hutton, M. et al., 1998, Nature 393(6686):702-5). Although physically and behaviorally, FTDP-17 patients can appear quite similar to Alzheimer's disease patients, at autopsy FTDP-17 brains lack the prominent AB plaque pathology of an AD brain (Gotz, J. et al., 2012, British Journal of Pharmacology 165(5):1246-59). Therapeutically targeting tau protein may ameliorate and prevent degenerative changes in the brain and potentially lead to improved cognitive ability.

As of today, there is no treatment to prevent, slow the progression, or cure FTDP-17. Medication may be prescribed to reduce aggressive, agitated or dangerous behavior. There remains a need for therapy affecting the underlying pathophysiology, such as RNAi therapies targeting tau protein.

In some embodiments, the vectored RNAi delivery of the present disclosure may be used to treat subjects suffering from FTDP-17. In some cases, methods of the present disclosure may be used to treat subjects suspected of developing FTDP-17.

### Chronic Traumatic Encephalopathy

Unlike the genetically linked tauopathies, chronic traumatic encephalopathy (CTE) is a degenerative tauopathy linked to repeated head injuries. The disease was first described in boxers who behaved “punch drunk” and has since been identified primarily in athletes that engage in American football, ice hockey, wrestling and other contact sports. The brains of those suffering from CTE are characterized by distinctive patterns of brain atrophy accompanied by accumulation of hyperphosphorylated species of aggregated tau in NFT. In CTE, pathological changes in tau are accompanied by a number of other pathobiological processes, such as inflammation (Daneshvar, D. H. et al., 2015 Mol Cell Neurosci 66 (Pt B): 81-90). Targeting the tau may provide reprieve from the progression of the disease and may allow cognitive improvement.

As of today, there is no medical therapy to treat or cure CTE. The condition is only diagnosed after death, due to lack of in vivo techniques to identify CTE specific biomarkers. There remains a need for therapy affecting the underlying pathophysiology, such as vectored RNAi therapies targeting tau protein.

In some embodiments, the vectored RNAi delivery methods of the present disclosure may be used to treat subjects suffering from CTE. In some cases, methods of the present disclosure may be used to treat subjects suspected of developing CTE.

### Prion Diseases

Prion diseases, also known as transmissible spongiform encephalopathies (TSEs), are a group of rare progressive conditions affecting the nervous system. The related conditions are rare and are typically caused by mutations in the PRNP gene which enables production of the prion protein. Gene mutations lead to an abnormally structured prion protein. Alternatively, the abnormal prion may be acquired by exposure from an outside source, e.g. by consumption of beef products containing the abnormal prion protein. Abnormal prions are misfolded, causing the brain tissue to degenerate rapidly. Prion diseases include, but are not limited to, Creutzfeldt-Jakob disease (CJD), Gerstmann-Sträussler-Scheinker syndrome (GSS), fatal insomnia (FFI), variably protcase-sensitive prionopathy (VPSPr), and kuru. Prion diseases are rare. Approximately 350 cascs of prion diseases are diagnosed in the US annually.

CJD is a degenerative brain disorder characterized by problems with muscular coordination, personality changes including mental impairment, impaired vision, involuntary muscle jerks, weakness and eventually coma. The most common categories of CJD are sporadic, hereditary (familial) due to a genetic mutation, and acquired. Sporadic CJD is the most common form affecting people with no known risk factors for the disease. The acquired form of CJD is transmitted by exposure of the brain and nervous system tissue to the prion. As an example, variant CJD (vCDJ) is linked to a bovine spongiform encephalopathy (BSE), also known as a ‘mad cow’ disease. CJD is fatal and patients typically die within one year of diagnosis.

Prion diseases are associated with an infectious agent consisting of an alternative conformational isoform of the prion protein, PrPSc. PrPSc replication is considered to occur through an induction of the infectious prion in the normal prion protein (PrPC). The replication occurs without a nucleic acid.

As of today, there is no therapy to manage or cure CJD, or other prion diseases. Typically, treatment is aimed at alleviating symptoms and increasing comfortability of the patient, e.g. with pain relievers. There remains a need for therapy affecting the underlying pathophysiology, such as vectored RNAi therapies targeting the prion protein.

In some embodiments, vectored RNAi delivery methods of the present disclosure may be used to treat subjects suffering from a prion disease. In some cases, methods of the present disclosure may be used to treat subjects suspected of developing a prion disease.

### Neurodegeneration and Stroke

Neurodegenerative diseases and other diseases of the nervous system share many common features. Neurodegenerative diseases, in particular, are a group of conditions characterized by progressive loss of neuronal structure and/or function, ultimately leading to neuronal cell death. Neurons are the building blocks of the nervous system(s) and are generally unable to reproduce and/or be replaced, and therefore neuron damage and/or death is especially devastating. Other, non-degenerating diseases that lead to neuronal cell loss, such as stroke, have similarly debilitating outcomes. Targeting molecules that contribute to the deteriorating cell structure or function may prove beneficial generally for treatment of nervous system diseases, neurodegenerative disease and/or stroke.

Many neurodegenerative diseases are associated with aggregation of misfolded proteins, including, but not limited to, alpha synuclein, tau, amyloid β, prion proteins, TDP-43, and huntingtin (see, e.g. De Genst et al., 2014, Biochim Biophys Acta; 1844(11):1907-1919, and Yu et al., 2013, Neurotherapeutics.; 10(3):459-472, references therein). The aggregation results from disease-specific conversion of soluble proteins to an insoluble, highly ordered fibrillary deposit. This conversion is thought to prevent the proper disposal or degradation of the misfolded protein, thereby leading to further aggregation. In some embodiments, the vectored RNAi delivery of the present disclosure is utilized to target the aforementioned antigens (e.g., misfolded or aggregated proteins).

AAV Particles and methods of using the AAV particles described in the present disclosure may be used to prevent, manage and/or treat tauopathies. As a non-limiting example, the AAV particles of the present disclosure comprise a nucleic acid sequence encoding at least one of the sequences described in Table 5.

### Dravet Syndrome and Epilepsy

Dravet syndrome (DS), also known as severe myoclonic epilepsy of infancy (SMEI), is a type of early-onset epilepsy that manifests as intractable epilepsy and neurodevelopmental delays, with genetic basis in the mutation of the voltage-gated calcium channel, alpha-1 subunit (SCNIA) gene on chromosome 2q24 (see, e.g., Anwar et al., Cureus. 2019 June; 11(6): e5006 and Claes et al., Am J Hum Genet. 2001 June; 68(6):1327-32. Epub 2001 May 15, the contents of each of which are incorporated herein by reference in their entirety). While more than 90% of these mutations are thought to be de novo (sporadic), familial mutations (e.g., missense) account for only 5-10% of cases. DS is rare, with incidence estimated at 1 in 15000 to 1 in 40,000, arising in males and females in equal proportion (see, e.g., Wu at al., Pediatrics. 2015 November; 136(5): e1310-e1315; Rosander et al., Dev Med Child Neurol. 2015 July; 57(7):628-633; and, Hurst Epilepsia. 1990 July-August; 31(4):397-400, the contents of each of which are incorporated herein by reference in their entirety).

Age of onset for DS typically occurs in the first year of life at ~6 months (1-18 months, range) with 10-20% mortality by the age of 10 (Shmuely et al., Epilepsy Behav. 2016 November; 64(Pt A):69-74, the contents of which are incorporated herein by reference in their entirety). Indeed, it is estimated that 14-20% of DS patients succumb to sudden unexpected death in epilepsy (SUDEP), the cause of which is largely unknown, but that may be associated with cardiac or respiratory complications (Genton et al., 2011, Epilepsia. 2011 April; 52 Suppl 2:44-9 the contents of which are incorporated herein by reference in their entirety). During adulthood, DS commonly manifests as persistent motor and cognitive functions (see, e.g., Anwar et al., Cureus. 2019 June; 11(6): e5006, the contents of which are incorporated herein by reference in their entirety). Several different seizure categories have been identified in DS patients such as, but not limited to, convulsive, myoclonic, absence, focal, obtundation status, and tonic seizures. In addition to pleomorphic seizure activity, motor dysfunction may include ataxia, tremors, dysarthria, pyramidal, and extrapyramidal signs. Most patients also exhibit cognitive, visual and visuomotor, and language impairment. Psychiatric conditions including aggressiveness, agitation, obsessiveness, perseveration, and hoarding behavior may also occur in DS patients.

Due to its intractable nature, treatment options for DS are somewhat limited, but may include anti-epileptic drugs, cannabinoids, ketogenic diet therapy, and surgery (e.g., deep brain stimulation). Despite the development of a number antiepileptic drugs over the course of the past few decades, the efficacy of such treatments has not substantially improved, in particular, as several patients suffer from seizures that are drug-refractory (Pérez-Pérez et al., Epilepsy Behav. 2019 Aug. 1:106430, the contents of which are incorporated herein by reference in their entirety). DS is among the most drug-resistant forms of epilepsy (see, e.g., Chiron Dev Med Child Neurol. 2011 April; 53 Suppl 2:16-8, the contents of which are incorporated herein by reference in their entirety). Thus, there remains a need in the art for effectively reducing, preventing, and/or treating epilepsies or variants thereof, such as, but not limited to, DS and other intractable childhood epilepsies.

Several lines of evidence suggest that antiepileptic effects of tau reduction and/or ablation may offer a new avenue for the treatment of drug-resistant epilepsy. Indeed, the genetic ablation of tau has been shown to reduces hyperexcitability in AD mouse lines, induced seizure models, and genetic in vivo models of epilepsy alike. For instance, a ~50% reduction of tau by has been shown to improve survival, reduce seizure frequency, decrease neuronal hyperexcitability, and rescue behavioral impairment in a mouse model of DS (Gheyara et al., Ann Neurol. 2014 September; 76(3):443-56, the contents of which are incorporated herein by reference in their entirety). In two chemically induced seizure mouse models, antisense oligonucleotide (ASO)-mediated reduction of tau tightly tracked with attenuation of seizure severity (Devos et al., J Neurosci. 2013 Jul. 31; 33(31):12887-97, the contents of which are incorporated herein by reference in their entirety). Levels of tau protein in cerebrospinal fluid (CSF) has also been linked to epilepsy severity, including in both AD and epileptic patients (see, e.g., Tábuas-Pereira et al., Epilepsy Behav. 2019 September; 98 (Pt A): 207-209, the contents of which are incorporated herein by reference in their entirety). Though as yet not fully understood, possible contributing mechanisms to support of tau (MAPT) knockdown as a genetic modifier for epilepsy, include: enhanced activity of inhibitory neurons; reduced N-methyl-D-aspartate receptor (NMDAR)-dependent excitotoxicity; and, reduced mossy fiber sprouting (Roberson et al., J Neurosci. 2011 Jan. 12; 31(2):700-11; Miyamoto et al., Mol Neurodegener. 2017 May 19; 12(1):41; and, Cavarsan et al., Front Neurol. 2018 Nov. 30; 9:1023, the contents of each of which are incorporated herein by reference in their entirety). Together, tau reduction may be therapeutically beneficial for DS and other epilepsies, including those that frequently occur in AD.

In some embodiments, the vectored RNAi delivery methods of the present disclosure may be used to treat subjects suffering from DS, or other epilepsy or variant thereof. In some cases, methods of the present disclosure may be used to treat subjects suspected of developing DS, or other epilepsy or variant thereof.

### Animal Models

Vectored RNAi delivery methods of the present disclosure (e.g., delivery of the siRNA or the AAV particles of the present disclosure) may be used to inhibit or suppress expression of MAPT transcript in a subject, including a non-human animal. In some embodiments, the non-human animal may be a rodent, such as, but not limited to, a mouse. As a non-limiting example, the mouse may be a transgenic mouse. Non-limiting examples of a transgenic mice include htau, P301S, rTg4510, and rTgTauEC transgenic mice.

In some embodiments, delivery of the siRNA or AAV particles of the present disclosure may be used to prevent, manage and/or treat tauopathy in the P301S mouse model of tauopathy, as described by Allen et al., J Neurosci. 2002 Nov. 1; 22(21):9340-51, the contents of which are incorporated herein by reference in their entirety; or the htau mouse model of tauopathy, as described in Andorfer et al., J Neurochem. 2003 August; 86(3):582-90 and WO199620218, the contents of which are incorporated herein by reference in their entirety). In some embodiments, sarkosyl insoluble paired helical filament (PHF) tau, or like substance, from Alzheimer's disease (AD) and/or other tauopathy disease brain or biofluid, may be introduced, e.g., via injection, into a brain area, e.g. the hippocampus, of htau mice, using methods similar to those previously described by Hu et al., Alzheimer's Dement. 2016 October; 12(10):1066-1077 and Gerson et al., J Neurotrauma. 2016 Nov. 15; 33(22):2034-2043, the contents of each of which are incorporated herein by reference in their entirety. In some embodiments, aged htau mice may be monitored for the onset of cognitive deficits as previously described by Polydoro et al., 2009 and Geiszler et al., 2016 (Polydoro et al., J Neurosci. 2009 Aug. 26; 29(34):10741-9 and Geiszler et al., Neuroscience. 2016 Aug. 4; 329:98-111, the contents of each of which are incorporated herein by reference in their entirety). In some embodiments, tau immunoreactivity may be measured in the seeding and in the first and secondary synaptic brain areas using anti-tau antibodies, such as but not limited to the AT100 antibody (pSer²¹²/pSer²¹⁴; ThermoFisher, Waltham, MA; described in U.S. Pat. No. 6,121,003, the contents of which are herein incorporated in their entirety).

In some embodiments, brain lysate from mice may be tested for tau seeding activity using a biosensor as described by, e.g., Gui et al., Sensors (Basel). 2017 July; 17(7):1623 and Frost et al., J Biol Chem. 2009 May 8; 284(19):12845-52, the contents of each of which are incorporated herein by reference in their entirety; or Frost et al., J Biol Chem. 2009 May 8:284(19):12845-52, the contents of which are incorporated herein by reference in their entirety.

In some embodiments, delivery of the siRNA or AAV particles of the present disclosure may be used to inhibit or suppress expression of MAPT transcript in a subject, including a non-human animal. The non-human animal may be a non-human primate (NHP), such as but not limited to, a rhesus macaque (Macaca mulatta). In some embodiments, delivery of the siRNA or AAV particles of the present disclosure may be used to inhibit or suppress expression of MAPT transcript in non-human animals, such as but not limited to, htau mice, P301S mice, and/or NHPs.

### V. DEFINITIONS

Unless stated otherwise, the following terms and phrases have the meanings described below. The definitions are not meant to be limiting in nature and serve to provide a clearer understanding of certain aspects of the present disclosure.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” and “consisting essentially thereof” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Adeno-associated virus (AAV): As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to an AAV capsid, e.g., an AAV capsid variant, and a polynucleotide, e.g., a viral genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Antisense strand: As used herein, the term “antisense strand” or “the first strand” or “the guide strand” of a siRNA molecule refers to a strand that is substantially complementary to a target sequence. In some embodiments, the antisense strand is substantially complementary to a section of about 10-50 nucleotides, e.g., about 15-30, 16-25, 18-23 or 19-22 nucleotides of the mRNA of the gene targeted for silencing. In some embodiments, the antisense strand is sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. When referring to a measurable value such as an amount, a temporal duration, and the like, the term is meant to encompass is meant to encompass variations of ±20% or in some instances±10%, or in some instances±5%, or in some instances ±1%, or in some instances±0.1% from the specified value, as such variations are appropriate to perform the disclosed methods.

Capsid: As used herein, the term “capsid” refers to the exterior, e.g., a protein shell, of a virus particle, e.g., an AAV particle, that is substantially (e.g., >50%, >90%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. The term “complementary” as used herein can encompass fully complementary, partially complementary, or substantially complementary. As used herein, the term “substantially complementary” means that the siRNA has a sequence (e.g., in the antisense strand) which is sufficient to bind the desired target mRNA, and to trigger the RNA silencing of the target mRNA. “Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isolcucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95% or less. For example, gaps or discontinuities may be present in the capsid so long as the viral genome is retained in the capsid, e.g., prior to entry into a cell.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats AD, an effective amount of an agent is, for example, an amount sufficient to achieve treatment, as defined herein, of AD, as compared to the response obtained without administration of the agent.

Expression: As used herein, “expression” of a nucleic acid sequence refers to production of an RNA template from a DNA sequence (e.g., by transcription). In some embodiments, expression further comprises one or more of: (1) processing of an RNA transcript (e.g., by splicing, editing, 5′ cap formation, and/or 3′ end processing); (2) translation of an RNA into a polypeptide or protein; and (3) post-translational modification of a polypeptide or protein.

Fragment: A “fragment,” as used herein, refers to a portion. For example, an antibody fragment may comprise a CDR, or a heavy chain variable region, or a scFv, etc. In some embodiments, a fragment is a nucleic acid fragment.

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Identity: As used herein, “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are identical at that position. The identity between two sequences is a direct function of the number of matching positions; e.g., if half (e.g., five positions in a polymer ten subunits in length) of the positions in two sequences are identical, the two sequences are 50% identical; if 90% of the positions (e.g., 9 of 10), are matched, the two sequences are 90% identical. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Isolated: As used herein, the term “isolated” refers to a substance or entity that is altered or removed from the natural state, e.g., altered or removed from at least some of the components with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant or may be incorporated into a vector.

MicroRNA (miRNA) binding site: As used herein, a “miR binding site” comprises a nucleic acid sequence (whether RNA or DNA, e.g., differ by “U” of RNA or “T” in DNA) that is capable of binding, or binds, in whole or in part to a microRNA (miR), e.g., through complete or partial hybridization. Typically, such binding occurs between the miR and the miR binding site in the reverse complement orientation. In some embodiments, the miR binding site is transcribed from the AAV viral genome encoding the miR binding site.

In some embodiments, a miR binding site may be encoded or transcribed in series. Such a “miR binding site series” or “miR BSs” may include two or more miR binding sites having the same or different nucleic acid sequence.

Spacer: As used here, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

Modulatory polynucleotide: As used herein, a “modulatory polynucleotide” is any nucleic acid sequence(s) which functions to modulate (either increase or decrease) the level or amount of a target gene expression, transcript expression and/or protein production.

Neurological disease: As used herein, a “neurological disease” is any disease associated with the central or peripheral nervous system and components thereof (e.g., neurons).

Nucleic Acid: “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994))

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA/DNA: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. In some embodiments, DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively).

RNA interfering: As used herein, the term “RNA interfering” or “RNAi” refers to a sequence specific regulatory mechanism that mediates cleavage of an RNA transcript, e.g., via an RNA-induced silencing complex (RISC) pathway. In some embodiments RNAi is mediated by RNA molecules which results in the inhibition or interfering or “silencing” of the expression of a corresponding protein-coding gene. In some embodiments, RNAi is controlled by the RNA-induced silencing complex (RISC) and is initiated by short/small dsRNA molecules in cell cytoplasm, where they interact with the catalytic RISC component argonaute. In some embodiments, dsRNA molecules can be introduced into cells exogenously. In some embodiments, exogenous dsRNA initiates RNAi by activating the ribonuclease protein Dicer, which binds and cleaves dsRNAs to produce double-stranded fragments of 21-25 base pairs with a few unpaired overhang bases on each end, e.g., small interfering RNAs (siRNAs).

Sample: As used herein, the term “sample” or “biological sample” refers to a subset of its tissues, cells, nucleic acids, or component parts (e.g., body fluids, including but not limited to blood, serum, mucus, lymphatic fluid, synovial fluid, cerebrospinal fluid, saliva, amniotic fluid, amniotic cord blood, urine, vaginal fluid, and semen).

Self-complementary viral particle: As used herein, a “self-complementary viral particle” is a particle included of at least two components, a protein capsid and a polynucleotide sequence encoding a self-complementary genome enclosed within the capsid.

Sense Strand: As used herein, the term “sense strand” or “second strand” or “passenger strand” or “sense sequence” of a siRNA molecule refers to a strand that is complementary to the antisense strand. In some embodiments, the antisense and sense strands of a siRNA molecule are hybridized to form a duplex structure, e.g., an siRNA.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule includes between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates such as chimpanzees and other apes and monkey species, and humans) and/or plants.

Substantially Complementary: As used herein, the term “substantially complementary” means that the siRNA has a sequence (e.g., in the antisense strand) which is sufficient to bind the desired target mRNA, and to trigger the RNA silencing of the target mRNA.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Tauopathy: As used herein, tauopathies and/or tau-associated diseases, refers to a heterogeneous group of neurodegenerative diseases characterized by the dysfunction and/or aggregation of the microtubule-associated protein tau (MAPT). MAPT may also be referred to as “Tau” herein.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Variant: As used herein, the term “variant” refers to a polypeptide or polynucleotide that has an amino acid or a nucleotide sequence that is substantially identical, e.g., having at least 70%, 75%, 80%, 85%, 90%, 95%, or 99% sequence identity to a reference sequence. In some embodiments, the variant is a functional variant.

Functional Variant: As used herein, the term “functional variant” refers to a polypeptide variant or a polynucleotide variant that has at least one activity of the reference sequence.

Vector: As used herein, the term “vector” refers to any molecule or moiety which transports, transduces, or otherwise acts as a carrier of a heterologous molecule. In some embodiments, vectors may be plasmids. Vectors of the present disclosure may be produced recombinantly. The heterologous molecule may be a polynucleotide and/or a polypeptide.

Viral Genome: As used herein, the term “viral genome” refers to the nucleic acid sequence(s) encapsulated in an AAV particle. A viral genome comprises a nucleic acid sequence with at least one payload region encoding a payload and at least one ITR.

The present disclosure is further illustrated by the following non-limiting examples.

### VI. EXAMPLES

### Example 1. Design and Synthesis of siRNA Sequences Targeting the Human MAPT Transcript

### Evaluation of MAPT Transcripts

siRNAs (19mers and 21mers) were generated from human microtubule associated protein tau (MAPT), transcript variant 4, NCBI RefSeq transcript sequence NM_016841.4 (SEQ ID NO: 3947). Subsequent analysis included: evaluation of full match (FM) 19mer and 17mer (without considering positions 1 and 19), and of 19mer and 17mer with single mismatch (SMM) for determination of cross-creativity with human (Hu), rhesus (Rh) and cynomolgus (Cyno) monkey, rat (Rt), and mouse (Ms) transcript (including miRNA seed); evaluation of predicted specificity in human, mouse, rat, and non-human primate (NHP), including separate analysis of sense and antisense strands; and, analysis of a human single nucleotide polymorphism (SNP) database (NCBI-DB-SNP) to identify siRNA targeting regions with known SNPs, including information of positions of SNPs within the target, as well as minor allele frequency (MAF) in available case data. A subset of siRNAs was selected for synthesis.

Cross-reactivity determination analysis was based on Human MAPT, transcript variant 4, NCBI RefSeq transcript sequence NM_016841.4 (SEQ ID NO: 3947). Analysis of siRNA cross-reactivity was performed for transcript variants and different species; for 19mers and 17mers with a full match (FM), and for 19mers and 17mers with a single mismatch (SMM).

An alignment was created by performing a BLASTn search with Human MAPT, transcript variant 4, NCBI RefSeq transcript sequence NM_016841.4 (SEQ ID NO: 3947) versus human, NHP (cynomolgus and rhesus), rat and mouse MAPT protein-coding transcript sequences. Table 9 provides information for MAPT transcript sequences corresponding to each species. Transcripts used for cross-reactivity determination analyses were limited to those longer than 5,000 bp. Sequences are identified in the Table 19 using NCBI RefSeq Accession and/or Ensembl ID identifier codes.

TABLE 19
MAPT transcripts

| | | 19mer cross-reactive | 19mer in | |
|---|---|---|---|---|
| Species | Transcript No. (Accession/ID) | with NM_016841.4 | common | Type |
| Hu | NM_001123066.3 | 5401 | 5120 | mRNA |
| Hu | NM_001123067.3 | 5448 | 5120 | mRNA |
| Hu | NM_001203251.1 | 5461 | 5120 | mRNA |
| Hu | NM_001203252.1 | 5460 | 5120 | mRNA |
| Hu | NM_005910.5 | 5447 | 5120 | mRNA |
| Hu | NM_016834.4 | 5463 | 5120 | mRNA |
| Hu | NM_016835.4 | 5418 | 5120 | mRNA |
| Hu | NM_016841.4 | 5476 | 5120 | mRNA |
| Hu | XM_005257362.4 | 5198 | 5120 | mRNA |
| Hu | XM_005257364.4 | 5119 | 5120 | mRNA |
| Hu | XM_005257365.4 | 5211 | 5120 | mRNA |
| Hu | XM_005257366.3 | 5256 | 5120 | mRNA |
| Hu | XM_005257367.4 | 5212 | 5120 | mRNA |
| Hu | XM_005257368.4 | 5225 | 5120 | mRNA |
| Hu | XM_005257369.4 | 5213 | 5120 | mRNA |
| Hu | XM_005257370.4 | 5278 | 5120 | mRNA |
| Hu | XM_005257371.4 | 5229 | 5120 | mRNA |
| Hu | ENST00000446361 | 5171 | 5120 | known PRT coding |
| Hu | ENST00000262410 | 5418 | 5120 | known PRT coding |
| Hu | ENST00000351559 | 5447 | 5120 | known PRT coding |
| Hu | ENST00000344290 | 5401 | 5120 | known PRT coding |
| Hu | ENST00000535772 | 5460 | 5120 | known PRT coding |
| Hu | ENST00000340799 | 5448 | 5120 | known PRT coding |
| Hu | ENST00000571311 | 289 | — | known PRT coding |
| Hu | ENST00000570299 | 856 | — | known PRT coding |
| Hu | ENST00000334239 | 1089 | — | known PRT coding |
| Hu | ENST00000420682 | 1013 | — | known PRT coding |
| Hu | ENST00000415613 | 966 | — | known PRT coding |
| Hu | ENST00000571987 | 983 | — | known PRT coding |
| Hu | ENST00000574436 | 1012 | — | known PRT coding |
| Hu | ENST00000431008 | 1025 | — | known PRT coding |
| Hu | ENST00000576518 | 1145 | — | known PRT coding |
| Hu | ENST00000572440 | 50 | — | known retained |
| Hu | ENST00000576238 | 0 | — | known retained |
| Hu | ENST00000577017 | 38 | — | known processed |
| Rh | XM_015119945.1 | 2184 | 2175 | mRNA |
| Rh | XM_015119946.1 | 2198 | 2175 | mRNA |
| Rh | XM_015119947.1 | 2193 | 2175 | mRNA |
| Rh | XM_015119948.1 | 2260 | 2175 | mRNA |
| Rh | XM_015119949.1 | 2207 | 2175 | mRNA |
| Rh | XM_015119950.1 | 2181 | 2175 | mRNA |
| Rh | XM_015119952.1 | 2185 | 2175 | mRNA |
| Rh | XM_015119953.1 | 2188 | 2175 | mRNA |
| Rh | XM_015119954.1 | 2286 | 2175 | mRNA |
| Rh | XM_015119955.1 | 2286 | 2175 | mRNA |
| Rh | XM_015119956.1 | 2299 | 2175 | mRNA |
| Rh | XM_015119957.1 | 2290 | 2175 | mRNA |
| Rh | XM_015119958.1 | 2299 | 2175 | mRNA |
| Rh | XM_015119959.1 | 2303 | 2175 | mRNA |
| Rh | XM_015119960.1 | 2166 | 2175 | mRNA |
| Rh | ENSMMUT00000068911 | 2184 | 2175 | novel PRT coding |
| Rh | ENSMMUT00000005862 | 2032 | 2175 | novel PRT coding |
| Rh | ENSMMUT00000005853 | 496 | — | known PRT coding |
| Rh | ENSMMUT00000001151 | 500 | — | known PRT coding |
| Rh | ENSMMUT00000005859 | 557 | — | known PRT coding |
| Rh | ENSMMUT00000005855 | 527 | — | known PRT coding |
| Cyno | NM_001123066.3* | 2105 | 2223 | mRNA |
| Cyno | NM_001123067.3* | 2148 | 2223 | mRNA |
| Cyno | NM_005910.5* | 2148 | 2223 | mRNA |
| Cyno | NM_016834.4* | 2152 | 2223 | mRNA |
| Cyno | NM_016835.4* | 2122 | 2223 | mRNA |
| Cyno | NM_016841.4* | 2165 | 2223 | mRNA |
| Cyno | XM_005584531.2 | 2178 | 2223 | mRNA |
| Cyno | XM_005584536.1 | 2227 | 2223 | mRNA |
| Cyno | XM_005584537.1 | 2244 | 2223 | mRNA |
| Cyno | XM_005584538.2 | 2178 | 2223 | mRNA |
| Cyno | XM_005584539.2 | 2182 | 2223 | mRNA |
| Cyno | XM_005584540.1 | 2270 | 2223 | mRNA |
| Cyno | XM_005584541.2 | 2270 | 2223 | mRNA |
| Cyno | XM_005584542.1 | 2283 | 2223 | mRNA |
| Cyno | XM_005584543.2 | 2274 | 2223 | mRNA |
| Cyno | XM_005584544.2 | 2283 | 2223 | mRNA |
| Cyno | XM_005584545.2 | 2287 | 2223 | mRNA |
| Cyno | XM_015437937.1 | 2195 | 2223 | mRNA |
| Cyno | XM_015437938.1 | 2191 | 2223 | mRNA |
| Cyno | XM_015437939.1 | 2208 | 2223 | mRNA |
| Cyno | XM_015437940.1 | 2189 | 2223 | mRNA |
| Cyno | XM_015437941.1 | 2206 | 2223 | mRNA |
| Cyno | XM_015437942.1 | 2219 | 2223 | mRNA |
| Cyno | XM_015437943.1 | 2193 | 2223 | mRNA |
| Rt | NM_017212.2 | 179 | 225 | mRNA |
| Rt | XM_008768268.2 | 177 | 225 | mRNA |
| Rt | XM_008768269.2 | 177 | 225 | mRNA |
| Rt | XM_008768270.2 | 179 | 225 | mRNA |
| Rt | XM_008768271.2 | 178 | 225 | mRNA |
| Rt | XM_008768272.2 | 177 | 225 | mRNA |
| Rt | XM_008768273.2 | 179 | 225 | mRNA |
| Rt | XM_008768277.2 | 176 | 225 | mRNA |
| Rt | XM_008768278.2 | 178 | 225 | mRNA |
| Rt | XM_008768279.2 | 178 | 225 | mRNA |
| Rt | XM_008768280.2 | 180 | 225 | mRNA |
| Rt | XM_008768281.2 | 181 | 225 | mRNA |
| Rt | XM_008768282.2 | 177 | 225 | mRNA |
| Rt | XM_017597175.1 | 176 | 225 | mRNA |
| Rt | ENSRNOT00000006947 | 179 | 225 | known PRT coding |
| Rt | ENSRNOT00000045127 | 71 | — | known PRT coding |
| Rt | ENSRNOT00000092428 | 0 | — | known PRT coding |
| Rt | ENSRNOT00000050070 | 66 | — | known PRT coding |
| Rt | ENSRNOT00000092483 | 5 | — | known processed |
| Rt | ENSRNOT00000042984 | 51 | — | known PRT coding |
| Ms | NM_001038609.2 | 213 | 218 | mRNA |
| Ms | NM_001285454.1 | 181 | 218 | mRNA |
| Ms | NM_001285455.1 | 213 | 218 | mRNA |
| Ms | NM_001285456.1 | 215 | 218 | mRNA |
| Ms | NM_010838.4 | 213 | 218 | mRNA |
| Ms | XM_006532407.3 | 213 | 218 | mRNA |
| Ms | XM_006532408.3 | 215 | 218 | mRNA |
| Ms | XM_006532409.2 | 215 | 218 | mRNA |
| Ms | XM_006532410.3 | 213 | 218 | mRNA |
| Ms | XM_017314315.1 | 207 | 218 | mRNA |
| Ms | ENSMUST00000106992 | 213 | 218 | known PRT coding |
| Ms | ENSMUST00000106988 | 69 | — | known PRT coding |
| Ms | ENSMUST00000106989 | 69 | — | known PRT coding |
| Ms | ENSMUST00000146353 | 0 | — | known PRT coding |
| Ms | ENSMUST00000145227 | 8 | — | known PRT coding |
| Ms | ENSMUST00000106993 | 100 | — | known PRT coding |
| Ms | ENSMUST00000100347 | 75 | — | known PRT coding |
| Ms | ENSMUST00000138384 | 8 | — | known PRT coding |
| Ms | ENSMUST00000144836 | 48 | — | known PRT coding |
| Ms | ENSMUST00000126820 | 46 | — | known PRT coding |
| Ms | ENSMUST00000132245 | 0 | — | known PRT coding |
| Ms | ENSMUST00000132977 | 0 | — | known PRT coding |

Without considering specificity prediction, 1891-3581 siRNAs were cross-reactive with human and NHP. For siRNAs predicted to be specific in human, 666-1382 siRNAs were cross-reactive with human and NHP. For siRNAs predicted to be specific in human and NHP, 517-1042 siRNAs were cross-reactive with human and NHP.

Evaluation of siRNA Candidates

siRNA off-target genes were predicted for human, rhesus monkey, cynomolgus monkey, mouse, rat, and dog. Off-target frequency was calculated as the number of predicted off-targets (genes) with 0, 1, 2, 3, or 4 mismatches relative to positions 2-18 of an siRNA, where 0 indicated no predicted off-target identified with the indicated number of mismatches. Criterion for off-target frequency was less than or equal to 20 (optimally less than or equal to 10) off-targets with 2 mismatches to the antisense strand. In general, the off-target frequency was used for refined ranking within siRNA candidate sets.

A specificity score was assigned to each siRNA strand. The specificity score accounted for the likelihood of unintended downregulation of any other transcript by full or partial complementarity of a siRNA strand (up to 4 mismatches within positions 2-18), and was based on the number and position of mismatches. Thus, the specificity score described the predicted most likely off-target(s) for all predicted off-targets corresponding to antisense and sense strands of each siRNA; where 0 indicated a perfect match. Criterion for the antisense and sense strand was a specificity score greater than or equal to 2 and greater than or equal to 1, respectively.

siRNA strands were analyzed for the presence of human, rhesus monkey, rat, mouse, rabbit, dog, and/or pig microRNA (miRNA) seed regions. siRNAs can function in a miRNA-like manner via base-pairing with complementary sequences within the 3′-UTR of mRNA molecules. The complementarity typically encompasses the 5′-bases 2-7 of the miRNA, which corresponds to the seed region. To circumvent siRNA action at functional miRNA binding sites, siRNA strands containing natural miRNA seed regions were excluded. Seed regions identified in miRNAs from human, rhesus monkey, dog, pig, mouse, and rat were classified as conserved. Mature miRNAs for Homo sapiens (2588), Macaca mulatta (914), Mus Musculus (1915), Rattus norvegicus (765), Canis familiaris (453), and Sus scrofa (411) were used for miRNA seed region conservation analyses. Oryctolagus cuniculus miRNAs were excluded as identified mature miRNAs (886) were not validated in depth.

Specificity categories were assigned to siRNAs (antisense [AS] and sense strand [SS], independently of each other) based on combined specificity score and miRNA seed analysis. For specificity category, lower numbers were selected, as 1 represented high specificity. Requisite specificity categories were AS1 or AS2, and SS1, SS2 or SS3

For assessment of SNPs, the total number of human MAPT SNPs described was 433. MAFs were assigned to 158 SNPs and 35 SNPs had a MAF greater than or equal to 1% in target sites (range, 0.0106-0.2025%).

Among the 19mers, 1891 human and NHP (cynomolgus) cross-reactive siRNAs were identified, of which 517 siRNAs were specific in human and NHP, and 19 siRNAs were highly specific in human. Among the 17mers, 2077 human and NHP cross-reactive siRNAs were identified, of which 564 siRNAs were specific in human and NHP, and 20 siRNAs were highly specific in human. The siRNAs were further filtered and sub-sorted by factors such as presence of human miRNA seeds, presence of rhesus monkey miRNA seeds, presence of conserved (human, mouse, rat) miRNA seeds, off-target frequency value, presence of SNPs with minor allele frequency (MAF) greater than or equal to 1% in target sites, and, predicted siRNA activity

siRNA Selection

Together, siRNAs were selected on the basis of several factors, including: cross-reactivity (with 19mer in human MAPT mRNA, and with 17mer/19mer in NHP MAPT mRNA); specificity category for human and NHP; miRNA seeds, which may not be identical to those of a known human miRNA, nor may it be conserved between human, mouse, and rat; off-target frequency (less than or equal to 15 human off-targets matched with 2 mismatches by antisense strand); and, human SNPs.

191 siRNA duplexes predicted to be specific in human, monkey and mouse, including several siRNAs predicted to be active, were selected. The siRNA duplexes and their corresponding sense and antisense strand sequences are provided in Table 4, where the antisense and sense strand sequence comprise dTdT overhangs at the 3′ end of each sequence; and in Table 5 where the sense and antisense strand sequences do not comprise a dTdT overhang. The target sites of these siRNAs were distributed along the human MAPT transcript. Position 1 of the antisense strand was engineered to a U and position 19 of the sense strand was engineered to a C, in order to unpair the duplex at this position.

### Example 2. Initial In Vitro siRNA Screening and Hit Identification

The MAPT siRNA duplexes designed in Example 1 and provided in Table 4 and 5, were screened for inhibition of tau (MAPT) expression (e.g., mRNA inhibition) in HEK293 cells at a duple concentration of either 0.1 nM or 10 nM. The 191 siRNA duplexes were designed to target the coding region of MAPT or the 3′ UTR region.

The percent MAPT mRNA remaining relative to control in the HEK293 cells post-treatment with the 191 siRNA duplexes at the 0.1 nM and 10 nM concentrations is provided in Table 20. As shown in Table 20, approximately 20 siRNA sequences reduced tau mRNA levels by approximately 60% at the 0.1 nM concentration and 80% at the 10 nM concentration. The siRNA duplexes of D-001 (SEQ ID NO: 4143 and 3952 or 4477 and 4687), D-002 (SEQ ID NO: 4144 and 3953 or 4478 and 4688), D-003 (SEQ ID NO: 4145 and 3954 or 4479 and 4689, D-004 (SEQ ID NO: 4146 and 3955 or 4480 and 4690), D-009 (SEQ ID NO: 4151 and 3956 or 4485 and 4691), D-014 (SEQ ID NO: 4156 and 3957 or 4490 and 4692), D-015 (SEQ ID NO: 4157 and 3958 or 4491 and 4693), D-016 (SEQ ID NO: 4158 and 3959 or 4492 and 4694), D-017 (SEQ ID NO: 4159 and 3960 or 4493 and 4695), D-018 (SEQ ID NO: 4160 and 3961 or 4494 and 4696), D-019 (SEQ ID NO: 4161 and 3692 or 4495 and 4697), D-026 (SEQ ID NO: 4168 and 3965 or 4502 and 4700), D-030 (SEQ ID NO: 4172 and 3966 or 4506 and 4701), D-035 (SEQ ID NO: 4177 and 3967 or 4511 and 4702), D-038 (SEQ ID NO: 4180 and 3970 or 4514 and 4705), D-043 (SEQ ID NO: 4185 and 3975 or 4519 and 4710), D-045 (SEQ ID NO: 4187 and 3977 or 4521 and 4712), D-047 (SEQ ID NO: 4189 and 3979 or 4523 and 4714), D-049 (SEQ ID NO: 4191 and 3981 or 4525 and 4716), and D-051 (SEQ ID NO: 4193 and 3983 or 4527 and 4718) resulted in the greatest knockdown of MAPT mRNA (Table 20). The sequences of having the indicated duplex IDs in Table 20 are provided in Table 4. Table 5 includes the corresponding siRNA sequences without the dTdT overhangs.

TABLE 20
Percentage of tau mRNA remaining following transfection of siRNA duplexes

| siRNA | % tau mRNA | % tau mRNA | siRNA | % tau mRNA | % tau mRNA |
|---|---|---|---|---|---|
| Duplex | remaining relative | remaining relative | Duplex | remaining relative | remaining relative |
| ID | to control, 0.1 nM | to control, 10 nM | ID | to control, 0.1 nM | to control, 10 nM |
| D-002 | 26 | 15 | D-071 | 64 | 60 |
| D-019 | 30 | 15 | D-107 | 64 | 60 |
| D-026 | 30 | 15 | D-120 | 64 | 60 |
| D-003 | 30 | 15 | D-208 | 64 | 60 |
| D-017 | 30 | 15 | D-161 | 64 | 60 |
| D-014 | 32 | 16 | D-066 | 64 | 61 |
| D-043 | 33 | 17 | D-143 | 65 | 61 |
| D-004 | 33 | 17 | D-065 | 65 | 61 |
| D-015 | 34 | 17 | D-091 | 65 | 62 |
| D-016 | 34 | 17 | D-101 | 65 | 62 |
| D-030 | 35 | 18 | D-210 | 66 | 62 |
| D-009 | 35 | 18 | D-112 | 66 | 63 |
| D-001 | 35 | 18 | D-138 | 66 | 63 |
| D-045 | 36 | 19 | D-094 | 66 | 63 |
| D-047 | 36 | 19 | D-149 | 66 | 64 |
| D-038 | 36 | 19 | D-083 | 66 | 64 |
| D-051 | 36 | 20 | D-102 | 66 | 64 |
| D-018 | 36 | 20 | D-067 | 66 | 64 |
| D-035 | 38 | 20 | D-132 | 66 | 64 |
| D-049 | 38 | 21 | D-172 | 66 | 65 |
| D-056 | 40 | 21 | D-164 | 67 | 65 |
| D-096 | 40 | 22 | D-139 | 67 | 65 |
| D-069 | 40 | 22 | D-198 | 67 | 65 |
| D-024 | 41 | 23 | D-113 | 67 | 66 |
| D-039 | 41 | 23 | D-140 | 67 | 66 |
| D-040 | 42 | 23 | D-133 | 67 | 66 |
| D-044 | 42 | 23 | D-158 | 67 | 66 |
| D-122 | 43 | 23 | D-068 | 68 | 67 |
| D-085 | 43 | 24 | D-144 | 68 | 67 |
| D-053 | 44 | 25 | D-146 | 69 | 67 |
| D-052 | 44 | 25 | D-199 | 69 | 68 |
| D-041 | 45 | 25 | D-177 | 69 | 68 |
| D-050 | 45 | 26 | D-165 | 70 | 69 |
| D-114 | 46 | 27 | D-180 | 70 | 69 |
| D-042 | 46 | 27 | D-196 | 70 | 69 |
| D-036 | 47 | 27 | D-075 | 70 | 70 |
| D-126 | 48 | 28 | D-203 | 70 | 71 |
| D-048 | 51 | 28 | D-108 | 71 | 71 |
| D-055 | 51 | 28 | D-159 | 71 | 71 |
| D-025 | 52 | 28 | D-191 | 71 | 73 |
| D-097 | 52 | 28 | D-193 | 71 | 73 |
| D-037 | 53 | 29 | D-154 | 71 | 74 |
| D-076 | 53 | 29 | D-088 | 72 | 74 |
| D-104 | 53 | 30 | D-109 | 72 | 74 |
| D-057 | 54 | 33 | D-197 | 72 | 74 |
| D-086 | 54 | 34 | D-202 | 73 | 74 |
| D-061 | 54 | 34 | D-092 | 73 | 75 |
| D-127 | 54 | 34 | D-121 | 74 | 75 |
| D-115 | 54 | 35 | D-173 | 74 | 75 |
| D-046 | 55 | 36 | D-150 | 74 | 75 |
| D-116 | 55 | 38 | D-194 | 74 | 76 |
| D-098 | 55 | 38 | D-155 | 74 | 76 |
| D-054 | 56 | 40 | D-178 | 75 | 76 |
| D-077 | 56 | 41 | D-124 | 75 | 78 |
| D-059 | 56 | 42 | D-186 | 75 | 78 |
| D-128 | 56 | 42 | D-151 | 75 | 78 |
| D-078 | 57 | 42 | D-134 | 75 | 78 |
| D-117 | 57 | 43 | D-166 | 75 | 78 |
| D-099 | 57 | 43 | D-181 | 75 | 79 |
| D-062 | 57 | 44 | D-152 | 75 | 79 |
| D-079 | 57 | 44 | D-141 | 75 | 79 |
| D-168 | 57 | 45 | D-084 | 76 | 79 |
| D-072 | 57 | 45 | D-182 | 76 | 81 |
| D-058 | 57 | 46 | D-183 | 76 | 81 |
| D-070 | 57 | 46 | D-187 | 77 | 82 |
| D-080 | 58 | 46 | D-162 | 77 | 83 |
| D-093 | 58 | 47 | D-142 | 77 | 83 |
| D-060 | 58 | 47 | D-188 | 77 | 84 |
| D-105 | 58 | 48 | D-200 | 77 | 84 |
| D-169 | 59 | 49 | D-095 | 78 | 85 |
| D-129 | 59 | 49 | D-204 | 81 | 85 |
| D-153 | 59 | 50 | D-179 | 81 | 85 |
| D-130 | 59 | 51 | D-103 | 81 | 85 |
| D-110 | 60 | 51 | D-209 | 83 | 85 |
| D-087 | 60 | 51 | D-205 | 84 | 85 |
| D-064 | 60 | 52 | D-201 | 85 | 86 |
| D-100 | 61 | 52 | D-184 | 85 | 88 |
| D-118 | 61 | 52 | D-192 | 86 | 90 |
| D-163 | 61 | 52 | D-135 | 87 | 90 |
| D-081 | 61 | 53 | D-125 | 87 | 90 |
| D-073 | 61 | 53 | D-195 | 88 | 91 |
| D-082 | 61 | 53 | D-189 | 88 | 92 |
| D-145 | 61 | 53 | D-156 | 89 | 92 |
| D-111 | 61 | 54 | D-174 | 90 | 93 |
| D-170 | 62 | 55 | D-190 | 91 | 94 |
| D-089 | 62 | 56 | D-160 | 91 | 94 |
| D-123 | 62 | 56 | D-063 | 92 | 96 |
| D-148 | 62 | 56 | D-207 | 93 | 96 |
| D-119 | 62 | 56 | D-147 | 93 | 97 |
| D-106 | 62 | 56 | D-185 | 97 | 97 |
| D-136 | 63 | 57 | D-157 | 97 | 101 |
| D-137 | 63 | 58 | D-206 | 98 | 101 |
| D-090 | 63 | 59 | D-175 | 100 | 104 |
| D-131 | 63 | 59 | D-167 | 106 | 112 |
| D-074 | 64 | 59 | D-176 | 111 | 124 |
| D-171 | 64 | 59 | | | |

Of the siRNAs designed and provided in Table 4 and tested as shown in Table 20, 10 siRNAs were selected for further evaluation in vitro, which included D-001 (SEQ ID NO: 4143 and 3952 or 4477 and 4687), D-004 (SEQ ID NO: 4146 and 3955 or 4480 and 4690), D-009 (SEQ ID NO: 4151 and 3956 or 4485 and 4691), D-016 (SEQ ID NO: 4158 and 3959 or 4492 and 4694), D-018 (SEQ ID NO: 4160 and 3961 or 4494 and 4696), D-019 (SEQ ID NO: 4161 and 3692 or 4495 and 4697), D-026 (SEQ ID NO: 4168 and 3965 or 4502 and 4700), D-030 (SEQ ID NO: 4172 and 3966 or 4506 and 4701), D-045 (SEQ ID NO: 4187 and 3977 or 4521 and 4712), D-051 (SEQ ID NO: 4193 and 3983 or 4527 and 4718). The IC50 values and maximum inhibition in five different cells lines were determined for each of these 10 siRNAs (Table 25). The five cell lines used were MCF-7 cells (breast cancer cells), HEK293 cells (epithelial like cells from the kidneys), BT-474 cells (cells that exhibit epithelial morphology that was isolated from a solid, invasive ductal carcinoma of the breast cancer), LNCap cells (androgen-sensitive human prostate adenocarcinoma cells derived from the left supraclavicular lymph node metastasis), and SH-SY5Y cells (cloned neuroblastoma cell line). High potency was observed in the BT-474 cells, with IC50 values in the 7-21 μM range and maximum inhibition values between 80-90% (Table 25). Similar data was observed in the HEK293 and LNCap cell lines (Table 25).

TABLE 25
IC50 and Maximum Inhibition of MAPT targeting siRNAs in HEK293 cells,
BT-474 cells, LNCap cells, SH-SY5Y cells, and MCF-7 cells

| | HEK293 Cells | | BT-474 Cells | | LNCap Cells | | MCF-7 Cells | |
|---|---|---|---|---|---|---|---|---|
| | IC50 | Maximum | IC50 | Maximum | IC50 | Maximum | IC50 | Maximum |
| Duplex | (nM) | Inhibition | (nM) | Inhibition | (nM) | Inhibition | (nM) | Inhibition |
| D-026 | 0.026 | 81.7 | 0.011 | 90.7 | 0.005 | 86.3 | 0.063 | 68.6 |
| D-019 | 0.098 | 76.1 | 0.007 | 86.5 | 0.010 | 81.3 | 0.058 | 65.8 |
| D-004 | 0.109 | 80.1 | 0.062 | 86.1 | 0.024 | 84.7 | 1.822 | 58.7 |
| D-016 | 0.023 | 76.2 | 0.035 | 88.0 | 0.016 | 84.8 | 1.072 | 65.6 |
| D-009 | 0.020 | 84.9 | 0.010 | 90.8 | 0.011 | 86.5 | 0.574 | 65.4 |
| D-030 | 0.078 | 74.8 | 0.115 | 83.8 | 0.082 | 80.5 | 124.980 | 51.7 |
| D-001 | 0.038 | 81.8 | 0.021 | 90.7 | 0.010 | 87.4 | 5.528 | 56.2 |
| D-045 | 0.064 | 76.7 | 0.019 | 86.6 | 0.030 | 83.4 | 13.662 | 56.1 |
| D-018 | 0.056 | 81.8 | 0.028 | 85.2 | 0.039 | 84.2 | 0.248 | 61.4 |
| D-051 | 0.144 | 77.7 | 0.040 | 83.7 | 0.025 | 83.6 | 2.676 | 56.8 |

### Example 3. Design and Evaluation of Vectorized miRNAs

Ten of the MAPT siRNA sequences demonstrating high potency (e.g., increased knockdown of MAPT mRNA) based on strong cross-cell activity and diversity as shown in Example 2 and Table 25, were selected for design of miRNA constructs comprising a passenger strand (sense) and a guide strand (antisense) as well as a scaffold comprising a 5′ flanking region, loop region, and a 3′ flanking region. The scaffolds used in these constructs included: (i) a 5′ flanking region of SEQ ID NO: 4354 (DNA) or 4879 (RNA), a loop region of SEQ ID NO: 4362 (DNA) or 4887 (RNA), and a 3′ flanking region of SEQ ID NO: 4373 (DNA) or 4898 (RNA); (ii) a 5′ flanking region of SEQ ID NO: 4354 (DNA) or 4879 (RNA), a loop region of SEQ ID NO: 4363 (DNA) or 4888 (RNA), and a 3′ flanking region of SEQ ID NO: 4373 (DNA) or 4898 (RNA); (iii) a 5′ flanking region of SEQ ID NO: 4354 (DNA) or 4879 (RNA), a loop region of SEQ ID NO: 4362 (DNA) or 4887 (RNA), and a 3′ flanking region of SEQ ID NO: 4374 (DNA) or 4899 (RNA); or (iv) a 5′ flanking region of SEQ ID NO: 4355 (DNA) or 4880 (RNA), a loop region of SEQ ID NO: 4366 (DNA) or 4891 (RNA), and a 3′ flanking region of SEQ ID NO: 4375 (DNA) or 4900 (RNA). The miRNA constructs also referred to herein as modulatory polynucleotides were designed and generated to have the scaffolds, passenger strand, and guide strand as indicated in Tables 9A and 9B above. From 5′ to 3′, these modulatory polynucleotides comprised the indicated 5′ flanking region, the indicated passenger strand, the indicated loop region, the indicated guide strand, and the indicated 3′ flanking region (Table 9A and 9B).

The modulatory polynucleotides VOYTaumiR-127-530 (SEQ ID NO: 4405), VOYTaumiR-127-556 (SEQ ID NO: 4406), VOYTaumiR-127-564 (SEQ ID NO: 4407), VOYTaumiR-127-579 (SEQ ID NO: 4408), and VOYTaumiR-127-586 (SEQ ID NO: 4409) were tested to determine guide and passenger strand activity using the DUAL-GLOR luciferase assay (Promega) in HEK293 cells. These constructs were vectorized in a self-complementary AAV genome under the control of a CBA promoter and transfected into cells with reporter plasmids that comprised the target regions of the guide and passenger strands being investigated arranged in tandem. Approximately 48-hours post transfection, cells were lysed and the Renilla and firefly luciferase activity was measured and relative activity was obtained by normalizing the Renilla luciferase level to the control firefly luciferase level (relative R/F luc activity % over amiR-NTC). A higher luciferase signal indicates low activity and a low luciferase signal indicates increased knockdown and activity. Of the constructs tested with respect to the guide strand, less than 50% normalized R/F luciferase activity was observed for the guide strands tested, with the guide strand of VOYTaumiR-127-530 showing the greatest knockdown and activity. The guide strands of VOYTaumiR-127-556 (SEQ ID NO: 4406), VOYTaumiR-127-564 (SEQ ID NO: 4407), VOYTaumiR-127-579 (SEQ ID NO: 4408), and VOYTaumiR-127-586 (SEQ ID NO: 4409) also all showed high, comparable levels of knockdown and activity. With respect to the passenger strand activity, VOYTaumiR-127-530 (SEQ ID NO: 4405), VOYTaumiR-127-564 (SEQ ID NO: 4407), VOYTaumiR-127-579 (SEQ ID NO: 4408), and VOYTaumiR-127-586 (SEQ ID NO: 4409), all showed low levels of passenger strand activity as indicated by a high normalized R/F luciferase activity. The ability of these modulatory polynucleotides to knockdown MAPT mRNA expression was also investigated. The percentage of MAPT mRNA remaining was quantified by qPCR and normalized to the TBP gene. VOYTaumiR-127-530 (SEQ ID NO: 4405) demonstrated a 92.3% knockdown (7.7% MAPT mRNA remaining), VOYTaumiR-127-556 (SEQ ID NO: 4406) demonstrated a 66% knockdown (34% MAPT mRNA remaining), VOYTaumiR-127-564 (SEQ ID NO: 4407) demonstrated a 90.4% knockdown (9.6% MAPT mRNA remaining), VOYTaumiR-127-579 (SEQ ID NO: 4408) demonstrated an 86.5% knockdown (13.5% MAPT mRNA remaining), and VOYTaumiR-127-586 (SEQ ID NO: 4409) demonstrated a 76.3% knockdown (23.7% MAPT mRNA remaining) of MAPT mRNA. The mRNA knockdown results correlated with the results observed in the guide/passenger strand activity assay.

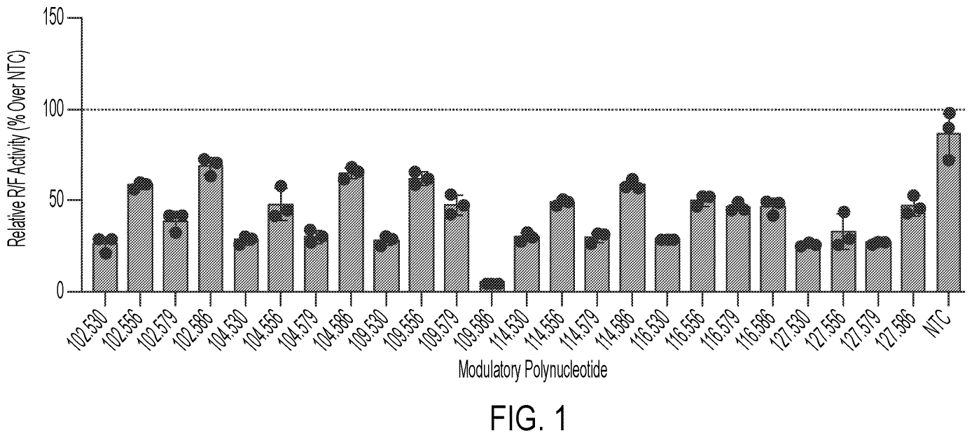

The modulatory polynucleotides VOYTaumiR-102-530 (SEQ ID NO: 4380), VOYTaumiR-102-556 (SEQ ID NO: 4381), VOYTaumiR-102-579 (SEQ ID NO: 4383), VOYTaumiR-102-586 (SEQ ID NO: 4384), VOYTaumiR-104-530 (SEQ ID NO: 4385), VOYTaumiR-104-556 (SEQ ID NO: 4386), VOYTaumiR-104-579 (SEQ ID NO: 4388), VOYTaumiR-104-586 (SEQ ID NO: 4389), VOYTaumiR-109-530 (SEQ ID NO: 4390), VOYTaumiR-109-556 (SEQ ID NO: 4391), VOYTaumiR-109-579 (SEQ ID NO: 4393), VOYTaumiR-109-586 (SEQ ID NO: 4394), VOYTaumiR-114-530 (SEQ ID NO: 4395), VOYTaumiR-114-556 (SEQ ID NO: 4396), VOYTaumiR-114-579 (SEQ ID NO: 4398), VOYTaumiR-114-586 (SEQ ID NO: 4399), VOYTaumiR-116-530 (SEQ ID NO: 4400), VOYTaumiR-116-556 (SEQ ID NO: 4401), VOYTaumiR-116-579 (SEQ ID NO: 4403), VOYTaumiR-116-586 (SEQ ID NO: 4404), VOYTaumiR-127-530 (SEQ ID NO: 4405), VOYTaumiR-127-556 (SEQ ID NO: 4406), VOYTaumiR-127-579 (SEQ ID NO: 4408), and VOYTaumiR-127-586 (SEQ ID NO: 4409) were also tested to determine guide strand activity using the DUAL-GLOR luciferase assay (Promega) in HEK293 cells, as compared to a non-targeting control (NTC) construct. These constructs were vectorized and transfected into cells with reporter plasmids that comprised the target regions of the guide strand being investigated arranged in tandem. Approximately 48-hours post transfection, cells were lysed and the Renilla and firefly luciferase activity was measured and relative activity was obtained by normalizing the Renilla luciferase level to the non-targeting control firefly luciferase level (relative R/F luc activity % over NTC). A higher luciferase signal indicates low activity and a low luciferase signal indicates increased knockdown and activity. As shown in FIG. 1, greater than 75% knockdown was observed for all the guide strands of the vectorized modulatory polynucleotides targeting MAPT tested and no knockdown was observed for the non-targeting control construct.

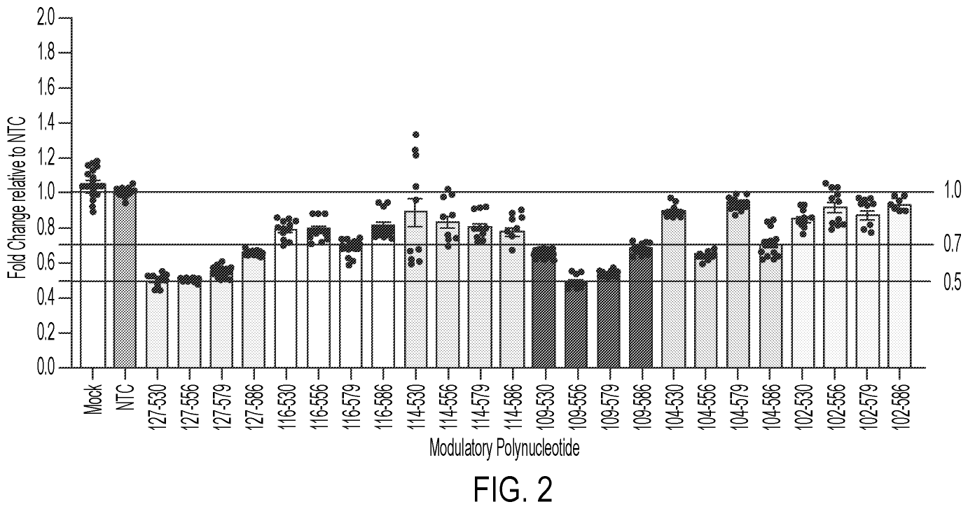

Vectorized modulatory polynucleotides VOYTaumiR-102-530 (SEQ ID NO: 4380), VOYTaumiR-102-556 (SEQ ID NO: 4381), VOYTaumiR-102-579 (SEQ ID NO: 4383), VOYTaumiR-102-586 (SEQ ID NO: 4384), VOYTaumiR-104-530 (SEQ ID NO: 4385), VOYTaumiR-104-556 (SEQ ID NO: 4386), VOYTaumiR-104-579 (SEQ ID NO: 4388), VOYTaumiR-104-586 (SEQ ID NO: 4389), VOYTaumiR-109-530 (SEQ ID NO: 4390), VOYTaumiR-109-556 (SEQ ID NO: 4391), VOYTaumiR-109-579 (SEQ ID NO: 4393), VOYTaumiR-109-586 (SEQ ID NO: 4394), VOYTaumiR-114-530 (SEQ ID NO: 4395), VOYTaumiR-114-556 (SEQ ID NO: 4396), VOYTaumiR-114-579 (SEQ ID NO: 4398), VOYTaumiR-114-586 (SEQ ID NO: 4399), VOYTaumiR-116-530 (SEQ ID NO: 4400), VOYTaumiR-116-556 (SEQ ID NO: 4401), VOYTaumiR-116-579 (SEQ ID NO: 4403), VOYTaumiR-116-586 (SEQ ID NO: 4404), VOYTaumiR-127-530 (SEQ ID NO: 4405), VOYTaumiR-127-556 (SEQ ID NO: 4406), VOYTaumiR-127-579 (SEQ ID NO: 4408), and VOYTaumiR-127-586 (SEQ ID NO: 4409) or a vectorized non-target control were also transfected into HEK293 cells at 1 μg/well to measure knockdown of MAPT mRNA by qPCR at 48 hours post-transfection. The amount of MAPT mRNA remaining was determined and the fold-change in MAPT mRNA remaining for each vectorized modulatory polynucleotide relative to the non-target control (NTC) was calculated as shown on the Y-axis of FIG. 2. A number of modulatory polynucleotides tested lead to a fold change of at least 0.5 in MAPT mRNA remaining relative to the non-target control, including VOYTaumiR-127-530 (SEQ ID NO: 4405), VOYTaumiR-127-556 (SEQ ID NO: 4406), VOYTaumiR-109-556 (SEQ ID NO: 4391), and VOYTaumiR-109-579 (SEQ ID NO: 4393) (FIG. 2). Several modulatory polynucleotides led to a fold change of at least 0.7 in MAPT mRNA remaining relative to the non-target control, including VOYTaumiR-127-579 (SEQ ID NO: 4408), VOYTaumiR-127-586 (SEQ ID NO: 4409), VOYTaumiR-116-556 (SEQ ID NO: 4401), VOYTaumiR-109-530 (SEQ ID NO: 4390), VOYTaumiR-109-586 (SEQ ID NO: 4394), VOYTaumiR-104-556 (SEQ ID NO: 4386), and VOYTaumiR-104-586 (SEQ ID NO: 4389) (FIG. 2).

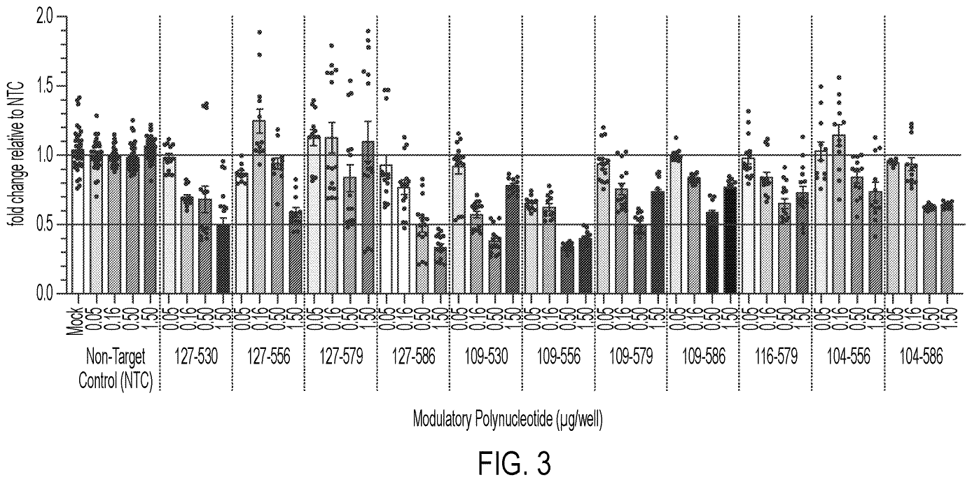

Several of the modulatory polynucleotides that demonstrated a fold change of at least 0.5-0.7 in MAPT mRNA remaining relative to the non-target control as shown in FIG. 2 were selected for subsequent testing in a dose-response study, including VOYTaumiR-127-530 (SEQ ID NO: 4405), VOYTaumiR-127-556 (SEQ ID NO: 4406), VOYTaumiR-127-579 (SEQ ID NO: 4408), VOYTaumiR-127-586 (SEQ ID NO: 4409), VOYTaumiR-109-530 (SEQ ID NO: 4390), VOYTaumiR-109-556 (SEQ ID NO: 4391), VOYTaumiR-109-579 (SEQ ID NO: 4393), VOYTaumiR-109-586 (SEQ ID NO: 4394), VOYTaumiR-116-579 (SEQ ID NO: 4403), VOYTaumiR-104-556 (SEQ ID NO: 4386), and VOYTaumiR-104-586 (SEQ ID NO: 4389). These modulatory polynucleotides or a non-target control construct were transfected into HEK293 cells at a concentration of 0.05 μg per well, 0.16 μg per well, 0.5 μg per well, or 1.5 μg per well. Cells were harvested at 48 hours post-transfection, and the MAPT mRNA levels remaining were determined. The fold change in MAPT mRNA levels remaining post-transfection with the vectorized modulatory polynucleotides relative to the non-targeting control (NTC) was calculated (FIG. 3). As provided in FIG. 3, a strong dose response in MAPT mRNA inhibition was observed for at least VOYTaumiR-127-530 (SEQ ID NO: 4405), VOYTaumiR-127-586 (SEQ ID NO: 4409), VOYTaumiR-109-530 (SEQ ID NO: 4390), VOYTaumiR-109-556 (SEQ ID NO: 4391), VOYTaumiR-109-579 (SEQ ID NO: 4393), and VOYTaumiR-109-586 (SEQ ID NO: 4394).

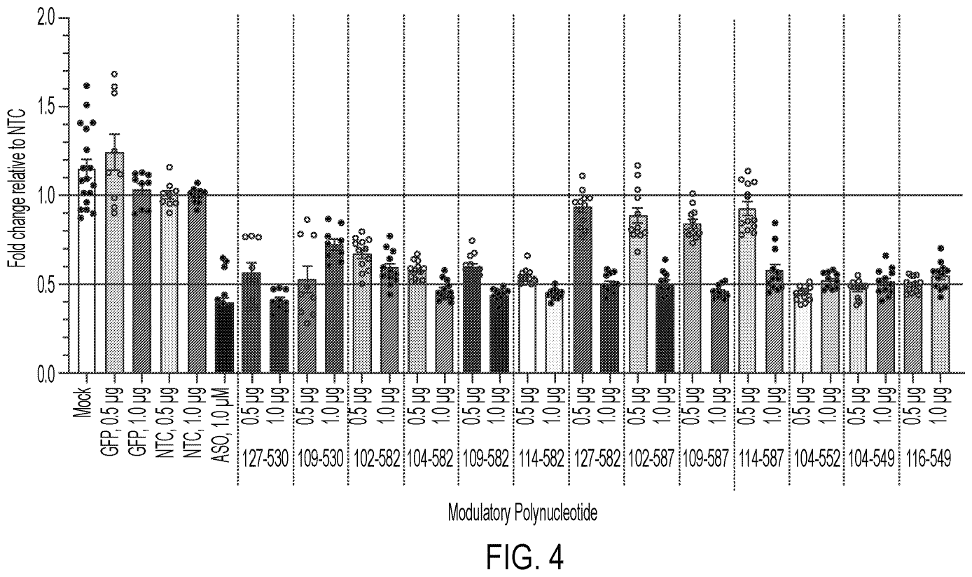

The ability of additional modulatory polynucleotides to inhibit MAPT mRNA was investigated in HEK293 cells, including VOYTaumiR-102-582 (SEQ ID NO: 5027), VOYTaumiR-104-582 (SEQ ID NO: 5031), VOYTaumiR-109-582 (SEQ ID NO: 5035), VOYTaumiR-114-582 (SEQ ID NO: 5039), VOYTaumiR-127-582 (SEQ ID NO: 5047), VOYTaumiR-102-587 (SEQ ID NO: 5028), VOYTaumiR-109-587 (SEQ ID NO: 5036), VOYTaumiR-114-587 (SEQ ID NO: 5040), VOYTaumiR-104-552 (SEQ ID NO: 5033), VOYTaumiR-104-549 (SEQ ID NO: 5034), and VOYTaumiR-116-549 (SEQ ID NO: 5046). These constructs were vectorized and transfected into cells at a concentration of 0.50 μg per well or 1 μg per well. VOYTaumiR-127-530 (SEQ ID NO: 4405), VOYTaumiR-109-530 (SEQ ID NO: 4390), and an antisense oligonucleotide (ASO) targeting MAPT were included as positive controls, and a plasmid encoding GFP or a non-targeting control (NTC) were included as negative controls (FIG. 4). Cells were harvested at 48 hours post-transfection, and the MAPT mRNA levels remaining were determined. The fold change in MAPT mRNA levels remaining post-transfection with the vectorized modulatory polynucleotides relative to the non-targeting control was also calculated (FIG. 4). As shown in FIG. 4, the eleven vectorized modulatory polynucleotides targeting MAPT (VOYTaumiR-102-582, VOYTaumiR-104-582, VOYTaumiR-109-582, VOYTaumiR-114-582, VOYTaumiR-127-582, VOYTaumiR-102-587, VOYTaumiR-109-587, VOYTaumiR-114-587, VOYTaumiR-104-552, VOYTaumiR-104-549, and VOYTaumiR-116-549) transfected at 1.0 μg per well, resulted in approximately a 50% knockdown of MAPT mRNA. Even when transfected at 0.5 μg per well, seven of the vectorized modulatory polynucleotides (VOYTaumiR-102-582, VOYTaumiR-104-582, VOYTaumiR-109-582, VOYTaumiR-114-582, VOYTaumiR-127-582, VOYTaumiR-104-552, VOYTaumiR-104-549, and VOYTaumiR-116-549) tested resulted in approximately 40-50% knockdown of MAPT mRNA.

Taken together, these data demonstrate several modulatory polynucleotides for targeting MAPT that demonstrated high knockdown of MAPT mRNA, high guide strand activity, and/or low passenger strand activity that could be used to treat tauopathies, e.g. tauopathies with aberrant expression of MAPT.

### Example 4. Viral Genomes Encoding siRNA Molecules

Modulatory polynucleotides comprising a molecular scaffold and encoding siRNA duplexes (siRNA molecules) comprising guide (antisense) strands targeting the MAPT transcript and passenger (sense) strands, were engineered into single stranded or self-complementary AAV viral genomes.

The single stranded AAV viral genomes in 5′ to 3′ order, comprised a 5′ inverted terminal repeat (ITR) comprising SEQ ID NO: 4469, a CMVie enhancer comprising SEQ ID NO: 4472, a CBA promoter comprising SEQ ID NO: 5199, an hBG intron comprising SEQ ID NO: 4475, a modulatory polynucleotide comprising any one of SEQ ID NOs: 4380-4409, a human growth hormone (hGH) polyadenylation (PolyA) signal sequence comprising SEQ ID NO: 4476, and a 3′ ITR comprising SEQ ID NO: 4470. The single stranded viral genomes comprise the nucleotide sequences of any one of SEQ ID NOs: 4439-4468 and are provided in Table 10A and Tables 11-18.

The self-complementary AAV viral genomes, in 5′ to 3′ order, comprised a 5′ inverted terminal repeat (ITR) comprising SEQ ID NO: 5197, a CMVie enhancer comprising SEQ ID NO: 4171, a CBA promoter comprising SEQ ID NO: 5199, an hBG intron comprising SEQ ID NO: 4475, a modulatory polynucleotide comprising any one of SEQ ID NOs: 4380-4409 or 5027-5050, a human growth hormone (hGH) polyadenylation (PolyA) signal sequence comprising SEQ ID NO: 4476, and a 3′ ITR comprising SEQ ID NO: 5200. The self-complementary viral genomes comprise the nucleotide sequences of any one of SEQ ID NOs: 5149-5196 and are provided in Table 10A, Table 10B, and Table 26.

### Example 5. Tau Knock-Down in In Vivo Animal Studies

### In Vivo Rodent Studies

To evaluate in vivo biological activity of AAV particles comprising vector genomes that encode MAPT siRNA, select AAV particles are delivered to rodents, such as transgenic mice (e.g., htau, P301S, rTg4510, rTgTauEC, SCN1a-A1783V) and non-transgenic (wild type) mice, via ICM, IM, IP, ICV, IPa (e.g., intrathalamic) or IT administration routes at a number of doses within a set dose range. A control group is treated with, e.g., vehicle, which can be PBS. The administration may include one or more injections over a period of time. Following test article administration, behavioral tests that may include the Morris water-, Y-, T- or radial arm-maze, or object recognition or open field tests, are performed at several time intervals across the lifespan. At a predetermined day post-dosing, animals are euthanized, and brain tissue punches (e.g., sampled from frontal and temporal lobes, basal ganglia, cerebellum, parietal lobe and brainstem) are collected and snap frozen. Tissue punches are homogenized, and the total RNA is purified. Relative MAPT expression (mRNA) is determined by quantification methods, e.g. qRT-PCR. MAPT expression is normalized to housekeeping gene expression, and then further normalized to the vehicle control group. Tissue samples, including brain and spinal cord, in addition to other peripheral organs, are also used to quantify tau protein, e.g., using immunohistochemical and western blot approaches.

### In Vivo Htau Mouse Studies

The htau mouse model of tauopathy, the natural history of which has been previously described in detail by Andorfer et al., 2003 (Andorfer et al., J Neurochem. 2003 August; 86(3):582-90, the contents of which are incorporated herein by reference in their entirety), is identified for studies, such as but not limited to, pharmacology and efficacy studies, due to both favorable cognitive and behavioral phenotypes.

The htau mouse transgene contains the coding sequence, intronic regions, and regulatory elements of the human microtubule-associated protein tau (MAPT) gene. Although no endogenous mouse MAPT is detected (the htau mouse was generated on a mouse tau knockout background), all six isoforms (including both 3R and 4R forms) of human MAPT are expressed. Htau mice develop tau pathology in a time course and distribution that is comparable to that occurring in the early stages of human Alzheimer's disease (AD). Hyperphosphorylated MAPT is detected in cell bodies and dendrites by 3 months of age. Accumulation of hyperphosphorylated tau begins by 6 months, but increases further by 13- and 15-months of age. Aggregated tau and PHFs are detectable in htau mice aged 9 months via sarkosyl-insolubility. The majority of tau pathology in htau mice is found in neocortex and hippocampus, while minimal tau pathology is found in brain stem and spinal cord. Consequently, there are no overt gross motor or behavioral disturbances. Tau pre-tangle, such as CP13 (pSer²⁰²) immunoreactivity can be observed in CA1 and CA3 at 4 months of age. At 12 months of age, htau mice display prominent hippocampal CP13 and PHF-1 (pSer³⁹⁶/pSer⁴⁰⁴; described in International Publication WO199620218, the contents of which are herein incorporated in their entirety) immunoreactivity, resembling that observed in human AD at a moderate stage of tau pathology. Htau mice also develop learning and memory deficits in an age-dependent manner. For instance, object recognition memory and spatial memory are impaired in the aged htau mice (12 months of age). These deficits have not been observed in younger (4-month) htau mice.

### Paired Helical Filament (PHF) Seeding

To circumvent an extended drug screening timeline resulting from prolonged disease progression in htau mice, which do not exhibit cognitive deficits until an advanced age (12 months old), sarkosyl insoluble paired helical filament (PHF) tau from Alzheimer's disease (AD) or other tauopathy disease brain or biofluid, is injected into the hippocampus of htau mice (n=10-15 mice), using methods similar to those previously described (see e.g., Hu et al., Alzheimer's Dement. 2016 October; 12(10):1066-1077 and Gerson et al., J Neurotrauma. 2016 Nov. 15; 33(22):2034-2043, the contents of each of which are incorporated herein by reference in their entirety).

The amount of PHF for injection and the time course (4, 8, and 12 weeks) for the development of seeding activity, in other words, the initiation of tau pathology, as well as for the development of cognitive deficits, such as those that may be measured by spatial memory (e.g., Morris water maze) and/or novel object recognition tests, is determined.

Age-matched htau and wild type (control) mice may be used. Aged htau mice (12 months old; n=10-15) are monitored for the onset of cognitive deficits as previously described by Polydoro et al., 2009 and Geiszler et al., 2016 (Polydoro et al., J Neurosci. 2009 Aug. 26; 29(34):10741-9 and Geiszler et al., Neuroscience. 2016 Aug. 4; 329:98-111, the contents of each of which are incorporated herein by reference in their entirety).

The htau PHF seeding model may be used to test whether tau miRNA administration may have a therapeutic effect post-symptomatically. As a nonlimiting example, the seeding may occur prior to miRNA administration.

### Olfactory Bulb Propagation

PHF tau isolated from AD and/or FTLD patients is injected into the olfactory bulb (seeding) in 4-month-old htau female mice. Propagation of tau pathology from olfactory bulb to the first (e.g., piriform cortex, entorhinal cortex) and second (e.g. medial thalamus, hippocampus) synaptic connections are assessed following a given time interval, e.g., 12-16 weeks post-injection. Tau immunoreactivity is measured in the seeding and in the first and secondary synaptic brain areas using anti-tau antibodies, such as but not limited to the AT100 antibody (pSer²¹²/pSer²¹⁴; ThermoFisher, Waltham, MA; described in U.S. Pat. No. 6,121,003, the contents of which are herein incorporated in their entirety). Pilot experiments have been performed using AD isolated PHF. This model may be used to test anti-tau miRNA of the present disclosure in therapeutic setting.

### In Vivo Target Engagement and Potency

Identified miRNAs, such as those described in Example 2, are evaluated in vivo for target engagement. Mice (htau; 10 per dose group) are injected via intra-cisterna magna (ICM) with or without additional direct intraparenchymal (IPa) delivery routes at 2 months of age. Initial doses may include 5×10⁸, 5×10⁹, and/or 5×10¹⁰ vg/mouse for ICM or intra-thalamic injections. Mice may be euthanized at set intervals following dosing, such as but not limited to, 4-, 8- and 12-weeks post-dose. Hippocampus, cortex, thalamus, olfactory bulb, brainstem, striatum, cerebellum, spinal cord and liver are collected, and human tau levels from the transgene are quantified by quantitative reverse transcription polymerase chain reaction (RT-qPCR) and/or branched DNA, enzyme-linked immunosorbent assays (ELISAs) and/or immunohistochemistry (IHC).

Constructs showing >50% decrease in tau mRNA/protein expression are tested in a time-response study. Briefly, htau mice (n=6-10 mice) are injected with an ICM or intra-thalamic injection of tau miRNA constructs, tissue is collected at various time-points (e.g. 4-, 8- and 12-weeks), and human tau mRNA and protein levels are quantified.

Dose and time-point for optimal knock-down/suppression of expression are determined.

### In Vivo Efficacy of AAV Particle Administration

AAV particles showing >50% decrease of human tau protein in brain areas such as hippocampus, cortex, thalamus, brainstem, cerebellum and olfactory bulb are tested in P301S, as described by Allen et al., 2002 (Allen et al., 2002, J Neurosci. 2002 Nov. 1; 22(21):9340-51, the contents of which are incorporated herein by reference in their entirety), and/or htau mice, e.g., htau seeding and/or propagation models, are selected for efficacy studies. Mice (n=8-12) are injected with AAV particles (ICM) with or without direct IPa administration into the thalamus) at various doses and evaluated for multiple efficacy outcomes. Nervous system tissue regions such as hippocampus, cortex, thalamus, olfactory bulb, brainstem, striatum, cerebellum, spinal cord, as well as the liver, are collected, and human tau levels are quantified by RT-qPCR and/or branched DNA, ELISAs and/or IHC. Brain lysate from mice in the treatment and control groups are tested for tau seeding activity using biosensor cells, as described by Frost et al., 2009 (Frost et al., J Biol Chem. 2009 May 8; 284(19):12845-52, the contents of where are incorporated herein by reference in their entirety). Versus the control group, no or minimal tau seeding activity (e.g., tau aggregation) may be observed in brain lysate isolated from treatment groups demonstrating efficacy in decreasing tau pathology.

A comparison of the in vivo efficacy of AAV anti-tau miRNAs versus anti-tau antisense oligonucleotides (ASOs) and/or vectorized anti-tau antibody efficacy is performed. Whether efficacy of AAV anti-tau miRNA treatment is equivalent, superior, or inferior compared to efficacy comparators, e.g., anti-tau ASO and/or vectorized anti-tau antibody, as described above (Example 1), and/or is additive with anti-tau ASO or vectorized anti-tau antibody in combination, is determined. Thus, anti-tau ASOs or vectorized anti-tau antibody will be run in parallel and together with AAV anti-tau miRNAs to provide a head-to-head comparator, as well as to inform whether co-treating provides synergy.

Based on in vivo efficacy data, up to 3 constructs are selected for additional studies. Data obtained in this example will include a minimally efficacious dose (MED) for in vivo efficacy, data related to therapeutic index (efficacious dose and tolerability findings). These data, in combination with target engagement data are used to drive additional experimental design and/or parameters, e.g., dose selection.

### In Vivo Non-Human Primate (NHP) Studies

In non-human primates, AAV particles or vehicle are delivered via IM, IP, ICV, IPa or IT administration routes at a number of doses within a set dose range. The administration may include one or more injections over a period of time. Clinical signs are documented and behavioral tests, e.g. the delayed response task, are performed. At a pre-set day and time following the administration, animals are sacrificed, and select tissues harvested for bioanalytical and histological evaluation. Brain regions included in evaluation may include frontal and temporal lobes, as well as many other brain regions, such as but not limited to, basal ganglia, cerebellum, parietal lobe and brainstem. Hence, entire brain regions would be assessed, including deep structures and cerebellum. Tau protein and mRNA levels are assessed for tau/MAPT suppression following administration of AAV particles comprising vector genomes that encode MAPT siRNA versus vehicle.

### Route of Administration (ROA) and Capsid Evaluation

Multiple routes of administration (ROA) using different capsids will be evaluated for comparison against AAV9 intra-cisterna magna (ICM) and thalamic intraparenchymal (IPa) administration.

Up to 4 different capsids, e.g., VOY101, AAV9, AVrh10, and AAV1 are selected. A single transgene tool construct is used for biodistribution studies. This may include a tool of vectorized anti-tau antibody and/or another well-validated transgene target.

NHP receive ICM and/or intrathalamic IPa injections of transgene tool construct at various doses. At a pre-selected time-point (e.g., 1-2 months post injection), NHPs are euthanized and brain, spinal cord, and peripheral tissues are collected. Tissue punches from multiple brain regions are collected from the left hemisphere and vector genome and anti-tau antibody or frataxin transgene mRNA levels are quantified from the same sample. Additionally, the right hemisphere is utilized for IHC (or in situ hybridization, ISH) analysis of tool construct expression.

### Example 6. Evaluation of Vectorized MAPT siRNAs In Vivo in Mice

This Example investigates the ability of the modulatory polynucleotide VOYTaumiR-127-530 (SEQ ID NO: 4405) and/or VOYTaumiR-127-579 (SEQ ID NO: 4408) to inhibit and reduce expression of MAPT (human tau) in vivo at varying doses. These constructs also demonstrated increased activity in vitro as shown in Example 3.

In a first study, AAV particles were generated with the VOY9P39 capsid (SEQ ID NO: 5147) or the VOY101 (SEQ ID NO: 1) encapsulating the VOYTaumiR-127-530 modulatory polynucleotide (SEQ ID NO: 4405) driven by a CBA promoter encoded by a self-complementary viral genome. The self-complementary viral genome comprised the nucleotide sequence of SEQ ID NO: 5173. The AAV particles comprising the VOY9P39 capsid encoding the VOYTaumiR-127-530 modulatory polynucleotide were intravenously administered to hTau mice (female; 12-13 weeks old, n=6) at increasing doses of 3e12 vg/kg (low), 1e13 vg/kg (mid), or 3e13 vg/kg (high). AAV particles comprising the VOY101 capsid encoding the VOYTaumiR-127-530 modulatory polynucleotide or an mCherry reporter control; or AAV particles comprising the VOY9P39 capsid encoding a non-targeting control or an mCherry reporter control were intravenously administered to hTau mice at a dose of 3e13 vg/kg. A group of mice also received a non treatment vehicle control (n=6). The in-life period was four weeks and brains, livers, spinal cord, serum, and/or CSF were collected from the mice to measure tau knockdown (mRNA and protein levels) and biodistribution. The body weight and cage side observations for all mice throughout the study were normal and no significant changes were observed.

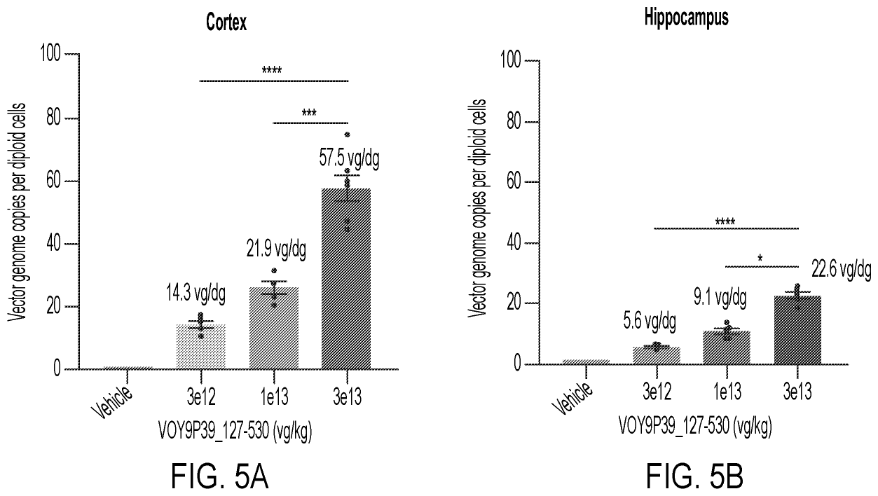

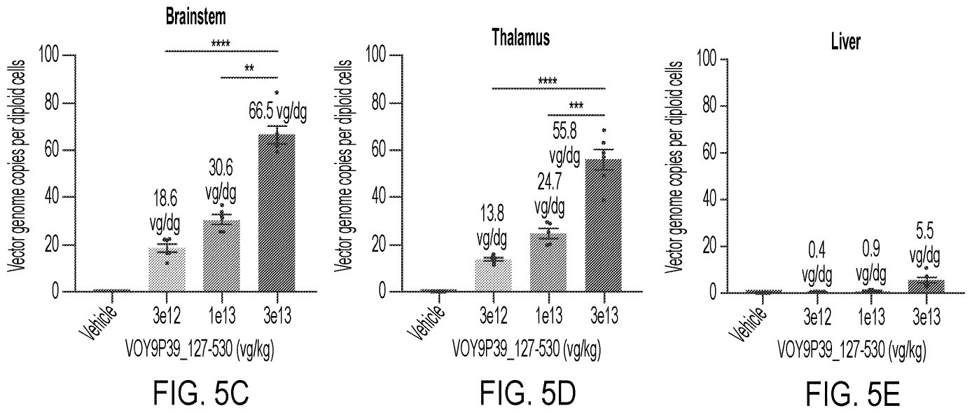

Table 21 and FIGS. 5A-5E, provide the AAV biodistribution observed in the cortex, hippocampus, brainstem, thalamus, and liver following intravenous administration of the AAV particles comprising the VOY9P39 or VOY101 capsid protein encoding the VOYTaumiR-127-530 modulatory polynucleotide, the non-targeting control (NTC), or the mCherry reporter control construct. Good biodistribution was observed in all brain regions investigated. Additionally, a dose dependent increase was observed in the biodistribution (vg copies per diploid cells (vg/dg)) in the cortex, hippocampus, brainstem, and thalamus in the mice that received the increasing doses of the AAV particles comprising the VOY9P39 capsid encoding the VOYTaumiR-127-530 modulatory polynucleotide (Table 21 and FIGS. 5A-5D). Low levels of vg/dg (biodistribution) were observed in the liver of the mice when administered the AAV particles comprising the VOY9P39 capsid (Table 21 and FIG. 5E).

TABLE 21
Biodistribution (vg/dg) in the brain of mice post-intravenous injection of AAV particles
comprising the VOY9P39 or VOY101 capsid protein encoding the VOYTaumiR-127-530 modulatory
polynucleotide, non-target control, or the mCherry reporter control

| | | Dose | VG/DG | | | | |
|---|---|---|---|---|---|---|---|
| Payload | Capsid | (vg/kg) | Cortex | Hippocampus | Brainstem | Thalamus | Liver |
| mCherry Reporter Control | VOY9P39 | 3e13 | 45.5 | 27.1 | 61.2 | 49.6 | 5.6 |
| Non-Target Control | VOY9P39 | 3e13 | 38.4 | 20.7 | 47.7 | 39.6 | 2.2 |
| VOYTaumiR-127-530 | VOY9P39 | 3e12 | 14.3 | 5.6 | 18.6 | 13.8 | 0.4 |
| VOYTaumiR-127-530 | VOY9P39 | 1e13 | 21.9 | 9.1 | 30.6 | 24.7 | 0.9 |
| VOYTaumiR-127-530 | VOY9P39 | 3e13 | 57.5 | 22.6 | 66.5 | 55.8 | 5.5 |
| mCherry Reporter Control | VOY101 | 3e13 | 63.3 | 33.3 | 74.0 | 66.5 | 22.0 |
| VOYTaumiR-127-530 | VOY101 | 3e13 | 46.5 | 28.0 | 59.5 | 48.5 | 27.4 |

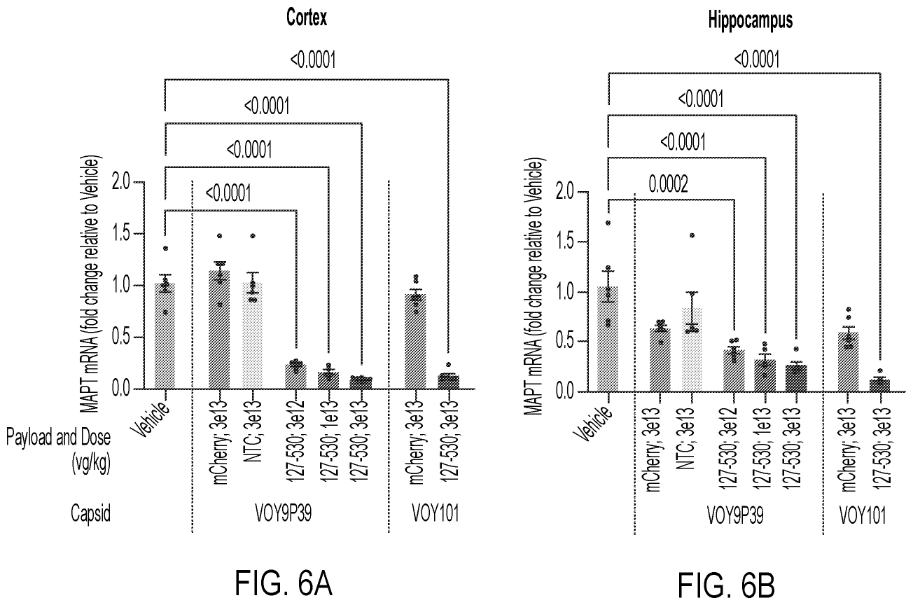

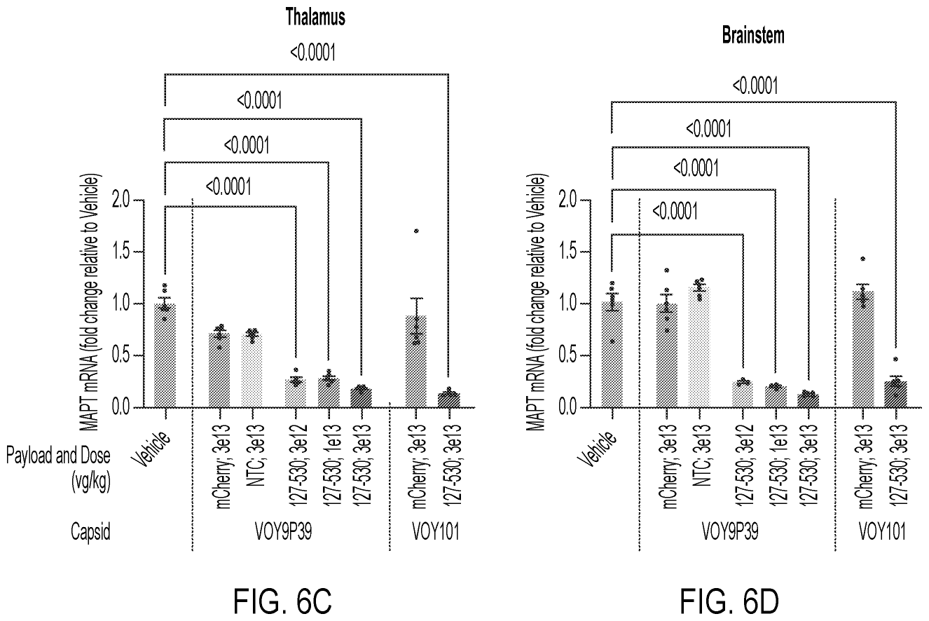

The ability of vectorized VOYTaumiR-127-530 modulatory polynucleotide (SEQ ID NO: 4405) to reduce MAPT mRNA expression was also determined in the cortex, hippocampus, thalamus, and brainstem of mice at 4 weeks post intravenous administration. The amount of MAPT mRNA remaining after treatment was measured by qPCR and normalized to a housekeeping gene (endogenous XPNPEP1) and the fold-change in MAPT mRNA remaining in the groups receiving the increasing doses of the VOYTaumiR-127-530 modulatory polynucleotide relative to the vehicle was determined. As shown in FIGS. 6A-6D, MAPT mRNA was reduced in all mice that received AAV particles encoding the VOYTaumiR-127-530 modulatory polynucleotide, relative to the groups that received the non-targeting control or the mCherry reporter control. Additionally, AAV particles encoding the VOYTaumiR-127-530 modulatory polynucleotide comprising the VOY9P39 capsid protein or the VOY1010 capsid protein led to comparable levels of MAPT mRNA knockdown in the brain tissues investigated (FIGS. 6A-6D). As shown in FIGS. 6A-6D, a trending dose response was observed in MAPT mRNA knockdown in the cortex, hippocampus, thalamus, and brainstem when mice were intravenously administered AAV particles comprising the VOY9P39 capsid and encoding the VOYTaumiR-127-530 modulatory polynucleotide at 3e12 vg/kg, 1e13 vg/kg, or 3e13 vg/kg. The fold-change in MAPT mRNA remaining in the groups receiving the increasing doses of the VOYTaumiR-127-530 modulatory polynucleotide relative to the vehicle or non-target control is shown in Table 22. The percent MAPT knockdown was calculated in the hippocampus, cortex and brainstem and is provided in Table 23. At least a 70% knockdown and up to a 90% knockdown was observed in the brain following treatment with the VOYTaumiR-127-530 modulatory polynucleotide targeting MAPT at the highest dose tested (3e13 vg/kg) (Table 23). Even at the low dose of 3e12 vg/kg, 44-76% knockdown of MAPT mRNA was observed in different regions of the mouse brain four weeks post treatment with vectorized VOYTaumiR-127-530, which targets MAPT (Table 23).

TABLE 22
Fold change in MAPT mRNA levels in the brain of mice post-intravenous
injection of AAV particles encoding the VOYTaumiR-127-530 modulatory
polynucleotide relative to MAPT mRNA levels in the brain of mice receiving
the vehicle control or AAV particles encoding a non-target control

| Payload | Capsid | Dose (vg/kg) | Cortex | Hippocampus | Thalamus | Brainstem |
|---|---|---|---|---|---|---|
| | | | Fold Change relative to Vehicle | | | |
| Vehicle | N/A | N/A | 1.00 | 1.00 | 1.00 | 1.00 |
| VOYTaumiR-127-530 | VOY9P39 | 3e12 | 0.24 | 0.42 | 0.27 | 0.25 |
| VOYTaumiR-127-530 | VOY9P39 | 1e13 | 0.17 | 0.32 | 0.28 | 0.21 |
| VOYTaumiR-127-530 | VOY9P39 | 3e13 | 0.10 | 0.27 | 0.18 | 0.13 |
| | | | Fold Change relative to Non-Target Control | | | |
| Non-Target Control | VOY9P39 | 3c13 | 1.00 | 1.00 | 1.00 | 1.00 |
| VOYTaumiR-127-530 | VOY9P39 | 3e12 | 0.24 | 0.53 | 0.38 | 0.21 |
| VOYTaumiR-127-530 | VOY9P39 | 1e13 | 0.17 | 0.41 | 0.40 | 0.18 |
| VOYTaumiR-127-530 | VOY9P39 | 3e13 | 0.10 | 0.34 | 0.26 | 0.11 |

TABLE 23
Percent knockdown of MAPT mRNA in the brain of mice post-intravenous
injection of AAV particles comprising the VOY9P39 capsid and
encoding the VOYTaumiR-127-530 modulatory polynucleotide

| | | Dose | Percent Knockdown of mRNA levels | | |
|---|---|---|---|---|---|
| Payload | Capsid | (vg/kg) | Cortex | Hippocampus | Brainstem |
| VOYTaumiR-127-530 | VOY9P39 | 3e12 | 76% | 44% | 75% |
| VOYTaumiR-127-530 | VOY9P39 | 1e13 | 84% | 64% | 79% |
| VOYTaumiR-127-530 | VOY9P39 | 3e13 | 90% | 69% | 87% |

A linear regression was performed to determine the relationship between viral genome levels and MAPT mRNA levels remaining relative to the vehicle in the brain regions at 4 weeks post-intravenous treatment with the AAV particles comprising the VOY9P39 capsid and encoding the VOYTaumiR-127-530 modulatory polynucleotide. A negative correlation was observed between the MAPT mRNA levels and the viral genome levels. The R² value in the cortex was 0.7172 (p<0.0001); the R² value in the hippocampus was 0.4143 (p=0.0053); the R² value in the thalamus was 0.4907 (p=0.0017); and the R² value in the brainstem was 0.8667 (p<0.0001).

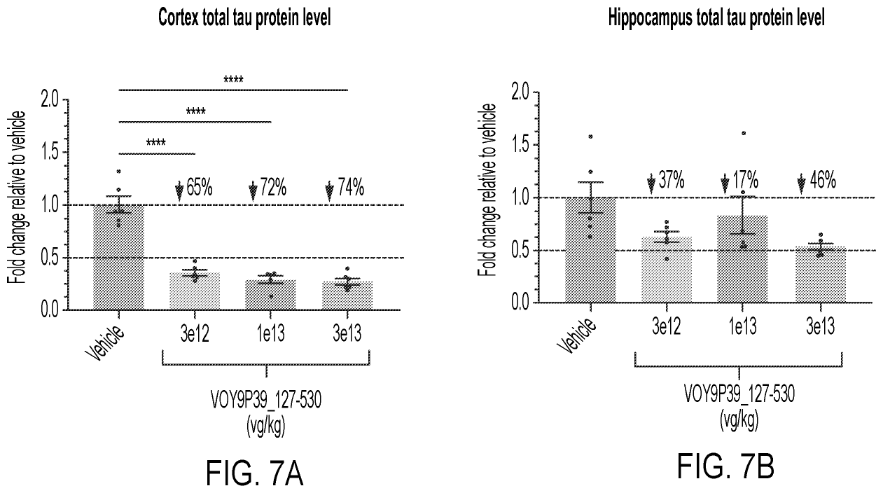

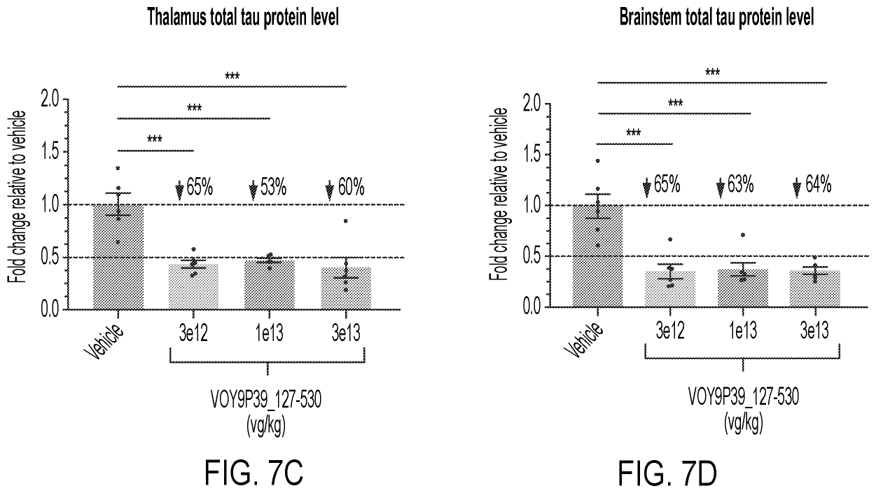

Total tau protein levels in the cortex, thalamus, and brainstem following treatment with the AAV particles comprising the VOY9P39 capsid and encoding the VOYTaumiR-127-530 modulatory polynucleotide were determined by AlphaLISA. As shown in FIGS. 7A-7D and Table 24, knockdown in total tau protein level was observed across all brain regions, with the cortex, thalamus, and brainstem showing significant reductions. This knockdown was specific for total tau protein, as no decrease in beta-actin protein levels were observed in the cortex by Western blot.

TABLE 24
Percent knockdown of MAPT protein and total MAPT protein remaining relative to the vehicle
control in the brain of mice post-intravenous injection of AAV particles comprising
the VOY9P39 capsid and encoding the VOYTaumiR-127-530 modulatory polynucleotide

| Payload | Capsid | Dose (vg/kg) | Cortex | Hippocampus | Thalamus | Brainstem |
|---|---|---|---|---|---|---|
| | | | Percent Knockdown of Protein | | | |
| VOYTaumiR-127-530 | VOY9P39 | 3e12 | 65% | 37% | 65% | 65% |
| VOYTaumiR-127-530 | VOY9P39 | 1e13 | 72% | 17% | 53% | 63% |
| VOYTaumiR-127-530 | VOY9P39 | 3e13 | 74% | 46% | 60% | 64% |
| | | | Tau Protein Levels Remaining relative to Vehicle Control | | | |
| Vehicle | N/A | N/A | 1.0 | 1.0 | 1.0 | 1.0 |
| VOYTaumiR-127-530 | VOY9P39 | 3e12 | 0.35 | 0.63 | 0.43 | 0.36 |
| VOYTaumiR-127-530 | VOY9P39 | 1e13 | 0.29 | 0.83 | 0.47 | 0.38 |
| VOYTaumiR-127-530 | VOY9P39 | 3e13 | 0.27 | 0.54 | 0.40 | 0.36 |

Small RNA processing analysis via high throughput small RNA deep sequencing to measure guide to passenger ratio, abundance of guide and passenger strands relative to the total endogenous pool of miRNAs (saturation level of the endogenous miRNA pathway), and precision of processing at the 5′-end of the guide strand was also performed on RNA isolated from the cortex 4 weeks post-intravenous treatment with the AAV particles comprising the VOY9P39 capsid and encoding the VOYTaumiR-127-530 modulatory polynucleotide. Table 25 shows the level of mature exogenous MAPT targeting polynucleotides relative to the total endogenous pool of miRNAs to ensure a lack of overharnessing of the endogenous miRNA biogenesis pathway, where a lower percentage is more favorable to prevent any effects related to endogenous miRNA pathway saturation. The guide to passenger strand level is also provided in Table 25. A higher guide to passenger strand ratio is more favorable for potency and selectivity and to minimize any passenger strand effects. Table 25 also shows the 5′ end processing of the modulatory polynucleotides evaluated. The 5′ end processing of the modulatory polynucleotides indicates that the constructs are being processed correctly at the 5′ end and a higher value is more favorable. As the seed region is used for recognition of the target mRNA and starts at a fixed position relative to the 5′ end of the guide strand, accurate processing of the 5′ end of the guide strand is needed. Both doses of VOYTaumiR-127-530 tested led to 89-90% in the 5′ end processing.

TABLE 25
Guide to passenger ratio, abundance of guide and passenger strands relative
to the total endogenous pool of miRNAs (% endogenous), and precision of processing
at the 5′-end of the guide strand for the VOYTaumiR-127-530 modulatory polynucleotides
in the cortex 4-weeks post-intravenous injection in mice

| | | Dose | | Guide/Passenger | 5′ Precision |
|---|---|---|---|---|---|
| Payload | Capsid | (vg/kg) | % Endogenous | Ratio | Processing (%) |
| VOYTaumiR-127- | VOY9P39 | 3e12 | 2.79% | 34.59 | 91.77% |
| 530 | | | | | |
| VOYTaumiR-127- | VOY9P39 | 3e13 | 5.67% | 29.60 | 89.16% |
| 530 | | | | | |
| Vehicle | N/A | N/A | 0.005% | N/A | 97.08% |

In a second study, AV particles were generated with the VOY9P39 capsid (SEQ ID NO: 5147) encapsulating the VOYTaumiR-127-530 modulatory polynucleotide (SEQ ID NO: 4405), the VOYTaumiR-127-579 (SEQ ID NO: 4408) modulatory polynucleotide, or a non-target control (NTC) modulatory polynucleotide driven by a CBA promoter encoded by a self-complementary viral genome. The self-complementary viral genome encoding VOYTaumiR-127-530 comprised the nucleotide sequence of SEQ ID NO: 5173; and the self-complementary viral genome encoding VOYTaumiR-127-579 comprised the nucleotide sequence of SEQ ID NO: 5195. The AAV particles comprising the VOY9P39 capsid encoding the VOYTaumiR-127-530 modulatory polynucleotide were intravenously administered to hTau mice (female; 17-18 weeks old, n=6) at a high dose of 1e13 vg/kg or a low dose of 1e12 vg/kg. The AAV particles comprising the VOY9P39 capsid encoding the VOYTaumiR-127-579 modulatory polynucleotide or the non-target control construct were intravenously administered to hTau mice (female; 17-18 weeks old, n=6) at the high dose of 1e13 vg/kg. The in-life period was four weeks and brains were collected from the mice to measure tau knockdown.

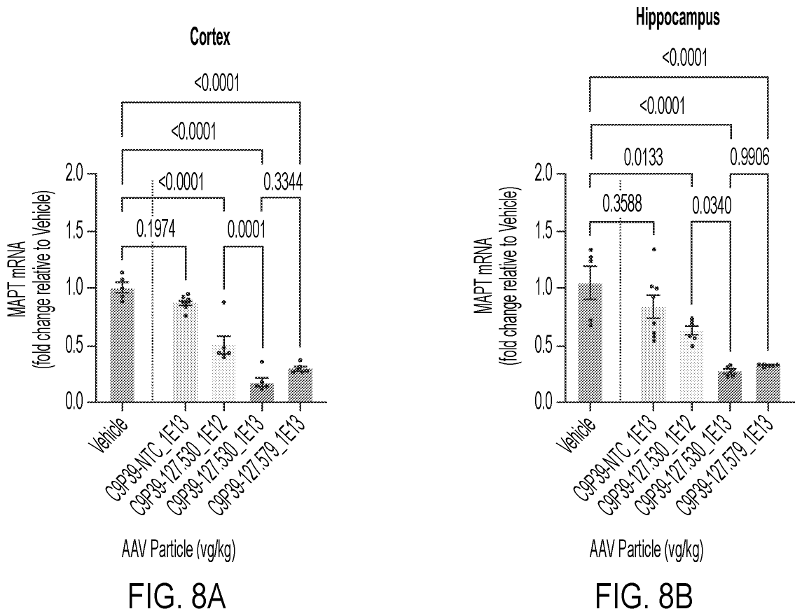

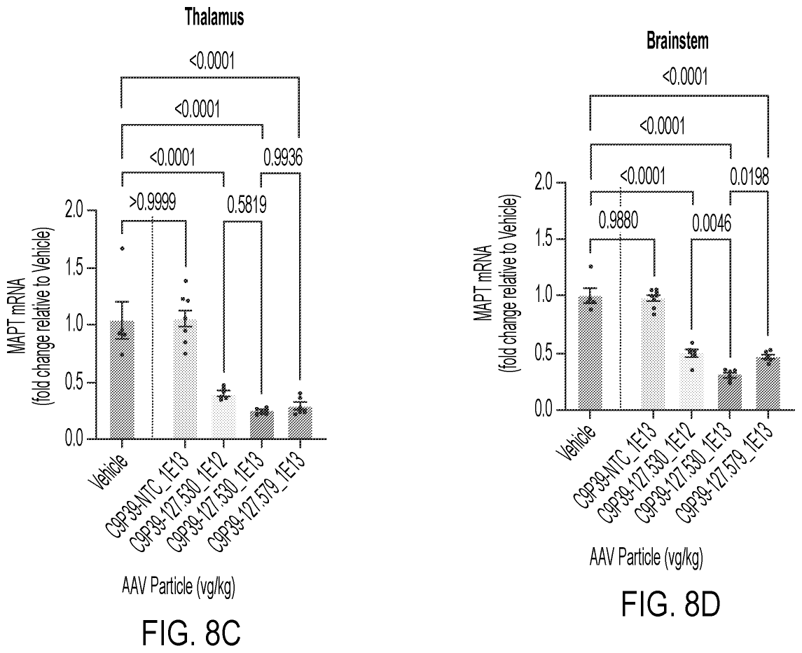

The amount of MAPT mRNA remaining after treatment was measured by qPCR and normalized to a housekeeping gene (endogenous XPNPEP1) in the cortex, hippocampus, thalamus, and brainstem. The fold-change in MAPT mRNA remaining in the groups receiving the VOYTaumiR-127-530 or VOYTaumiR-127-579 modulatory polynucleotide relative to the vehicle was determined (FIGS. 8A-8D). As shown in FIGS. 8A-8D, MAPT mRNA was reduced across all brain regions investigated, including the cortex, hippocampus, thalamus, and brainstem, at 4 weeks post-treatment with AAV particles encoding the VOYTaumiR-127-530 or VOYTaumiR-127-579 modulatory polynucleotide. Additionally, a dose response was observed in these brain regions in the mice that received the high and low dose of AAV particles encoding the VOYTaumiR-127-530 modulatory polynucleotide (FIGS. 8A-8D).

Taken together, these in vivo data demonstrate good biodistribution and/or knock down of MAPT following intravenous administration of AAV particles encoding the VOYTaumiR-127-530 modulatory polynucleotide or the VOYTaumiR-127-579 modulatory polynucleotide which target MAPT.

### VII. EQUIVALENTS AND SCOPE

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

We claim:

1. A short interfering ribonucleic acid (siRNA) for inhibiting expression of microtubule-associated protein tau (MAPT) which comprises a sense strand sequence and an antisense strand sequence, wherein:
(i) the antisense strand sequence comprises at least 20 or 21 contiguous nucleotides of any one of SEQ ID NOs: 4908, 4920, 4924, 5057, 4916, 4912, 5080, 5104, or 5128; and
(ii) the sense strand sequence comprises at least 20 or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 5054, 5060, 5064, 5066, 5070, 5074, 4914, 4934, 4954, 4974, 4994, 5014, 4910, 4930, 4950, 4970, 4990, 5010, 5077, 5084, 5088, 5092, 5096, 5098, 5101, 5108, 5112, 5116, 5120, 5122, 5125, 5132, 5136, 5140, 5144, or 5146;
wherein the sense strand sequence and the antisense strand sequence comprise a region of complementarity of at least 15, 16, 17, 18, 19, or 20 nucleotides.

2. An adeno-associated virus (AAV) viral genome comprising a nucleotide sequence positioned between two inverted terminal repeats (ITRs), wherein the nucleotide sequence encodes an siRNA for inhibiting MAPT, wherein the siRNA comprises a sense strand sequence and an antisense strand sequence, wherein:
(i) the antisense strand sequence comprises at least 19, 20, or 21 contiguous nucleotides of any one of SEQ ID NOs: 4908, 4920, 4924, 5057, 4916, 4912, 5080, 5104, or 5128; and
(ii) the sense strand sequence comprises at least 19, 20, or 21 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 5054, 5060, 5064, 5066, 5070, 5074, 4914, 4934, 4954, 4974, 4994, 5014, 4910, 4930, 4950, 4970, 4990, 5010, 5077, 5084, 5088, 5092, 5096, 5098, 5101, 5108, 5112, 5116, 5120, 5122, 5125, 5132, 5136, 5140, 5144, or 5146;
wherein the sense strand sequence and the antisense strand sequence comprise a region of complementarity of at least 15, 16, 17, 18, 19, or 20 nucleotides.

3. The siRNA of claim 1, or the AAV viral genome of claim 2, wherein:
(i) the antisense strand comprises at least 20 contiguous nucleotides of any one of SEQ ID NOS:
4908, 4920, 4924, or 4912; and/or the sense strand comprises at least 20 contiguous nucleotides of any one of SEQ ID NOs: 4906, 4926, 4946, 4966, 4986, 5006, 4918, 4938, 4958, 4978, 4998, 4922, 4942, 4962, 4982, 5002, 5022, 4910, 4930, 4950, 4970, 4990, or 5010;
(ii) the antisense strand sequence comprises at least 20 contiguous nucleotides from the nucleotide sequence of SEQ ID NO: 4908 and the sense strand sequence comprises at least 20 contiguous nucleotides from the nucleotide sequence of any one of SEQ ID NOs: 4906, 4926, 4946, 4966, 4986, or 5006;
(iii) the antisense strand sequence comprises at least 20 contiguous nucleotides from the nucleotide sequence of SEQ ID NO: 4920 and the sense strand sequence comprises at least 20 contiguous nucleotides from the nucleotide sequence of any one of SEQ ID NOs: 4918, 4938, 4958, 4978, or 4998;
(iv) the antisense strand sequence comprises at least 20 contiguous nucleotides from the nucleotide sequence of SEQ ID NO: 4924 and the sense strand sequence comprises at least 20 contiguous nucleotides from the nucleotide sequence of any one of SEQ ID NOs: 4922, 4942, 4962, 4982, 5002, or 5022;
(v) the antisense strand sequence comprises at least 20 contiguous nucleotides from the nucleotide sequence of SEQ ID NO: 4912 and the sense strand sequence comprises at least 20 contiguous nucleotides from the nucleotide sequence of any one of SEQ ID NOs: 4910, 4930, 4950, 4970, 4990, or 5010;
(vi) the antisense strand sequence comprises at least 20 contiguous nucleotides from the nucleotide sequence of SEQ ID NO: 4916 and the sense strand sequence comprises at least 20 contiguous nucleotides from the nucleotide sequence of any one of SEQ ID NOs: 4914, 4934, 4954, 4974, 4994, or 5014;
(vii) the antisense strand sequence comprises at least 20 contiguous nucleotides from the nucleotide sequence of SEQ ID NO: 5104; and the sense strand sequence comprises at least 20 contiguous nucleotides from the nucleotide sequence of any one of SEQ ID NOs: 5101, 5108, 5112, 5116, 5120, or 5122;
(viii) the antisense strand sequence comprises at least 20 contiguous nucleotides from the nucleotide sequence of SEQ ID NO: 5128; and the sense strand sequence comprises at least 20 contiguous nucleotides from the nucleotide sequence of any one of SEQ ID NOs: 5125, 5132, 5136, 5140, 5144, or 5146;
(ix) the antisense strand sequence comprises at least 20 contiguous nucleotides from the nucleotide sequence of SEQ ID NO: 5080; and the sense strand sequence comprises at least 20 contiguous nucleotides from the nucleotide sequence of any one of SEQ ID NOs: 5077, 5084, 5088, 5092, 5096, or 5098; or
(x) the antisense strand sequence comprises at least 20 contiguous nucleotides from the nucleotide sequence of SEQ ID NO: 5057; and the sense strand sequence comprises at least 20 contiguous nucleotides from the nucleotide sequence of any one of SEQ ID NOs: 5054, 5060, 5064, 5066, 5070, or 5074.

4. The siRNA of claim 1 or 3, or the AAV viral genome of claim 2 or 3, wherein:
(i) the antisense strand sequence comprises at least 20 contiguous nucleotides from the nucleotide sequence of SEQ ID NO: 4908 and the sense strand sequence comprises at least 20 contiguous nucleotides from the nucleotide sequence of SEQ ID NO: 4906;
(ii) the antisense strand sequence comprises at least 20 contiguous nucleotides from the nucleotide sequence of SEQ ID NO: 4920 and the sense strand sequence comprises at least 20 contiguous nucleotides from the nucleotide sequence of SEQ ID NO: 4918;
(iii) the antisense strand sequence comprises at least 20 contiguous nucleotides from the nucleotide sequence of SEQ ID NO: 4924 and the sense strand sequence comprises at least 20 contiguous nucleotides from the nucleotide sequence of SEQ ID NO: 4922;
(iv) the antisense strand sequence comprises at least 20 contiguous nucleotides from the nucleotide sequence of SEQ ID NO: 4908 and the sense strand sequence comprises at least 20 contiguous nucleotides from the nucleotide sequence of SEQ ID NO: 4966;
(v) the antisense strand sequence comprises at least 20 contiguous nucleotides from the nucleotide sequence of SEQ ID NO: 4912 and the sense strand sequence comprises at least 20 contiguous nucleotides from the nucleotide sequence of SEQ ID NO: 4970;
(vi) the antisense strand sequence comprises at least 20 contiguous nucleotides from the nucleotide sequence of SEQ ID NO: 4920 and the sense strand sequence comprises at least 20 contiguous nucleotides from the nucleotide sequence of SEQ ID NO: 4978;
(vii) the antisense strand sequence comprises at least 20 contiguous nucleotides from the nucleotide sequence of SEQ ID NO: 4924 and the sense strand sequence comprises at least 20 contiguous nucleotides from the nucleotide sequence of SEQ ID NO: 4982;
(viii) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4910;
(ix) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4914;
(x) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5054;
(xi) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 4926;
(xii) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4930;
(xiii) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4934;
(xiv) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4938;
(xv) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 4942;
(xvi) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5060;
(xvii) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 4946;
(xviii) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4950;
(xix) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4954;
(xx) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4958;
(xxi) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 4962;
(xxii) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5064;
(xxiii) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4974;
(xxiv) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5066;
(xxv) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 4986;
(xxvi) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 4990;
(xxvii) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 4994;
(xxviii) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4998;
(xxix) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 5002;
(xxx) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5070;
(xxxi) the antisense strand sequence comprises at least 20 contiguous nucleotides from the nucleotide sequence of SEQ ID NO: 4908; and the sense strand sequence comprises at least 20 contiguous nucleotides from the nucleotide sequence of SEQ ID NO: 5006;
(xxxii) the antisense strand sequence comprises at least 20 contiguous nucleotides from the nucleotide sequence of SEQ ID NO: 4912; and the sense strand sequence comprises at least 20 contiguous nucleotides from the nucleotide sequence of SEQ ID NO: 5010;
(xxxiii) the antisense strand sequence comprises at least 20 contiguous nucleotides from the nucleotide sequence of SEQ ID NO: 4916; and the sense strand sequence comprises at least 20 contiguous nucleotides from the nucleotide sequence of SEQ ID NO: 5014;
(xxxiv) the antisense strand sequence comprises at least 20 contiguous nucleotides from the nucleotide sequence of SEQ ID NO: 4920; and the sense strand sequence comprises at least 20 contiguous nucleotides from the nucleotide sequence of SEQ ID NO: 4958;
(xxxv) the antisense strand sequence comprises at least 20 contiguous nucleotides from the nucleotide sequence of SEQ ID NO: 4924; and the sense strand sequence comprises at least 20 contiguous nucleotides from the nucleotide sequence of SEQ ID NO: 5022;
(xxxvi) the antisense strand sequence comprises at least 20 contiguous nucleotides from the nucleotide sequence of SEQ ID NO: 5057; and the sense strand sequence comprises at least 20 contiguous nucleotides from the nucleotide sequence of SEQ ID NO: 5074;
(xxxvii) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5077;
(xxxviii) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5084;
(xxxix) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5088;
(xl) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5092;
(xli) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5096;
(xlii) the antisense strand sequence comprises at least 20 contiguous nucleotides from the nucleotide sequence of SEQ ID NO: 5080; and the sense strand sequence comprises at least 20 contiguous nucleotides from the nucleotide sequence of SEQ ID NO: 5098;
(xliii) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5101;
(xliv) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5108;
(xlv) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5112;
(xlvi) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5116;
(xlvii) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5120;
(xlviii) the antisense strand sequence comprises at least 20 contiguous nucleotides from the nucleotide sequence of SEQ ID NO: 5104; and the sense strand sequence comprises at least 20 contiguous nucleotides from the nucleotide sequence of SEQ ID NO: 5122;
(xlix) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5125;
(l) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5132;
(li) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5136;
(lii) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5140;
(liii) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5144; or
(liv) the antisense strand sequence comprises at least 20 contiguous nucleotides from the nucleotide sequence of SEQ ID NO: 5128; and the sense strand sequence comprises at least 20 contiguous nucleotides from the nucleotide sequence of SEQ ID NO: 5146.

5. The siRNA of any one of claim 1, 3, or 4, or the AAV viral genome of any one of claims 2-4, wherein:
(i) the antisense strand comprises the nucleotide sequence of any one of SEQ ID NOs: 4908, 4920, 4924, or 4912; and/or the sense strand comprises at the nucleotide sequence of any one of SEQ ID NOs: 4906, 4926, 4946, 4966, 4986, 5006, 4918, 4938, 4958, 4978, 4998, 4922, 4942, 4962, 4982, 5002, 5022, 4910, 4930, 4950, 4970, 4990, or 5010;
(ii) the antisense strand sequence comprises the nucleotide sequence of SEQ ID NO: 4908 and the sense strand sequence comprises the nucleotide sequence of any one of SEQ ID NOs: 4906, 4926, 4946, 4966, 4986, or 5006;
(iii) the antisense strand sequence comprises the nucleotide sequence of SEQ ID NO: 4920 and the sense strand sequence comprises the nucleotide sequence of any one of SEQ ID NOs: 4918, 4938, 4958, 4978, or 4998;
(iv) the antisense strand sequence comprises the nucleotide sequence of SEQ ID NO: 4924 and the sense strand sequence comprises the nucleotide sequence of any one of SEQ ID NOs: 4922, 4942, 4962, 4982, 5002, or 5022;
(v) the antisense strand sequence comprises the nucleotide sequence of SEQ ID NO: 4912 and the sense strand sequence comprises the nucleotide sequence of any one of SEQ ID NOs: 4910, 4930, 4950, 4970, 4990, or 5010;
(vi) the antisense strand sequence comprises the nucleotide sequence of SEQ ID NO: 4916 and the sense strand sequence comprises the nucleotide sequence of any one of SEQ ID NOs: 4914, 4934, 4954, 4974, 4994, or 5014;
(vii) the antisense strand sequence comprises the nucleotide sequence of SEQ ID NO: 5104; and the sense strand sequence comprises the nucleotide sequence of any one of SEQ ID NOs: 5101, 5108, 5112, 5116, 5120, or 5122;
(viii) the antisense strand sequence comprises the nucleotide sequence of SEQ ID NO: 5128; and the sense strand sequence comprises the nucleotide sequence of any one of SEQ ID NOs: 5125, 5132, 5136, 5140, 5144, or 5146;
(ix) the antisense strand sequence comprises the nucleotide sequence of SEQ ID NO: 5080; and the sense strand sequence comprises the nucleotide sequence of any one of SEQ ID NOs: 5077, 5084, 5088, 5092, 5096, or 5098; or
(x) the antisense strand sequence comprises the nucleotide sequence of SEQ ID NO: 5057; and the sense strand sequence comprises the nucleotide sequence of any one of SEQ ID NOs: 5054, 5060, 5064, 5066, 5070, or 5074.

6. The siRNA of any one of claim 1 or 3-5, or the AAV viral genome of any one of claims 2-5, wherein:
(i) the antisense strand sequence comprises the nucleotide sequence of SEQ ID NO: 4908 and the sense strand sequence comprises the nucleotide sequence of SEQ ID NO: 4906;
(ii) the antisense strand sequence comprises the nucleotide sequence of SEQ ID NO: 4920 and the sense strand sequence comprises the nucleotide sequence of SEQ ID NO: 4918;
(iii) the antisense strand sequence comprises the nucleotide sequence of SEQ ID NO: 4924 and the sense strand sequence comprises the nucleotide sequence of SEQ ID NO: 4922;
(iv) the antisense strand sequence comprises the nucleotide sequence of SEQ ID NO: 4908 and the sense strand sequence comprises the nucleotide sequence of SEQ ID NO: 4966;
(v) the antisense strand sequence comprises the nucleotide sequence of SEQ ID NO: 4912 and the sense strand sequence comprises the nucleotide sequence of SEQ ID NO: 4970;
(vi) the antisense strand sequence comprises the nucleotide sequence of SEQ ID NO: 4920 and the sense strand sequence comprises the nucleotide sequence of SEQ ID NO: 4978;
(vii) the antisense strand sequence comprises the nucleotide sequence of SEQ ID NO: 4924 and the sense strand sequence comprises the nucleotide sequence of SEQ ID NO: 4982;
(viii) the antisense strand sequence comprises the nucleotide sequence of SEQ ID NO: 4912; and the sense strand sequence comprises the nucleotide sequence of SEQ ID NO: 4910;
(ix) the antisense strand sequence comprises the nucleotide sequence of SEQ ID NO: 4916; and the sense strand sequence comprises the nucleotide sequence of SEQ ID NO: 4914;
(x) the antisense strand sequence comprises the nucleotide sequence of SEQ ID NO: 5057; and the sense strand sequence comprises the nucleotide sequence of SEQ ID NO: 5054;
(xi) the antisense strand sequence comprises the nucleotide sequence of SEQ ID NO: 4908; and the sense strand sequence comprises the nucleotide sequence of SEQ ID NO: 4926;
(xii) the antisense strand sequence comprises the nucleotide sequence of SEQ ID NO: 4912; and the sense strand sequence comprises the nucleotide sequence of SEQ ID NO: 4930;
(xiii) the antisense strand sequence comprises the nucleotide sequence of SEQ ID NO: 4916; and the sense strand sequence comprises the nucleotide sequence of SEQ ID NO: 4934;
(xiv) the antisense strand sequence comprises the nucleotide sequence of SEQ ID NO: 4920; and the sense strand sequence comprises the nucleotide sequence of SEQ ID NO: 4938;
(xv) the antisense strand sequence comprises the nucleotide sequence of SEQ ID NO: 4924; and the sense strand sequence comprises the nucleotide sequence of SEQ ID NO: 4942;
(xvi) the antisense strand sequence comprises the nucleotide sequence of SEQ ID NO: 5057; and the sense strand sequence comprises the nucleotide sequence of SEQ ID NO: 5060;
(xvii) the antisense strand sequence comprises the nucleotide sequence of SEQ ID NO: 4908; and the sense strand sequence comprises the nucleotide sequence of SEQ ID NO: 4946;
(xviii) the antisense strand sequence comprises the nucleotide sequence of SEQ ID NO: 4912; and the sense strand sequence comprises the nucleotide sequence of SEQ ID NO: 4950;
(xix) the antisense strand sequence comprises the nucleotide sequence of SEQ ID NO: 4916; and the sense strand sequence comprises the nucleotide sequence of SEQ ID NO: 4954;
(xx) the antisense strand sequence comprises the nucleotide sequence of SEQ ID NO: 4920; and the sense strand sequence comprises the nucleotide sequence of SEQ ID NO: 4958;
(xxi) the antisense strand sequence comprises the nucleotide sequence of SEQ ID NO: 4924; and the sense strand sequence comprises the nucleotide sequence of SEQ ID NO: 4962;
(xxii) the antisense strand sequence comprises the nucleotide sequence of SEQ ID NO: 5057; and the sense strand sequence comprises the nucleotide sequence of SEQ ID NO: 5064;
(xxiii) the antisense strand sequence comprises the nucleotide sequence of SEQ ID NO: 4916; and the sense strand sequence comprises the nucleotide sequence of SEQ ID NO: 4974;
(xxiv) the antisense strand sequence comprises the nucleotide sequence of SEQ ID NO: 5057; and the sense strand sequence comprises the nucleotide sequence of SEQ ID NO: 5066;
(xxv) the antisense strand sequence comprises the nucleotide sequence of SEQ ID NO: 4908; and the sense strand sequence comprises the nucleotide sequence of SEQ ID NO: 4986;
(xxvi) the antisense strand sequence comprises the nucleotide sequence of SEQ ID NO: 4912; and the sense strand sequence comprises the nucleotide sequence of SEQ ID NO: 4990;
(xxvii) the antisense strand sequence comprises the nucleotide sequence of SEQ ID NO: 4916; and the sense strand sequence comprises the nucleotide sequence of SEQ ID NO: 4994;
(xxviii) the antisense strand sequence comprises the nucleotide sequence of SEQ ID NO: 4920; and the sense strand sequence comprises the nucleotide sequence of SEQ ID NO: 4998;
(xxix) the antisense strand sequence comprises the nucleotide sequence of SEQ ID NO: 4924; and the sense strand sequence comprises the nucleotide sequence of SEQ ID NO: 5002;
(xxx) the antisense strand sequence comprises the nucleotide sequence of SEQ ID NO: 5057; and the sense strand sequence comprises the nucleotide sequence of SEQ ID NO: 5070;
(xxxi) the antisense strand sequence comprises the nucleotide sequence of SEQ ID NO: 4908; and the sense strand sequence comprises the nucleotide sequence of SEQ ID NO: 5006;
(xxxii) the antisense strand sequence comprises the nucleotide sequence of SEQ ID NO: 4912; and the sense strand sequence comprises the nucleotide sequence of SEQ ID NO: 5010;
(xxxiii) the antisense strand sequence comprises the nucleotide sequence of SEQ ID NO: 4916; and the sense strand sequence comprises the nucleotide sequence of SEQ ID NO: 5014;
(xxxiv) the antisense strand sequence comprises the nucleotide sequence of SEQ ID NO: 4920; and the sense strand sequence comprises the nucleotide sequence of SEQ ID NO: 4958;
(xxxv) the antisense strand sequence comprises the nucleotide sequence of SEQ ID NO: 4924; and the sense strand sequence comprises the nucleotide sequence of SEQ ID NO: 5022;
(xxxvi) the antisense strand sequence comprises the nucleotide sequence of SEQ ID NO: 5057; and the sense strand sequence comprises the nucleotide sequence of SEQ ID NO: 5074;
(xxxvii) the antisense strand sequence comprises the nucleotide sequence of SEQ ID NO: 5080; and the sense strand sequence comprises the nucleotide sequence of SEQ ID NO: 5077;
(xxxviii) the antisense strand sequence comprises the nucleotide sequence of SEQ ID NO: 5080; and the sense strand sequence comprises the nucleotide sequence of SEQ ID NO: 5084;
(xxxix) the antisense strand sequence comprises the nucleotide sequence of SEQ ID NO: 5080; and the sense strand sequence comprises the nucleotide sequence of SEQ ID NO: 5088;
(xl) the antisense strand sequence comprises the nucleotide sequence of SEQ ID NO: 5080; and the sense strand sequence comprises the nucleotide sequence of SEQ ID NO: 5092;
(xli) the antisense strand sequence comprises the nucleotide sequence of SEQ ID NO: 5080; and the sense strand sequence comprises the nucleotide sequence of SEQ ID NO: 5096;
(xlii) the antisense strand sequence comprises the nucleotide sequence of SEQ ID NO: 5080; and the sense strand sequence comprises the nucleotide sequence of SEQ ID NO: 5098;
(xliii) the antisense strand sequence comprises the nucleotide sequence of SEQ ID NO: 5104; and the sense strand sequence comprises the nucleotide sequence of SEQ ID NO: 5101;
(xliv) the antisense strand sequence comprises the nucleotide sequence of SEQ ID NO: 5104; and the sense strand sequence comprises the nucleotide sequence of SEQ ID NO: 5108;
(xlv) the antisense strand sequence comprises the nucleotide sequence of SEQ ID NO: 5104; and the sense strand sequence comprises the nucleotide sequence of SEQ ID NO: 5112;
(xlvi) the antisense strand sequence comprises the nucleotide sequence of SEQ ID NO: 5104; and the sense strand sequence comprises the nucleotide sequence of SEQ ID NO: 5116;
(xlvii) the antisense strand sequence comprises the nucleotide sequence of SEQ ID NO: 5104; and the sense strand sequence comprises the nucleotide sequence of SEQ ID NO: 5120;
(xlviii) the antisense strand sequence comprises the nucleotide sequence of SEQ ID NO: 5104; and the sense strand sequence comprises the nucleotide sequence of SEQ ID NO: 5122;
(xlix) the antisense strand sequence comprises the nucleotide sequence of SEQ ID NO: 5128; and the sense strand sequence comprises the nucleotide sequence of SEQ ID NO: 5125;
(l) the antisense strand sequence comprises the nucleotide sequence of SEQ ID NO: 5128; and the sense strand sequence comprises the nucleotide sequence of SEQ ID NO: 5132;
(li) the antisense strand sequence comprises the nucleotide sequence of SEQ ID NO: 5128; and the sense strand sequence comprises the nucleotide sequence of SEQ ID NO: 5136;
(lii) the antisense strand sequence comprises the nucleotide sequence of SEQ ID NO: 5128; and the sense strand sequence comprises the nucleotide sequence of SEQ ID NO: 5140;
(liii) the antisense strand sequence comprises the nucleotide sequence of SEQ ID NO: 5128; and the sense strand sequence comprises the nucleotide sequence of SEQ ID NO: 5144; or
(liv) the antisense strand sequence comprises the nucleotide sequence of SEQ ID NO: 5128; and the sense strand sequence comprises the nucleotide sequence of SEQ ID NO: 5146.

7. The siRNA of any one of claim 1 or 3-6, or the AAV viral genome of any one of claims 2-6, wherein:
(i) the nucleotide sequence encoding the antisense strand sequence comprises the nucleotide sequence of SEQ ID NO: 4907 and the nucleotide sequence encoding the sense strand sequence comprises the nucleotide sequence of SEQ ID NO: 4905;
(ii) the nucleotide sequence encoding the antisense strand sequence comprises the nucleotide sequence of SEQ ID NO: 4919 and the nucleotide sequence encoding the sense strand sequence comprises the nucleotide sequence of SEQ ID NO: 4917;
(iii) the nucleotide sequence encoding the antisense strand sequence comprises the nucleotide sequence of SEQ ID NO: 4923 and the nucleotide sequence encoding the sense strand sequence comprises the nucleotide sequence of SEQ ID NO: 4921;
(iv) the nucleotide sequence encoding the antisense strand sequence comprises the nucleotide sequence of SEQ ID NO: 4907 and the nucleotide sequence encoding the sense strand sequence comprises the nucleotide sequence of SEQ ID NO: 4965;
(v) the nucleotide sequence encoding the antisense strand sequence comprises the nucleotide sequence of SEQ ID NO: 4911 and the nucleotide sequence encoding the sense strand sequence comprises the nucleotide sequence of SEQ ID NO: 4969;
(vi) the nucleotide sequence encoding the antisense strand sequence comprises the nucleotide sequence of SEQ ID NO: 4919 and the nucleotide sequence encoding the sense strand sequence comprises the nucleotide sequence of SEQ ID NO: 4977; or
(vii) the nucleotide sequence encoding the antisense strand sequence comprises the nucleotide sequence of SEQ ID NO: 4923 and the nucleotide sequence encoding the sense strand sequence comprises the nucleotide sequence of SEQ ID NO: 4981.

8. The siRNA of any one of claim 1 or 3-7, or the AAV viral genome of any one of claims 2-7, wherein:
(i) the sense strand sequence, the antisense strand sequence, or both the sense strand sequence and the antisense strand sequence comprise at least 15-30, 19-21, or 25-30 nucleotides in length, e.g., 17, 18, 20, 21, 22, 25, or 30 nucleotides in length;
(ii) the sense strand sequence, the antisense strand sequence, or both the sense strand sequence and the antisense strand sequence comprise an overhang, e.g., an overhang at the 5′ end of the sense strand sequence and/or at the 3′ end of the antisense strand sequence, of 1 or 2 nucleotides;
(iii) the sense strand and the antisense strand comprise one mismatch; and/or
(iv) the antisense strand and the target sequence comprise one mismatch.

9. A modulatory polynucleotide comprising the siRNA of any one of claim 1 or 3-8.

10. The AAV viral genome of any one of claims 2-8, wherein the viral genome further encodes a modulatory polynucleotide comprising the sense and antisense strand of the siRNA.

11. The modulatory polynucleotide of claim 9 or the AAV viral genome of claim 10, wherein the modulatory polynucleotide further comprises one, two, three or all of:
(i) a 5′ flanking region;
(ii) a loop region; and
(iii) a 3′ flanking region.

12. The modulatory polynucleotide of claim 11 or the AAV viral genome of claim 11, wherein:
(A) (i) the 5′ flanking region comprises the nucleotide sequence of any one of SEQ ID NOs: 4878-4886; a nucleotide sequence at least 85%, 90%, 92%, 95%, 96%, 97%, 98%, or 99% identical thereto: a nucleotide sequence comprising one, two, three, four, five, six, or seven, but no more than ten different nucleotides relative to the nucleotide sequence of any of SEQ ID NOs: 4878-4886; or a nucleotide sequence comprising one, two, three, four, five, six, or seven, but no more than ten modifications relative to the nucleotide sequence of any of SEQ ID NOs: 4878-4886;
(ii) the loop region comprises the nucleotide sequence of any of SEQ ID NOs: 4887-4896; a nucleotide sequence at least 85%, 90%, 92%, 95%, 96%, 97%, 98%, or 99% identical thereto; a nucleotide sequence comprising one, two, three, or four, but no more than five different nucleotides relative to the nucleotide sequence of any of SEQ ID NOs: 4887-4896; or a nucleotide sequence comprising one, two, three, or four, but no more than five modifications relative to the nucleotide sequence of any of SEQ ID NOs: 4887-4896; and/or
(iii) the 3′ flanking region comprises the nucleotide sequence of any one of SEQ ID NOs: 4897-4904; a nucleotide sequence at least 85%, 90%, 92%, 95%, 96%, 97%, 98%, or 99% identical thereto; a nucleotide sequence comprising one, two, three, four, five, six, or seven, but no more than ten modifications relative to the nucleotide sequence of any of SEQ ID NOs: 4897-4904; or a nucleotide sequence comprising one, two, three, four, five, six, or seven, but no more than ten modifications relative to the nucleotide sequence of any of SEQ ID NOs: 4897-4904; and/or
(B) (i) the nucleotide sequence encoding the 5′ flanking region comprises the nucleotide sequence of any of SEQ ID NOs: 4353-4361, a nucleotide sequence at least 85%, 90%, 92%, 95%, 96%, 97%, 98%, or 99% identical thereto; a nucleotide sequence comprising one, two, three, four, five, six, or seven, but no more than ten different nucleotides relative to the nucleotide sequence of any of SEQ ID NOs: 4353-4361; or a nucleotide sequence comprising one, two, three, four, five, six, or seven, but no more than ten modifications relative to the nucleotide sequence of any of SEQ ID NOs: 4353-4361;
(ii) the nucleotide sequence encoding the loop region comprises the nucleotide sequence of any of SEQ ID NOs: 4362-4371; a nucleotide sequence at least 85%, 90%, 92%, 95%, 96%, 97%, 98%, or 99% identical thereto; a nucleotide sequence comprising one, two, three, or four, but no more than five different nucleotides relative to the nucleotide sequence of any of SEQ ID NOs: 4362-4371; or a nucleotide sequence comprising one, two, three, or four, but no more than five modifications relative to the nucleotide sequence of any of SEQ ID NOs: 4362-4371; and/or
(iii) the nucleotide sequence encoding the 3′ flanking region comprises the nucleotide sequence of any of SEQ ID NOs: 4372-4379; a nucleotide sequence at least 85%, 90%, 92%, 95%, 96%, 97%, 98%, or 99% identical thereto; a nucleotide sequence comprising one, two, three, four, five, six, or seven, but no more than ten different nucleotides relative to the nucleotide sequence of any of SEQ ID NOs:
4372-4379; or a nucleotide sequence comprising one, two, three, four, five, six, or seven, but no more than ten modifications relative to the nucleotide sequence of any of SEQ ID NOs: 4372-4379.

13. The modulatory polynucleotide of any one of claim 9, 11, or 12, or the AAV viral genome of any one of claims 10-12, wherein the modulatory polynucleotide comprises:
(A) (i) a 5′ flanking region comprising the nucleotide sequence of SEQ ID NO: 4879, a nucleotide sequence at least 85%, 90%, 92%, 95%, 96%, 97%, 98%, or 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten different nucleotides relative to the nucleotide sequence of SEQ ID NO: 4879;
(ii) a loop region comprising the nucleotide sequence of SEQ ID NO: 4887, a nucleotide sequence at least 85%, 90%, 92%, 95%, 96%, 97%, 98%, or 99% identical to SEQ ID NO: 4887, a nucleotide sequence comprising one, two, three, or four, but no more than five different nucleotides relative to the nucleotide sequence of SEQ ID NO: 4887; or a nucleotide sequence comprising one, two, three, or four, but no more than five different nucleotides relative to the nucleotide sequence of SEQ ID NO: 4887; and
(iii) a 3′ flanking region of SEQ ID NO: 4898, a nucleotide sequence at least 85%, 90%, 92%, 95%, 96%, 97%, 98%, or 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898;
(B) (i) a 5′ flanking region comprising the nucleotide sequence of SEQ ID NO: 4879, a nucleotide sequence at least 85%, 90%, 92%, 95%, 96%, 97%, 98%, or 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) a loop region comprising the nucleotide sequence of SEQ ID NO: 4888, a nucleotide sequence at least 85%, 90%, 92%, 95%, 96%, 97%, 98%, or 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888; and
(iii) a 3′ flanking region of SEQ ID NO: 4898, a nucleotide sequence at least 85%, 90%, 92%, 95%, 96%, 97%, 98%, or 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898;
(C) (i) a 5′ flanking region comprising the nucleotide sequence of SEQ ID NO: 4879, a nucleotide sequence at least 85%, 90%, 92%, 95%, 96%, 97%, 98%, or 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) a loop region comprising the nucleotide sequence of SEQ ID NO: 4887, a nucleotide sequence at least 85%, 90%, 92%, 95%, 96%, 97%, 98%, or 99% identical to SEQ ID NO: 4887, a nucleotide sequence comprising one, two, three, or four, but no more than five different nucleotides relative to the nucleotide sequence of SEQ ID NO: 4887; or a nucleotide sequence comprising one, two, three, or four, but no more than five modifications relative to the nucleotide sequence of SEQ ID NO: 4887; and
(iii) a 3′ flanking region of SEQ ID NO: 4899, a nucleotide sequence at least 85%, 90%, 92%, 95%, 96%, 97%, 98%, or 99% identical to SEQ ID NO: 4899, a nucleotide sequence comprising one, two, three, four, five, six, or seven, but no more than ten different nucleotides relative to the nucleotide sequence of SEQ ID NO: 4899, a nucleotide sequence comprising one, two, three, four, five, six, or seven, but no more than ten modifications relative to the nucleotide sequence of SEQ ID NO: 4899; or
(D) (i) a 5′ flanking region comprising the nucleotide sequence of SEQ ID NO: 4880, a nucleotide sequence at least 85%, 90%, 92%, 95%, 96%, 97%, 98%, or 99% identical to SEQ ID NO: 4880, a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) a loop region comprising the nucleotide sequence of SEQ ID NO: 4891, a nucleotide sequence at least 85%, 90%, 92%, 95%, 96%, 97%, 98%, or 99% identical to SEQ ID NO: 4891, a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891; and
(iii) a 3′ flanking region of SEQ ID NO: 4900, a nucleotide sequence at least 85%, 90%, 92%, 95%, 96%, 97%, 98%, or 99% identical to SEQ ID NO: 4900, a nucleotide sequence comprising one, two, three, four, five, six, or seven, but no more than ten different nucleotides relative to the nucleotide sequence of SEQ ID NO: 4900, or a nucleotide sequence comprising one, two, three, four, five, six, or seven, but no more than ten modifications relative to SEQ ID NO: 4900.

14. The modulatory polynucleotide of any one of claim 9 or 11-13 or the AAV viral genome of any one of claims 10-13, wherein the modulatory polynucleotide comprises in 5′ to 3′ order:
(i) the 5′ flanking region, sense strand sequence, loop region, antisense strand sequence, and 3′ flanking region; or
(ii) the 5′ flanking region, antisense strand sequence, loop region, sense strand sequence, and 3′ flanking region.

15. The modulatory polynucleotide of any one of claim 9 or 11-14 or the AAV viral genome of any one of claims 10-14, wherein the modulatory polynucleotide comprises in 5′ to 3′ order:
(A) (i) a 5′ flanking region comprising the nucleotide sequence of SEQ ID NO: 4880; a nucleotide sequence at least 85%, 90%, 95% 96%, 97%, 98%, 99% identical to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) the sense strand sequence of SEQ ID NO: 4906;
(iii) a loop region comprising the nucleotide sequence of SEQ ID NO: 4891; a nucleotide sequence at least 85%, 90%, 95% 96%, 97%, 98%, 99% identical to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891;
(iv) the antisense strand sequence of SEQ ID NO: 4908; and
(v) a 3′ flanking region comprising the nucleotide sequence of SEQ ID NO: 4900; a nucleotide sequence at least 85%, 90%, 95% 96%, 97%, 98%, 99% identical to SEQ ID NO: 4900; a nucleotide sequence comprising one, two, three, four, five, six, or seven, but no more than ten different nucleotides relative to the nucleotide sequence of SEQ ID NO: 4900; or a nucleotide sequence comprising one, two, three, four, five, six, or seven, but no more than ten modifications relative to SEQ ID NO: 4900;
(B) (i) a 5′ flanking region comprising the nucleotide sequence of SEQ ID NO: 4880; a nucleotide sequence at least 85%, 90%, 95% 96%, 97%, 98%, 99% identical to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) the sense strand sequence of SEQ ID NO: 4918;
(iii) a loop region comprising the nucleotide sequence of SEQ ID NO: 4891; a nucleotide sequence at least 85%, 90%, 95% 96%, 97%, 98%, 99% identical to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891;
(iv) the antisense strand sequence of SEQ ID NO: 4920; and
(v) a 3′ flanking region comprising the nucleotide sequence of SEQ ID NO: 4900; a nucleotide sequence at least 85%, 90%, 95% 96%, 97%, 98%, 99% identical to SEQ ID NO: 4900; a nucleotide sequence comprising one, two, three, four, five, six, or seven, but no more than ten different nucleotides relative to the nucleotide sequence of SEQ ID NO: 4900; or a nucleotide sequence comprising one, two, three, four, five, six, or seven, but no more than ten modifications relative to SEQ ID NO: 4900;
(C) (i) a 5′ flanking region comprising the nucleotide sequence of SEQ ID NO: 4880; a nucleotide sequence at least 85%, 90%, 95% 96%, 97%, 98%, 99% identical to SEQ ID NO: 4880; a nucleotide sequence comprising one, two, three, four, five, six, or seven, but no more than ten different nucleotides relative to the nucleotide sequence of SEQ ID NO: 4880; or a nucleotide sequence comprising one, two, three, four, five, six, or seven, but no more than ten modifications relative to the nucleotide sequence of SEQ ID NO: 4880;
(ii) the sense strand sequence of SEQ ID NO: 4922;
(iii) a loop region comprising the nucleotide sequence of SEQ ID NO: 4891; a nucleotide sequence at least 85%, 90%, 95% 96%, 97%, 98%, 99% identical to SEQ ID NO: 4891; a nucleotide sequence comprising one, two, three, or four, but no more than five different nucleotides relative to the nucleotide sequence of SEQ ID NO: 4891; or a nucleotide sequence comprising one, two, three, or four, but no more than five modifications relative to the nucleotide sequence of SEQ ID NO: 4891;
(iv) the antisense strand sequence of SEQ ID NO: 4924; and
(v) a 3′ flanking region comprising the nucleotide sequence of SEQ ID NO: 4900; a nucleotide sequence at least 85%, 90%, 95% 96%, 97%, 98%, 99% identical to SEQ ID NO: 4900; a nucleotide sequence comprising one, two, three, four, five, six, or seven, but no more than ten different nucleotides relative to the nucleotide sequence of SEQ ID NO: 4900; or a nucleotide sequence comprising one, two, three, four, five, six, or seven, but no more than ten modifications relative to SEQ ID NO: 4900;
(D) (i) a 5′ flanking region comprising the nucleotide sequence of SEQ ID NO: 4879, a nucleotide sequence at least 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4966;
(iii) a loop region comprising the nucleotide sequence of SEQ ID NO: 4888, a nucleotide sequence at least 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4908; and
(v) a 3′ flanking region of SEQ ID NO: 4898, a nucleotide sequence at least 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898;
(E) (i) a 5′ flanking region comprising the nucleotide sequence of SEQ ID NO: 4879, a nucleotide sequence at least 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4970;
(iii) a loop region comprising the nucleotide sequence of SEQ ID NO: 4888, a nucleotide sequence at least 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4912; and
(v) a 3′ flanking region of SEQ ID NO: 4898, a nucleotide sequence at least 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898;
(F) (i) a 5′ flanking region comprising the nucleotide sequence of SEQ ID NO: 4879, a nucleotide sequence at least 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4978;
(iii) a loop region comprising the nucleotide sequence of SEQ ID NO: 4888, a nucleotide sequence at least 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4920; and
(v) a 3′ flanking region of SEQ ID NO: 4898, a nucleotide sequence at least 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898; or
(G) (i) a 5′ flanking region comprising the nucleotide sequence of SEQ ID NO: 4879, a nucleotide sequence at least 85%, 90%, 95% 96%, 97%, 98%, 99% identical to SEQ ID NO: 4879, a nucleotide sequence comprising one, two, three, four, five, six, or seven, but no more than ten different nucleotides relative to the nucleotide sequence of SEQ ID NO: 4879; or a nucleotide sequence comprising one, two, three, four, five, six, or seven, but no more than ten modifications relative to the nucleotide sequence of SEQ ID NO: 4879;
(ii) the sense strand sequence of SEQ ID NO: 4982;
(iii) a loop region comprising the nucleotide sequence of SEQ ID NO: 4888, a nucleotide sequence at least 85%, 90%, 95% 96%, 97%, 98%, 99% identical to SEQ ID NO: 4888, a nucleotide sequence comprising one, two, three, or four, but no more than five different nucleotides relative to the nucleotide sequence of SEQ ID NO: 4888; or a nucleotide sequence comprising one, two, three, or four, but no more than five modifications relative to the nucleotide sequence of SEQ ID NO: 4888;
(iv) the antisense strand sequence comprises the nucleotide sequence of SEQ ID NO: 4924; and
(v) a 3′ flanking region of SEQ ID NO: 4898, a nucleotide sequence at least 85%, 90%, 95% 96%, 97%, 98%, 99% identical to SEQ ID NO: 4898, a nucleotide sequence comprising one, two, three, four, five, six, or seven, but no more than ten different nucleotides relative to the nucleotide sequence of SEQ ID NO: 4898, a nucleotide sequence comprising one, two, three, four, five, six, or seven, but no more than ten modifications relative to the nucleotide sequence of SEQ ID NO: 4898.

16. The modulatory polynucleotide of any one of claim 9 or 11-15 or the AAV viral genome of any one of claims 10-15, wherein the nucleotide sequence encoding the modulatory polynucleotide comprises in 5′ to 3′ order:
(A) (i) a 5′ flanking region comprising the nucleotide sequence of SEQ ID NO: 4355; a nucleotide sequence at least 85%, 90%, 95% 96%, 97%, 98%, 99% identical to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355;
(ii) the sense strand sequence of SEQ ID NO: 4905;
(iii) a loop region comprising the nucleotide sequence of SEQ ID NO: 4366; a nucleotide sequence at least 85%, 90%, 95% 96%, 97%, 98%, 99% identical to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366;
(iv) the antisense strand sequence of SEQ ID NO: 4907; and
(v) a 3′ flanking region comprising the nucleotide sequence of SEQ ID NO: 4375; a nucleotide sequence at least 85%, 90%, 95% 96%, 97%, 98%, 99% identical to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to SEQ ID NO: 4375;
(B) (i) a 5′ flanking region comprising the nucleotide sequence of SEQ ID NO: 4355; a nucleotide sequence at least 85%, 90%, 95% 96%, 97%, 98%, 99% identical to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355;
(ii) the sense strand sequence of SEQ ID NO: 4917;
(iii) a loop region comprising the nucleotide sequence of SEQ ID NO: 4366; a nucleotide sequence at least 85%, 90%, 95% 96%, 97%, 98%, 99% identical to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366;
(iv) the antisense strand sequence of SEQ ID NO: 4919; and
(v) a 3′ flanking region comprising the nucleotide sequence of SEQ ID NO: 4375; a nucleotide sequence at least 85%, 90%, 95% 96%, 97%, 98%, 99% identical to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to SEQ ID NO: 4375;
(C) (i) a 5′ flanking region comprising the nucleotide sequence of SEQ ID NO: 4355; a nucleotide sequence at least 85%, 90%, 95% 96%, 97%, 98%, 99% identical to SEQ ID NO: 4355; a nucleotide sequence comprising one, two, three, four, five, six, or seven, but no more than ten different nucleotides relative to the nucleotide sequence of SEQ ID NO: 4355; or a nucleotide sequence comprising one, two, three, four, five, six, or seven, but no more than ten modifications relative to the nucleotide sequence of SEQ ID NO: 4355;
(ii) the sense strand sequence of SEQ ID NO: 4921;
(iii) a loop region comprising the nucleotide sequence of SEQ ID NO: 4366; a nucleotide sequence at least 85%, 90%, 95% 96%, 97%, 98%, 99% identical to SEQ ID NO: 4366; a nucleotide sequence comprising one, two, three, or four, but no more than five different nucleotides relative to the nucleotide sequence of SEQ ID NO: 4366; or a nucleotide sequence comprising one, two, three, or four, but no more than five modifications relative to the nucleotide sequence of SEQ ID NO: 4366;
(iv) the antisense strand sequence of SEQ ID NO: 4923; and
(v) a 3′ flanking region comprising the nucleotide sequence of SEQ ID NO: 4375; a nucleotide sequence at least 85%, 90%, 95% 96%, 97%, 98%, 99% identical to SEQ ID NO: 4375; a nucleotide sequence comprising one, two, three, four, five, six, or seven, but no more than ten different nucleotides relative to the nucleotide sequence of SEQ ID NO: 4375; or a nucleotide sequence comprising one, two, three, four, five, six, or seven, but no more than ten modifications relative to SEQ ID NO: 4375;
(D) (i) a 5′ flanking region comprising the nucleotide sequence of SEQ ID NO: 4354, a nucleotide sequence at least 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4965;
(iii) a loop region comprising the nucleotide sequence of SEQ ID NO: 4363, a nucleotide sequence at least 85%, 90%, 95% 96%, 97%, 98%, 99% identical to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4907; and
(v) a 3′ flanking region of SEQ ID NO: 4373, a nucleotide sequence at least 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373;
(E) (i) a 5′ flanking region comprising the nucleotide sequence of SEQ ID NO: 4354, a nucleotide sequence at least 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4969;
(iii) a loop region comprising the nucleotide sequence of SEQ ID NO: 4363, a nucleotide sequence at least 85%, 90%, 95% 96%, 97%, 98%, 99% identical to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4911; and
(v) a 3′ flanking region of SEQ ID NO: 4373, a nucleotide sequence at least 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373;
(F) (i) a 5′ flanking region comprising the nucleotide sequence of SEQ ID NO: 4354, a nucleotide sequence at least 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4977;
(iii) a loop region comprising the nucleotide sequence of SEQ ID NO: 4363, a nucleotide sequence at least 85%, 90%, 95% 96%, 97%, 98%, 99% identical to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4919; and
(v) a 3′ flanking region of SEQ ID NO: 4373, a nucleotide sequence at least 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373; or
(G) (i) a 5′ flanking region comprising the nucleotide sequence of SEQ ID NO: 4354, a nucleotide sequence at least 85%, 90%, 95% 96%, 97%, 98%, 99% identical to SEQ ID NO: 4354, a nucleotide sequence comprising one, two, three, four, five, six, or seven, but no more than ten different nucleotides relative to the nucleotide sequence of SEQ ID NO: 4354; or a nucleotide sequence comprising one, two, three, four, five, six, or seven, but no more than ten modifications relative to the nucleotide sequence of SEQ ID NO: 4354;
(ii) the sense strand sequence of SEQ ID NO: 4981;
(iii) a loop region comprising the nucleotide sequence of SEQ ID NO: 4363, a nucleotide sequence at least 85%, 90%, 95% 96%, 97%, 98%, 99% identical to SEQ ID NO: 4363, a nucleotide sequence comprising one, two, three, or four, but no more than five different nucleotides relative to the nucleotide sequence of SEQ ID NO: 4363; or a nucleotide sequence comprising one, two, three, or four, but no more than five modifications relative to the nucleotide sequence of SEQ ID NO: 4363;
(iv) the antisense strand sequence comprises the nucleotide sequence of SEQ ID NO: 4923; and
(v) a 3′ flanking region of SEQ ID NO: 4373, a nucleotide sequence at least 85%, 90%, 95% 96%, 97%, 98%, 99% identical to SEQ ID NO: 4373, a nucleotide sequence comprising one, two, three, four, five, six, or seven, but no more than ten different nucleotides relative to the nucleotide sequence of SEQ ID NO: 4373, a nucleotide sequence comprising one, two, three, four, five, six, or seven, but no more than ten modifications relative to the nucleotide sequence of SEQ ID NO: 4373.

17. The modulatory polynucleotide of any one of claim 9 or 11-16, or the AAV viral genome of any one of claims 10-16, wherein the nucleotide sequence encoding the modulatory polynucleotide comprises the nucleotide sequence of any one of SEQ ID NOs: 4405, 4408, 4409, 4390, 4391, 4393, 4394, 4380-4389, 4392, 4395-4404, 4406, or 4407, or nucleotide sequence at least 85%, 90%, 92%, 95%, 96%, 97%, 98%, or 99% identical thereto.

18. An AAV viral genome comprising a promoter operably linked to a nucleic acid encoding the modulatory polynucleotide of any one of claim 9 or 11-17 or the siRNA of any one of claim 1 or 3-8.

19. The AAV viral genome of any one of claim 3-8 or 10-17, wherein the AAV viral genome further comprises a promoter operably linked to the nucleotide sequence encoding the sense strand sequence and the antisense strand sequence or the nucleotide sequence encoding the modulatory polynucleotide.

20. The AAV viral genome of claim 18 or 19, wherein the promoter:
(i) is a ubiquitous promoter;
(ii) is a cell or tissue specific promoter;
(iii) is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light chain (NFL) or neurofilament heavy chain (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a functional fragment, e.g., a truncation, or a functional variant thereof;
(iii) is a CBA promoter or variant thereof;
(iv) comprises the nucleotide sequence of any one of the nucleotide sequences provided in Table 2; a nucleotide sequence comprising at least one, two, three, four, five, six, or seven but no more than ten modifications, e.g., substitutions, insertions, or deletions, relative to any one of the nucleotide sequences provided in Table 2; or a nucleotide sequence with at least 85%, 90%, 92%, 95%, 96%, 97%, 98%, or 99% sequence identity to any one of the nucleotide sequences provided in Table 2; or
(v) comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence with at least 85%, 90%, 92%, 95%, 96%, 97%, 98%, or 99% sequence identity to SEQ ID NO: 5199.

21. The AAV viral genome of any one of claim 2-8 or 10-20, wherein the AAV viral genome further comprises:
(i) at least one or two inverted terminal repeat (ITR) sequences, optionally wherein the AAV viral genome comprises an ITR sequence positioned 5′ relative to the nucleic acid encoding the modulatory polynucleotide or siRNA and/or an ITR sequence positioned 3′ relative to the nucleic acid encoding the modulatory polynucleotide or siRNA;
(ii) a polyadenylation (polyA) signal sequence;
(iii) an enhancer, a Kozak sequence, an intron region, and/or an exon region; and/or
(iv) a nucleotide sequence encoding a microRNA (miR) binding site, e.g., a miR binding site that modulates, e.g., reduces expression of payload encoded by the viral genome in a cell or tissue where the corresponding miRNA is expressed; optionally wherein the encoded miR binding site modulates, e.g., reduces expression of the encoded payload in a cell or tissue of the DRG, liver, heart, hematopoietic lineage, or a combination thereof.

22. The AAV viral genome of claim 21, wherein:
(i) the ITR positioned 5′ relative to the nucleic acid encoding the modulator polynucleotide or siRNA comprises the nucleotide sequence of SEQ ID NO: 5197 or 4469; a nucleotide sequence comprising at least one, two, three, four, five, six, or seven but no more than ten modifications, e.g., substitutions, insertions, or deletions, relative to SEQ ID NO: 5197 or 4469; or a nucleotide sequence with at least 80%, 85%, 90%, 95%, 96%, 97%, 98%, or 99% sequence identity to SEQ ID NO: 5197 or 4469;
(ii) the ITR positioned 3′ relative to the nucleic acid encoding the modulator polynucleotide or siRNA comprises the nucleotide sequence of SEQ ID NO: 5200 or 4470; a nucleotide sequence comprising at least one, two, three, four, five, six, or seven but no more than ten modifications, e.g., substitutions, insertions, or deletions, relative to SEQ ID NO: 5200 or 4470; or a nucleotide sequence with at least 80%, 85%, 90%, 95%, 96%, 97%, 98%, or 99% sequence identity to SEQ ID NO: 5200 or 4470;
(iii) the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence with at least 80% (e.g., 85%, 90%, 95%, 96%, 97%, 98%, or 99%) sequence identity to SEQ ID NO: 4476;
(iv) the enhancer comprises a CMVie enhancer or variant thereof;
(v) the enhancer comprises the nucleotide sequence of SEQ ID NO: 4471 or 4472; a nucleotide sequence comprising at least one, two, three, four, five, six, or seven but no more than ten modifications, e.g., substitutions, insertions, or deletions, relative to SEQ ID NO: 4471 or 4472; or a nucleotide sequence with at least 80%, 85%, 90%, 95%, 96%, 97%, 98%, or 99% sequence identity to SEQ ID NO: 4471 or 4472; and/or
(vi) the intron comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence with at least 80%, 85%, 90%, 95%, 96%, 97%, 98%, or 99% sequence identity to SEQ ID NO: 4475.

23. The AAV viral genome of any one of claim 2-8 or 10-22, wherein the AAV viral genome is self-complementary or single stranded.

24. The AAV viral genome of any one of claim 2-8 or 10-23, wherein the AAV viral genome further comprises a nucleotide sequence encoding a Rep protein, e.g., a non-structural protein, wherein the Rep protein comprises a Rep78 protein, a Rep68, Rep52 protein, and/or a Rep40 protein (e.g., a Rep78 and a Rep52 protein).

25. The AAV viral genome of any one of claim 2-8 or 10-24, which comprises in 5′ to 3′ order:
(A) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises any one of SEQ ID NOs: 4380-4409 or 5027-5050, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO:
4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200; or
(B) (i) an ITR, wherein the ITR comprises the nucleotide sequence of SEQ ID NO: 4469; a nucleotide sequence at least 80%, 85%, 90%, 92%, 95%, 96%, 97%, 98%, or 99% identical to SEQ ID NO: 4469; or a nucleotide sequence comprising at least one, two, three, four, five, six, or seven but no more than ten modifications, e.g., substitutions, insertions, or deletions, relative to SEQ ID NO: 4469;
(ii) an enhancer, wherein the enhancer comprises the nucleotide sequence of SEQ ID NO: 4472; a nucleotide sequence comprising at least one, two, three, four, five, six, or seven but no more than ten modifications, e.g., substitutions, insertions, or deletions, relative to SEQ ID NO: 4472; or a nucleotide sequence at least 80%, 85%, 90%, 92%, 95%, 96%, 97%, 98%, or 99% identical to SEQ ID NO: 4472;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises any one of SEQ ID NOs: 4380-4409 or 5027-5050, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 4470; a nucleotide sequence at least 80%, 85%, 90%, 92%, 95%, 96%, 97%, 98%, or 99% identical to SEQ ID NO: 4470; or a nucleotide sequence comprising at least one, two, three, four, five, six, or seven but no more than ten modifications, e.g., substitutions, insertions, or deletions, relative to SEQ ID NO: 4470.

26. The AAV viral genome of any one of claim 2-8 or 10-25, which comprises in 5′ to 3′ order:
(A) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4405, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200;
(B) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4408, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200;
(C) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4409, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200;
(D) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4390, or nucleotide sequence at least 80%, 85%, 90%, 92%, 95%, 96%, 97%, 98%, or 99% identical thereto;
(vi) a polyA signal sequence, wherein the poly A signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200;
(E) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4391, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200;
(F) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4393, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200; or
(G) (i) an ITR, wherein the ITR comprises the nucleotide sequence of SEQ ID NO: 5197; a nucleotide sequence at least 80%, 85%, 90%, 92%, 95%, 96%, 97%, 98%, or 99% identical to SEQ ID NO: 5197; or a nucleotide sequence comprising at least one, two, three, four, five, six, or seven but no more than ten modifications, e.g., substitutions, insertions, or deletions, relative to SEQ ID NO: 5197;
(ii) an enhancer, wherein the enhancer comprises the nucleotide sequence of SEQ ID NO: 4471; a nucleotide sequence comprising at least one, two, three, four, five, six, or seven but no more than ten modifications, e.g., substitutions, insertions, or deletions, relative to SEQ ID NO: 4471; or a nucleotide sequence at least 80%, 85%, 90%, 92%, 95%, 96%, 97%, 98%, or 99% identical to SEQ ID NO: 4471;
(iii) a promoter, wherein the promoter comprises the nucleotide sequence of SEQ ID NO: 5199; a nucleotide sequence comprising at least one, two, three, four, five, six, or seven but no more than ten modifications, e.g., substitutions, insertions, or deletions, relative to SEQ ID NO: 5199; or a nucleotide sequence at least 80%, 85%, 90%, 92%, 95%, 96%, 97%, 98%, or 99% identical to SEQ ID NO: 5199;
(iv) an intron, wherein the intro comprises the nucleotide sequence of SEQ ID NO: 4475; a nucleotide sequence comprising at least one, two, three, four, five, six, or seven but no more than ten modifications, e.g., substitutions, insertions, or deletions, relative to SEQ ID NO: 4475; or a nucleotide sequence at least 80%, 80%, 85%, 90%, 92%, 95%, 96%, 97%, 98%, or 99% identical to SEQ ID NO: 4475;
(v) a nucleotide sequence encoding a modulatory polynucleotide, wherein the nucleotide sequence comprises SEQ ID NO: 4394, or nucleotide sequence at least 80%, 85%, 90%, 92%, 95%, 96%, 97%, 98%, or 99% identical thereto;
(vi) a polyA signal sequence, wherein the polyA signal sequence comprises the nucleotide sequence of SEQ ID NO: 4476; a nucleotide sequence comprising at least one, two, three, four, five, six, or seven but no more than ten modifications, e.g., substitutions, insertions, or deletions, relative to SEQ ID NO: 4476; or a nucleotide sequence at least 80%, 85%, 90%, 92%, 95%, 96%, 97%, 98%, or 99% identical to SEQ ID NO: 4476; and
(vii) an ITR, wherein the ITR comprises the nucleotide sequence of SEQ ID NO: 5200; a nucleotide sequence at least 80%, 85%, 90%, 92%, 95%, 96%, 97%, 98%, or 99% identical to SEQ ID NO: 5200; or a nucleotide sequence comprising at least one, two, three, four, five, six, or seven but no more than ten modifications, e.g., substitutions, insertions, or deletions, relative to SEQ ID NO: 5200.

27. The AAV viral genome of any one of claim 2-8 or 10-26, which comprises the nucleotide sequence of any one of SEQ ID NOs: 5173, 5195, 5196, 5182, 5183, 5184, 5185, 5149-5172, 5174-5181, 5186-5194 or 4439-4468, or a nucleotide sequence at least 70%, 75%, 80%, 85%, 90%, 95%, 99% or 100% identical to any one of SEQ ID NOs: 5173, 5195, 5196, 5182, 5183, 5184, 5185, 5149-5172, 5174-5181, 5186-5194 or 4439-4468.

28. An AAV particle comprising the AAV viral genome of any one of claim 2-8 or 10-27, an AAV viral genome encoding the modulatory polynucleotide of any one of claim 9 or 11-17, or a viral genome encoding the siRNA of any one of claim 1 or 3-8, and an AAV capsid protein.

29. The AAV particle of claim 28, wherein the AAV capsid protein comprises:
(i) an AAV5 capsid protein or a variant thereof, an AAV9 capsid protein or a variant thereof, VOY101 capsid protein or a variant thereof, a PHPeB capsid protein or a variant thereof, a PHP.B capsid protein or a variant thereof, a PHP.N capsid protein or a variant thereof, an AAV1 capsid protein or a variant thereof, an AAV2 capsid protein or a variant thereof, an AAVrh10 capsid protein or a variant thereof, or VOY9P39 capsid protein or a variant thereof;
(ii) an AAV9 capsid protein or a variant thereof;
(iii) an AAV5 capsid protein or variant thereof;
(iv) an amino acid sequence provided in Table 1 or an amino acid sequence at least 85%, 90%, 92%, 95%, 97%, 98%, or 99% identical thereto; or
(v) the nucleotide sequence encoding the AAV capsid protein comprises any one of the nucleotide sequences in Table 1, or a nucleotide sequence at least 85%, 90%, 92%, 95%, 97%, 98%, or 99% identical thereto.

30. A vector comprising the AAV viral genome of any one of claim 2-8 or 10-27, a nucleotide sequence encoding the modulatory polynucleotide of any one of claim 9 or 11-17, or a nucleotide sequence encoding the siRNA of any one of claim 1 or 3-8.

31. A cell comprising the AAV particle of claim 28 or 29, the AAV viral genome of any one of claim 2-8 or 10-27, the modulatory polynucleotide of any one of claim 9 or 11-17, or the siRNA of any one of claim 1 or 3-8, optionally wherein, the cell is:
(a) a mammalian cell or an insect cell;
(b) a cell of a brain region or a spinal cord region (e.g., a CNS cell);
(c) a cell of the cortex, hippocampus, thalamus, or brain stem; or
(d) a neuron, a glial cell, or an oligodendrocyte.

32. A method of making an AAV particle, comprising:
(i) providing a host cell comprising the AAV viral genome of any one of claim 2-8 or 10-27, an AAV viral genome encoding the modulatory polynucleotide of any one of claim 9 or 11-17, or a viral genome encoding the siRNA of any one of claim 1 or 3-8;
(ii) incubating the host cell under conditions suitable to enclose the viral genome in an AAV capsid protein;
thereby making the AAV particle.

33. A pharmaceutical composition comprising the AAV particle of claim 28 or 29, the AAV viral genome of any one of claim 2-8 or 10-27, the modulatory polynucleotide of any one of claim 9 or 11-17, or the siRNA of any one of claim 1 or 3-8, and a pharmaceutically acceptable excipient.

34. A method of delivering an siRNA for inhibiting MAPT expression to a cell, comprising administering to the cell an effective amount of the pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, thereby delivering the siRNA for inhibiting MAPT expression to the cell.

35. The method of claim 34, wherein the cell is:
(a) a mammalian cell or an insect cell:
(b) a cell of a brain region or a spinal cord region (e.g., a CNS cell);
(c) a cell of the cortex, hippocampus, thalamus, or brain stem;
(d) a neuron, a glial cell, or an oligodendrocyte; and/or
(e) in a subject.

36. The method of claim 35, wherein the subject has or has been diagnosed with having a genetic disorder (e.g., a monogenic disorder or a polygenic disorder); a neurological disorder (e.g., a neurodegenerative disorder); a disorder associated with tau expression, e.g., aberrant tau expression; and/or a tauopathy.

37. A method of treating a subject having or diagnosed with having a genetic disorder (e.g., a monogenic disorder or a polygenic disorder); a neurological disorder (e.g., a neurodegenerative disorder); a disorder associated with tau expression, e.g., aberrant tau expression; and/or a tauopathy, the method comprising administering to the subject an effective amount of the pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8.

38. The method of claim 36 or 37, wherein the genetic disorder, neurological disorder (e.g., neurodegenerative disorder), disorder associated with tau expression (e.g., aberrant tau expression), and/or tauopathy is Alzheimer's disease (AD), Frontotemporal dementia (FTD), frontotemporal dementia and parkinsonism linked to chromosome 17 (FTDP-17), frontotemporal lobar degeneration (FTLD), Dravet syndrome (DS), neurodegenerative disease, traumatic brain injury (TBI), chronic traumatic encephalopathy (CTE), progressive supranuclear palsy (PSP), Down's syndrome, Pick's disease, corticobasal degeneration (CBD), corticobasal syndrome, amyotrophic lateral sclerosis (ALS), Prion diseases, CJD, Multiple system atrophy, mild cognitive impairment, Tangle-only dementia, or Progressive subcortical gliosis.

39. The method of claim 37 or 38, wherein treating comprises prevention of progression of the genetic disorder, neurological disorder (e.g., neurodegenerative disorder), disorder associated with tau expression (e.g., aberrant tau expression), and/or tauopathy in the subject in the subject.

40. The method of any one of claims 37-39, further comprising performing a blood test, an imaging test (e.g., a PET scan or a PET scan in combination with biomarker, e.g., serum biomarker staining), a CNS biopsy sample, or an aqueous cerebral spinal fluid biopsy, optionally wherein the blood test, imaging test, biopsy sample, or aqueous cerebral spinal fluid biopsy is performed prior to, during, or after treatment with the AAV particle, modulatory polynucleotide, siRNA, or pharmaceutical composition.

41. The method of any one of claims 37-4, wherein the pharmaceutical composition, AAV particle, modulatory polynucleotide, and siRNA are administered to the subject:
(i) intravenously, via intra-cisterna magna injection (ICM), intracerebrally, intrathecally, intracerebroventricularly, via intraparenchymal administration, or intramuscularly;
(ii) via focused ultrasound (FUS), e.g., coupled with the intravenous administration of microbubbles (FUS-MB), or MRI-guided FUS coupled with intravenous administration;
(iii) intravenously.

42. The pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, for use in a method of delivering an siRNA for inhibiting MAPT expression to a cell.

43. The pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, for use in the manufacture of a medicament.

44. The pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, for use in the treatment of a genetic disorder, neurological disorder (e.g., neurodegenerative disorder), disorder associated with tau expression (e.g., aberrant tau expression), and/or tauopathy.

45. Use of the pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, in the manufacture of a medicament for delivering an siRNA for inhibiting MAPT expression to a cell.

46. Use of the pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, in the manufacture of a medicament.

47. Use of the pharmaceutical composition of claim 33, the AAV particle of claim 28 or 29, the AAV viral genome of any one of claim 2-8 or 10-27, the modulatory polynucleotide of any one of claim 9 or 11-17, or the siRNA of any one of claim 1 or 3-8, in the manufacture of a medicament for treating a genetic disorder, neurological disorder (e.g., neurodegenerative disorder), disorder associated with tau expression (e.g., aberrant tau expression), and/or tauopathy.
